(19) United States
(12) Patent Application Publication
(10) Pub. No.: US 2010/0256148 A1
(43) Pub. Date: Oct. 7, 2010

(54) USE OF CFMS INHIBITOR FOR TREATING OR PREVENTING BONE CANCER AND THE BONE LOSS AND BONE PAIN ASSOCIATED WITH BONE CANCER

(76) Inventor: Carl L. Manthey, Dowingtown, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/741,118

(22) PCT Filed: Oct. 29, 2008
(86) PCT No.: PCT/US2008/081501

§ 371 Date: May 3, 2010

Related U.S. Application Data
(60) Provisional application No. 60/984,978, filed on Nov. 2, 2007.

(51) Int. Cl. A61K 31/5355; A61P 35/00; A61K 31/496; A61K 31/454

(52) U.S. Cl. 514/235.8; 514/254.1; 514/326

(57) ABSTRACT

The present invention provides therapeutic methods for treating a subject having, and prophylactic methods for preventing in a subject at risk of (or susceptible to) developing, bone cancer and the bone loss and bone pain associated with bone cancer, said method comprising the administration of a compound of Formula I:

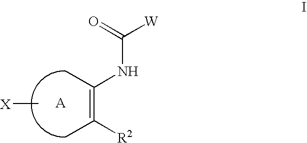

or a solvate, hydrate, tautomer or pharmaceutically acceptable salt thereof.

### FIELD OF THE INVENTION

The present invention is directed to methods for the treatment or prevention of bone cancers and bone metastases from other primary sites, and for preventing and treating bone loss and bone pain associated with cancer metastases.

### BACKGROUND OF THE INVENTION

Bone cancer is a relatively rare disease in which cancer cells grow in the bone tissue. Cancer may form in the bone or spread to the bone from another site in the body. When cancer starts in bone tissue, it is called primary bone cancer. When cancer cells travel to the bone from elsewhere, it is called secondary or metastatic cancer to the bone. Types of bone cancer include: Osteosarcoma, a cancerous tumor of the bone, usually of the arms, legs, or pelvis (the most common primary cancer); Chrondrosarcoma, cancer of the cartilage (the second most common primary cancer); Ewing's Sarcoma, tumors that usually develop in the cavity of the leg and arm bones, Fibrosarcoma and Malignant Fibrous Histiocytoma, cancers that develop in soft tissues such as the tendons, ligaments, fat, muscle and move to the bones of the legs, arms, and jaw; Giant Cell Tumor, a primary bone tumor that is malignant only about 10% of the time and is most common in the arm or leg bones; and Chordoma, a primary bone tumor that usually occurs in the skull or spine.

Bone metastases arise with high frequency in patients with solid tumor malignancies including end-stage breast, prostate, and lung carcinoma. (Mundy G. R., Nat Rev Cancer 2002; 2:584-93.) Metastatic tumors in the skeleton lead to serious morbidity including fracture, hypercalcemia and pain. The pain often develops resistance to opiate therapy and arises from two principal sources; impingement of the growing tumor on the innervated periosteum, and skeletal instability. (Sabino M A, et al., J Support Oncol 2005; 3:15-24.) The latter is a byproduct of bone erosion mediated by osteoclasts.

In this setting, two therapeutic approaches in common practice include radiotherapy to shrink tumor mass, and high dose bisphosphonates (viz., pamidronate and zoledronic acid) to deplete osteoclasts and reduce osteolysis. (Saarto T, et al., Eur J Pain 2002; 6:323 330; and Mystakidou K, et al., Cancer Treat Rev 2005; 31:303 311.)

Radiotherapy does not address soft tissue metastases that often accompany bone metastasis. Additionally bisphosphonates delay (˜35%) but do not prevent skeletal events in most patients, and are accompanied with significant bone toxicities (e.g., osteonecrosis). Also, most existing chemotherapeutics target tumor cells directly, but have variable efficacy in many cancers and are often associated with debilitating side effects, and cannot be chronically administered. In addition, advanced cancers are prone to develop resistance to chemotherapy due to their inherent genetic instability. For these reasons, there is increasing interest to understand and exploit the dependence of tumor on the host microenvironment (Joyce J., Cancer Cell 2005; 7:513-520.)

An important emerging concept is that tumor-associated macrophages may facilitate tumor growth and metastasis. (See, e.g., Pollard J W., Nature Reviews Cancer 2004; 4:71-78; and Bingle L, et al., J Pathol 2002; 196:254-265.)

Macrophages comprise between five and fifty percent of cells in most tumors, and have long been considered a component of tumor immunity. (Wood G W, et al., J Natl Cancer Inst 1977; 59:1081-7; and Kelly P M A et al., Br J Cancer 1988; 57:174-177.) However, numerous recent studies (see, Bingle L, et al., J Pathol 2002; 196:254-265 and Valkovic T, et al., Virchows Arch 2002; 440:583 588.) demonstrating a direct correlation between macrophage numbers, angiogenesis and tumor progression have forced a reconsideration of the potential role of macrophages in the tumor microenvironment.

There is now a growing body of evidence that tumor-associated macrophages (TAMs) promote tumor angiogenesis and growth. In response to the tumor microenvironment, TAMs are “alternatively activated” to elaborate growth factors and cytokines that support tumor growth including VEGF, platelet derived growth factor (PDGF), basic fibroblast growth factor (bFGF), and TGF-131, matrix metalloprotease-9 (MMP-9) and urokinase plasminogen activator (uPA). (Mantovani A. et al., Trends in Immunology 2002; 23:549-555.) Significantly, TAMs have been identified in some tumors as the major source of epidermal growth factor (EGF).

Indeed, several studies have shown that chemical or genetic depletion of TAMs results in tumor growth suppression in preclinical models (see, e.g., van Rooijen N et al., Methods Enzymol 2003; 373:3-16; De Palma M et al., Cancer Cell 2005; 8:211-226; Nowickki A, et al., Int J Cancer 1996; 65: 112-119; Aharinejad S, et al., Cancer Res 2002; 62:5317-5324; Aharinejad S, et al., Cancer Res 2004; 64:5378-5384; and Paulus P et al., Cancer Res 2006; 66:4349-56).

Macrophage lineage is dependent, in part, on colony stimulating factor-1 (CSF-1) (see, e.g., Pollard, J. W. et al., Adv in Devel Biochem 1995; 4:153-193.) FMS is the class III receptor tyrosine kinase responsible for all cell-signaling by the macrophage lineage growth factor, colony stimulating factor-1 (CSF-1). Because CSF-1 plays a critical role in tumor-induced osteoclastogenesis, inhibition of FMS is anticipated to provide a mechanism for the prevention of osteolysis in metastatic bone disease. Further, mouse genetics more specifically implicate CSF-1 in tumor growth and progression. Lewis lung carcinomas grew poorly in CSF-1-deficient mice. (Nowicki A, et al., Int J Cancer 1996; 65:112 119.) Further, systemically delivered CSF-1 antisense and neutralizing anti-CSF-1 antibody were shown to reduce the growth rate of several human tumor xenografts. (Paulus P, et al., Cancer Res 2006; 66:4349 4356.) Growth suppression was associated with reduced TAMs and lower microvessel density.

Between 50% and 85% of patients suffering late stage breast and prostate cancer will be diagnosed with bone metastases (Roodman G D., NEJM 2004; 350:1655-64.). Bone metastases rates in late stage lung cancer patients are somewhat lower (ca. 30%) only because of rapid progression and death from this disease. Most patients with bone metastases will experience a skeletal event (e.g., severe bone pain, bone fracture, or hypercalcemia) despite bisphosphonate therapy.

Metastatic bone lesions may be lytic or sclerotic in nature depending upon whether increased osteoclastic or osteoblastic activity predominates; if both processes are equally active, they are termed mixed lesions. Bone metastases in breast cancer patients usually involve osteolytic disease, where normal bone homeostasis is disrupted and skewed towards excessive resorption of bone (Coleman R E, Cancer Treat Rev. 27(3), 165-76 (2001)).

Tumor-associated bone erosion is exacerbated by a microenvironment favoring osteoclastogenesis and osteoclast activation (Roodman G D., Biology of osteoclast activation in cancer. J Clin Oncol 2001; 19:3562-3571.) CSF-1 is expressed by tumors and is a critical differentiation factor for osteoclasts as exemplified by the near complete absence of osteoclasts in young CSF-1-deficient mice (Pollard, J. W. et al., Adv in Devel Biochem 1995; 4:153-193.)

CSF-1 not only drives the proliferation and differentiation of osteoclast precursors (i.e., macrophages), but it is required for the differentiation of osteoclast precursors into osteoclasts, in part, by enhanced expression of RANK (Kitaura H et al., J Clin Invest; 2005; 115: 3418-27.)

Although activating FMS mutations are rare in human cancers, ectopic expression of FMS might drive proliferation in some tumors. Histological examination of primary tumors indicated high FMS expression on many breast, prostate, ovarian, uterine, endometrial, hepatocellular, and squamous cell carcinomas. (Kascinski B., Cancer Treat Res 2002; 107: 285 292.) Lung and breast carcinoma tumor lines that expressed FMS were more invasive, and FMS expression in breast cancer has been correlated with poor prognosis. (Kluger H M, et al., Clin Cancer Res 2004; 10: 173 177.)

Patients with bone metastases experience considerable morbidity, including bone pain, pathological fractures, hypercalcaemia, reduced mobility and spinal cord or nerve root compression. Despite the importance of these clinical problems, there are few available treatments for bone loss associated with cancer metastasis. Thus, there remains a need in the art to identify new agents and methods for preventing or treating cancer metastasis, including bone metastases, and the associated bone loss and bone pain. (see, e.g., WO 2007/081879).

### SUMMARY OF THE INVENTION

The present invention is directed to methods of treating or preventing bone cancer and bone metastases from other primary sites, and for preventing and treating bone loss and bone pain associated with cancer metastases, utilizing certain compounds described in WO 2006/047277 (filed Oct. 20, 2005, as PCT/US2005/037868), in particular 4-Cyano-1H-imidazole-2-carboxylic acid {2-cyclohex-1-enyl-4-[1-(2-dimethylamino-acetyl)-piperidin-4-yl]-phenyl}-amide, or a solvate, hydrate, tautomer or pharmaceutically acceptable salt thereof, described as Example 38a in WO 2006/047277 and as JNJ-141 herein, the disclosure of which is hereby incorporated by reference in its entirety.

### DESCRIPTION OF THE DRAWINGS

FIG. 1. Structure and cell activity of JNJ-141. A: Structure of JNJ-141. B: A stable HEK cell-line with recombinant CSF-1R expression was pretreated 30 minutes with graded concentrations of JNJ-141 and treated ten minutes with 25 ng/ml CSF-1. Cells were lysed and lysates evaluated for phosphorylated CSF-1R and total CSF-1R by immunoblot analysis as described in the experimentals herein.

FIG. 2. JNJ-141 inhibits CSF-1R in vivo. B6C3R1 mice were dosed orally with JNJ-141 eight hours prior to a tail-vein injection of 0.8 micrograms (μg) of recombinant CSF-1. Fifteen minutes later, the mice were sacrificed and c-fos mRNA was measured in spleen lysates as described in the experimentals herein. JNJ-141 dose dependently suppressed CSF-1-induced c-fos mRNA induction in mice. FIG. 3. JNJ-141 reduced the growth of H460 human lung tumor xenografts in nude mice. Three days following s.c inoculation of nude mice with 1×10⁶ H460 cells oral dosing was initiated twice-daily (except once daily on weekends) with vehicle or JNJ-141 at 25, 50 or 100 mg/kg. A: Tumor volumes were determined by caliper measurement. B: On day 28, mice were sacrificed and tumors were excised and weighed. C: Mouse weights were determined on days indicated. All values represent means and standard errors. * p<0.05 vs the vehicle control. ** p<0.01 vs the vehicle control.

FIG. 4. JNJ-141 reduced tumor associated macrophages and microvascularity. Day 28 tumors were harvested from vehicle-treated mice (A and C) or mice treated with 100 mg/kg JNJ-141 (B and D). Tumors were fixed in formalin, and paraffin-embedded sections were probed for F4/80⁺ macrophages (A and B) or frozen and cryostat sections probed for CD31⁺ microvasculature (C and D) as described in the experimentals herein.

FIG. 5. JNJ-141 prevented bone erosions in tibiae with MRMT-1 tumors. Saline (A) or 3×10⁴ rat syngeneic MRMT mammary carcinoma cells (B-D) were inoculated into the left tibia of rats. Starting on day 3, rats were dosed bid with vehicle (B), or with 20 mg/kg JNJ-141 (C), or QOD s.c. with 30 μg/kg zoledronate (D). On day 17, rats were sacrificed and tibiae assessed by micro-computed tomography.

FIG. 6. JNJ-141 prevented bone erosions and eliminated osteoclasts in tibiae with MRMT-1 tumors. Saline (A) or 3×10⁴ rat syngeneic MRMT mammary carcinoma cells (B-G) were inoculated into the left tibia of rats. Starting on day 3, rats were dosed bid with vehicle (B and E) or 20 mg/kg JNJ-141 (C and F) or god s.c. with 30 μg/kg zoledronate (D and G). On day 17, rats were sacrificed and left hindlimbs were excised, fixed and decalcified, and paraffin-embedded sections were stained for TRAP⁺ cells and counterstained lightly in H&E. Representative photomicrographs (40× originals) of the epiphyseal trabecular bone (A-D) were photographed in dark field for optimal visualization of the trabecular bone below the growth plate. Representative photomicrographs (200× originals) of periosteal tumor (E-G) are provided. Note the nearly complete loss of trabecular bone in the vehicle-treated tumor bearing rats and the protection afforded by JNJ-141 and zoledronate (A-D). Although both agents depleted osteoclasts from the trabecular bones, note that multinucleated, tumor-associated osteoclasts are still present in zoledronate-treated rats (G) but are absent rats treated with JNJ-141 (F).

FIG. 7. JNJ-141 prevented onset of metastatic bone pain. Inoculation of MRMT-1 cells into the proximal tibia significantly increased mechanical allodynia in animals inoculated with MRMT-1 cells compared to animals inoculated with media at the final time point; p<0.01. Treatment of affected animals with morphine reversed allodynia from the 2nd time point forward, while treatment with either 20 mpk or 60 mpk of JNJ-141 decreased allodynia compared to tumor-inoculated animals at the final time point (p, 0.05 and 0.01, respectively). Zoledronate treatment also decreased allodynia compared to tumor-inoculated animals but this effect did not reach statistical significance. Values in figure represent group means±SEM.

Other features and advantages of the invention will be apparent from the following detailed description of the invention and from the claims.

### DETAILED DESCRIPTION OF THE INVENTION

The terms “comprising”, “including”, and “containing” are used herein in their open, non-limited sense.

### Abbreviations

As used herein, the following abbreviations are intended to have the following meanings (additional abbreviations are provided where needed throughout the Specification):
ATP adenosine triphosphate
Boc or BOC tert-butoxycarbonyl
DCM dichloromethane
DMF dimethylformamide
DMSO dimethylsulfoxide
DIEA diisopropylethylamine
EDCI 1-(3-dimethylaminopropyl)-3-ethylcarbodiimide hydrochloride
EDTA ethylenediaminetetraaceticacid
EtOAc ethyl acetate
FP fluorescence polarization
HOBT or HOBt 1-hydroxybenzotriazole hydrate
LC/MS (ESI) Liquid chromatography/mass spectrum (electrospray ionization)
MeOH Methyl alcohol
NMR nuclear magnetic resonance
RT room temperature
TFA trifluoroacetic acid
THF tetrahydrofuran
TLC thin layer chromatography

### DEFINITIONS

The term “alkyl” refers to both linear and branched chain radicals of up to 12 carbon atoms, preferably up to 6 carbon atoms, unless otherwise indicated, and includes, but is not limited to, methyl, ethyl, propyl, isopropyl, butyl, isobutyl, sec-butyl, tert-butyl, pentyl, isopentyl, hexyl, isohexyl, heptyl, octyl, 2,2,4-trimethylpentyl, nonyl, decyl, undecyl and dodecyl.

The term “hydroxyalkyl” refers to both linear and branched chain radicals of up to 6 carbon atoms, in which one hydrogen atom has been replaced with an OH group.

The term “hydroxyalkylamino” refers to an hydroxyalkyl group in which one hydrogen atom from the carbon chain has been replaced with an amino group, wherein the nitrogen is the point of attachment to the rest of the molecule.

The term “cycloalkyl” refers to a saturated or partially unsaturated ring composed of from 3 to 8 carbon atoms. Up to four alkyl substituents may optionally be present on the ring. Examples include cyclopropyl, 1,1-dimethyl cyclobutyl, 1,2,3-trimethylcyclopentyl, cyclohexyl, cyclopentenyl, cyclohexenyl, and 4,4-dimethyl cyclohexenyl.

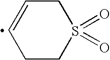

The term “dihydrosulfonopyranyl” refers to the following radical:

The term “hydroxyalkyl” refers to at least one hydroxyl group bonded to any carbon atom along an alkyl chain.

The term “aminoalkyl” refers to at least one primary or secondary amino group bonded to any carbon atom along an alkyl chain, wherein an alkyl group is the point of attachment to the rest of the molecule.

The term “alkylamino” refers to an amino with one alkyl substituent, wherein the amino group is the point of attachment to the rest of the molecule.

The term “dialkylamino” refers to an amino with two alkyl substituents, wherein the amino group is the point of attachment to the rest of the molecule.

The term “heteroaromatic” or “heteroaryl” refers to 5- to 7-membered mono- or 8- to 10-membered bicyclic aromatic ring systems, any ring of which may consist of from one to four heteroatoms selected from N, O or S where the nitrogen and sulfur atoms can exist in any allowed oxidation state. Examples include benzimidazolyl, benzothiazolyl, benzothienyl, benzoxazolyl, furyl, imidazolyl, isothiazolyl, isoxazolyl, oxazolyl, pyrazinyl, pyrazolyl, pyridyl, pyrimidinyl, pyrrolyl, quinolinyl, thiazolyl and thienyl.

The term “heteroatom” refers to a nitrogen atom, an oxygen atom or a sulfur atom wherein the nitrogen and sulfur atoms can exist in any allowed oxidation states.

The term “alkoxy” refers to straight or branched chain radicals of up to 12 carbon atoms, unless otherwise indicated, bonded to an oxygen atom. Examples include methoxy, ethoxy, propoxy, isopropoxy and butoxy.

The term “aryl” refers to monocyclic or bicyclic aromatic ring systems containing from 6 to 12 carbons in the ring. Alkyl substituents may optionally be present on the ring. Examples include benzene, biphenyl and napththalene.

The term “aralkyl” refers to a C₁₋₆ alkyl group containing an aryl substituent. Examples include benzyl, phenylethyl or 2-naphthylmethyl.

The term “sulfonyl” refers to the group —S(O)₂Rₐ, where Rₐ is hydrogen, alkyl, cycloalkyl, haloalkyl, aryl, aralkyl, heteroaryl and heteroaralkyl. A “sulfonylating agent” adds the —S(O)₂Rₐ group to a molecule.

### Formula I

The present invention comprises methods of using the compounds of Formula I (referred to herein as “the compounds of the present invention”):

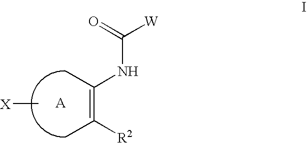

or a solvate, hydrate, tautomer or pharmaceutically acceptable salt thereof, wherein:

### A is

phenyl or pyridyl, either of which may be substituted with one of chloro, fluoro, methyl, —N₃, —NH₂, —NH(alkyl), —N(alkyl)₂, —S(alkyl), —O(alkyl), or 4-aminophenyl;

### W is

pyrrolyl (including 1H-pyrrol-2-yl), imidazolyl, (including 1H-imidazol-2-yl), isoxazolyl, oxazolyl, 1,2,4 triazolyl, or furanyl (including furan-2-yl), any of which may be connected through any carbon atom, wherein the pyrrolyl, imidazolyl, isoxazolyl, oxazolyl, 1,2,4 triazolyl, or furanyl may contain one —Cl, —CN, —NO₂, —OMe, or —CF₃ substitution, connected to any other carbon;

### R² is

cycloalkyl (including cyclohexenyl, cyclopentenyl), thiophenyl, dihydrosulfonopyranyl, phenyl, furanyl, tetrahydropyridyl, or dihydropyranyl, any of which may be independently substituted with one or two of each of the following: chloro, fluoro, and C₍₁₋₃₎alkyl (including 4,4-dimethyl cyclohexenyl, 4-methyl cyclohexenyl, 2-methyl thiophenyl, 3-methyl thiophenyl), with the proviso that tetrahydropyridyl is connected to the ring A through a carbon-carbon bond;

### X is

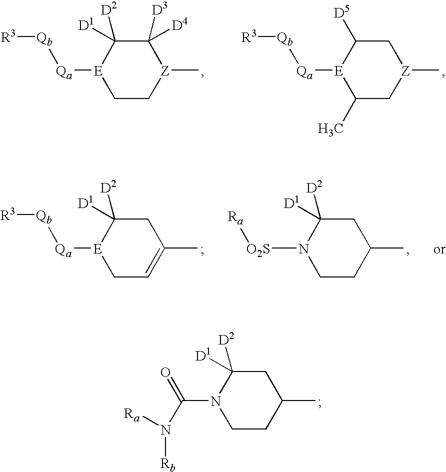 [0065] Z is
   CH or N;
D¹ and D² are
   each hydrogen or taken together form a double bond to an oxygen;
D³ and D⁴ are
   each hydrogen or taken together form a double bond to an oxygen;
D⁵ is
   hydrogen or —CH₃, wherein said —CH₃ may be relatively oriented syn or anti;
Rₐ and R_{b} are independently
   hydrogen, cycloalkyl, haloalkyl, aryl, aralkyl, heteroaryl, or heteroaralkyl;
E is
   N, S, O, SO or SO₂, with the proviso that E may not be N if the following three conditions are simultaneously met: Qₐ is absent, Q_{b} is absent, and R³ is an amino group or cyclic amino radical wherein the point of attachment to E is N;
Qₐ is
   absent, —CH₂—, —CH₂CH₂—, or C(O);
Q_{b} is
   absent, —NH—, —CH₂—, —CH₂CH₂—, or C(O), with the proviso that Q_{b} may not be C(O) if Qₐ is C(O), and further provided that Q_{b} may not be —NH— if E is N and Qₐ is absent, further provided that Q_{b} may not be —NH— if R³ is an amino group or cyclic amino radical wherein the point of attachment to Q_{b} is N;
R³ is
   hydrogen, phenyl, hydroxyalkylamino (including 2-hydroxy ethylamino), (hydroxyalkyl)₂amino, hydroxyalkyl(alkyl)amino (including 1-hydroxyeth-2-yl(methyl)amino), alkylamino (including methylamino), aminoalkyl (including 2-amino isopropyl), dihydroxyalkyl (including 1,3-dihydroxy isopropyl, 1,2-dihydroxy ethyl), alkoxy (including methoxy), dialkylamino (including dimethylamino), hydroxyalkyl (including 1-hydroxy eth-2-yl), —COOH, —CONH₂, —CN, —SO₂-alkyl-R⁴ (including —SO₂CH₃), —NH₂, or a 5 or six membered ring which contains at least one heteroatom N and may optionally contain an additional heteromoiety selected from S, SO₂, N, and O, and the 5 or 6 membered ring may be saturated, partially unsaturated or aromatic (including piperidinyl, morpholinyl, imidazolyl, and pyridyl) wherein aromatic nitrogen in the 5 or 6 membered ring may be present as N-oxide (including pyridyl N-oxide), and the 5 or 6 membered ring may be optionally substituted with methyl, halogen, alkylamino, or alkoxy (including 1 methyl imidazolyl); R³ may also be absent, with the proviso that R³ is not absent when E is nitrogen;
R⁴ is
   hydrogen, —OH, alkoxy, carboxy, carboxamido, or carbamoyl.

### Embodiments

Embodiments of the present invention include a compound of Formula I wherein:

### a) A is

phenyl or pyridyl, either of which may be substituted with one of chloro, fluoro, methyl, —N₃, —NH₂, —NH(alkyl), —N(alkyl)₂, —S(alkyl), —O(alkyl), or 4-aminophenyl;

### b) A is

phenyl;

### c) W is

pyrrolyl (including 1H-pyrrol-2-yl), imidazolyl, (including 1H-imidazol-2-yl), isoxazolyl, oxazolyl, 1,2,4 triazolyl, or furanyl (including furan-2-yl), any of which may be connected through any carbon atom, wherein the pyrrolyl, imidazolyl, isoxazolyl, oxazolyl, 1,2,4 triazolyl, or furanyl may contain one —Cl, —CN, —NO₂, —OMe, or —CF₃ substitution, connected to any other carbon;

### d) W is

furan-2-yl, 1H-pyrrol-2-yl, or 1H-imidazol-2-yl, any of which may be substituted at the 4 or 5 carbons with —CN;

### e) W is

3H-2-imidazolyl-4-carbonitrile or 5-cyano-1H-pyrrol-2-yl;

### f) W is

3H-2-imidazolyl-4-carbonitrile;

### g) R² is

cycloalkyl (including cyclohexenyl, cyclopentenyl), thiophenyl, dihydrosulfonopyranyl, phenyl, furanyl, tetrahydropyridyl, or dihydropyranyl, any of which may be independently substituted with one or two of each of the following: chloro, fluoro, and C₍₁₋₃₎alkyl (including 4,4-dimethyl cyclohexenyl, 4-methyl cyclohexenyl, 2-methyl thiophenyl, 3-methyl thiophenyl), with the proviso that tetrahydropyridyl is connected to the ring A through a carbon-carbon bond;

### h) R² is

cycloalkyl (including cyclohexenyl, cyclopentenyl), which may substituted with one or two C₍₁₋₃₎alkyl (including 4,4-dimethyl cyclohexenyl, 4-methyl cyclohexenyl);

### i) R² is

cyclohexenyl, which may substituted with one or two C₍₁₋₃₎alkyl:

### j) R² is

cyclohexenyl, 4,4-dimethyl cyclohexenyl, or 4-methyl cyclohexenyl;

### k) R² is

cyclohexenyl;

### l) X is

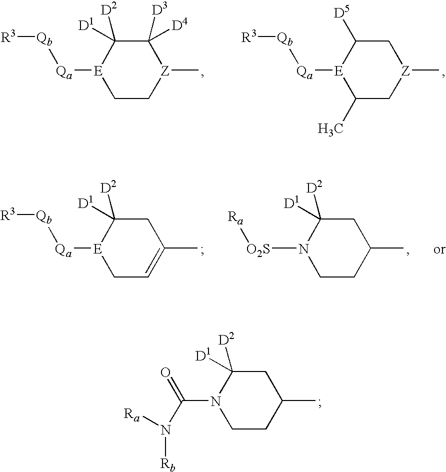

### m) X is

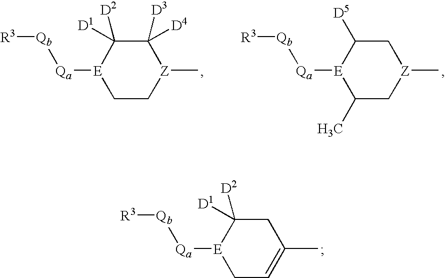

### n) X is

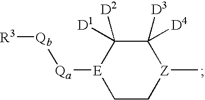

### o) Z is

CH or N;

### p) Z is

CH;

### q) D¹ and D² are

each hydrogen or taken together form a double bond to an oxygen;

### r) D¹ and D² are

each hydrogen;

### s) D³ and D⁴ are

each hydrogen or taken together form a double bond to an oxygen;

### t) D³ and D⁴ are

each hydrogen;

### u) D⁵ is

hydrogen or —CH₃, wherein said —CH₃ may be relatively oriented syn or anti;
   v) Rₐ and R_{b} are independently
hydrogen, cycloalkyl, haloalkyl, aryl, aralkyl, heteroaryl, or heteroaralkyl;

### w) E is

N, S, O, SO or SO₂, with the proviso that E may not be N if the following three conditions are simultaneously met: Qₐ is absent, Q_{b} is absent, and R³ is an amino group or cyclic amino radical wherein the point of attachment to E is N;

### x) E is

N, with the proviso that E may not be N if the following three conditions are simultaneously met: Qₐ is absent, Q_{b} is absent, and R³ is an amino group or cyclic amino radical wherein the point of attachment to E is N;

### y) Qₐ is

absent, —CH₂—, —CH₂CH₂—, or C(O);

### z) Qₐ is

absent, —CH₂CH₂—, or C(O);

### aa) Qₐ is

absent, or C(O);

### bb) Qₐ is

C(O);

### cc) Q_{b} is

absent, —NH—, —CH₂—, —CH₂CH₂—, or C(O), with the proviso that Q_{b} may not be C(O) if Qₐ is C(O), and further provided that Q_{b} may not be —NH— if E is N and Qₐ is absent, further provided that Q_{b} may not be —NH— if R³ is an amino group or cyclic amino radical wherein the point of attachment to Q_{b} is N;

### dd) Q_{b} is

absent, —CH₂CH₂—, or C(O), with the proviso that Q_{b} may not be C(O) if Qₐ is C(O);

### ee) Q_{b} is

absent, or C(O), with the proviso that Q_{b} may not be C(O) if Qₐ is C(O);

### ff) R³ is

hydrogen, phenyl, hydroxyalkylamino (including 2-hydroxy ethylamino), (hydroxyalkyl)₂amino, hydroxyalkyl(alkyl)amino (including 1-hydroxyeth-2-yl(methyl)amino), alkylamino (including methylamino), aminoalkyl (including 2-amino isopropyl), dihydroxyalkyl (including 1,3-dihydroxy isopropyl, 1,2-dihydroxy ethyl), alkoxy (including methoxy), dialkylamino (including dimethylamino), hydroxyalkyl (including 1-hydroxy eth-2-yl), —COOH, —CONH₂, —CN, —SO₂-alkyl-R⁴ (including —SO₂CH₃), —NH₂, or a 5 or six membered ring which contains at least one heteroatom N and may optionally contain an additional heteromoiety selected from S, SO₂, N, and O, and the 5 or 6 membered ring may be saturated, partially unsaturated or aromatic (including piperidinyl, morpholinyl, imidazolyl, and pyridyl) wherein aromatic nitrogen in the 5 or 6 membered ring may be present as N-oxide (including pyridyl N-oxide), and the 5 or 6 membered ring may be optionally substituted with methyl, halogen, alkylamino, or alkoxy (including 1 methyl imidazolyl); R³ may also be absent, with the proviso that R³ is not absent when E is nitrogen;

### gg) R³ is

hydrogen, phenyl, 2-hydroxy ethylamino, 1-hydroxyeth-2-yl(methyl)amino, methylamino, 2-amino isopropyl, 1,3-dihydroxy isopropyl, 1,2-dihydroxy ethyl, methoxy, dimethylamino, 1-hydroxy eth-2-yl, —COOH, —CONH₂, —CN, —SO₂—, —SO₂CH₃), —NH₂, piperidinyl, morpholinyl, imidazolyl, pyridyl, pyridyl N-oxide), or 1 methyl imidazolyl;

### hh) R³ is

alkylamino (including methylamino), dialkylamino (including dimethylamino), or —SO₂-alkyl-R⁴ (including —SO₂CH₃);

### ii) R³ is

methylamino, dimethylamino, or —SO₂CH₃;

### jj) R³ is

dimethylamino;

### kk) R⁴ is

hydrogen, —OH, alkoxy, carboxy, carboxamido, or carbamoyl; and

### ll) R⁴ is

hydrogen;
   and all combinations of a) to ll), inclusive, herein above.

Other preferred embodiments of Formula I are those wherein:

### A is

phenyl or pyridyl, either of which may be substituted with one of chloro, fluoro, methyl, —N₃, —NH₂, —NH(alkyl), —N(alkyl)₂, —S(alkyl), —O(alkyl), or 4-aminophenyl;

### W is

pyrrolyl, imidazolyl, isoxazolyl, oxazolyl, 1,2,4 triazolyl, or furanyl, any of which may be connected through any carbon atom, wherein the pyrrolyl, imidazolyl, isoxazolyl, oxazolyl, 1,2,4 triazolyl, or furanyl may contain one —Cl, —CN, —NO₂, —OMe, or —CF₃ substitution, connected to any other carbon;

### R² is

cycloalkyl, thiophenyl, dihydrosulfonopyranyl, phenyl, furanyl, tetrahydropyridyl, or dihydropyranyl, any of which may be independently substituted with one or two of each of the following: chloro, fluoro, and C₍₁₋₃₎alkyl, with the proviso that tetrahydropyridyl is connected to the ring A through a carbon-carbon bond;

### X is

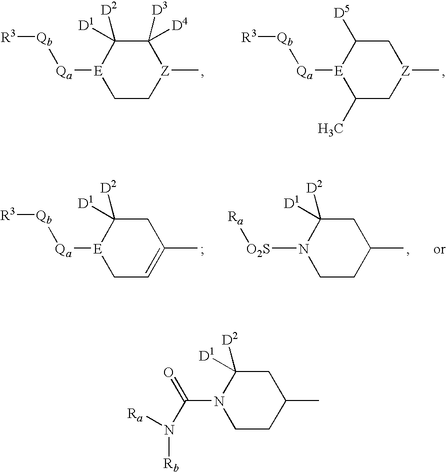

and is oriented para with respect to —NHCO—W;
Z is
   CH or N;
D¹ and D² are
   each hydrogen or taken together form a double bond to an oxygen;
D³ and D⁴ are
   each hydrogen or taken together form a double bond to an oxygen;
D⁵ is
   hydrogen or —CH₃, wherein said —CH₃ may be relatively oriented syn or anti;
Rₐ and R_{b} are independently
   hydrogen, cycloalkyl, haloalkyl, aryl, aralkyl, heteroaryl, or heteroaralkyl;
E is
   N, S, O, SO or SO₂, with the proviso that E may not be N if the following three conditions are simultaneously met: Qₐ is absent, Q_{b} is absent, and R³ is an amino group or cyclic amino radical wherein the point of attachment to E is N;
Qₐ is
   absent, —CH₂—, —CH₂CH₂—, or C(O);
Q_{b} is
   absent, —NH—, —CH₂—, —CH₂CH₂—, or C(O), with the proviso that Q_{b} may not be C(O) if Qₐ is C(O), and further provided that Q_{b} may not be —NH— if E is N and Qₐ is absent, further provided that Q_{b} may not be —NH— if R³ is an amino group or cyclic amino radical wherein the point of attachment to Q_{b} is N;
R³ is
   hydrogen, hydroxyalkylamino, (hydroxyalkyl)₂amino, alkylamino, aminoalkyl, dihydroxyalkyl, alkoxy, dialkylamino, hydroxyalkyl, —COOH, —CONH₂, —CN, —SO₂-alkyl-R⁴, —NH₂, or a 5 or six membered ring which contains at least one heteroatom Nand may optionally contain an additional heteromoiety selected from S, SO₂, N, and O, and the 5 or 6 membered ring may be saturated, partially unsaturated or aromatic, wherein aromatic nitrogen in the 5 or 6 membered ring may be present as N-oxide, and the 5 or 6 membered ring may be optionally substituted with methyl, halogen, alkylamino, or alkoxy; R³ may also be absent, with the proviso that R³ is not absent when E is nitrogen;
R⁴ is
   hydrogen, —OH, alkoxy, carboxy, carboxamido, or carbamoyl.

Other preferred embodiments of Formula I are those wherein:

### A is

phenyl or pyridyl;

### W is

pyrrolyl, imidazolyl, isoxazolyl, oxazolyl, 1,2,4 triazolyl, or furanyl, any of which may be connected through any carbon atom, wherein the pyrrolyl, imidazolyl, isoxazolyl, oxazolyl, 1,2,4 triazolyl, or furanyl may contain one —Cl, —CN, —NO₂, —OMe, or —CF₃ substitution, connected to any other carbon;

### R² is

cycloalkyl, thiophenyl, dihydrosulfonopyranyl, phenyl, furanyl, tetrahydropyridyl, or dihydropyranyl, any of which may be independently substituted with one or two of each of the following: chloro, fluoro, and C₍₁₋₃₎alkyl, with the proviso that tetrahydropyridyl is connected to the ring A through a carbon-carbon bond;

### X is

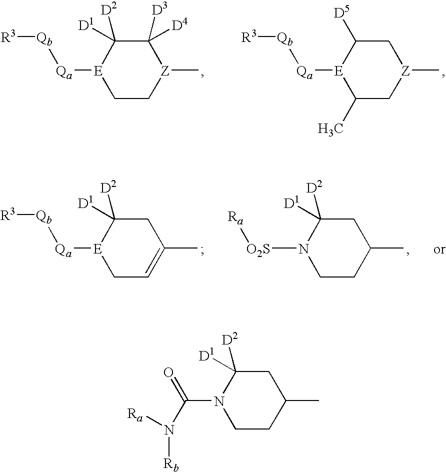

and is oriented para with respect to —NHCO—W;
Z is
   CH or N;
D¹ and D² are
   each hydrogen or taken together form a double bond to an oxygen;
D³ and D⁴ are
   each hydrogen or taken together form a double bond to an oxygen;
D⁵ is
   hydrogen or —CH₃, wherein said —CH₃ may be relatively oriented syn or anti;
Rₐ and R_{b} are independently
   hydrogen, cycloalkyl, haloalkyl, aryl, aralkyl, heteroaryl, or heteroaralkyl;
E is
   N, S, O, SO or SO₂, with the proviso that E may not be N if the following three conditions are simultaneously met: Qₐ is absent, Q_{b} is absent, and R³ is an amino group or cyclic amino radical wherein the point of attachment to E is N;
Qₐ is
   absent, —CH₂—, —CH₂CH₂—, or C(O);
Q_{b} is
   absent, —NH—, —CH₂—, —CH₂CH₂—, or C(O), with the proviso that Q_{b} may not be C(O) if Qₐ is C(O), and further provided that Q_{b} may not be —NH— if E is N and Qₐ is absent, further provided that Q_{b} may not be —NH— if R³ is an amino group or cyclic amino radical wherein the point of attachment to Q_{b} is N;
R³ is
   hydrogen, hydroxyalkylamino, (hydroxyalkyl)₂amino, alkylamino, aminoalkyl, dihydroxyalkyl, alkoxy, dialkylamino, hydroxyalkyl, —COOH, —CONH₂, —CN, —SO₂-alkyl-R⁴, —NH₂, or a 5 or six membered ring which contains at least one heteroatom Nand may optionally contain an additional heteromoiety selected from S, SO₂, N, and O, and the 5 or 6 membered ring may be saturated, partially unsaturated or aromatic, wherein aromatic nitrogen in the 5 or 6 membered ring may be present as N-oxide, and the 5 or 6 membered ring may be optionally substituted with methyl, halogen, alkylamino, or alkoxy; R³ may also be absent, with the proviso that R³ is not absent when E is nitrogen;
R⁴ is
   hydrogen, —OH, alkoxy, carboxy, carboxamido, or carbamoyl.

Other preferred embodiments of Formula I are those wherein:

### A is

phenyl or pyridyl;

### W is

3H-2-imidazolyl-4-carbonitrile;

### R² is

cycloalkyl, thiophenyl, dihydrosulfonopyranyl, phenyl, furanyl, tetrahydropyridyl, or dihydropyranyl, any of which may be independently substituted with one or two of each of the following: chloro, fluoro, and C₍₁₋₃₎alkyl, with the proviso that tetrahydropyridyl is connected to the ring A through a carbon-carbon bond;

### X is

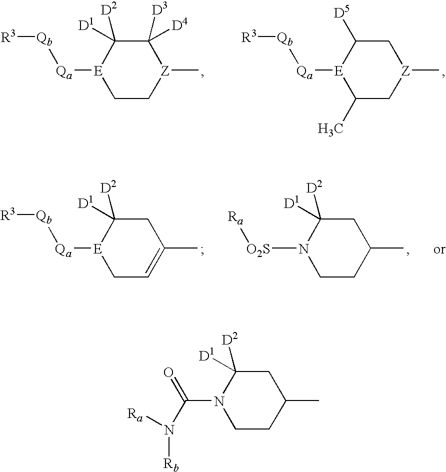

and is oriented para with respect to —NHCO—W;
Z is
   CH or N;
D¹ and D² are
   each hydrogen or taken together form a double bond to an oxygen;
D³ and D⁴ are
   each hydrogen or taken together form a double bond to an oxygen;
D⁵ is
   hydrogen or —CH₃, wherein said —CH₃ may be relatively oriented syn or anti;
Rₐ and R_{b} are independently
   hydrogen, cycloalkyl, haloalkyl, aryl, aralkyl, heteroaryl, or heteroaralkyl;
E is
   N, S, O, SO or SO₂, with the proviso that E may not be N if the following three conditions are simultaneously met: Qₐ is absent, Q_{b} is absent, and R³ is an amino group or cyclic amino radical wherein the point of attachment to E is N;
Qₐ is
   absent, —CH₂—, —CH₂CH₂—, or C(O);
Q_{b} is
   absent, —NH—, —CH₂—, —CH₂CH₂—, or C(O), with the proviso that Q_{b} may not be C(O) if Qₐ is C(O), and further provided that Q_{b} may not be —NH— if E is N and Qₐ is absent, further provided that Q_{b} may not be —NH— if R³ is an amino group or cyclic amino radical wherein the point of attachment to Q_{b} is N;
R³ is
   hydrogen, hydroxyalkylamino, (hydroxyalkyl)₂amino, alkylamino, aminoalkyl, dihydroxyalkyl, alkoxy, dialkylamino, hydroxyalkyl, —COOH, —CONH₂, —CN, —SO₂-alkyl-R⁴, —NH₂, or a 5 or six membered ring which contains at least one heteroatom Nand may optionally contain an additional heteromoiety selected from S, SO₂, N, and O, and the 5 or 6 membered ring may be saturated, partially unsaturated or aromatic, wherein aromatic nitrogen in the 5 or 6 membered ring may be present as N-oxide, and the 5 or 6 membered ring may be optionally substituted with methyl, halogen, alkylamino, or alkoxy; R³ may also be absent, with the proviso that R³ is not absent when E is nitrogen;
R⁴ is
   hydrogen, —OH, alkoxy, carboxy, carboxamido, or carbamoyl.

Other preferred embodiments of Formula I are those wherein:

### A is

phenyl or pyridyl;

### W is

3H-2-imidazolyl-4-carbonitrile;

### R² is

cyclohexenyl which may be substituted with one or two methyl groups;

### X is

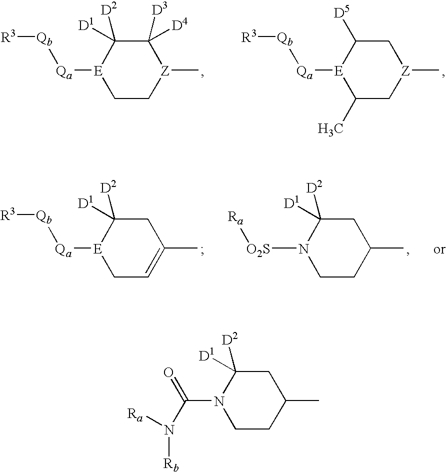

and is oriented para with respect to —NHCO—W;
Z is
   CH or N;
D¹ and D² are
   each hydrogen or taken together form a double bond to an oxygen;
D³ and D⁴ are
   each hydrogen or taken together form a double bond to an oxygen;
D⁵ is
   hydrogen or —CH₃, wherein said —CH₃ may be relatively oriented syn or anti;
Rₐ and R_{b} are independently
   hydrogen, cycloalkyl, haloalkyl, aryl, aralkyl, heteroaryl, or heteroaralkyl;
E is
   N, S, O, SO or SO₂, with the proviso that E may not be N if the following three conditions are simultaneously met: Qₐ is absent, Q_{b} is absent, and R³ is an amino group or cyclic amino radical wherein the point of attachment to E is N;
Qₐ is
   absent, —CH₂—, —CH₂CH₂—, or C(O);
Q_{b} is
   absent, —NH—, —CH₂—, —CH₂CH₂—, or C(O), with the proviso that Q_{b} may not be C(O) if Qₐ is C(O), and further provided that Q_{b} may not be —NH— if E is N and Qₐ is absent, further provided that Q_{b} may not be —NH— if R³ is an amino group or cyclic amino radical wherein the point of attachment to Q_{b} is N;
R³ is
   hydrogen, hydroxyalkylamino, (hydroxyalkyl)₂amino, alkylamino, aminoalkyl, dihydroxyalkyl, alkoxy, dialkylamino, hydroxyalkyl, —COOH, —CONH₂, —CN, —SO₂-alkyl-R⁴, —NH₂, or a 5 or six membered ring which contains at least one heteroatom N and may optionally contain an additional heteromoiety selected from S, SO₂, N, and O, and the 5 or 6 membered ring may be saturated, partially unsaturated or aromatic, wherein aromatic nitrogen in the 5 or 6 membered ring may be present as N-oxide, and the 5 or 6 membered ring may be optionally substituted with methyl, halogen, alkylamino, or alkoxy; R³ may also be absent, with the proviso that R³ is not absent when E is nitrogen;
R⁴ is
   hydrogen, —OH, alkoxy, carboxy, carboxamido, or carbamoyl.

Other preferred embodiments of Formula I are those wherein:

### A is

phenyl or pyridyl;

### W is

3H-2-imidazolyl-4-carbonitrile;

### R² is

cyclohexenyl which may be substituted with one or two methyl groups;

### X is

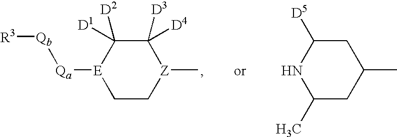

and is oriented para with respect to —NHCO—W;
Z is
   CH;
D¹ and D² are
   each hydrogen;
D³ and D⁴ are
   each hydrogen;
D⁵ is
   —CH₃, wherein said —CH₃ may be relatively oriented syn or anti;
E is
   N;
Qₐ is
   absent, —CH₂—, —CH₂CH₂—, or C(O);
Q_{b} is
   absent, —CH₂—, —CH₂CH₂—, or C(O), with the proviso that Q_{b} may not be C(O) if Qₐ is C(O), further provided that Q_{b} may not be —NH— if R³ is an amino group or cyclic amino radical wherein the point of attachment to Q_{b} is N;
R³ is
   hydrogen, hydroxyalkylamino, (hydroxyalkyl)₂amino, alkylamino, aminoalkyl, dihydroxyalkyl, alkoxy, dialkylamino, hydroxyalkyl, —COOH, —CONH₂, —CN, —SO₂—CH₃, —NH₂, pyridyl, pyridyl-N-oxide, or morpholinyl.

Other preferred embodiments of Formula I are those wherein:

### A is

phenyl or pyridyl;

### W is

3H-2-imidazolyl-4-carbonitrile;

### R² is

cyclohexenyl which may be substituted with one or two methyl groups;

### X is

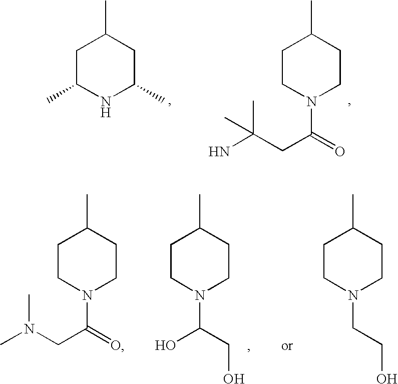

and is oriented para with respect to —NHCO—W.

Examples of compounds of Formula I include:
5-cyano-furan-2-carboxylic acid [4-(4-methyl-piperazin-1-yl)-2-(3-methyl-thiophen-2-yl)-phenyl]-amide, and
5-cyano-furan-2-carboxylic acid [4-(4-methyl-piperazin-1-yl)-2-(2-methyl-thiophen-3-yl)-phenyl]-amide,
   and solvates, hydrates, tautomers and pharmaceutically acceptable salts thereof.

Additional examples of compounds of Formula I include:
4-cyano-1H-imidazole-2-carboxylic acid [4-(1-acetyl-piperidin-4-yl)-2-(1,2,5,6-tetrahydro-pyri din-3-yl)-phenyl]-amide,
4-cyano-1H-imidazole-2-carboxylic acid [2-cyclohex-1-enyl-4-(1,1-dioxo-hexahydro-1λ⁶-thiopyran-4-yl)-phenyl]-amide,
5-cyano-furan-2-carboxylic acid [2-cyclohex-1-enyl-4-(4-methyl-piperazin-1-yl)-phenyl]-amide,
5-cyano-furan-2-carboxylic acid [2-(3,6-dihydro-2H-pyran-4-yl)-4-(4-methyl-piperazin-1-yl)-phenyl]-amide,
4-cyano-1H-imidazole-2-carboxylic acid [2-(1,1-dioxo-1,2,3,6-tetrahydro-1λ⁶-thiopyran-4-yl)-4-piperidin-4-yl-phenyl]-amide,
4-cyano-1H-imidazole-2-carboxylic acid [4-(1-acetyl-piperidin-4-yl)-2-(1,1-dioxo-1,2,3,6-tetrahydro-1λ⁶-thiopyran-4-yl)-phenyl]-amide,
5-cyano-furan-2-carboxylic acid [2′-methyl-5-(4-methyl-piperazin-1-yl)-biphenyl-2-yl]-amide, and
5-cyano-furan-2-carboxylic acid [2′-fluoro-5-(4-methyl-piperazin-1-yl)-biphenyl-2-yl]-amide,
   and solvates, hydrates, tautomers and pharmaceutically acceptable salts thereof.

Further examples of compounds of Formula I are:
(4-{4-[(4-cyano-1H-imidazole-2-carbonyl)-amino]-3-cyclohex-1-enyl-phenyl}-piperidin-1-yl)-acetic acid,
4-cyano-1H-imidazole-2-carboxylic acid [4-(1-carbamoylmethyl-piperidin-4-yl)-2-cyclohex-1-enyl-phenyl]-amide,
4-cyano-1H-imidazole-2-carboxylic acid [2-(4-methyl-cyclohex-1-enyl)-4-piperidin-4-yl-phenyl]-amide,
4-cyano-1H-imidazole-2-carboxylic acid {2-cyclohex-1-enyl-4-[1-(2-hydroxy-ethyl)-piperidin-4-yl]-phenyl}-amide,
4-cyano-1H-imidazole-2-carboxylic acid [2-(4-methyl-cyclohex-1-enyl)-4-(1-pyridin-2-ylmethyl-piperidin-4-yl)-phenyl]-amide,
4-cyano-1H-imidazole-2-carboxylic acid {2-cyclohex-1-enyl-4-[1-(2-hydroxy-1-hydroxymethyl-ethyl)-piperidin-4-yl]-phenyl}-amide,
4-cyano-1H-imidazole-2-carboxylic acid {4-[1-(2-cyano-ethyl)-piperidin-4-yl]-2-cyclohex-1-enyl-phenyl}-amide,
4-cyano-1H-imidazole-2-carboxylic acid {2-cyclohex-1-enyl-4-[1-(2-morpholin-4-yl-ethyl)-piperidin-4-yl]-phenyl}-amide,
4-cyano-1H-imidazole-2-carboxylic acid (2-cyclohex-1-enyl-4-piperidin-4-yl-phenyl)-amide,
4-cyano-1H-imidazole-2-carboxylic acid {2-cyclohex-1-enyl-4-[1-(2-methanesulfonyl-ethyl)-piperidin-4-yl]-phenyl}-amide,
4-cyano-1H-imidazole-2-carboxylic acid [2-cyclohex-1-enyl-4-(1-pyridin-2-ylmethyl-piperidin-4-yl)-phenyl]-amide,
4-cyano-1H-imidazole-2-carboxylic acid {2-cyclopent-1-enyl-4-[1-(1-methyl-1H-imidazol-2-ylmethyl)-piperidin-4-yl]-phenyl}-amide,
4-cyano-1H-imidazole-2-carboxylic acid (2-cyclopent-1-enyl-4-piperidin-4-yl-phenyl)-amide,
4-cyano-1H-pyrrole-2-carboxylic acid (2-cyclohex-1-enyl-4-piperidin-4-yl-phenyl)-amide,
4-cyano-1H-imidazole-2-carboxylic acid [2-cyclohex-1-enyl-4-(3,4,5,6-tetrahydro-2H-[1,2′]bipyridinyl-4-yl)-phenyl]-amide, and
4-cyano-1H-pyrrole-2-carboxylic acid [4-(1-acetyl-piperidin-4-yl)-2-cyclohex-1-enyl-phenyl]-amide,
   and solvates, hydrates, tautomers and pharmaceutically acceptable salts thereof.

Other examples of compounds of Formula I are:
4-cyano-1H-imidazole-2-carboxylic acid {2-cyclohex-1-enyl-4-[1-(1-oxy-pyridine-3-carbonyl)-piperidin-4-yl]-phenyl}-amide,
4-cyano-1H-imidazole-2-carboxylic acid {2-cyclohex-1-enyl-4-[1-(1-oxy-pyridine-4-carbonyl)-piperidin-4-yl]-phenyl}-amide,
4-cyano-1H-imidazole-2-carboxylic acid {2-cyclohex-1-enyl-4-[1-(3-morpholin-4-yl-propionyl)-piperidin-4-yl]-phenyl}-amide,
4-{4-[(4-cyano-1H-imidazole-2-carbonyl)-amino]-3-cyclohex-1-enyl-phenyl}-piperidine-1-carboxylic acid amide,
4-cyano-1H-imidazole-2-carboxylic acid {2-cyclohex-1-enyl-4-[1-(pyridine-3-carbonyl)-piperidin-4-yl]-phenyl}-amide,
4-{4-[(4-cyano-1H-imidazole-2-carbonyl)-amino]-3-cyclohex-1-enyl-phenyl}-piperidine-1-carboxylic acid (2-hydroxy-ethyl)-amide,
4-cyano-1H-imidazole-2-carboxylic acid {2-cyclohex-1-enyl-4-[1-(2-3H-imidazol-4-yl-acetyl)-piperidin-4-yl]-phenyl}-amide,
4-cyano-1H-imidazole-2-carboxylic acid {2-cyclohex-1-enyl-4-[1-(2-pyridin-4-yl-acetyl)-piperidin-4-yl]-phenyl}-amide,
4-cyano-1H-imidazole-2-carboxylic acid (2-cyclohex-1-enyl-4-{1-[2-(1-methyl-1H-imidazol-4-yl)-acetyl]-piperidin-4-yl}-phenyl)-amide,
4-cyano-1H-imidazole-2-carboxylic acid {2-cyclohex-1-enyl-4-[1-(2-pyridin-3-yl-acetyl)-piperidin-4-yl]-phenyl}-amide,
4-cyano-1H-imidazole-2-carboxylic acid {2-cyclohex-1-enyl-4-[1-(2-methanesulfonyl-acetyl)-piperidin-4-yl]-phenyl}-amide,
4-cyano-1H-imidazole-2-carboxylic acid {2-cyclohex-1-enyl-4-[1-(2-pyridin-2-yl-acetyl)-piperidin-4-yl]-phenyl}-amide, and
4-cyano-1H-imidazole-2-carboxylic acid [4-(1-acetyl-piperidin-4-yl)-2-cyclohex-1-enyl-phenyl]-amide,
   and solvates, hydrates, tautomers and pharmaceutically acceptable salts thereof.

Another example compound of Formula I is:
4-cyano-1H-imidazole-2-carboxylic acid [2-cyclohex-1-enyl-4-(1-{2-[(2-hydroxy-ethyl)-methyl-amino]-acetyl}-piperidin-4-yl)-phenyl]-amide,
   and solvates, hydrates, tautomers and pharmaceutically acceptable salts thereof.

Another example compound of Formula I is:
4-cyano-1H-imidazole-2-carboxylic acid {2-cyclohex-1-enyl-4-[1-(2-dimethylamino-acetyl)-piperidin-4-yl]-phenyl}-amide,
   and solvates, hydrates, tautomers and pharmaceutically acceptable salts thereof.

Another example compound of Formula I is:
4-cyano-1H-imidazole-2-carboxylic acid {2-cyclohex-1-enyl-4-[1-(2-morpholin-4-yl-acetyl)-piperidin-4-yl]-phenyl}-amide,
   and solvates, hydrates, tautomers and pharmaceutically acceptable salts thereof.

Still other example compounds of formula I are:
4-Cyano-1H-imidazole-2-carboxylic acid {4-[1-(3-amino-3-methyl-butyryl)-piperidin-4-yl]-2-cyclohex-1-enyl-phenyl}-amide trifluoroacetic acid salt,
4H-[1,2,4]-triazole-3-carboxylic acid (2-cyclohex-1-enyl-4-piperidin-4-yl-phenyl)-amide bis trifluoroacetic acid salt,
5-Chloro-4H-[1,2,4]-triazole-3-carboxylic acid (2-cyclohex-1-enyl-4-piperidin-4-yl-phenyl)-amide trifluoroacetic acid salt,
5-Cyano-1H-imidazole-2-carboxylic acid [2-cyclohex-1-enyl-4-(cis-2,6-dimethyl-piperidin-4-yl)-phenyl]-amide bis trifluoroacetic acid salt,
5-cyano-1H-imidazole-2-carboxylic acid [2-cyclohex-1-enyl-4-(trans-2,6-dimethyl-piperidin-4-yl)-phenyl]-amide bis trifluoroacetic acid salt,
5-Cyano-1H-imidazole-2-carboxylic acid {2-cyclohex-1-enyl-4-[1-(R)-(+)-(2,3-dihydroxy-propionyl)-piperidin-4-yl]-phenyl}-amide,
5-Cyano-1H-imidazole-2-carboxylic acid [2-cyclohex-1-enyl-4-(1-methoxy-piperidin-4-yl)-phenyl]-amide trifluoroacetic acid salt,
4-Cyano-1H-imidazole-2-carboxylic acid [6-(4,4-dimethyl-cyclohex-1-enyl)-1′,2′,3′,4′,5′,6′-hexahydro-[2,4′]bipyridinyl-5-yl]-amide trifluoroacetic acid salt,
5-Cyano-1H-imidazole-2-carboxylic acid {4-[1-(2-amino-2-methyl-propionyl)-piperidin-4-yl]-2-cyclohex-1-enyl-phenyl}-amide trifluoroacetic acid salt, and
5-Cyano-1H-imidazole-2-carboxylic acid [6-cyclohex-1-enyl-1′-(2-methanesulfonyl-ethyl)-1′,2′,3′,4′,5′,6′-hexahydro-[2,4]bipyridinyl-5-yl]-amide,
   and solvates, hydrates, tautomers and pharmaceutically acceptable salts thereof.

Additional example compounds of Formula I are:
4-Cyano-1H-imidazole-2-carboxylic acid {2-cyclohex-1-enyl-4-[1-(2-methylamino-acetyl)-piperidin-4-yl]-phenyl}-amide,
4-Cyano-1H-imidazole-2-carboxylic acid [1′-(2-dimethylamino-acetyl)-6-(4,4-dimethyl-cyclohex-1-enyl)-1′,2′,3′,4′,5′,6′-hexahydro-[2,4′]bipyridinyl-5-yl]-amide trifluoroacetic acid salt, and
4-Cyano-1H-imidazole-2-carboxylic acid [6-(4,4-dimethyl-cyclohex-1-enyl)-1′-(2-methanesulfonyl-ethyl)-1′,2′,3′,4′,5′,6′-hexhydro-[2,4′]bipyridinyl-5-yl]-amide trifluoroacetic acid salt,
   and solvates, hydrates, tautomers and pharmaceutically acceptable salts thereof.

As used herein, the term “the compounds of the present invention” shall also include solvates, hydrates, tautomers or pharmaceutically acceptable salts thereof.

### Pharmaceutically Acceptable Salts

As stated, the compounds of the present invention may also be present in the form of pharmaceutically acceptable salts.

For use in medicines, the salts of the compounds of the present invention refer to non-toxic “pharmaceutically acceptable salts.” FDA approved pharmaceutically acceptable salt forms (Ref. International J. Pharm. 1986, 33, 201-217; J. Pharm. Sci., 1977, January, 66(1), p 1) include pharmaceutically acceptable acidic/anionic or basic/cationic salts.

Pharmaceutically acceptable acidic/anionic salts include, and are not limited to acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate and triethiodide. Organic or inorganic acids also include, and are not limited to, hydriodic, perchloric, sulfuric, phosphoric, propionic, glycolic, methanesulfonic, hydroxyethanesulfonic, oxalic, 2-naphthalenesulfonic, p-toluenesulfonic, cyclohexanesulfamic, saccharinic or trifluoroacetic acid.

Pharmaceutically acceptable basic/cationic salts include, and are not limited to aluminum, 2-amino-2-hydroxymethyl-propane-1,3-diol (also known as tris(hydroxymethyl)aminomethane, tromethane or “TRIS”), ammonia, benzathine, t-butylamine, calcium, calcium gluconate, calcium hydroxide, chloroprocaine, choline, choline bicarbonate, choline chloride, cyclohexylamine, diethanolamine, ethylenediamine, lithium, LiOMe, L-lysine, magnesium, meglumine, NH₃, NH₄OH, N-methyl-D-glucamine, piperidine, potassium, potassium-t-butoxide, potassium hydroxide (aqueous), procaine, quinine, sodium, sodium carbonate, sodium-2-ethylhexanoate (SEH), sodium hydroxide, or zinc.

### Prodrugs

The present invention also includes within its scope, prodrugs of the compounds of the present invention. In general, such prodrugs will be functional derivatives of the compounds which are readily convertible in vivo into an active compound. Thus, in the methods of treatment of the present invention, the term “administering” shall encompass the means for treating, ameliorating or preventing a syndrome, disorder or disease described herein with the compounds of the present invention or a prodrug thereof, which would obviously be included within the scope of the invention albeit not specifically disclosed any given compound. Conventional procedures for the selection and preparation of suitable prodrug derivatives are described in, for example, “Design of Prodrugs”, ed. H. Bundgaard, Elsevier, 1985.

### Stereochemical Isomers

One skilled in the art will recognize that some compounds of the present invention have one or more asymmetric carbon atoms in their structure. It is intended that the present invention include within its scope single enantiomer forms of the compounds of the present invention, racemic mixtures, and mixtures of enantiomers in which an enantiomeric excess is present.

The term “single enantiomer” as used herein defines all the possible homochiral forms which the compounds of the present invention and their N-oxides, addition salts, quaternary amines, and physiologically functional derivatives may possess.

Stereochemically pure isomeric forms may be obtained by the application of art known principles. Diastereoisomers may be separated by physical separation methods such as fractional crystallization and chromatographic techniques, and enantiomers may be separated from each other by the selective crystallization of the diastereomeric salts with optically active acids or bases or by chiral chromatography. Pure stereoisomers may also be prepared synthetically from appropriate stereochemically pure starting materials, or by using stereoselective reactions.

The term “isomer” refers to compounds that have the same composition and molecular weight but differ in physical and/or chemical properties. Such substances have the same number and kind of atoms but differ in structure. The structural difference may be in constitution (geometric isomers) or in an ability to rotate the plane of polarized light (enantiomers).

The term “stereoisomer” refers to isomers of identical constitution that differ in the arrangement of their atoms in space. Enantiomers and diastereomers are examples of stereoisomers.

The term “chiral” refers to the structural characteristic of a molecule that makes it impossible to superimpose it on its mirror image.

The term “enantiomer” refers to one of a pair of molecular species that are mirror images of each other and are not superimposable.

The term “diastereomer” refers to stereoisomers that are not mirror images.

The symbols “R” and “S” represent the configuration of substituents around a chiral carbon atom(s).

The term “racemate” or “racemic mixture” refers to a composition composed of equimolar quantities of two enantiomeric species, wherein the composition is devoid of optical activity.

The term “homochiral” refers to a state of enantiomeric purity.

The term “optical activity” refers to the degree to which a homochiral molecule or nonracemic mixture of chiral molecules rotates a plane of polarized light.

It is to be understood that the various substituent stereoisomers, geometric isomers and mixtures thereof used to prepare the compounds of the present invention are either commercially available, can be prepared synthetically from commercially available starting materials or can be prepared as isomeric mixtures and then obtained as resolved isomers using techniques well-known to those of ordinary skill in the art.

The isomeric descriptors “R,” and “S” are used as described herein for indicating atom configuration(s) relative to a core molecule and are intended to be used as defined in the literature (IUPAC Recommendations for Fundamental Stereochemistry (Section E), Pure Appl. Chem., 1976, 45:13-30).

The compounds of the present invention may be prepared as an individual isomer by either isomer-specific synthesis or resolved from an isomeric mixture. Conventional resolution techniques include forming the free base of each isomer of an isomeric pair using an optically active salt (followed by fractional crystallization and regeneration of the free base), forming an ester or amide of each of the isomers of an isomeric pair (followed by chromatographic separation and removal of the chiral auxiliary) or resolving an isomeric mixture of either a starting material or a final product using preparative TLC (thin layer chromatography) or a chiral HPLC column.

### Polymorphs and Solvates

Furthermore, the compounds of the present invention may have one or more polymorph or amorphous crystalline forms and as such are intended to be included in the scope of the invention. In addition, the compounds may form solvates, for example with water (i.e., hydrates) or common organic solvents. As used herein, the term “solvate” means a physical association of the compounds of the present invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. The term “solvate” is intended to encompass both solution-phase and isolatable solvates. Non-limiting examples of suitable solvates include ethanolates, methanolates, and the like.

It is intended that the present invention include within its scope solvates of the compounds of the present invention. Thus, in the methods of treatment of the present invention, the term “administering” shall encompass the means for treating, ameliorating or preventing a syndrome, disorder or disease described herein with the compounds of the present invention or a solvate thereof, which would obviously be included within the scope of the invention albeit not specifically disclosed.

### N-Oxides

The compounds of the present invention may be converted to the corresponding N-oxide form following art-known procedures for converting a trivalent nitrogen into its N-oxide form. Said N-oxidation reaction may generally be carried out by reacting the starting material with an appropriate organic or inorganic peroxide. Appropriate inorganic peroxides comprise, for example, hydrogen peroxide, alkali metal or earth alkaline metal peroxides, e.g. sodium peroxide, potassium peroxide; appropriate organic peroxides may comprise peroxy acids such as, for example, benzenecarboperoxoic acid or halo substituted benzenecarboperoxoic acid, e.g. 3-chlorobenzenecarboperoxoic acid, peroxoalkanoic acids, e.g. peroxoacetic acid, alkylhydroperoxides, e.g. tbutyl hydro-peroxide. Suitable solvents are, for example, water, lower alcohols, e.g. ethanol and the like, hydrocarbons, e.g. toluene, ketones, e.g. 2-butanone, halogenated hydrocarbons, e.g. dichloromethane, and mixtures of such solvents.

### Tautomeric Forms

The compounds of the present invention may also exist in their tautomeric forms. Such forms although not explicitly indicated in the present application are intended to be included within the scope of the present invention.

### Preparation of the Compounds of the Present Invention

During any of the processes for preparation of the compounds of the present invention, it may be necessary and/or desirable to protect sensitive or reactive groups on any of the molecules concerned. This may be achieved by means of conventional protecting groups, such as those described in Protecting Groups, P. Kocienski, Thieme Medical Publishers, 2000; and T. W. Greene & P. G. M. Wuts, Protective Groups in Organic Synthesis, 3^{rd} ed. Wiley Interscience, 1999. The protecting groups may be removed at a convenient subsequent stage using methods known in the art.

### Methods of Preparation

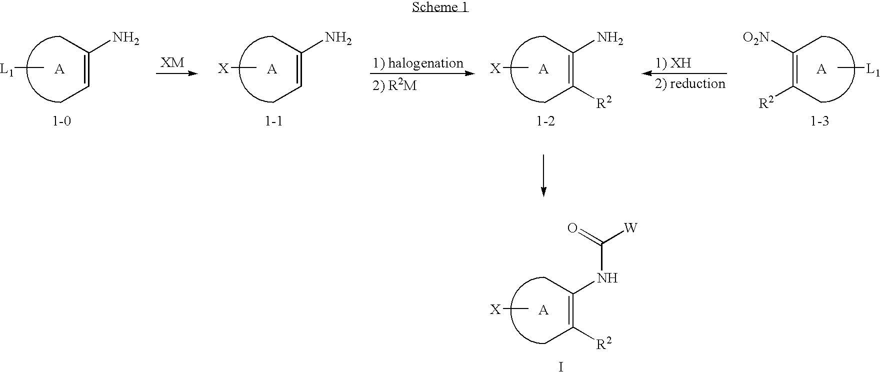

Scheme 1 illustrates general methodology for the preparation of compounds of Formula I. Compounds of Formula 1-2 can be obtained by ortho-halogenation, preferably bromination, of amino compounds of Formula 1-1 followed by metal-catalyzed coupling reactions with boronic acids or boronate esters (Suzuki reactions, where R²M is R²B(OH)₂ or a boronic ester) or tin reagents (Stille reactions, where R²M is R²Sn(alkyl)₃) (for reviews, see N. Miyaura, A. Suzuki, Chem. Rev., 95:2457 (1995), J. K. Stille, Angew. Chem., Int. Ed. Engl., 25: 508024 (1986) and A. Suzuki in Metal-Catalyzed Coupling Reactions, F. Deiderich, P. Stang, Eds., Wiley-VCH, Weinheim (1988)). Compounds of formula 1-1 may be commercially available, or the above palladium mediated cross-coupling reactions described above may be used to generate compounds of Formula 1-1 from starting material 1-0.

Preferred conditions for the bromination of 1-1 are N-bromosuccinimide (NBS) in a suitable solvent such as N,N-dimethylformamide (DMF), dichloromethane (DCM) or acetonitrile. Metal-catalyzed couplings, preferably Suzuki reactions, can be performed according to standard methodology, preferably in the presence of a palladium catalyst such as tetrakis(triphenylphosphine)palladium(0) (Pd(PPh₃)₄), an aqueous base such aq. Na₂CO₃, and a suitable solvent such as toluene, ethanol, dimethoxyethane (DME), or DMF.

Compounds of Formula I can be prepared by reaction of compounds of Formula 1-2 with carboxylic acids WCOOH according to standard procedures for amide bond formation (for a review, see: M. Bodansky and A. Bodansky, The Practice of Peptide Synthesis, Springer-Verlag, NY (1984)) or by reaction with acid chlorides WCOCl or activated esters WCO₂Rq (where Rq is a leaving group such as pentafluorophenyl or N-succinimide). The preferred reaction conditions for coupling with WCOOH are: when W is a furan, oxalyl chloride in DCM with DMF as a catalyst to form the acid chloride WCOCl and then coupling in the presence of a trialkylamine such as DIEA; when W is a pyrrole, 1-(3-dimethylaminopropyl)-3-ethylcarbodiimide hydrochloride (EDCI) and 1-hydroxybenzotriazole-6-sulfonamidomethyl hydrochloride (HOBt); and when W is an imidazole, the preferred conditions are bromotripyrrolidinophosphonium hexafluorophosphate (PyBrOP) and diisopropylethylamine (DIEA) in DCM.

It is understood that the optional substitution present on ring A in Formula I may be present in the starting materials 1-1 or 1-3 and, in such cases, would be carried through the synthesis outlined in Scheme 1. Alternatively various substituents on compounds of Formula I may be introduced in a number of ways described below to provide the optional substitution listed for Formula I. The leaving group “L₁” present on ring A in Formula 1-0 or 1-3, can be substituted before or at any step during Scheme 1. When such leaving groups (preferably fluoro or chloro) are activated by the nitro group of Formula 1-3 for nucleophilic attack, they can undergo direct nucleophilic aromatic substitution by ammonia and azide anion or by amines, alcohols, thiols and other nucleophiles in the presence of a suitable base such as K₂CO₃, N,N-diisopropylethylamine (DIEA) or NEt₃. When the leaving group is suitable for metal-catalyzed couplings (preferably bromo or trifluoromethanesulfonyloxy), a number of cross-coupling reactions (such as Suzuki or Stille reactions as discussed above for the introduction of R²) may be performed. Other metal-catalyzed coupling reactions that can be employed include aromatic and heteroaromatic amination and amidation (for reviews, see: S. L. Buchwald, et al, Top. Curr. Chem., 219:131-209 (2001) and J. F. Hartwig in “Organopalladium Chemistry for Organic Synthesis,” Wiley Interscience, NY (2002). Additional metal catalyzed cross coupling reactions with 2,4,6-trimethyl-cyclotriboroxane may be employed if L₁ is bromo, iodo, or chloro activated by nitro to generate optional methyl substitution (see M. Gray, et al, Tetrahedron Lett., 41: 6237-40 (2000)).

In some cases, the initial substituents can be further derivatized as described below to provide the final substitution of Formula I.

An alternative method for the introduction of nitrogen-containing heterocyclic substituents onto ring A is to form the heterocycle from an amino group on ring A. The amino group may be originally present in the starting material in a protected or unprotected form or may result from the reduction of a nitro group which also can be either originally present in the starting material or attached by a nitration reaction. In addition, the amino group may be formed by reduction of an azide group which can be present in the starting material or may result from nucleophilic aromatic substitution of an activated halide by azide anion as mentioned above. The amino group may also result from nucleophilic aromatic substitution of an activated halide (m, for example a nitrohalo compound) by ammonia or by the anion of a protected ammonia equivalent, for example, t-butyl carbamate. If introduced in protected form, the amine can be deprotected according to standard literature methods. (For examples of amine protecting groups and deprotection methods see: Theodora W. Greene and Peter G. M. Wuts, John Wiley and Sons, Inc., NY (1991).) The ring-forming reaction involves treatment of the aniline amino group with a suitable optionally substituted di-electrophile, preferably a dihalide or dicarbonyl compound, which results in two substitutions on the amino group to form an optionally substituted heterocycle. In the case of dihalides, any of a number of suitable bases can be added as an acid scavenger such as potassium carbonate, sodium hydroxide, or, a trialkylamine such as triethylamine. Thus, treatment with a bis(2-haloethyl)amine such as bis(2-chloroethyl)amine or bis(2-bromoethyl)amine would afford a piperazine ring (see, for example, J. Med. Chem., 29: 640-4 (1986) and J. Med. Chem., 46: 2837 (2003)). Optional substitution on the amine nitrogen of the reagent would incorporate optional substitution on the terminal amine of the piperazine. For example, treatment with N,N-bis(2-chloroethyl)aniline would give an N-phenylpiperazino group. Treatment with a bis(2-haloethyl)ether or bis(2-haloethyl)thioether would afford a morpholine or thiomorpholine ring, respectively.

Another alternative method to direct substitution to introduce heterocyclic substituents onto ring A is to form the heterocycle from an aldehyde (i.e. from a formyl group on ring A). The formyl group may be originally present in the starting material in a protected or unprotected form or may result from or any of a number of formylation reactions known in the literature including a Vilsmeier-Haack reaction (for a review of formylation chemistry, see: G. A. Olah, et al, Chem. Rev., 87: (1987)) or by para-formylation of nitroaromatics (see: A. Katritsky and L. Xie, Tetrahedron Lett., 37:347-50 (1996)).

Finally it is understood that compounds of Formula I may be further derivatized. Protecting groups on compounds of Formula I can be removed according to standard synthetic methodologies (Theodora W. Greene and Peter G. M. Wuts, John Wiley and Sons, Inc., NY (1991)) and can be then subjected to further derivatization. Examples of further derivatization of compounds of I include, but are not limited to: when compounds of Formula I contain a primary or secondary amine, the amine may be reacted with aldehydes or ketones in the presence of a reducing agent such as sodium triacetoxyborohydride (see Abdel-Magid J. Org. Chem. 61, pp. 3849-3862, (1996)) to reductively alkylate; with acid chlorides or carboxylic acids and an amide bond forming reagent as described above to form amides; with sulfonyl chlorides to form sulfonamides; with isocyanates to form ureas; with aryl- or heteroaryl-halides in the presence of a palladium catalyst as described above (see Buchwald and Hartwig references above) to form aryl and heteroarylamines In addition, when compounds of Formulae I contain an aryl halide or heteroaryl halide, these compounds may be subjected to metal-catalyzed reactions with boronic acids (for example, Suzuki or Stille couplings as described above), or, amines or alcohols (Buchwald- or Hartwig-type couplings, see Buchwald and Hartwig references above). When compounds of Formulae I contain a cyano group, this group may be hydrolyzed to amides or acids under acid or basic conditions. Basic amines may be oxidized to N-oxides and conversely N-oxides may be reduced to basic amines. When compounds of Formula I contain a sulfide, either acyclic or cyclic, the sulfide can be further oxidized to the corresponding sulfoxides or sulfones. Sulfoxides can be obtained by oxidation using an appropriate oxidant such as one equivalent of (meta-chloroperbenzoicacid) MCPBA or by treatment with NaIO₄ (see, for example, J. Regan, et al, J. Med. Chem., 46: 4676-86 (2003)) and sulfones can be obtained using two equivalents of MCPBA or by treatment with 4-methylmorpholine N-oxide and catalytic osmium tetroxide (see, for example, PCT application WO 01/47919).

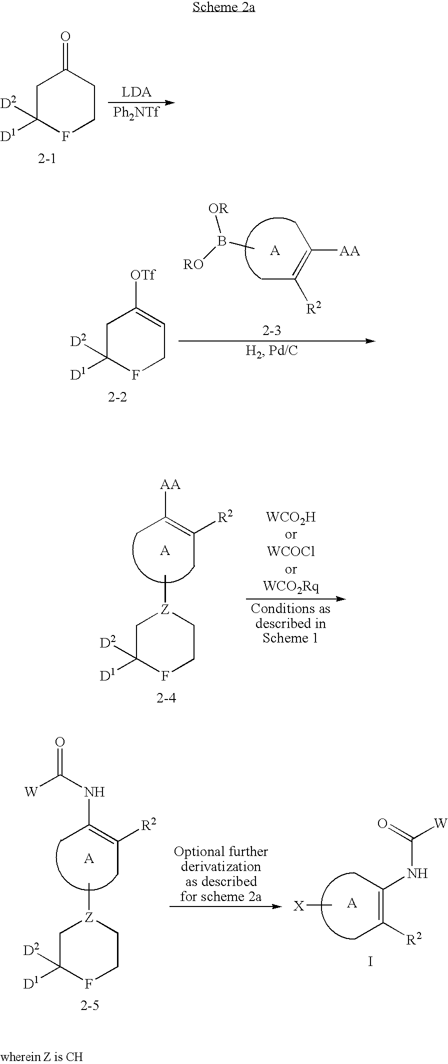

Scheme 2a illustrates a route to compounds of Formula I. F represents —NQₐQ_{b}R³—, —O—, S, SO, or SO₂, and AA represents —NH₂ or —NO₂. D¹ and D² are shown for illustrative purposes only; it is recognized by those skilled in art that D⁵ D⁶ D⁷ D⁸ may also be present. Ketones of formula 2-1 can be converted to a vinyl triflate of formula 2-2 by treatment with a non-nucleophilic base such as LDA and then trapping of the resulting enolate with a triflating reagent such as trifluoromethanesulfonic anhydride or preferably N-phenyltrifluoromethanesulfonimide. Suzuki coupling of boronic acids or boronate esters of formula 2-3 to vinyl triflates of formula 2-2 can provide compounds of formula 2-4 where Z is C (Synthesis, 993 (1991)).

For compounds of formula 2-4 treatment with Pd/C can reduce both the olefin (and the nitro if AA is NO₂) to give Z is CH, AA is NH₂. Compounds of formula 2-4 where F represents —SO₂ can be prepared from compounds of formula 2-4 where AA is —NO₂ and F is a sulfide (F is —S—) by oxidation with MCPBA or other methods described in Scheme 1. The nitro group may then be reduced with Pd/C to reduce both the nitro and the olefin.

Compounds of formula 2-4 (AA is NH₂) are then converted to compounds of Formula 2-5 (which also represent compounds of Formulae I if no further modifications are required) as described in Scheme 1.

Compounds of formula 2-5 may be further modified to provide additional compounds of Formula I. For example, in cases where F is —NQₐQ_{b}R³—, QₐQ_{b} is a direct bond, and R₃ represents a BOC protecting group (CO₂tBu), the BOC group may be removed according to standard methodology such as trifluoroactic acid (TFA) in DCM (Greene and Wuts, ibid.) to provide a secondary amine that can then be further derivatized to provide compounds of Formula I. Further derivatization includes, but is not limited to: reactions with aldehydes or ketones in the presence of a reducing agent such as sodium triacetoxyborohydride to provide compounds of Formula II where F is —NCH₂R³ (A. F. Abdel-Magid, ibid.); with acid chlorides or with carboxylic acids and an amide bond forming reagent (as described in Scheme 1) to provide compounds of Formula II where F is —NCOR³; with sulfonyl chlorides (as described in Scheme 1) to provide compounds of Formula I where F is —NSO₂Rₐ; with isocyanates (as described in Scheme 1) to provide compounds of Formula II where F is —NCONRₐR_{b}; or subjected to metal-catalyzed substitution reactions as outlined in Scheme 1 to provide compounds of Formula I where F is —NR³. (S. L. Buchwald, et al, ibid.; J. H. Hartwig, ibid.) For the above example, Rₐ and R_{b} are independently hydrogen, alkyl, cycloalkyl, haloalkyl, aryl, aralkyl, heteroaryl and heteroaralkyl.

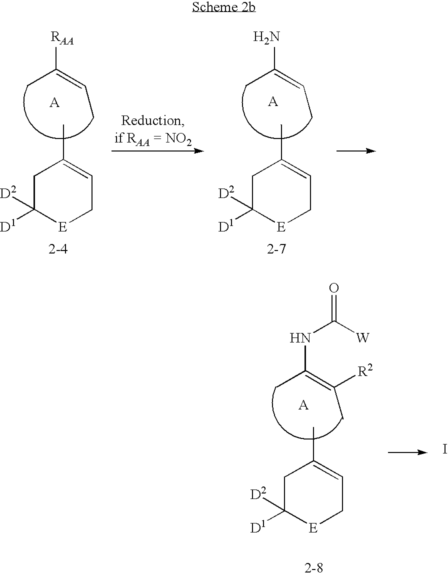

Scheme 2b illustrates a modification of Scheme 2a to synthesize partially unsaturated compounds of Formula I. E represents —NQₐQ_{b}R³—, —O— (D¹ and D² are H), —S— (D¹ and D² are H), -(D¹ and D² are H), or —SO₂— (D¹ and D² are H), and R_{AA} represents —NH₂ or —NO₂. Compounds of formula 2-4 are prepared as shown in Scheme 2. If R_{AA} is —NO₂, the nitro group must be reduced by a method that does not reduce olefins, such as iron and ammonium chloride. If R_{AA} of formula 2-4 is an amino group then no step is necessary and compounds of formula 2-4 are also compounds of formula 2-7. To prepared compounds of formula 2-7 where E is —SO₂— or —SO—, the oxidation of the sulfide must be performed on compound 2-4 where R_{AA} is —NO₂ as described above, followed by nitro reduction.

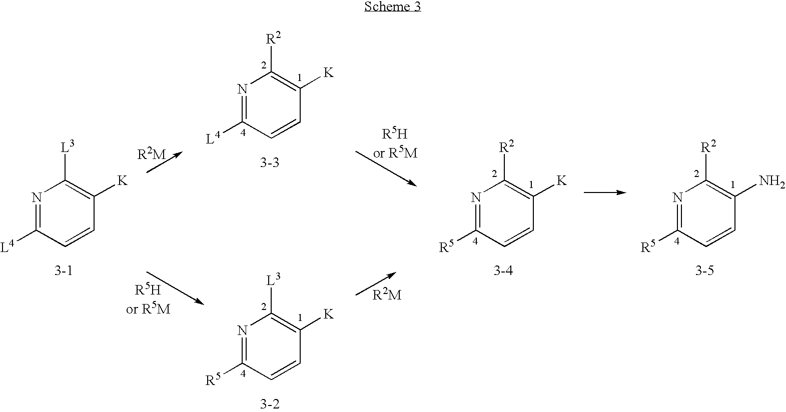

Scheme 3 illustrates the preparation of intermediates for the synthesis of compounds of Formula I, where ring A is pyridyl, and R⁵ is the optional substitution on ring A or one of the heterocyclic substituents as defined in Formula I. K is NH₂ or other functional groups such as NO₂, COOH or COOR which can eventually be converted to amino group by known literature methods such as reductions for NO₂ (as discussed for Scheme 1) or Curtius rearrangement for COOH (for a review, see Organic Reactions, 3: 337 (1947)). L³ and L⁴ are halogens. (K is COOH can also be formed from K is COOR by simple base- or acid-catalyzed hydrolysis.) In general, the selectivity and order in introducing R² and R⁵ can be achieved by the relative reactivity of the halogens L³ and L⁴ chosen in compound (3-1), the intrinsic selectivity of the heterocycle and/or the reaction conditions employed. An example of using the relative reactivity of the halogens L³ and L⁴ in selectively introducing R² and R⁵ would include the situation where, in compounds of Formula 3-1 where L³ is a fluoro group and L⁴ is a bromo group, selective displacement of the fluoro group by a nucleophile can be achieved followed by substitution of the remaining bromo group by metal-catalyzed substitution chemistry (such as Suzuki or Stille cross-coupling reactions as further outlined below). Similarly in compounds of Formula 3-1 where one of L³ and L⁴ is an iodo group and the other is a bromo or chloro group, selective metal-catalyzed substitution chemistry (such as Suzuki or Stille cross-coupling reactions or Buchwald/Hartwig aminations as further discussed below) on the iodo group can be achieved followed by replacement of the remaining bromo or chloro group by another metal-catalyzed substitution reaction.

As illustrated in Scheme 3, leaving group L³ in Formula 3-1 can be first substituted to obtain compounds of Formula 3-3 or leaving group L⁴ can be first substituted to obtain compound of Formula 3-2. Compounds 3-2 or 3-3 can then be reacted to displace L³ or L⁴ to furnish the compound of Formula 3-4.

Thus, a direct nucleophilic displacement or metal-catalyzed amination of compound of Formula 3-1 with a secondary amine, ammonia or a protected amine such as tert-butyl carbamate (for review, see Modern Amination Methods: Ricci, A., Ed.; Wiley-VCH: Weinheim, 2000), can be used to introduce R⁵ in Formulae 3-2 or 3-3 where R⁵ is a primary or secondary amine, amino group (NH₂), and amine equivalent or a protected amino group. Metal-catalyzed coupling of compound 3-1 with boronic acids or boronates esters (Suzuki reaction, M is boronic acid group or boronate ester group) or with organotin compounds (Stille reaction, M is SnR₃, where R is alkyl and the other substituents as defined above, as described in Scheme 1 can provide compounds of Formulae 3-2 or 3-3.

Compound 3-2 can be further converted to compound 3-4 by a metal-catalyzed Suzuki or Stille coupling as described above. L⁴ in compound 3-3 also subsequently can be substituted with R⁵ to obtain compounds of Formula 3-4, again, by a direct nucleophilic substitution or metal-catalyzed reaction with a nucleophile or by the same metal-catalyzed cross-coupling reaction as described above. When R⁵ in the formulae (3-2, 3-3 or 3-4) is a protected amine and K not an amino group, it can be deprotected to unmask the amino functionality. This amino functionality can then be further derivatized as described in Scheme 1. When the K group in Formula 3-4 is not an amino group (such as functionality described above), it can be converted to an amino group according to known literature methods (see, for example Comprehensive Organic Transformations Larock, R. S.; Wiley and Sons Inc., USA, 1999) and the resulting amine 3-5 can be employed in amide bond formation reactions as described in Scheme (1) to obtain the compounds in Formula I. When K in Formula 3-4 is an amino group it can be directly used in amide coupling as described above.

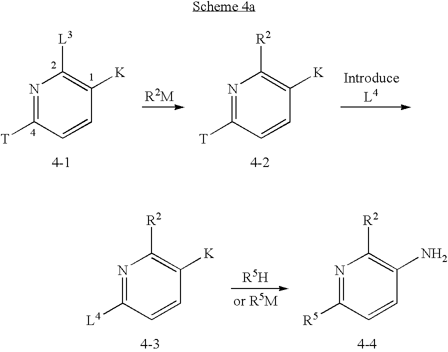

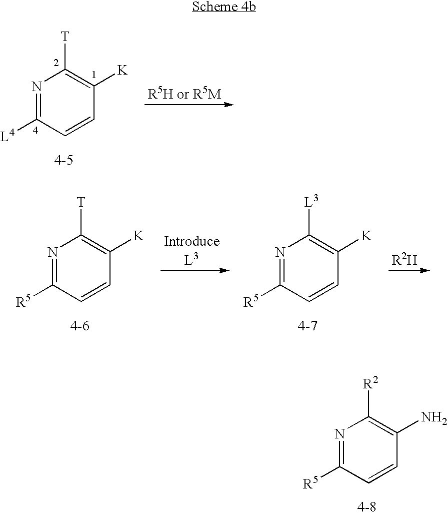

Schemes 4a and 4b illustrate the preparation of intermediates to be further modified according to Scheme 3 starting from a monohalo-substituted compound of Formulae 4-1 and 4-5 by introducing the second leaving group after the replacement of the first one has been completed. These can also be used for the synthesis of compounds of Formula I where ring A is a pyridine and R⁵ is either the optional substitution on Ring A or one of the heterocyclic substituents. As in Scheme 3, the remaining positions on the pyridine ring can be substituted as described in Formula I. K is NH₂ or other functional groups such as NO₂, COOH or COOR which can eventually be converted to amino group by known literature methods such as reductions or Curtius rearrangement as described in Scheme 3. L³ and L⁴ are halogens. In these compounds, T is either H or is a functional group such as OH that can be converted to leaving groups L³ or L⁴ such as halogen, triflate or mesylate by known literature methods (see, for example, Nicolai, E., et al., J. Heterocyclic Chemistry, 31, (73), (1994)). Displacement of L³ in compound of Formula 4-1 or L⁴ in Formula 4-5 by methods described in Scheme 3, can yield compounds of Formulae 4-2 and 4-6. At this point, the substituent T of compounds 4-2 or 4-6 can be converted to a leaving group L⁴ or L³ (preferably a halogen) by standard methods to provide compounds of Formulae 4-3 and 4-5. For example, when T is OH, the preferred reagents to effect this transformation are thionyl chloride, PCl₅, POCl₃ or PBr₃ (see, for examples, Kolder, den Hertog., Recl. Tray. Chim. Pays-Bas; 285, (1953), and Iddon, B, et. al., J. Chem. Soc. Perkin Trans. 1., 1370, (1980)). When T is H, it can be directly halogenated (preferably brominated) to provide compounds of Formulae 4-3 or 4-7 (see, for example, Canibano, V. et al., Synthesis, 14, 2175, (2001)). The preferred conditions for bromination are NBS in a suitable solvent such as DCM or acetonitrile.

The compounds of Formulae 4-3 or 4-7 can be converted to compounds of Formulae 4-4 or 4-8 by introduction of the remaining groups R² or R⁵, respectively, by the methods described above and then on to compounds of Formula I, by the methods described in Scheme 3 for conversion of compounds of Formulae 3-4 and 3-5 to compounds of Formula I.

Representative compounds of the present invention and their synthesis are presented in the following chart and examples thereafter. The following are for exemplary purposes only and are in no way meant to limit the invention. Preferred compounds of the present invention are Examples 14, 17, 34, 35, 38a, 38b, 40, 51a, 51b, 55 and 56; most preferred is compound 38a.

| | Name | Structure |
|---|---|---|
| 4 | 5-Cyano-furan-2-carboxylic acid [4-(4-methyl-piperazin-1-yl)-2- (3-methyl-thiophen-2-yl)-phenyl]-amide | 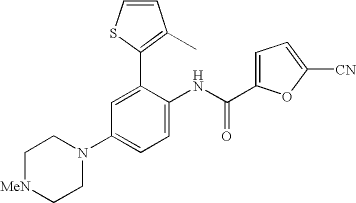 |
| 5 | 5-Cyano-furan-2-carboxylic acid [4-(4-methyl-piperazin-1-yl)-2- (4-methyl-thiophen-3-yl)- phenyl]-amide | 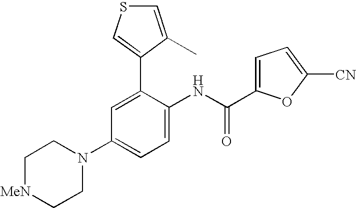 |
| 6 | 4-Cyano-1H-imidazole-2- carboxylic acid {2-cyclohex-1- enyl-4-[1-(2-hydroxy-1- hydroxymethyl-ethyl)-piperidin- 4-yl]-phenyl}-amide trifluoroacetic acid salt | 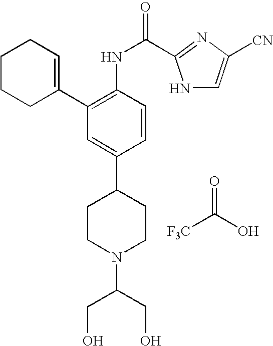 |
| 7 | 4-Cyano-1H-imidazole-2- carboxylic acid {2-cyclohex-1- enyl-4-[1-(2-morpholin-4-yl- acetyl)-piperidin-4-yl]-phenyl}- amide | 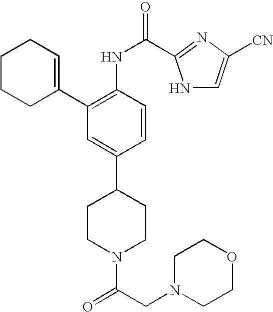 |
| 8 | 4-Cyano-1H-imidazole-2- carboxylic acid {2-cyclohex-1- enyl-4-[1-(3-morpholin-4-yl- propionyl)-piperidin-4-yl]- phenyl}-amide | 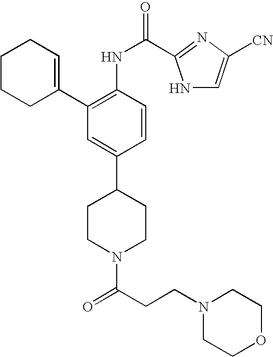 |
| 9 | 5-Cyano-furan-2-carboxylic acid [2’-methyl-5-(4-methyl-piperazin- 1-yl)-biphenyl-2-yl]-amide | 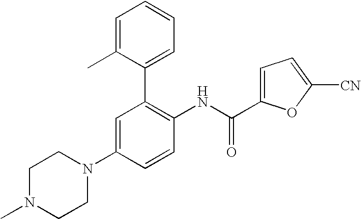 |
| 10 | 5-Cyano-furan-2-carboxylic acid [2’-fluoro-5-(4-methyl-piperazin- 1-yl)-biphenyl-2-yl]-amide | 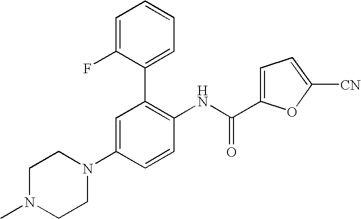 |
| 11 | 5-Cyano-furan-2-carboxylic acid [2-cyclohex-1-enyl-4-(4-methyl- piperazin-1-yl)-phenyl]-amide | 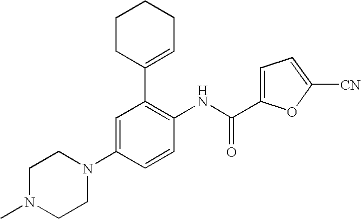 |
| 12 | 5-Cyano-fhran-2-carboxylic acid[2-(3,6-dihydro-2H-pyran-4- yl)-4-(4-methyl-piperazin-1-yl)- phenyl-amide | 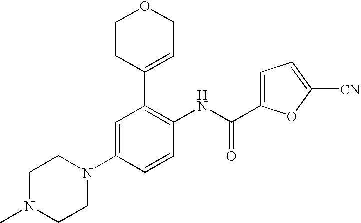 |
| 13 | 4-Cyano-1H-pyrrole-2-carboxylic acid (2-cyclohex-1-enyl-4- piperidin-4-yl-phenyl)-amide trifluoroacetic acid salt | 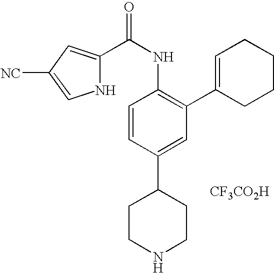 |
| 14 | 4-Cyano-1H-imidazole-2- carboxylic acid (2-cyclohex-1- enyl-4-piperidin-4-yl-phenyl)- amide trifluoroacetic acid salt | 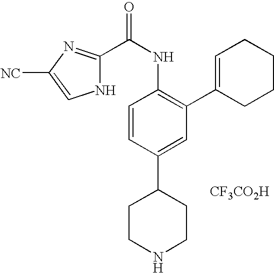 |
| 15 | 4-Cyano-1H-pyrrole-2-carboxylic acid [4-(1-acetyl-piperidin-4-yl)- 2-cyclohex-1-enyl-phenyl]-amide | 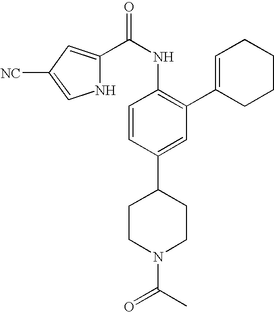 |
| 16 | 4-Cyano-1H-imidazole-2- carboxylic acid [4-(1-acetyl- piperidin-4-yl)-2-cyclohex-1- enyl-phenyl]-amide | 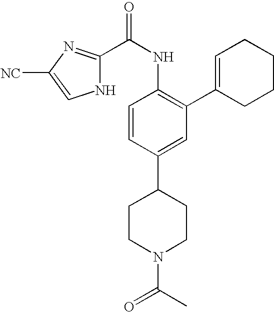 |
| 17 | 4-Cyano-1H-imidazole-2- carboxylic acid [2-(4-methyl- cyclohex-1-enyl)-4-piperidin-4- yl-phenyl]-amide trifluoroacetic acid salt | 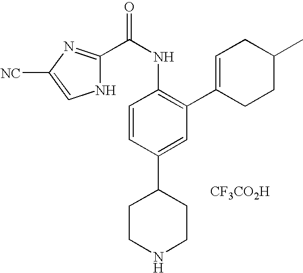 |
| 18 | 4-Cyano-1H-imidazole-2- carboxylic acid (2-cyclopent-1- enyl-4-piperidin-4-yl-phenyl)- amide trifluoroacetic acid salt | 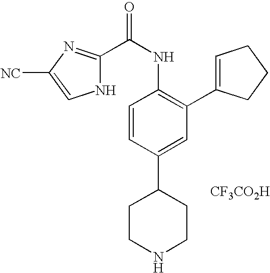 |
| 20 | 4-Cyano-1H-imidazole-2- carboxylic acid {2-cyclohex-1- enyl-4-[1-(2-methanesulfonyl- acetyl)-piperidin-4-yl]-phenyl}- amide | 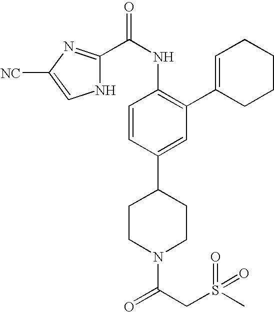 |
| 21 | 4-Cyano-1H-imidazole-2- carboxylic acid [2-cyclohex-1- enyl-4-(1-pyridin-2-ylmethyl- piperidin-4-yl)-phenyl]-amide trifluoroacetic acid salt | 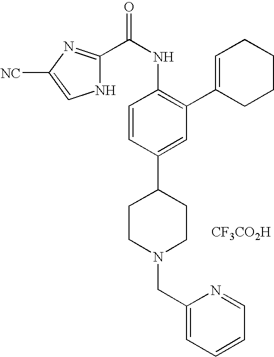 |
| 22 | 4-Cyano-1H-imidazole-2- carboxylic acid [2-(4-methyl- cyclohex-1-enyl)-4-(1-pyridin-2- ylmethyl-piperidin-4-yl)-phenyl]- amide trifluoroacetic acid salt | 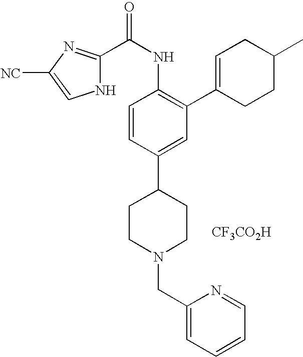 |
| 23 | 4-Cyano-1H-imidazole-2- carboxylic acid {2-cyclopent-1- enyl-4-[1-(1-methyl-1H-imidazol- 2-ylmethyl)-piperidin-4-yl]- phenyl}-amide trifluoroacetic acid salt | 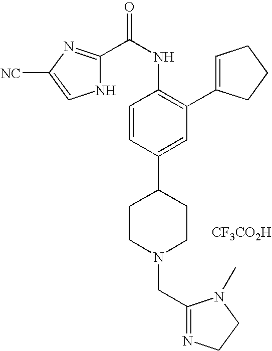 |
| 24 | 4-{4-[(4-Cyano-1H-imidazole-2- carbonyl)-amino]-3-cyclohex-1- enyl-phenyl}-piperidine-1- carboxylic acid amide | 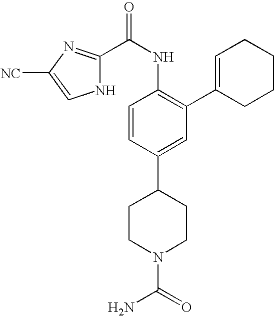 |
| 25 | 4-Cyano-1H-imidazole-2- carboxylic acid [2-cyclohex-1- enyl-4-(3,4,5,6-tetrahydro-2H- [1,2’]bipyridinyl-4-yl)-phenyl]- amide trifluoroacetic acid salt | 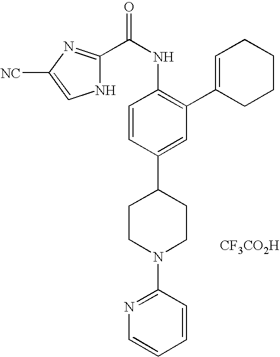 |
| 26 | 4-Cyano-1H-imidazole-2- carboxylic acid {2-cyclohex-1- enyl-4-[1-(2-hydroxy-ethyl)- piperidin-4-yl]-phenyl}-amide trifluoroacetic acid salt | 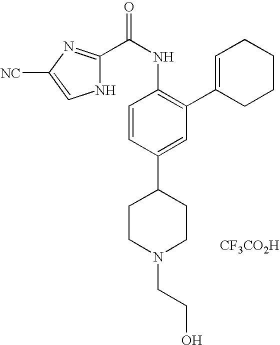 |
| 27 | 4-Cyano-1H-imidazole-2- carboxylic acid {4-[1-(2-cyano- ethyl)-piperidin-4-yl]-2-cyclohex- 1-enyl-phenyl}-amide trifluoroacetic acid salt | 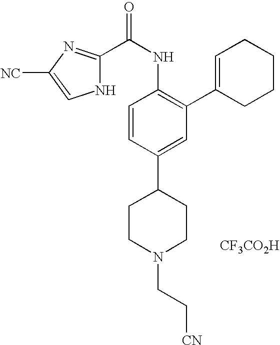 |
| 28 | 4-Cyano-1H-imidazole-2- carboxylic acid [4-(1- carbamoylmethyl-piperidin-4-yl)- 2-cyclohex-1-enyl-phenyl]-amide trifluoroacetic acid salt | 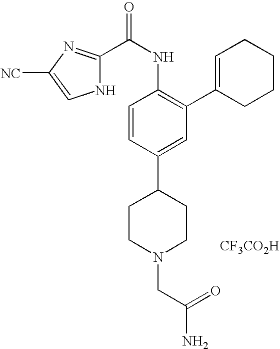 |
| 29 | 4-Cyano-1H-imidazole-2- carboxylic acid {2-cyclohex-1- enyl-4-[1-(2-pyridin-2-yl-acetyl)- piperidin-4-yl]-phenyl}-amide trifluoroacetic acid salt | 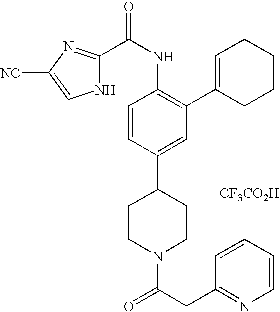 |
| 30 | 4-Cyano-1H-imidazole-2- carboxylic acid {2-cyclohex-1- enyl-4-[1-(2-pyridin-3-yl-acetyl)- piperidin-4-yl]-phenyl}-amide trifluoroacetic acid salt | 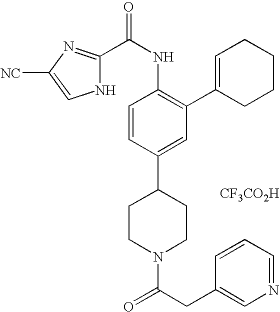 |
| 31 | 4-Cyano-1H-imidazole-2- carboxylic acid {2-cyclohex-1- enyl-4-[1-(2-pyridin-4-yl-acetyl)- piperidin-4-yl]-phenyl}-amide trifluoroacetic acid salt | 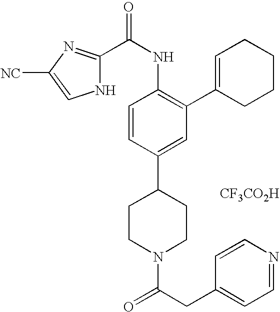 |
| 32 | 4-Cyano-1H-imidazole-2- carboxylic acid (2-cyclohex-1- enyl-4-{1-[2-(1-methyl-1H- imidazol-4-yl)-acetyl]-piperidin- 4-yl}-phenyl)-amide trifluoroacetic acid salt | 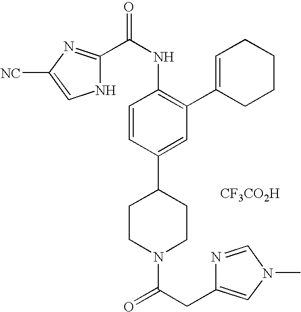 |
| 33 | 4-Cyano-1H-imidazole-2- carboxylic acid {2-cyclohex-1- enyl-4-[1-(2-1H-imidazol-4-yl- acetyl)-piperidin-4-yl]-phenyl}- amide trifluoroacetic acid salt | 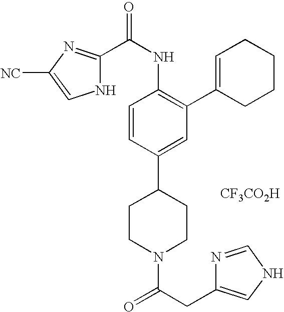 |
| 34 | 4-Cyano-1H-imidazole-2- carboxylic acid {2-cyclohex-1- enyl-4-[1-(2-morpholin-4-yl- ethyl) piperidin-4-yl]-phenyl}- amide di-trifluoroacetic acid salt | 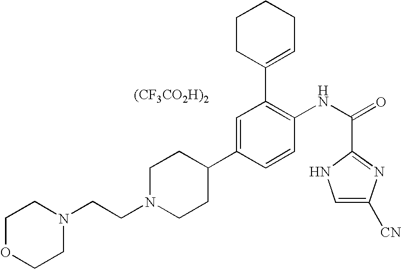 |
| 35 | 4-Cyano-1H-imidazole-2- carboxylic acid [2-(1,1-dioxo- 1,2,3,6-tetrahydro-1λ⁶-thiopyran- 4-yl)-4-piperidin-4-yl-phenyl]- amide | 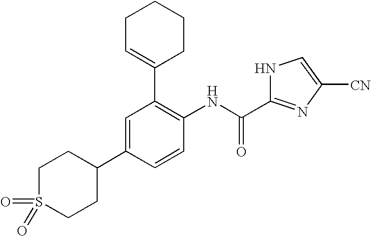 |
| 36 | 4-Cyano-1H-imidazole-2- carboxylic acid [2-(1,1-dioxo- 1,2,3,6-tetrahydro-1λ⁶-thiopyran- 4-yl)-4-piperidin-4-yl-phenyl]- amide trifluoroacetic acid salt | 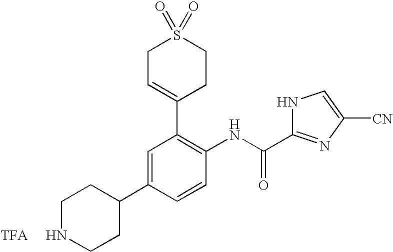 |
| 37 | 4-Cyano-1H-imidazole-2- carboxylic acid [4-(1-acetyl- piperidin-4-yl)-2-(1,1-dioxo 1,2,3,6-tetrahydro-1λ⁶-thiopyran- 4-yl)-phenyl]-amide | 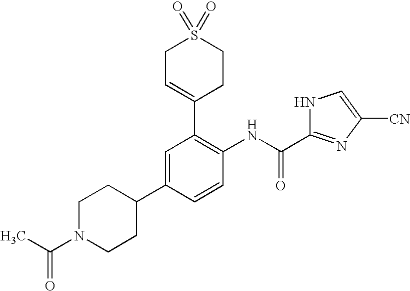 |
| 38a | 4-Cyano-1H-imidazole-2- carboxylic acid {2-cyclohex-1- enyl-4-[1-(2-dimethylamino- acetyl)-piperidin-4-yl]-phenyl}- amide | 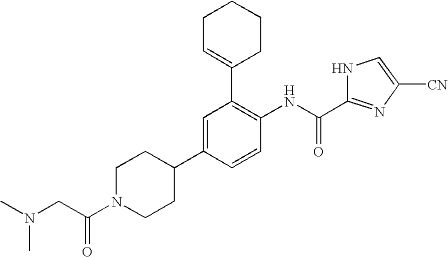 |
| 38b | 4-Cyano-1H-imidazole-2- carboxylic acid {2-cyclohex-1- enyl-4-[1-(2-methylamino- acetyl)-piperidin-4-yl]-phenyl}- amide | 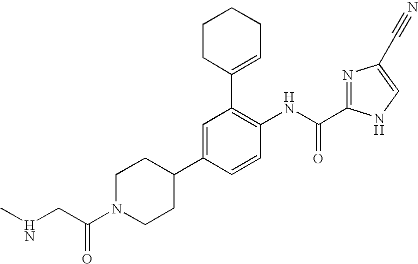 |
| 39 | 4-{4-[(4-Cyano-1H-imidazole-2- carbonyl)-amino]-3-cyclohex-1- enyl-phenyl}-piperidine-1- carboxylic acid (2-hydroxy- ethyl)-amide | 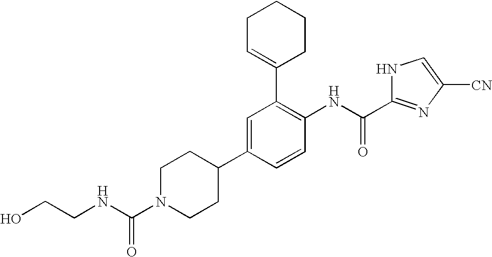 |
| 40 | 4-Cyano-1H-imidazole-2- carboxylic acid {2-cyclohex-1- enyl-4-[1-(2-methanesulfonyl- ethyl)-piperidin-4-yl]-phenyl}- amide | 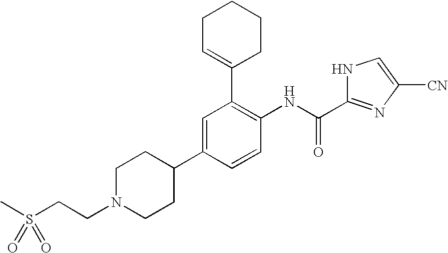 |
| 41 | 4-Cyano-1H-imidazole-2- carboxylic acid {2-cyclohex-1- enyl-4-[1-(1-oxy-pyridine-4- carbonyl)-piperidin-4-yl]- phenyl}-amide | 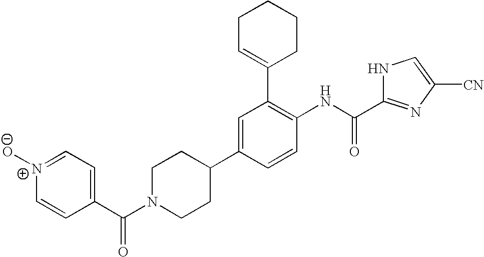 |
| 42 | 4-Cyano-1H-imidazole-2- carboxylic acid {2-cyclohex-1- enyl-4-[1-(1-oxy-pyridine-3- carbonyl)-piperidin-4-yl]- phenyl}-amide | 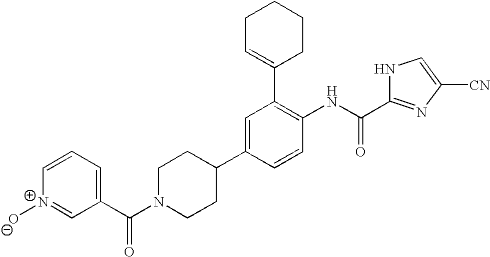 |
| 43 | 4-Cyano-1H-imidazole-2- carboxylic acid{2-cyclohex-1- enyl-4-[1-(pyridine-3-carbonyl)- piperidin-4-yl]-phenyl}-amide | 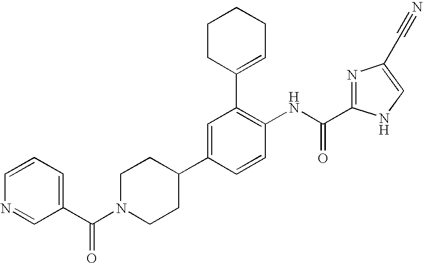 |
| 44 | 4-Cyano-1H-imidazole-2- carboxylic acid (2-cyclohex-1- enyl-4-{1-[2-(2-hydroxy- ethylamino)-acetyl]-pipendin-4- yl}-phenyl)-amide trifluoroacetic acid salt | 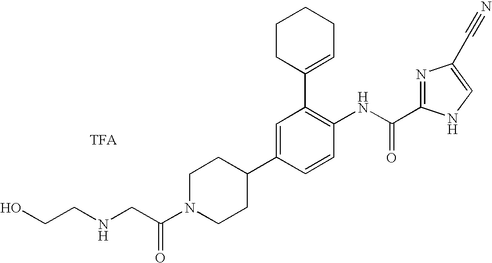 |
| 45 | 4-Cyano-1H-imidazole-2- carboxylic acid (2-cyclohex-1- enyl-4-{1-[2-(2-hydroxy-ethyl)- methyl-amino-acetyl]-piperidin- 4-yl}-phenyl)-amide trifluoroacetic acid salt | 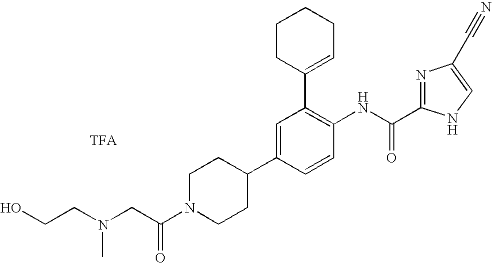 |
| 46 | 4-Cyano-1H-imidazole-2- carboxylic acid [4-(1-acetyl- piperidin-4-yl)-2-(1,2,5,6- tetrahydro-pyridin-3-yl)-phenyl]- amide trifluoroacetic acid salt | 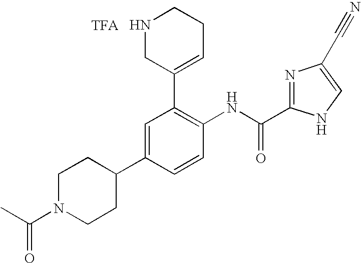 |
| 47 | (4-{4-[(4-Cyano-1H-imidazole-2- carbonyl)-amino]-3-cyclohex-1- enyl-phenyl}-piperidin-l-yl)- acetic acid trifluoroacetic acid salt | 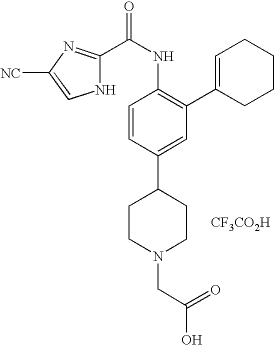 |
| 48 | 4-Cyano-1H-imidazole-2- carboxylic acid {4-[1-(3-amino-3- methyl-butyryl)-piperidin-4-yl]-2- cyclohex-1-enyl-phenyl}-amide trifluoroacetic acid salt | 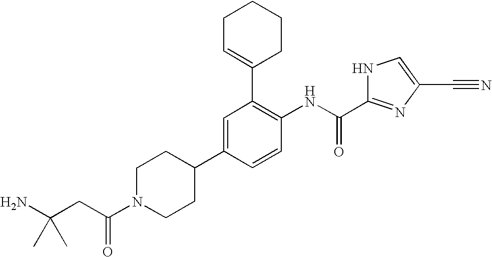 |
| 49 | 4H-[1,2,4]-triazole-3-carboxylic acid (2-cyclohex-1-enyl-4- piperidin-4-yl-phenyl)-amide bis trifluoroacetic acid salt | 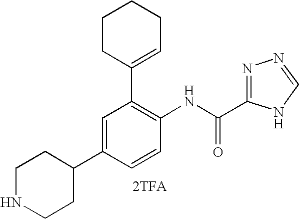 |
| 50 | 5-Chloro-4H-[1,2,4]-triazole-3- carboxylic acid (2-cyclohex-1- enyl-4-piperidin-4-yl-phenyl)- amide trifluoroacetic acid salt | 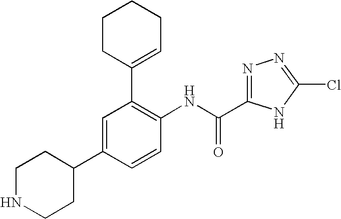 |
| 51a | 5-Cyano-1H-imidazole-2- carboxylic acid [2-cyclohex-1- enyl-4-(cis-2,6-dimethyl- piperidin-4-yl)-phenyl]-amide bis trifluoroacetic acid salt | 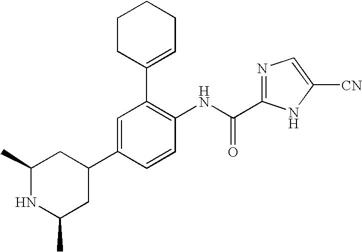 |
| 51b | 5-cyano-1H-imidazole-2- carboxylic acid [2-cyclohex-1- enyl-4-(trans-2,6-dimethyl- piperidin-4-yl)-phenyl]-amide bis trifluoroacetic acid salt | 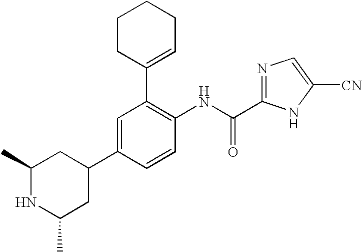 |
| 52 | 5-Cyano-1H-imidazole-2- carboxylic acid {2-cyclohex-1- enyl-4-[1-(R)-(+)-(2,3-dihydroxy- propionyl)-piperidin-4-yl]- phenyl}-amide | 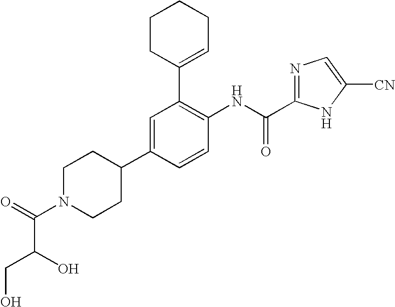 |
| 53 | 5-Cyano-1H-imidazole-2- carboxylic acid [2-cyclohex-1- enyl-4-(1-methoxy-piperidin-4- yl)-phenyl]-amide trifluoroacetic acid salt | 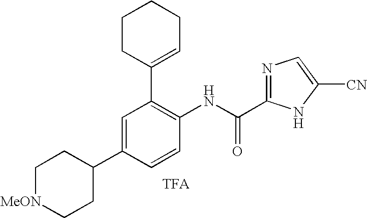 |
| 54 | 4-Cyano-1H-imidazole-2- carboxylic acid [6-(4,4-dimethyl- cyclohex-1-enyl)-1',2',3',4',5',6'- hexahydro-[2,4']bipyridinyl-5- yl]-amide trifluoroacetic acid salt | 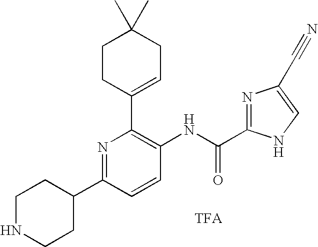 |
| 55 | 4-Cyano-1H-imidazole-2- carboxylic acid [1’-(2- dimethylamino-acetyl)-6-(4,4- dimethyl-cyclohex-1-enyl) 1',2',3',4',5',6'-hexahydro- [2,4']bipyridinyl-5-yl]-amide trifluoroacetic acid salt | 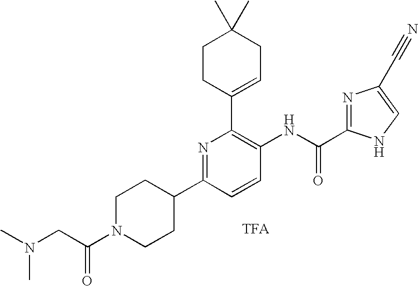 |
| 56 | 4-Cyano-1H-imidazole-2- carboxylic acid [6-(4,4-dimethyl- cyclohex-1-enyl)-1'-(2- methanesulfonyl-ethyl)- 1',2',3',4',5',6'-hexhydro- [2,4']bipyridinyl-5-yl]-amide trifluoroacetic acid salt | 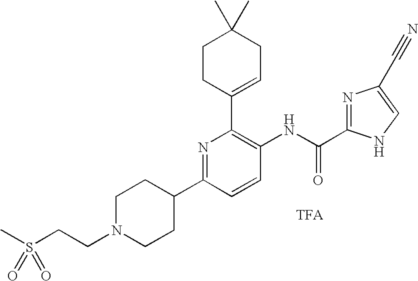 |
| 57 | 5-Cyano-1H-imidazole-2- carboxylic acid {4-[1-(2-amino-2- methyl-propionyl)-piperidin-4- yl]-2-cyclohex-1-enyl-phenyl}- amide trifluoroacetic acid salt | 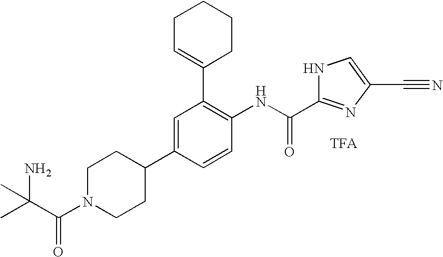 |
| 58 | 5-Cyano-1H-imidazole-2- carboxylic acid [6-cyclohex-1- enyl-1'-(2-methanesulfonyl- ethyl)-1',2',3',4',5',6'-hexahydro- [2,4']bipyridinyl-5-yl]-amide | 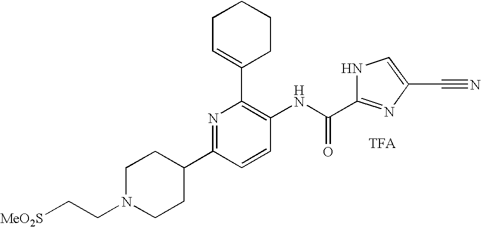 |

### Example 1

### 5-Cyano-furan-2-carboxylic acid

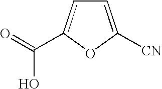

To a flask with a stir bar and Vigreaux column under Ar was added 2-formyl-5-furancarboxylic acid (2.8 g, 20 mmol), hydroxylamine hydrochloride (2.7 g, 40 mmol), and dry pyridine (50 mL). The mixture was heated to 85° C., acetic anhydride (40 mL) was added and the mixture was stirred for 3 h. After cooling to 60° C., water (250 mL) was added and the mixture was stirred at RT for 70 h. The mixture was acidified to pH 2 with concentrated hydrochloric acid and extracted with 3:1 dichloromethane-isopropanol (8×100 mL). The combined organic layers were washed with water (100 mL), brine (100 mL), dried over anh sodium sulfate and concentrated in vacuo to afford the title compound as a tan solid (1.26 g, 46%). ¹H-NMR (CD₃OD; 400 MHz): δ 14.05 (br s, 1H), 7.74 (d, 1H, J=3.8 Hz), 7.42 (d, 1H, J=3.8 Hz).

### Example 2

### 4-Cyano-1H-pyrrole-2-carboxylic acid

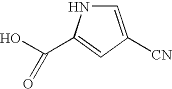

The title compound was prepared by the literature procedure (Loader and Anderson, Canadian J. Chem. 59: 2673 (1981)). ¹H-NMR (CDCl₃; 400 MHz): δ 12.70 (br s, 1H), 7.78 (s, 1H), 7.13 (s, 1H).

### Example 3

### 4-Cyano-1-(2-trimethylsilanyl-ethoxymethyl)-1H-imidazole-2-carboxylate potassium salt

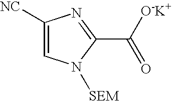

### a) 1-(2-Trimethylsilanyl-ethoxymethyl)-1H-imidazole-4-carbonitrile

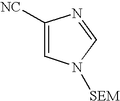

A flask charged with imidazole-4-carbonitrile (0.5 g, 5.2 mmol) (Synthesis, 677, 2003), 2-(trimethylsilyl)ethoxymethyl chloride (SEMCl) (0.95 mL, 5.3 mmol), K₂CO₃ (1.40 g, 10.4 mmol), and acetone (5 mL) was stirred for 10 h at RT. The mixture was diluted with EtOAc (20 mL) and washed with water (20 mL) and brine (20 mL) and the organic layer dried over MgSO₄. The crude product was eluted from a 20-g SPE cartridge (silica) with 30% EtOAc/hexane to give 0.80 g (70%) of the title compound as a colorless oil. Mass spectrum (CI (CH₄), m/z) Calcd. for C₁₀H₁₇N₃OSi, 224.1 (M+H), found 224.1.

### b) 2-Bromo-1-(2-trimethylsilanyl-ethoxymethyl)-1H-imidazole-4-carbonitrile

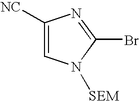

To a solution of 1-(2-trimethylsilanyl-ethoxymethyl)-1H-imidazole-4-carbonitrile (0.70 g, 3.1 mmol) (as prepared in the previous step) in CCl₄ (10 mL) was added NBS (0.61 g, 3.4 mmol) and AIBN (cat), and the mixture heated at 60° C. for 4 h. The reaction was diluted with EtOAc (30 mL) and washed with NaHCO₃ (2×30 mL) and brine (30 mL) and the organic layer was dried over Na₂SO₄ and then concentrated. The title compound was eluted from a 20-g SPE cartridge (silica) with 30% EtOAc/hexane to give 0.73 g (77%) of a yellow solid. Mass spectrum (CI (CH₄), m/z) Calcd. for C₁₀H₁₆BrN₃OSi, 302.0/304.0 (M+H), found 302.1/304.1.

### c) 4-Cyano-1-(2-trimethylsilanyl-ethoxymethyl)-1H-imidazole-2-carboxylic acid ethyl ester

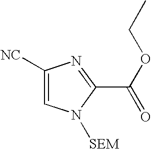

To a solution of 2-bromo-1-(2-trimethylsilanyl-ethoxymethyl)-1H-imidazole-4-carbonitrile (0.55 g, 1.8 mmol) (as prepared in the previous step) in THF (6 mL) at −40° C. was added drop wise a solution of 2M i-PrMgCl in THF (1 mL). The reaction was allowed to stir for 10 min at −40° C. and then cooled to −78° C., and ethyl cyanoformate (0.3 g, 3.0 mmol) was added. The reaction allowed to attain RT and stirred for 1 h. The reaction was quenched with satd aq NH₄Cl, diluted with EtOAc (20 mL) and washed with brine (2×20 mL), and the organic layer was dried over Na₂SO₄ and then concentrated. The title compound was eluted from a 20-g SPE cartridge (silica) with 30% EtOAc/hexane to give 0.4 g (74%) of a colorless oil. Mass spectrum (ESI, m/z): Calcd. for C₁₃H₂₁N₃O₃Si, 296.1 (M+H), found 296.1.

### d) 4-Cyano-1-(2-trimethylsilanyl-ethoxymethyl)-1H-imidazole-2-carboxylate potassium salt

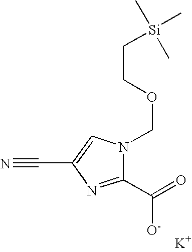

To a solution of 4-cyano-1-(2-trimethylsilanyl-ethoxymethyl)-1H-imidazole-2-carboxylic acid ethyl ester (0.4 g, 1.3 mmol) (as prepared in the previous step) in ethanol (3 mL) was added a solution of 6M KOH (0.2 mL) and the reaction was stirred for 10 min and then concentrated to give 0.40 g (100%) of the title compound as a yellow solid. ¹H-NMR (400 MHz, CD₃OD) δ 7.98 (s, 1H), 5.92 (s, 2H), 3.62 (m, 2H), 0.94 (m, 2H), 0.00 (s, 9H). Mass spectrum (ESI-neg, m/z) Calcd. for C₁₁H₁₇N₃O₃Si, 266.1 (M−H), found 266.0.

### Example 4

### 5-Cyano-furan-2-carboxylic acid [4-(4-methyl-piperazin-1-yl)-2-(3-methyl-thiophen-2-yl)-phenyl]-amide

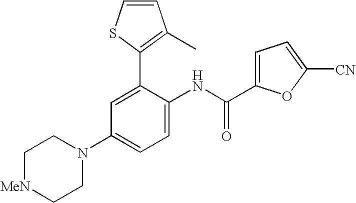

### a) 1-(3-Bromo-4-nitro-phenyl)-4-methyl-piperazine

2-Bromo-4-fluoronitrobenzene (949 mg, 4.31 mmol) was added in two portions to neat N-methypiperazine (8 mL) at 0° C. and allowed to warm to room temperature. The reaction was heated to 60° C. for 1 h, and then it was diluted with 50 mL of EtOAc and poured into H₂O (50 mL). The layers were separated and the organic layer was washed with satd aq NaHCO₃, dried (Na₂SO₄), and concentrated in vacuo to afford 580 mg (45%) of the title compound as a yellow solid: Mass spectrum (ESI, m/z): Calcd. for C₁₁H₁₄BrN₃O₂, 300.0 (M+H), found 300.1.

### b) 4,4,5,5-Tetramethyl-2-(3-methyl-thiophen-2-yl)-[1,3,2]dioxaborolane

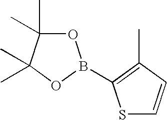

To a stirred solution of 2-bromo-3-methythiophene (337 mg, 1.9 mmol) in 8 mL of THF at −40° C. was added n-BuLi (0.8 mL, 2.5 M/hexanes), and the reaction was allowed to stir for 30 min. At this time 2-isopropoxy-4,4,5,5-tetramethyl-[1,3,2]dioxaborolane (775 μL, 3.8 mmol) was added, and the reaction was allowed to warm to ambient temperature, and stirring was continued for 1 h. The reaction was then cooled to 0° C. and quenched with satd aq NaHCO₃ (10 mL). The mixture was poured into EtOAc (100 mL), washed with H₂O (2×50 mL), dried (Na₂SO₄) and concentrated in vacuo. Purification of the residue by silica gel preparative thin layer chromatography (20% EtOAc-hexanes) afforded 224 mg (53%) of the title compound as an oil. ¹H-NMR (CDCl₃; 400 MHz): δ 1.36 (s, 12H), 2.5 (s, 3H), 6.99 (d, 1H, J=4.8 Hz), 7.50 (d, 1H, J=4.8 Hz).

### c) 1-Methyl-4-[3-(3-methyl-thiophen-2-yl)-4-nitro-phenyl]-piperazine

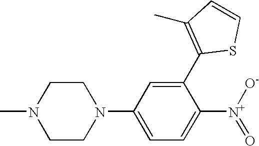

To a flask containing 1-(3-bromo-4-nitro-phenyl)-4-methyl-piperazine (68 mg, 0.2 mmol, as prepared in Example 4, step (a)), 4,4,5,5-tetramethyl-2-(3-methyl-thiophen-2-yl)-[1,3,2]dioxaborolane (61 mg, 0.27 mmol, as prepared in the previous step) and Pd(PPh₃)₄ (14 mg, 6 mol %) was charged toluene (3 mL), ethanol (3 mL) and 2M Na₂CO₃ (4 mL). The resultant mixture was heated at 80° C. for 2 h and then poured into EtOAc (25 mL). The organic layer was separated, dried (Na₂SO₄) and concentrated in vacuo. Purification by silica gel preparative thin layer chromatography (EtOAc) afforded 40 mg (63%) of the title compound as a light yellow solid. Mass spectrum (ESI, m/z): Calcd. for C₁₆H₁₉N₃O₂S, 318.1 (M+H), found 318.2.

### d) 5-Cyano-furan-2-carboxylic acid [4-(4-methyl-piperazin-1-yl)-2-(3-methyl-thiophen-2-yl)-phenyl]-amide

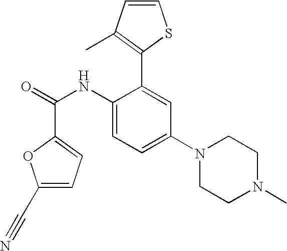

1-Methyl-4-[3-(3-methyl-thiophen-2-yl)-4-nitro-phenyl]-piperazine (60 mg, 0.18 mmol, as prepared in the previous step) was stirred with 40 mg 5% Pd—C in MeOH (5 mL) under H₂ (1 atm) for 2 h. The reaction was filtered through Celite and concentrated in vacuo to afford 40 mg (72%) of 4-(4-methyl-piperazin-1-yl)-2-(3-methyl-thiophen-2-yl)-phenylamine as a brown solid, which was used immediately without further purification. Using a procedure similar to Example 9, step (c), 4-(4-methyl-piperazin-1-yl)-2-(3-methyl-thiophen-2-yl)-phenylamine (40 mg, 0.13 mmol) was allowed to react with 5-cyano-furan-2-carbonyl chloride (30 mg, 0.19 mmol, as prepared in Example 9, step (c)) in the presence of DIEA (61 μL, 0.34 mmol) to afford 18.9 mg (36%) of the title compound as a yellow solid. ¹H-NMR (CDCl₃; 400 MHz): δ 2.13 (s, 3H), 2.38 (s, 3H), 2.59-2.62 (m, 4H), 3.24-3.27 (m, 4H), 6.92 (d, 1H, J=2.8 Hz), 7.06 (d, 1H, J=5.1 Hz), 7.15 (d, 1H, J=3.7 Hz), 7.19 (d, 1H, J=3.7 Hz), 7.02 (dd, 1H, J=2.8, 9.0 Hz), 7.42 (d, 1H, J=5.1 Hz), 8.11 (s, 1H), 8.34 (d, 1H, J=9.0 Hz); Mass spectrum (ESI, m/z): Calcd. for C₂₂H₂₂N₄O₂S, 407.1 (M+H), found 407.1.

### Example 5

### 5-Cyano-furan-2-carboxylic acid [4-(4-methyl-piperazin-1-yl)-2-(4-methyl-thiophen-3-yl)-phenyl]-amide

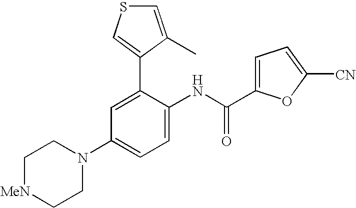

a) 4,4,5,5-Tetramethyl-2-(2-methyl-thiophen-3-yl)-[1,3,2]dioxaborolane

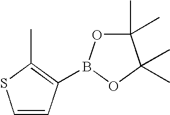

Using a procedure similar to Example 4, step (b), 3-bromo-4-methylthiophene (571 mg, 3.2 mmol) was treated with n-BuLi (1.41 mL, 2.5M/hexanes) and then allowed to react with 2-isopropoxy-4,4,5,5-tetramethyl-[1,3,2]dioxaborolane (775 μL, 3.8 mmol) to afford 189 mg (26%) of the title compound as a colorless oil. ¹H-NMR (CDCl₃; 400 MHz): δ 1.32 (s, 12H), 2.42 (s, 3H), 6.90-6.91 (m, 1H), 7.84 (d, 1H, J=2.9 Hz).

### b) 1-Methyl-4-[3-(4-methyl-thiophen-3-yl)-4-nitro-phenyl]-piperazine

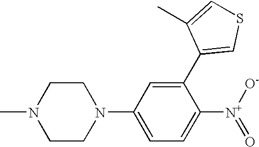

Using a procedure similar to Example 4, step (c), 1-(3-bromo-4-nitro-phenyl)-4-methyl-piperazine (162 mg, 0.54 mmol), 4,4,5,5-tetramethyl-2-(2-methyl-thiophen-3-yl)-[1,3,2]dioxaborolane (145 mg, 0.64 mmol) and Pd(PPh₃)₄ (37 mg, 6 mol %) were allowed to react to afford 108 mg (71%) of the title compound as a yellow solid. ¹H-NMR (CDCl₃; 400 MHz): δ 2.02 (s, 3H), 2.37 (s, 3H), 2.55-2.57 (m, 4H), 3.42-3.45 (m, 4H), 6.66 (d, 1H, J=2.8 Hz), 6.87 (s, 1H), 6.99-7.00 (m, 1H), 7.09 (d, 1H, J=3.2 Hz), 8.13 (d, 1H, J=9.2 Hz).

### c) 4-(4-Methyl-piperazin-1-yl)-2-(4-methyl-thiophen-3-yl)-phenylamine

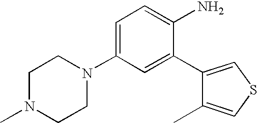

Using a procedure similar to Example 4, step (d), 1-methyl-4-[3-(4-methyl-thiophen-3-yl)-4-nitro-phenyl]-piperazine (100 mg, 0.32 mmol) was stirred with 80 mg 5% Pd—C under H₂ to afford 82 mg (89%) of the title compound as a dark oil, which was used immediately without further purification spectrum (ESI, m/z): Calcd. for C₁₆H₂₁N₃S, 288.15 (M+H), found 288.1.

### d) 5-Cyano-furan-2-carboxylic acid [4-(4-methyl-piperazin-1-yl)-2-(4-methyl-thiophen-3-yl)-phenyl]-amide

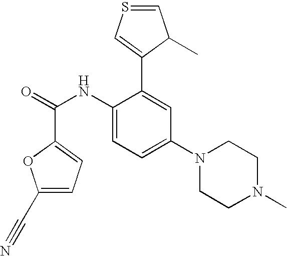

Using a procedure similar to Example 9, step (c), 5-cyano-furan-2-carbonyl chloride (64 mg, 0.41 mmol, as prepared in Example 9, step (c)) was allowed to react with 4-(4-methyl-piperazin-1-yl)-2-(4-methyl-thiophen-3-yl)-phenylamine (80 mg, 0.27 mmol, as prepared in the previous step) in the presence of DIEA (0.10 mL, 0.59 mmol) to afford 25.8 mg (24%) of the title compound as a yellow solid. ¹H-NMR (CDCl₃; 400 MHz): δ 2.09 (s, 3H), 2.37 (s, 3H), 2.59-2.60 (m, 4H), 3.24-3.26 (m, 4H), 6.83 (d, 1H, J=2.9 Hz), 6.98-7.06 (m, 2H), 7.14-7.21 (m, 3H), 7.96 (s, 1H), 8.32 (d, 1H, J=9.0 Hz). Mass spectrum (ESI, m/z): Calcd. for C₂₂H₂₂N₄O₂S, 407.1 (M+H), found 407.1.

### Example 6

### 4-Cyano-1H-imidazole-2-carboxylic acid {2-cyclohex-1-enyl-4-[1-(2-hydroxy-1-hydroxymethyl-ethyl)-piperidin-4-yl]-phenyl}-amide trifluoroacetic acid salt

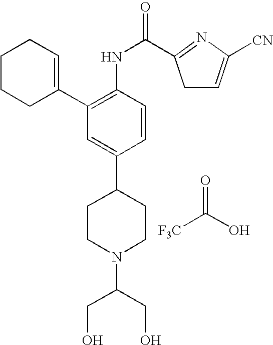

### a) 4-Cyano-1H-imidazole-2-carboxylic acid {2-cyclohex-1-enyl-4-[1-(2,2-dimethyl-[1,3]dioxan-5-yl)-piperidin-4-yl]-phenyl}-amide

To a slurry of 4-cyano-1H-imidazole-2-carboxylic acid (2-cyclohex-1-enyl-4-piperidin-4-yl-phenyl)-amide trifluoroacetic acid salt (81 mg, 0.16 mmol, as prepared in Example 14, step (b)) in CH₂Cl₂ (3 mL) was added NEt₃ (33 μL, 0.24 mmol). The solution was then treated with 2,2-dimethyl-[1,3]dioxan-5-one (31 mg, 0.24 mmol) and the reaction was allowed to stir for 3 h. At this time NaBH(OAc)₃ (51 mg, 0.24 mmol) was added in one portion, and the reaction was allowed to stir for an additional 4 h. The reaction was diluted with H₂O (10 mL) and extracted with EtOAc (2×25 mL). The organic extracts were dried (Na₂SO₄) and concentrated in vacuo. Purification by silica gel preparative thin layer chromatography (10% MeOH—CHCl₃) afforded 22 mg (28%) of the title compound as an off-white semi-solid. Mass spectrum (ESI, m/z): Calcd. for C₂₈H₃₅N₅O₃, 490.2 (M+H), found 490.6.

### b) 4-Cyano-1H-imidazole-2-carboxylic acid {2-cyclohex-1-enyl-4-[1-(2-hydroxy-1-hydroxymethyl-ethyl)-piperidin-4-yl]-phenyl}-amide trifluoro-acetic acid

To a solution of 4-cyano-1H-imidazole-2-carboxylic acid {2-cyclohex-1-enyl-4-[1-(2,2-dimethyl-[1,3]dioxan-5-yl)-piperidin-4-yl]-phenyl}-amide (22 mg, 0.04 mmol, as prepared in the previous step) in THF-H₂O (1 mL, 4:1 v/v) was added TFA (0.4 mL), and the reaction was allowed to stir for 1 h. Removal of the solvent under vacuum afforded 14 mg (60%) of the title compound as an amber foam. ¹H-NMR (CD₃OD, 400 MHz): δ 1.78-1.90 (m, 4H), 2.03-2.16 (m, 3H), 2.29 (br s, 4H), 2.88-2.96 (m, 1H), 3.37-3.40 (m, 1H), 3.46-3.53 (m, 2H), 3.74-3.78 (m, 3H), 5.83 (s, 1H), 7.13 (d, 1H, J=2.0 Hz), 7.22 (dd, 1H, J=2.0, 8.4 Hz), 8.03 (s, 1H), 8.17 (d, 1H, J=8.4 Hz); Mass spectrum (ESI, m/z): Calcd. for C₂₅H₃₁N₅O₃, 450.2 (M+H), found 450.2.

### Example 7

### 4-Cyano-1H-imidazole-2-carboxylic acid {2-cyclohex-1-enyl-4-[1-(2-morpholin-4-yl-acetyl)-piperidin-4-yl]-phenyl}-amide

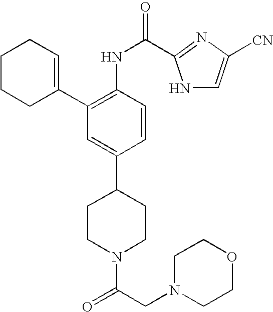

To a solution of morpholin-4-yl-acetic acid ethyl ester (117 mg, 0.67 mmol) in ethanol (4 mL) was added 6N KOH (110 μL, 0.67 mmol) via syringe and stirring was continued for 3 h. Concentration in vacuo afforded 122 mg (100%) of morpholin-4-yl-acetic acid potassium salt. To a mixture of morpholin-4-yl-acetic acid potassium salt (29 mg, 0.15 mmol), 4-cyano-1H-imidazole-2-carboxylic acid (2-cyclohex-1-enyl-4-piperidin-4-yl-phenyl)-amide trifluoroacetic acid salt (65.1 mg, 0.13 mmol, as prepared in Example 14, step (b)) and PyBroP (93 mg, 0.19 mmol) in CH₂Cl₂ (4 mL) was added DIEA (51 μL, 0.29 mmol) and the reaction was allowed to stir overnight. The reaction was diluted with CH₂Cl₂ (50 mL), washed with H₂O (2×25 mL), dried (Na₂SO₄) and concentrated in vacuo. Purification of the crude product by silica gel preparative TLC afforded 8.1 mg (12%) of the title compound as a white solid. ¹H-NMR (CDCl₃; 400 MHz): δ 1.68-2.04 (m, 5H), 2.20-2.29 (m, 4H), 2.53-2.78 (m, 5H), 3.09-3.23 (m, 6H), 3.35-3.40 (m, 1H), 3.72 (br s, 4H), 4.16-4.22 (m, 1H), 4.73-4.77 (m, 1H), 5.82 (s, 1H), 7.00 (s, 1H), 7.12 (dd, 1H, J=0.6, 8.0 Hz), 7.73 (s, 1H), 8.27 (d, 1H, J=8.1 Hz), 9.48 (s, 1H); Mass spectrum (ESI, m/z): Calcd. for C₂₈H₃₄N₆O₃, 503.27 (M+H), found 503.1.

### Example 8

### 4-Cyano-1H-imidazole-2-carboxylic acid {2-cyclohex-1-enyl-4-[1-(3-morpholin-4-yl-propionyl)-piperidin-4-yl]-phenyl}-amide

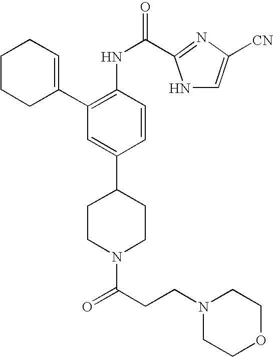

To a flask containing 3-morpholin-4-yl-propionic acid potassium salt (94 mg, 0.47 mmol, prepared from 3-morpholin-4-yl-propionic acid ethyl ester exactly as described in Example 7, 4-cyano-1H-imidazole-2-carboxylic acid (2-cyclohex-1-enyl-4-piperidin-4-yl-phenyl)-amide trifluoroacetic acid salt (179 mg, 0.36 mmol, as prepared in Example 14 (b)), EDCI (83 mg, 0.43 mmol), and HOBT (68 mg, 0.5 mmol) was added DMF (4 mL). To the stirred slurry was added DIEA (157 μL, 0.9 mmol) and the reaction was allowed to stir overnight. The reaction was diluted with H₂O (10 mL) and extracted with EtOAc (2×25 mL). The combined organic extracts were dried (Na₂SO₄), concentrated in vacuo and the crude product was purified by silica gel preparative TLC to afford 10.4 mg (6%) of the title compound as a white solid. ¹H-NMR (CDCl₃; 400 MHz): δ 1.49-1.93 (m, 5H), 2.22-2.31 (m, 3H), 2.52 (br s, 4H), 2.58-2.63 (m, 3H), 2.74-2.76 (m, 4H), 3.10-3.17 (m, 2H), 3.72 (br s, 4H), 3.97-4.02 (m, 2H), 4.76-4.81 (m, 2H), 5.81-5.82 (m, 1H), 6.81-6.82 (m, 1H), 6.99-7.00 (m, 1H), 7.09-7.13 (m, 1H), 7.70 (s, 1H), 8.26 (d, 1H, J=8.2 Hz), 9.51 (s, 1H); Mass spectrum (ESI, m/z): Calcd. for C₂₉H₃₆N₆O₃, 517.28 M+H), found 517.3.

### Example 9

### 5-Cyano-furan-2-carboxylic acid [2′-methyl-5-(4-methyl-piperazin-1-yl)-biphenyl-2-yl]-amide

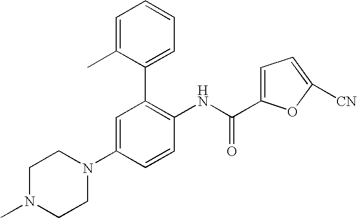

### a) 1-(3-Bromo-4-nitro-phenyl)-4-methyl-piperazine

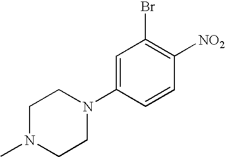

To a cooled (0° C.) solution of 1.00 g (4.55 mmol) of 2-bromo-4-fluoronitrobenzene (Oakwood) in 12 mL of EtOH was added 1.52 mL (13.7 mmol) of piperidine. The solution was stirred at 0° C. for 0.5 h and then at 60° C. for 4 h. The mixture was concentrated in vacuo, dissolved in EtOAc (60 mL), washed with water (3×100 mL) and brine (100 mL), and dried (Na₂SO₄). Concentration in vacuo and chromatography on a 50-g silica SPE column with 1-3% MeOH-dichloromethane afforded 1.06 g (77%) of the title compound as a tannish yellow solid. Mass spectrum (ESI, m/z): Calcd. for C₁₁H₁₄BrN₃O₂, 300.0 (M+H, ⁷⁹Br), found 300.1.

### b) 1-Methyl-4-(2′-methyl-6-nitro-biphenyl-3-yl)-piperazine

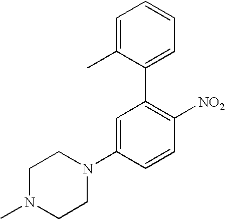

A mixture of 200 mg (0.666 mmol) 1-(3-bromo-4-nitro-phenyl)-4-methyl-piperazine (as prepared in the previous step), 136 mg (0.999 mmol) and 77.0 mg (0.0666 mmol) of tetrakis(triphenylphosphine)palladium (0) under Ar was added 4.0 mL of degassed dimethoxyethane (DME) and 400 μL (0.799 mmol) of 2.0 M aq Na₂CO₃. The mixture was heated with stirring under Ar at 80° C. for 14 h. The cooled (RT) mixture was concentrated and chromatographed on a 10-g silica SPE column with 1-5% MeOH in dichloromethane-hexane (1:1). The product fractions were treated with 80 mg of decolorizing carbon, filtered, concentrated, and then rechromatographed on a similar column with 1-3% EtOH-dichloromethane to afford 265 mg of the title compound as a yellow resin (75% purity by ¹H-NMR as a mixture with triphenylphosphine) that was used in the following reaction without further purification: Mass spectrum (ESI, m/z): Calcd. for C₁₁H₂₁N₃O₃, 312.2 (M+H), found 312.2.

### c) 5-Cyano-furan-2-carboxylic acid [2′-methyl-5-(4-methyl-piperazin-1-yl)-biphenyl-2-yl]-amide

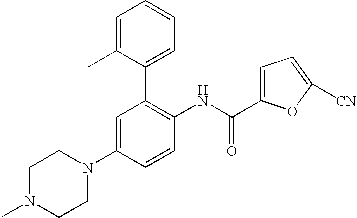

A mixture of 140 mg (0.337 mmol based on 75% purity) of 1-methyl-4-(2′-methyl-6-nitro-biphenyl-3-yl)-piperazine (as prepared in the previous step) and 70 mg of 10% palladium on carbon (Degussa type E101-NE/W, Aldrich, 50% by weight water) in 5 mL of THF was stirred vigorously under a balloon of hydrogen for 1 h. The mixture was filtered (Celite), washed with dichloromethane (2×2 mL), and the solution of the resulting aniline was placed under Ar and used immediately in the following reaction. Simultaneously to the above reduction, 55.4 mg (0.404 mmol) of 5-cyanofuran-2-carboxylic acid (as prepared in Example 1) in 2.5 mL of anh dichloromethane under a CaSO₄ drying tube was treated with 52.9 μL (0.606 mmol) of oxalyl chloride followed by 10 μL of anh DMF. The solution was stirred for 25 min and quickly concentrated in vacuo at 20-25° C. The resulting 5-cyano-furan-2-carbonyl chloride was placed under high vacuum for 2-3 min and then immediately placed under Ar, cooled to 0° C. in an ice bath, and treated with the aniline solution produced above followed by 141 μL (0.808 mmol) of N,N-diisopropylethylamine (DIEA). After stirring for 30 min at RT, the mixture was concentrated in vacuo, and the resulting residue was chromatographed on a 20-g silica SPE column with 2-10% EtOH-dichloromethane to give a yellow resin (which was crystallized from EtOAc-hexane) to afford 17.2 mg (13%) of the pure title compound as a yellow solid along with 70.3 mg of impure title compound. The impure fraction was dissolved in 50 mL of EtOAc, washed with satd aq NaHCO₃-1M K₂CO₃ (1:1, 2×20 mL) and brine (20 mL), dried (Na₂SO₄) and concentrated to afford 43.4 mg (32%) additional title compound as a crystalline yellow solid (total yield 45%). ¹H-NMR (CDCl₃; 400 MHz): δ 8.32 (d, 1H, J=9.0 Hz), 7.73 (br s, 1H), 7.34-7.54 (m, 3H), 7.25 (d, 1H, J=7.7 Hz), 7.12, 7.14 (AB q, 2H, J=3.7 Hz), 7.01 (dd, 1H, J=9.0, 2.8 Hz), 3.25-3.27 (m, 4H), 2.59-2.62 (m, 4H), 2.38 (s, 3H), and 2.15 (s, 3H). Mass spectrum (ESI, m/z): Calcd. for C₂₁H₂₄N₄O₃, 401.2 (M+H), found 401.1.

### Example 10

### 5-Cyano-furan-2-carboxylic acid [2′-fluoro-5-(4-methyl-piperazin-1-yl)-biphenyl-2-yl]-amide

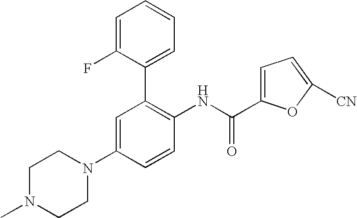

### a) 1-(2′-Fluoro-6-nitro-biphenyl-3-yl)-4-methyl-piperazine

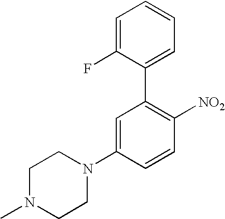

The procedure of Example 9, step (b) was followed using 75.0 mg (0.250 mmol) 1-(3-bromo-4-nitro-phenyl)-4-methyl-piperazine (as prepared in Example 9, step (a)), 136 mg (0.999 mmol) 2-fluorophenylboronic acid, 26.8 mg (0.0232 mmol) of tetrakis(triphenylphosphine)palladium (0) and 400 μL (0.799 mmol) of 2.0 M aq Na₂CO₃ in DME except the mixture was heated for 22 h. Chromatography on a 5-g silica SPE column with 1-5% MeOH in dichloromethane-hexane (1:1) afforded 95.0 mg of the title compound (76% purity by ¹H-NMR as a mixture with triphenylphosphine) as a yellow resin that was used in the following reaction without further purification. Mass spectrum (ESI, m/z): Calcd. for C₁₂H₁₈FN₃O₃, 316.1 (M+H), found 316.2.

### b) 5-Cyano-furan-2-carboxylic acid [2′-fluoro-5-(4-methyl-piperazin-1-yl)-biphenyl-2-yl]-amide

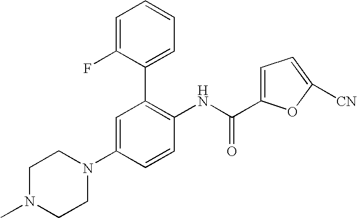

The procedure of Example 9, step (c) was followed using 93.2 mg (0.225 mmol based on 76% purity) of 1-(2′-fluoro-6-nitro-biphenyl-3-yl)-4-methyl-piperazine (as prepared in the previous step), 46 mg of 10% palladium on carbon, 37.0 mg (0.270 mmol) of 5-cyanofuran-2-carboxylic acid (as prepared in Example 1), 35.3 μL (0.405 mmol) of oxalyl chloride, 5.0 μL of anh DMF, and 94.1 μL (0.540 mmol) of DIEA. Chromatography on a 5-g silica SPE column with 1-4% MeOH-dichloromethane afforded 69.8 mg (77%) of the title compound as a yellow resin. ¹H-NMR (CDCl₃; 400 MHz): δ 8.04 (d, 1H, J=9.0 Hz), 7.93 (br s, 1H), 7.434-7.48 (m, 1H), 7.37 (td, 1H, J=7.5, 1.8 Hz), 7.22-7.31 (m, 2H), 7.13, 7.18 (AB q, 2H, J=3.7 Hz), 7.02 (dd, 1H, J=9.0, 2.9 Hz), 6.88 (d, 1H, J=2.9 Hz), 3.24-3.27 (m, 4H), 2.57-2.60 (m, 4H), and 2.36 (s, 3H). Mass spectrum (ESI, m/z): Calcd. for C₂₃H₂₁FN₄O₂, 405.2 (M+H), found 405.2.

### Example 11

### 5-Cyano-furan-2-carboxylic acid [2-cyclohex-1-enyl-4-(4-methyl-piperazin-1-yl)-phenyl]-amide

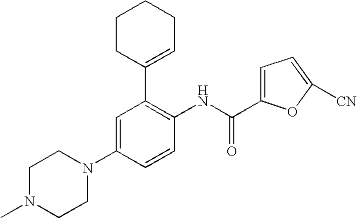

### a) 1-(3-Cyclohex-1-enyl-4-nitro-phenyl)-4-methyl-piperazine

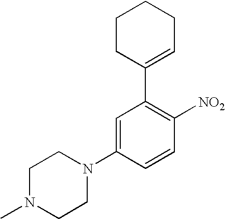

A mixture of 102 mg (0.340 mmol) 1-(3-bromo-4-nitro-phenyl)-4-methyl-piperazine (as prepared in Example 9, step (a)), 59.7 mg (0.474 mmol) cyclohexen-1-ylboronic acid, 43.8 mg (0.0379 mmol) of tetrakis(triphenylphosphine)palladium (0) under Ar was treated with 206 μL (0.412 mmol) of 2.0 M degassed aq Na₂CO₃, 0.6 mL degassed anh toluene and 0.2 mL degassed anh EtOH and the mixture was heated at 100° C. for 21 h. After cooling to RT, the mixture was poured into EtOAc (10 mL), washed with brine (10 mL), dried (Na₂SO₄) and concentrated in vacuo. Chromatography on a 5-g silica SPE column with 1-3% EtOH in dichloromethane afforded 126 mg of the title compound (74% purity by RP-HPLC (C18 column) as a mixture with triphenylphosphine) as a yellow oil that was used in the following reaction without further purification. Mass spectrum (ESI, m/z): Calcd. for C₁₇H₂₃N₃O₃, 302.2 (M+H), found 302.2.

### b) 5-Cyano-furan-2-carboxylic acid [2-cyclohex-1-enyl-4-(4-methyl-piperazin-1-yl)-phenyl]-amide

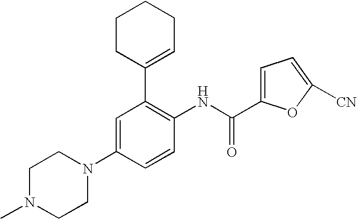

To 122 mg (0.299 mmol based on 74% purity) of 1-(3-cyclohex-1-enyl-4-nitro-phenyl)-4-methyl-piperazine (as prepared in the previous step) in 5.0 mL of EtOH-water (2:1) was added 83.8 mg (1.50 mmol) of iron powder and 160 mg (2.99 mmol) of NH₄Cl and the mixture refluxed under Ar for 12 h. An additional 83.8 mg (1.50 mmol) of iron powder was added, and the mixture was refluxed for 1 h. The mixture was poured into EtOAc (12 mL), filtered (Celite), washed with EtOAc (2×4 mL), concentrated in vacuo and dissolved in anh THF (4.0 mL). The resulting aniline solution was placed under Ar and used immediately in the following reaction. 61.6 mg (0.449 mmol) of 5-cyanofuran-2-carboxylic acid (as prepared in Example 1) in 2.5 mL of anh dichloromethane under a CaSO₄ drying tube was treated with 60.0 μL (0.688 mmol) of oxalyl chloride followed by 10 μL of anh DMF. The solution was stirred for 25 min and quickly concentrated in vacuo at 20-25° C. The residue was placed under high vacuum for 2-3 min and then immediately placed under Ar, cooled to 0° C. in an ice bath and treated with the aniline solution produced above followed by 104 μL (0.598 mmol) of DIEA. After stirring 30 min at RT, the mixture was concentrated in vacuo, dissolved in EtOAc (20 mL), washed with 1M K₂CO₃ (2×10 mL) and brine (10 mL), dried (Na₂SO₄) and concentrated in vacuo. The resulting residue was chromatographed on a 10-g silica SPE column with 1-4% MeOH-dichloromethane to give a yellow resin which was then crystallized from Et₂O-hexane to afford 84.7 mg (72%) of the title compound as a crystalline yellow solid. ¹H-NMR (CDCl₃; 400 MHz): δ 8.57 (br s, 1H), 8.26 (d, 1H, J=9.0 Hz), 7.20, 7.23 (AB q, 2H, J=3.7 Hz), 6.86 (dd, 1H, J=9.0, 2.9 Hz), 6.74 (d, 1H, J=2.9 Hz), 5.84-5.85 (m, 1H), 3.20-3.22 (m, 4H), 2.57-2.59 (m, 4H), 2.36 (s, 3H), 2.23-2.30 (m, 4H) and 1.79-1.84 (m, 4H). Mass spectrum (ESI, m/z): Calcd. for C₂₃H₂₆N₄O₂, 391.2 (M+H), found 391.2.

### Example 12

### 5-Cyano-furan-2-carboxylic acid [2-(3,6-dihydro-2H-pyran-4-yl)-4-(4-methyl-piperazin-1-yl)-phenyl-amide

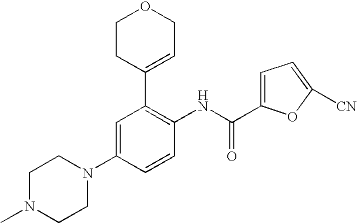

### a) 1-[3-(3,6-Dihydro-2H-pyran-4-yl)-4-nitro-phenyl]-4-methyl-piperazine

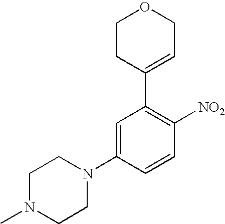

1-(3-Bromo-4-nitro-phenyl)-4-methyl-piperazine (as prepared in Example 9, step (a)) (225.1 mg, 0.79 mmol), K₂CO₃ (310.9 mg, 2.25 mmol) and 4-(4,4,5,5-tetramethyl-[1,3,2]dioxaborolan-2-yl)-3,6-dihydro-2H-pyran (Murata, M., et al, Synthesis, 778, (2000)) (157 mg, 0.75 mmol) in dioxane (5 mL) was heated at 80° C. overnight under Ar. The reaction mixture was allowed to cool to RT, concentrated, and the resulting residue was chromatographed on silica (10% EtOAc/hexane-20% MeOH/EtOAc) to obtain the title compound (82 mg, 36%). ¹H-NMR (CDCl₃; 400 MHz): δ 8.04 (d, 1H, J=9.4 Hz), 6.78 (dd, 1H, J=9.4, 2.6 Hz), 6.58 (m, 1H, J=2.6 Hz), 5.58 (m, 1H), 4.34 (m, 2H), 3.95 (t, 2H, J=5.3 Hz), 3.46 (m, 4H), 2.57 (m, 4H), 2.38 (s, 3H), 2.30 (m, 2H).

### b) 5-Cyano-furan-2-carboxylic acid [2-(3,6-dihydro-2H-pyran-4-yl)-4-(4-methyl-piperazin-1-yl)-phenyl-amide

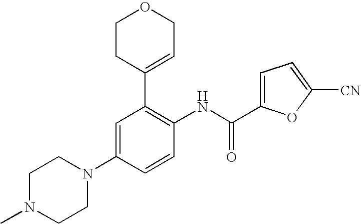

1-[3-(3,6-Dihydro-2H-pyran-4-yl)-4-nitro-phenyl]-4-methyl-piperazine (as prepared in previous step) (80 mg, 0.26 mmol) was converted to the corresponding amine using a procedure similar to Example 4, step (d), and coupled with 5-cyano-furan-2-carbonyl chloride as prepared in Example 9, step (c) (obtained from 137 mg, 1.00 mmol of 5-cyano-furan-2-carboxylic acid as prepared in Example 1) in CH₂Cl₂ (2 mL) at 0° C. The product was isolated by flash chromatography on silica (50% EtOAc/hexane-10% MeOH/EtOAc) to obtain the title compound (62.2 mg, 60%). ¹H-NMR (CDCl₃; 400 MHz): δ 8.35 (br s, 1H), 8.12 (d, 1H each, J=8.76 Hz), 7.24 (d, 1H, J=5.08 Hz), 7.19 (d, 1H, J=5.08 Hz), 6.88 (dd, 1H, J=8.76, 2.7 Hz), 6.73 (d, 1H, J=2.7 Hz), 5.88 (br s, 1H), 4.34 (m, 2H), 3.94 (t, 2H, J=5.3 Hz), 3.23 (m, 4H), 2.59 (m, 4H), 2.38 (br s, 5H). LC-MS (ESI, m/z): Calcd. for C₂₂H₂₄N₄O₃, 393.1 (M+H), found 393.2.

### Example 13

### 4-Cyano-1H-pyrrole-2-carboxylic acid (2-cyclohex-1-enyl-4-piperidin-4-yl-phenyl)-amide trifluoroacetic acid salt

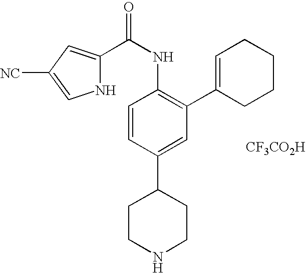

### a) 4-(4-Amino-phenyl)-3,6-dihydro-2H-pyridine-1-carboxylic acid tert-butyl ester

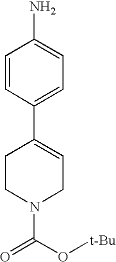

The title compound was prepared by Suzuki coupling of 4-(4,4,5,5-tetramethyl-[1,3,2]dioxaborolan-2-yl)-phenylamine with 4-trifluoromethanesulfonyloxy-3,6-dihydro-2H-pyridine-1-carboxylic acid tert-butyl ester (Synthesis, 993, (1991)) according to the procedure in Example 35, step (b). Mass spectrum (ESI, m/z): Calcd. for C₁₆H₂₂N₂O₂, 275.2 (M+H), found 275.1.

### b) 4-(4-Amino-phenyl)-piperidine-1-carboxylic acid tert-butyl ester

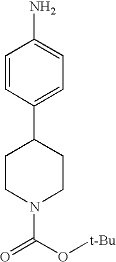

A solution of 4-(4-amino-phenyl)-3,6-dihydro-2H-pyridine-1-carboxylic acid tert-butyl ester (0.35 g, 1.2 mmol) (as prepared in the previous step) in methanol was hydrogenated over 10% Pd/C at 20 psi for 1 h. The solution was filtered and concentrated to give 0.35 g (100%) of the title compound as a yellow solid: Mass spectrum (ESI, m/z): Calcd. for C₁₆H₂₄N₂O₂, 277.2 (M+H), found 277.1.

### c) 4-(4-Amino-3-bromo-phenyl)-piperidine-1-carboxylic acid tert-butyl ester

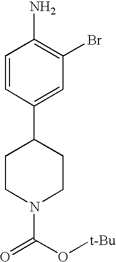

To a solution of 4-(4-amino-phenyl)-piperidine-1-carboxylic acid tert-butyl ester (0.20 g, 0.71 mmol) (as prepared in the previous step) in DCM (3 mL) was added N-bromosuccinimide (NBS) (0.13 g, 0.71 mmol), and the reaction stirred at RT for 10 h. The reaction was diluted with EtOAc (10 mL) and washed with NaHCO₃ (2×10 mL) and brine (10 mL). Concentration of the organic layer gave 0.26 g (100%) of the title compound as a yellow foam. Mass spectrum (ESI, m/z): Calcd. for C₁₆H₂₃BrN₂O₂, 355.1 (M+H), found 355.1.

### d) 4-(4-Amino-3-cyclohex-1-enyl-phenyl)-piperidine-1-carboxylic acid tert-butyl ester

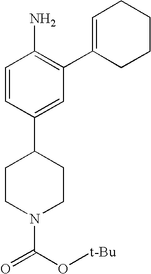

A flask was charged with 4-(4-amino-3-bromo-phenyl)-piperidine-1-carboxylic acid tert-butyl ester (0.13 g, 0.36 mmol) (as prepared in the previous step), cyclohex-1-enyl boronic acid (0.060 g, 0.48 mmol), Pd(PPh₃)₄ (0.04 g, 10 mol %), aqueous 2M Na₂CO₃ (1.5 mL), ethanol (1.5 mL), and toluene (3 mL), and heated at 80° C. for 3 h. The reaction was diluted EtOAc (10 mL), washed with NaHCO₃ (2×10 mL) and brine (10 mL), and the organic layer was dried over Na₂SO₄ and then concentrated. The title compound was eluted from a 20-g SPE cartridge (silica) with 30%

EtOAc/hexane to give 0.10 g (85%) of the title compound as a yellow oil. Mass spectrum (ESI, m/z): Calcd. for C₂₂H₃₂N₂O₂, 357.2 (M+H), found 357.1.

### e) 4-Cyano-1H-pyrrole-2-carboxylic acid (2-cyclohex-1-enyl-4-piperidin-4-yl-phenyl)-amide trifluoroacetic acid salt

A flask was charged with 4-(4-amino-3-cyclohex-1-enyl-phenyl)-piperidine-1-carboxylic acid tert-butyl ester (0.050 g, 0.14 mmol) (as prepared in the previous step), 4-cyano-1H-pyrrole-2-carboxylic acid (0.019 g, 0.14 mmol) (as prepared in Example 2), EDCI (0.040 g, 0.21 mmol), HOBt (0.019 g, 0.14 mmol), DIEA (0.073 mL, 0.42 mmol), and DCM (0.5 mL) and stirred at 25° C. for 10 h. The reaction was loaded directly on a 10-g solid phase extraction (SPE) cartridge (silica) and the resulting intermediate was eluted with 30% EtOAc/hexane. This compound was stirred at RT for 1 h in 50 TFA/DCM (2 mL) and then concentrated and purified by RP-HPLC (C18), eluting with 30-50% CH₃CN in 0.1% TFA/H₂O over 12 min to give the title compound (0.052 g, 77%). ¹H-NMR (400 MHz, CD₃OD): δ 7.59 (s, 1H), 7.50 (d, 1H), 7.22 (d, 1H), 7.16 (m, 2H), 5.74 (m, 1H), 3.54 (m, 2H), 3.16 (m, 2H), 2.94 (m, 1H), 2.29 (m, 2H), 2.15 (m, 4H), 1.92 (m, 2H), 1.72 (m, 4H). Mass spectrum (ESI, m/z): Calcd. for C₂₃H₂₆N₄O, 375.2 (M+H), found 375.1.

### Example 14

### 4-Cyano-1H-imidazole-2-carboxylic acid (2-cyclohex-1-enyl-4-piperidin-4-yl-phenyl)-amide trifluoroacetic acid salt

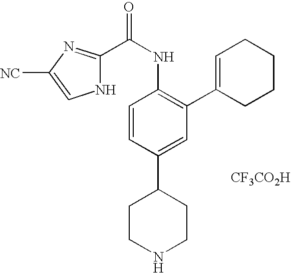

### a) 4-(4-{[4-Cyano-1-(2-trimethylsilanyl-ethoxymethyl)-1H-imidazole-2-carbonyl]-amino}-3-cyclohex-1-enyl-phenyl)-piperidine-1-carboxylic acid tert-butyl ester

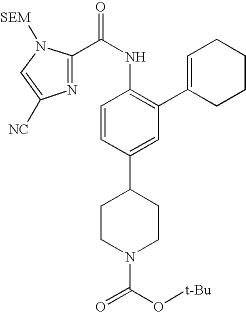

To a solution of 4-cyano-1-(2-trimethylsilanyl-ethoxymethyl)-1H-imidazole-2-carboxylate potassium salt (3.34 g, 10.9 mmol) (as prepared in Example 3, step (d)) in 20 mL DCM was added DIEA (3.8 mL, 21.8 mmol) and PyBroP (5.6 g, 12.0 mmol), and the reaction stirred at 25° C. for 15 min. A solution of 4-(4-amino-3-cyclohex-1-enyl-phenyl)-piperidine-1-carboxylic acid tert-butyl ester (3.9 g, 10.9 mmol) (as prepared in Example 13, step (d)) in 10 mL DCM was added and the reaction stirred for 8 h at 25° C. The reaction was diluted EtOAc (60 mL) and washed with NaHCO₃ (2×60 mL) and brine (100 mL) and the organic layer was dried over Na₂SO₄ and then concentrated. The title compound was purified by flash chromatography (silica gel, 2% EtOAc/DCM) to give 5.5 g (85%) of the title compound as a yellow oil. Mass spectrum (ESI, m/z): Calcd. for C₃₃H₄₇N₅O₄Si, 606.2 (M+H), found 606.2.

### b) 4-Cyano-1H-imidazole-2-carboxylic acid (2-cyclohex-1-enyl-4-piperidin-4-yl-phenyl)-amide trifluoroacetic acid salt

To a solution of 4-(4-{[4-cyano-1-(2-trimethylsilanyl-ethoxymethyl)-1H-imidazole-2-carbonyl]-amino}-3-cyclohex-1-enyl-phenyl)-piperidine-1-carboxylic acid tert-butyl ester (1.5 g, 2.5 mmol) (as prepared in the previous step) in 10 mL of DCM and 0.3 mL EtOH was added 3 mL of TFA and the solution stirred for 3 h at 25° C. The reaction was diluted with 5 mL of EtOH and then concentrated. The residue was crystallized from methanol and ethyl ether to give 0.85 g (70%) of the title compound as a white solid. ¹H-NMR (400 MHz, CD₃OD) δ 8.18 (d, 1H), 8.04 (s, 1H), 7.22 (dd, 1H), 7.12 (d, 1H), 5.76 (m, 1H), 3.54 (m, 2H), 3.16 (m, 2H), 2.92 (m, 1H), 2.30 (m, 4H), 2.10 (m, 2H), 1.75 (m, 6H). Mass spectrum (ESI, m/z): Calcd. for C₂₂H₂₅N₅O, 376.2 (M+H), found 376.2.

### Example 15

### 4-Cyano-1H-pyrrole-2-carboxylic acid [4-(1-acetyl-piperidin-4-yl)-2-cyclohex-1-enyl-phenyl]-amide

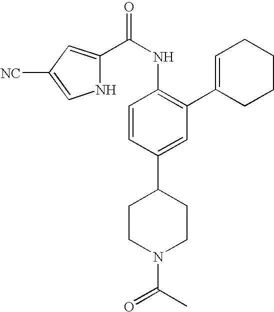

The title compound was prepared from 4-cyano-1H-pyrrole-2-carboxylic acid (2-cyclohex-1-enyl-4-piperidin-4-yl-phenyl)-amide trifluoroacetic acid salt (as prepared in Example 13, step (e)) according to the procedure in Example 37. ¹H-NMR (400 MHz, CDCl₃) δ 10.82 (s, 1H), 8.28 (d, 1H), 8.18 (s, 1H), 7.48 (d, 1H), 7.16 (dd, 1H), 7.02 (s, 1H), 6.72 (s, 1H), 5.88 (m, 1H), 4.82 (m, 1H), 3.98 (m, 1H), 3.20 (m, 1H), 2.70 (m, 2H), 2.29 (m, 4H), 2.18 (s, 3H), 1.80 (m, 8H). Mass spectrum (ESI, m/z): Calcd. for C₂₅H₂₈N₄O₂, 417.2 (M+H), found 417.1.

### Example 16

### 4-Cyano-1H-imidazole-2-carboxylic acid [4-(1-acetyl-piperidin-4-yl)-2-cyclohex-1-enyl-phenyl]-amide

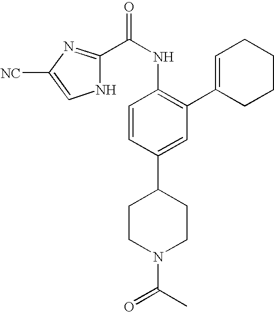

The title compound was prepared from 4-cyano-1H-imidazole-2-carboxylic acid (2-cyclohex-1-enyl-4-piperidin-4-yl-phenyl)-amide trifluoroacetic acid salt (as prepared in Example 13, step (b)) according to the procedure in Example 37: ¹H-NMR (400 MHz, CDCl₃) δ 13.12 (br s, 1H), 9.58 (s, 1H), 8.34 (d, 1H), 7.76 (s, 1H), 7.21 (dd, 1H), 7.05 (d, 1H), 5.86 (s, 1H), 4.84 (m, 2H), 4.00 (m, 1H), 3.22 (m, 1H), 2.72 (m, 2H), 2.30 (m, 4H), 2.21 (s, 3H), 1.80 (m, 8H). Mass spectrum (ESI, m/z): Calcd. for C₂₄H₂₇N₅O₂, 418.2 (M+H), found 418.1.

### Example 17

### 4-Cyano-1H-imidazole-2-carboxylic acid [2-(4-methyl-cyclohex-1-enyl)-4-piperidin-4-yl-phenyl]-amide trifluoroacetic acid salt

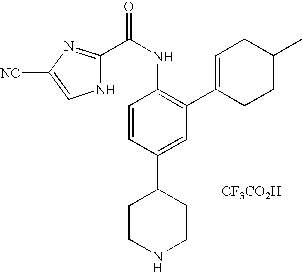

The title compound was prepared from 4-cyano-1-(2-trimethylsilanyl-ethoxymethyl)-1H-imidazole-2-carboxylate potassium salt (as prepared in Example 3, step (d)) and 4-[4-amino-3-(4-methyl-cyclohex-1-enyl)-phenyl]-piperidine-1-carboxylic acid tert-butyl ester (prepared according to the procedure in Example 13, step (d), substituting 4-methyl-1-cyclohex-1-enyl boronic acid for cyclohex-1-enyl boronic acid) according to the procedure for Example 14: ¹H-NMR (400 MHz, CD₃OD): δ 8.18 (d, 1H), 8.04 (s, 1H), 7.22 (dd, 1H), 7.12 (d, 1H), 5.80 (m, 1H), 3.54 (m, 2H), 3.18 (m, 2H), 2.94 (m, 1H), 2.30 (m, 3H), 2.12 (m, 2H), 1.92 (m, 5H), 1.54 (m, 1H), 1.12 (d, 3H). Mass spectrum (ESI, m/z): Calcd. for C₂₃H₂₇N₅O, 390.2 (M+H), found 390.2.

### Example 18

### 4-Cyano-1H-imidazole-2-carboxylic acid (2-cyclopent-1-enyl-4-piperidin-4-yl-phenyl)-amide trifluoroacetic acid salt

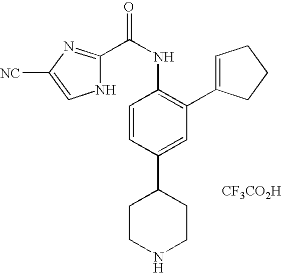

The title compound was prepared from 4-cyano-1-(2-trimethylsilanyl-ethoxymethyl)-1H-imidazole-2-carboxylate potassium salt (as prepared in Example 3, step (d)) and 4-(4-amino-3-cyclopent-1-enyl-phenyl)-piperidine-1-carboxylic acid tert-butyl ester (prepared according to the procedure in Example 13, step (d), substituting cyclopenten-1-yl boronic acid for cyclohex-1-enyl boronic acid) according to the procedure for Example 14. ¹H-NMR (400 MHz, DMSO-d₆) δ 14.25 (br s, 1H), 10.00 (s, 1H), 8.36 (s, 1H), 7.72 (d, 1H), 7.18 (m, 2H), 6.06 (s, 1H), 4.12 (m, 1H), 3.42 (m, 2H), 3.18 (m, 2H), 3.00 (m, 3H), 2.80 (m, 2H), 1.92 (m, 5H). Mass spectrum (ESI, m/z): Calcd. for C₂M₂₃N₅O, 362.2 (M+H), found 362.2.

### Example 19

An alternate method for the synthesis of the intermediate described in Example 1 is described below.

### 5-Cyano-furan-2-carboxylic acid

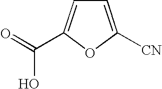

A 250-mL, three-neck, round-bottom flask equipped with a mechanical stirrer, a heating mantle, and a condenser was charged with 5-formyl-2-furancarboxylic acid (9.18 g, 65.6 mmol) and pyridine (60 mL). Hydroxylamine hydrochloride (5.01 g, 72.2 mmol) was added and the mixture was heated to 85° C. Acetic anhydride (40 mL) was added and the reaction was stirred at 85° C. for 3 h, after which time the solvent was evaporated at 40° C. under reduced pressure. The residue was dissolved in water, basified with 2.0 N NaOH solution to pH 9, and extracted with 4:1 dichloromethane/2-propanol until the pyridine was completely removed (5×200 mL). The aqueous solution was then acidified with 2.0 N HCl solution to pH 2, saturated with solid NaCl, and extracted with 4:1 dichloromethane/2-propanol (5×200 mL). The combined organic extracts were dried over Na₂SO₄ and concentrated in vacuo to dryness. The residue was crystallized from dichloromethane to give 6.80 g of the title compound as a white solid (76%). Mass spectrum (ESI-neg, m/z) Calcd. for C₆H₃NO₃, 136.0 (M−H), found 136.1. The ¹H NMR spectrum was consistent with the assigned structure.

### Example 20

### 4-Cyano-1H-imidazole-2-carboxylic acid {2-cyclohex-1-enyl-4-[1-(2-methanesulfonyl-acetyl)-piperidin-4-yl]-phenyl}-amide

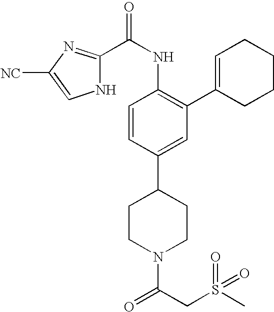

A flask was charged with methanesulfonyl-acetic acid (14 mg, 0.10 mmol), EDCI (30 mg, 0.15 mmol), HOBt (14 mg, 0.10 mmol), DIEA (36 μL, 0.20 mmol) and 0.5 mL DCM and stirred at 25° C. After 10 min, a solution containing 4-cyano-1H-imidazole-2-carboxylic acid (2-cyclohex-1-enyl-4-piperidin-4-yl-phenyl)-amide TFA salt (40 mg, 0.08 mmol) (as prepared in Example 20, step (b)) and NEt₃ (14 μL, 0.09 mmol) in 0.5 mL DCM was added and the reaction allowed to proceed for 10 h at 25° C. The reaction mixture was loaded on a 5-g SPE cartridge (silica) and the title compound was eluted with 10% EtOH/EtOAc to give 10 mg (25%) of a white solid. ¹H-NMR (400 MHz, CDCl₃): δ 11.60 (br s, 1H), 9.52 (s, 1H), 8.30 (d, 1H), 7.74 (s, 1H), 7.60 (dd, 1H), 7.03 (d, 1H), 5.86 (m, 1H), 4.84 (m, 1H), 4.18 (s, 2H), 4.12 (m, 1H), 3.32 (m, 1H), 3.20 (s, 3H), 2.82 (m, 2H), 2.30 (m, 4H), 1.98 (m, 2H), 1.84 (m, 5H), 1.72 (m, 1H). Mass spectrum (ESI, m/z): Calcd. for C₂₅H₂₉N₅O₄S, 496.2 (M+H), found 496.2.

### Example 21

### 4-Cyano-1H-imidazole-2-carboxylic acid [2-cyclohex-1-enyl-4-(1-pyridin-2-ylmethyl-piperidin-4-yl)-phenyl]-amide trifluoroacetic acid salt

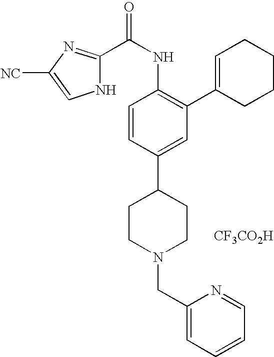

A flask was charged with 4-cyano-1H-imidazole-2-carboxylic acid (2-cyclohex-1-enyl-4-piperidin-4-yl-phenyl)-amide TFA salt (88 mg, 0.18 mmol) (as prepared in Example 14, step (b)), pyridine-2-carbaldehyde (17 μL, 0.21 mmol), NEt₃ (30 μL, 0.21 mmol), sodium triacetoxyborohydride (56 mg, 0.25 mmol) and 0.8 mL of 1,2-dichloroethane and stirred for 10 h at 25° C. The solvent was evaporated, and the title compound was purified by RP-HPLC (C18), eluting with 30-50% CH₃CN in 0.1% TFA/H₂O over 20 min to give 81 mg (78%) of a white solid. ¹H-NMR (400 MHz, DMSO-d₆): δ 14.25 (br s, 1H), 9.90 (br s, 1H), 9.79 (s, 1H), 8.72 (s, 1H), 8.36 (s, 1H), 7.98 (m, 1H), 7.88 (dd, 1H), 7.58 (d, 1H), 7.52 (m, 1H), 7.20 (m, 1H), 7.12 (d, 1H), 5.76 (m, 1H), 4.56 (s, 2H), 3.40 (m, 2H), 3.18 (m, 2H), 2.88 (m, 1H), 2.20 (m, 4H), 2.00 (m, 4H), 1.72 (m, 4H). Mass spectrum (ESI, m/z): Calcd. for C₂₈H₃₀N₆O, 467.2 (M+H), found 467.2.

### Example 22

### 4-Cyano-1H-imidazole-2-carboxylic acid [2-(4-methyl-cyclohex-1-enyl)-4-(1-pyridin-2-ylmethyl-piperidin-4-yl)-phenyl]-amide trifluoroacetic acid salt

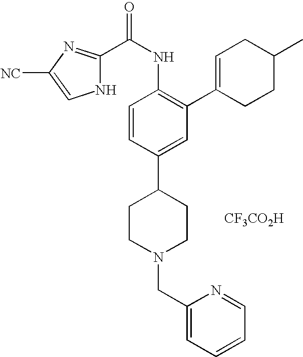

This compound was prepared according to the procedure in Example 21 from 4-cyano-1H-imidazole-2-carboxylic acid [2-(4-methyl-cyclohex-1-enyl)-4-piperidin-4-yl-phenyl]-amide (as prepared in Example 17) and pyridine-2-carbaldehyde. ¹H-NMR (400 MHz, DMSO-d₆): δ 14.25 (br s, 1H), 9.90 (br s, 1H), 9.79 (s, 1H), 8.72 (s, 1H), 8.36 (s, 1H), 7.98 (m, 1H), 7.86 (dd, 1H), 7.54 (d, 1H), 7.52 (m, 1H), 7.20 (m, 1H), 7.12 (d, 1H), 5.74 (m, 1H), 4.56 (s, 2H), 3.40 (m, 2H), 3.18 (m, 2H), 2.88 (m, 1H), 2.48-2.22 (m, 3H), 2.18-2.06 (m, 4H), 1.98-1.82 (m, 3H), 1.52 (m, 1H), 1.02 (s, 3H). Mass spectrum (ESI, m/z): Calcd. for C₂₈H₃₂N₆O, 481.2 (M+H), found 481.2.

### Example 23

### 4-Cyano-1H-imidazole-2-carboxylic acid {2-cyclopent-1-enyl-4-[1-(1-methyl-1H-imidazol-2-ylmethyl)-piperidin-4-yl]-phenyl}-amide trifloroacetic acid salt

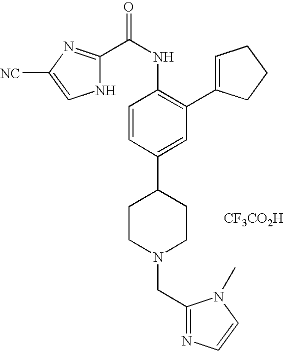

This compound was prepared from 4-cyano-1H-imidazole-2-carboxylic acid (2-cyclopent-1-enyl-4-piperidin-4-yl-phenyl)-amide TFA salt (as prepared in Example 18) and 1-methyl-1H-imidazole-2-carbaldehyde according to the procedure in Example 21. ¹H-NMR (400 MHz, CD₃OD): δ 8.03 (m, 2H), 7.50 (d, 1H), 7.42 (s, 1H), 7.20 (m, 2H), 6.02 (m, 1H), 4.22 (s, 2H), 3.96 (s, 3H), 3.30 (m, 2H), 2.82-2.40 (m, 7H), 2.13-1.84 (m, 6H). Mass spectrum (ESI, m/z): Calcd. for C₂₆H₂₉N₇O, 456.2 (M+H), found 456.2.

### Example 24

### 4-{4-[(4-Cyano-1H-imidazole-2-carbonyl)-amino]-3-cyclohex-1-enyl-phenyl}-piperidine-1-carboxylic acid amide

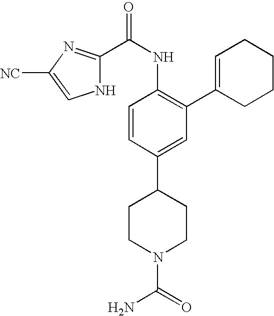

A flask was charged with 4-cyano-1H-imidazole-2-carboxylic acid (2-cyclohex-1-enyl-4-piperidin-4-yl-phenyl)-amide TFA salt (51 mg, 0.10 mmol) (as prepared in Example 14, step (b)), NEt₃ (22 μL, 0.15 mmol), trimethylsilyl isocyanate (16 μL, 0.11 mmol) and 1.0 mL of DCM and stirred for 10 h at 25° C. The solvent was evaporated and the title compound was purified by RP-HPLC (C18), eluting with 35-60% CH₃CN in 0.1% TFA/H₂O over 11 min to give 30 mg (70%) of a white solid. ¹H-NMR (400 MHz, DMSO-d₆): δ 14.28 (br s, 1H), 9.76 (s, 1H), 8.34 (s, 1H), 7.84 (d, 1H), 7.18 (dd, 1H), 7.08 (d, 1H), 6.00 (br s, 2H), 5.72 (m, 1H), 4.18 (m, 2H), 2.80-2.60 (m, 3H), 2.24-2.10 (m, 4H), 1.80-1.60 (m, 6H), 1.50 (m, 2H). Mass spectrum (ESI, m/z): Calcd. for C₂₃H₂₆N₆O, 419.2 (M+H), found 419.0.

### Example 25

### 4-Cyano-1H-imidazole-2-carboxylic acid [2-cyclohex-1-enyl-4-(3,4,5,6-tetrahydro-2H-[1,2′]bipyridinyl-4-yl)-phenyl]-amide trifluoroacetic acid salt

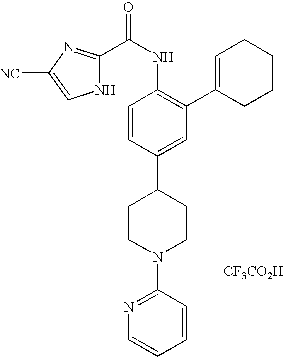

A flask was charged with 4-cyano-1H-imidazole-2-carboxylic acid (2-cyclohex-1-enyl-4-piperidin-4-yl-phenyl)-amide TFA salt (75 mg, 0.15 mmol) (as prepared in Example 14, step (b)), K₂CO₃ (84 mg, 0.60 mmol), 2-fluoropyridine (27 μL, 0.30 mmol) and 0.3 mL of N,N-dimethylacetamide and stirred for 8 h at 120° C. The reaction was diluted with 3 mL of H₂O and the title compound was purified by RP-HPLC (C18), eluting with 30-50% CH₃CN in 0.1% TFA/H₂O over 9 min to give 50 mg (75%) of a white solid. ¹H-NMR (400 MHz, CD₃OD): δ 8.18 (d, 1H), 8.06 (m, 1H), 8.02 (s, 1H), 7.94 (dd, 1H), 7.48 (d, 2H), 7.22 (dd, 1H), 7.12 (d, 1H), 6.98 (t, 1H), 5.82 (m, 1H), 4.32 (m, 2H), 3.46 (m, 2H), 3.00 (m, 1H), 2.30 (m, 4H), 2.18 (m, 2H), 1.96-1.74 (m, 6H). Mass spectrum (ESI, m/z): Calcd. for C₂₂H₂₈N₆O, 453.2 (M+H), found 453.2.

### Example 26

### 4-Cyano-1H-imidazole-2-carboxylic acid {2-cyclohex-1-enyl-4-[1-(2-hydroxy-ethyl)-piperidin-4-yl]-phenyl}-amide trifluoroacetic acid salt

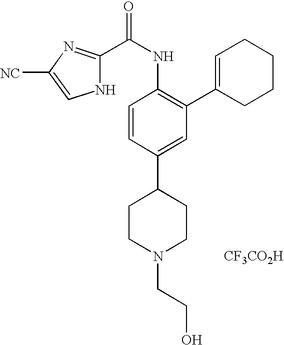

The title compound was prepared from 4-cyano-1H-imidazole-2-carboxylic acid (2-cyclohex-1-enyl-4-piperidin-4-yl-phenyl)-amide TFA salt (as prepared in Example 14, step (b)), and hydroxy-acetaldehyde according to the procedure in Example 21. ¹H-NMR (400 MHz, CD₃OD): δ 8.18 (d, 1H), 8.02 (s, 1H), 7.22 (dd, 1H), 7.14 (d, 2H), 5.82 (m, 1H), 3.94 (m, 2H), 3.74 (m, 2H), 3.30 (m, 2H), 3.18 (t, 2H), 2.92 (m, 1H), 2.30 (m, 4H), 2.20-1.98 (m, 4H), 1.96-1.74 (m, 4H). Mass spectrum (ESI, m/z): Calcd. for C₂₄H₂₉N₅O₂, 420.2 (M+H), found 420.2.

### Example 27

### 4-Cyano-1H-imidazole-2-carboxylic acid {4-[1-(2-cyano-ethyl)-ethyl)-4-yl]-2-cyclohex-1-enyl-phenyl}-amide trifluoroacetic acid salt

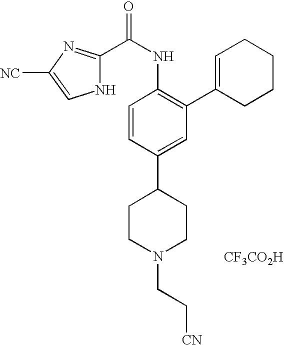

A flask was charged with 4-cyano-1H-imidazole-2-carboxylic acid (2-cyclohex-1-enyl-4-piperidin-4-yl-phenyl)-amide TFA salt (77 mg, 0.16 mmol) (as prepared in Example 14, step (b)), NEt₃ (24 μL, 0.16 mmol), acrylonitrile (12 μL, 0.18 mmol), 0.1 mL MeOH and 1.0 mL of 1,2-dichloroethane and stirred for 1 h at 80° C. The reaction was concentrated and the title compound was purified by RP-HPLC (C18), eluting with 30-50% CH₃CN in 0.1% TFA/H₂O over 12 min to give 83 mg (95%) of a white solid. ¹H-NMR (400 MHz, CD₃OD): δ 8.18 (d, 1H), 8.06 (m, 1H), 7.22 (dd, 1H), 7.12 (d, 1H), 5.82 (m, 1H), 3.76 (m, 2H), 3.60 (m, 2H), 3.28 (t, 2H), 3.12 (t, 2H), 2.92 (m, 1H), 2.30 (m, 4H), 2.18-1.98 (m, 4H), 1.92-1.74 (m, 4H). Mass spectrum (ESI, m/z): Calcd. for C₂₅H₂₈N₆O, 429.2 (M+H), found 429.2.

### Example 28

### 4-Cyano-1H-imidazole-2-carboxylic acid [4-(1-carbamoylmethyl-piperidin-4-yl)-2-cyclohex-1-enyl-phenyl]-amide trifluoroacetic acid salt

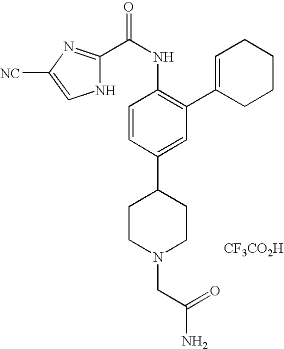

A flask was charged with 4-cyano-1H-imidazole-2-carboxylic acid (2-cyclohex-1-enyl-4-piperidin-4-yl-phenyl)-amide TFA salt (50 mg, 0.10 mmol) (as prepared in Example 14, step (b)), NEt₃ (32 μL, 0.23 mmol), 2-bromoacetamide (16 mg, 0.12 mmol), and 0.5 mL of DCM and stirred for 4 h at 25° C. The reaction was concentrated and the title compound was purified by RP-HPLC (C18), eluting with 30-50% CH₃CN in 0.1% TFA/H₂O over 12 min to give 42 mg (75%) of a white solid. ¹H-NMR (400 MHz, DMSO-d₆): δ 14.28 (br s, 1H), 9.78 (s, 1H), 9.50 (br s, 1H), 8.34 (s, 1H), 8.00 (s, 1H), 7.88 (d, 1H), 7.72 (s, 1H), 7.18 (dd, 1H), 7.10 (d, 1H), 5.76 (m, 1H), 3.94 (s, 2H), 3.58 (m, 2H), 3.12 (m, 2H), 2.80 (m, 1H), 2.20 (m, 4H), 1.98 (m, 4H), 1.80 (m, 4H). Mass spectrum (ESI, m/z): Calcd. for C₂₄H₂₈N₆O₂, 433.2 (M+H), found 433.2.

### Example 29

### 4-Cyano-1H-imidazole-2-carboxylic acid {2-cyclohex-1-enyl-4-[1-(2-pyridin-2-yl-acetyl)-piperidin-4-yl]-phenyl}-amide trifluoroacetic acid salt

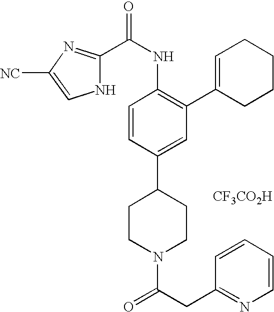

A flask was charged with 4-cyano-1H-imidazole-2-carboxylic acid (2-cyclohex-1-enyl-4-piperidin-4-yl-phenyl)-amide TFA salt (25 mg, 0.05 mmol) (as prepared in Example 14, step (b)), pyridin-2-yl-acetic acid hydrochloride (10 mg, 0.06 mmol), EDCI (12 mg, 0.06 mmol), HOBt (8.0 mg, 0.06 mmol), DIEA (36 μL, 0.20 mmol) and 0.2 mL DMF and stirred at 25° C. for 10 h. The reaction was diluted with 2 mL of H₂O and the title compound was purified by RP-HPLC (C18), eluting with 30-50% CH₃CN in 0.1% TFA/H₂O over 9 min to give 22 mg (70%) of a white solid. ¹H-NMR (400 MHz, CD₃OD): δ 8.82 (d, 1H), 8.52 (t, 1H), 8.14 (d, 1H), 8.04 (s, 1H), 7.96 (m, 3H), 7.20 (dd, 1H), 7.10 (d, 1H), 5.82 (m, 1H), 4.68 (m, 1H), 4.32 (m, 2H), 4.18 (m, 1H), 3.40 (m, 1H), 2.88 (m, 2H), 2.30 (m, 4H), 2.06-1.60 (m, 8H). Mass spectrum (ESI, m/z): Calcd. for C₂₉H₃₀N₆O₂, 495.2.2 (M+H), found 495.2.

### Example 30

### 4-Cyano-1H-imidazole-2-carboxylic acid {2-cyclohex-1-enyl-4-[1-(2-pyridin-3-yl-acetyl)-piperidin-4-yl]-phenyl}-amide trifluoroacetic acid salt

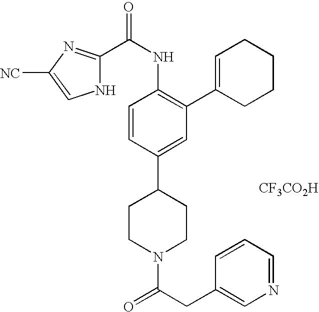

The title compound was prepared from 4-cyano-1H-imidazole-2-carboxylic acid (2-cyclohex-1-enyl-4-piperidin-4-yl-phenyl)-amide TFA salt (as prepared in Example 14, step (b)), according to the procedure in Example 29 using pyridin-3-yl-acetic acid. ¹H-NMR (400 MHz, CD₃OD): δ 8.80 (m, 2H), 8.54 (d, 1H), 8.10 (d, 1H), 8.06 (t, 1H), 7.98 (s, 1H), 7.18 (dd, 1H), 7.08 (d, 1H), 5.78 (m, 1H), 4.68 (m, 1H), 4.20 (m, 1H), 4.18 (s, 2H), 3.36 (m, 1H), 2.84 (m, 2H), 2.28 (m, 4H), 2.06-1.70 (m, 7H), 1.62 (m, 1H). Mass spectrum (ESI, m/z): Calcd. for C₂₉H₃₀N₆O₂, 495.2 (M+H), found 495.2.

### Example 31

### 4-Cyano-1H-imidazole-2-carboxylic acid {2-cyclohex-1-enyl-4-[1-(2-pyridin-4-yl-acetyl)-piperidin-4-yl]-phenyl}-amide trifluoroacetic acid salt

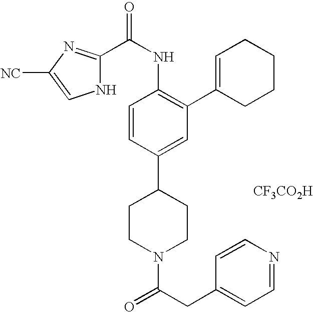

The title compound was prepared from 4-cyano-1H-imidazole-2-carboxylic acid (2-cyclohex-1-enyl-4-piperidin-4-yl-phenyl)-amide TFA salt (as prepared in Example 14, step (b)), according to the procedure in Example 29 using pyridin-4-yl-acetic acid. ¹H-NMR (400 MHz, CD₃OD): δ 8.78 (d, 2H), 8.12 (d, 1H), 8.00 (m, 3H), 7.18 (dd, 1H), 7.08 (d, 1H), 5.80 (m, 1H), 4.66 (m, 1H), 4.22 (s, 2H), 4.18 (m, 1H), 3.34 (m, 1H), 2.84 (m, 2H), 2.24 (m, 4H), 2.00-1.70 (m, 7H), 1.64 (m, 1H). Mass spectrum (ESI, m/z): Calcd. for C₂₉H₃₀N₆O₂, 495.2 (M+H), found 495.2.

### Example 32

### 4-Cyano-1H-imidazole-2-carboxylic acid (2-cyclohex-1-enyl-4-{1-[2-(1-methyl-1H-imidazol-4-yl)-acetyl]-piperidin-4-yl}-phenyl)-amide trifluoroacetic acid salt

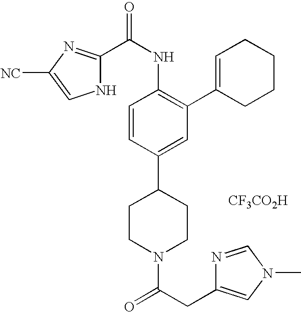

The title compound was prepared from 4-cyano-1H-imidazole-2-carboxylic acid (2-cyclohex-1-enyl-4-piperidin-4-yl-phenyl)-amide TFA salt (as prepared in Example 14, step (b)), according to the procedure in Example 29 using (1-methyl-1H-imidazol-4-yl)-acetic acid. ¹H-NMR (400 MHz, CD₃OD): δ 8.82 (s, 1H), 8.10 (d, 1H), 8.00 (s, 1H), 7.42 (s, 1H), 7.16 (dd, 1H), 7.06 (d, 1H), 5.80 (m, 1H), 4.66 (m, 1H), 4.12 (m, 1H), 4.04 (m, 2H), 3.92 (s, 3H), 3.28 (m, 1H), 2.82 (m, 2H), 2.26 (m, 4H), 2.00-1.70 (m, 7H), 1.64 (m, 1H). Mass spectrum (ESI, m/z): Calcd. for C₂₈H₃₁N₇O₂, 498.2 (M+H), found 498.2.

### Example 33

### 4-Cyano-1H-imidazole-2-carboxylic acid {2-cyclohex-1-enyl-4-[1-(2-1H-imidazol-4-yl-acetyl)-piperidin-4-yl]-phenyl}-amide trifluoroacetic acid salt

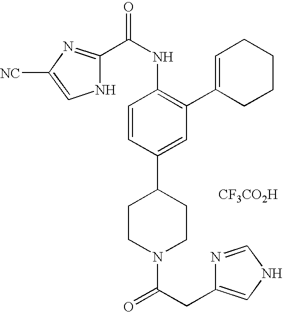

The title compound was prepared from 4-cyano-1H-imidazole-2-carboxylic acid (2-cyclohex-1-enyl-4-piperidin-4-yl-phenyl)-amide TFA salt (as prepared in Example 14, step (b)), according to the procedure in Example 29 using (1-methyl-1H-imidazol-4-yl)-acetic acid. ¹H-NMR (400 MHz, CD₃OD): δ 8.88 (s, 1H), 8.12 (d, 1H), 8.02 (s, 1H), 7.44 (s, 1H), 7.20 (dd, 1H), 7.10 (d, 1H), 5.82 (m, 1H), 4.70 (m, 1H), 4.18 (m, 1H), 4.06 (m, 2H), 3.36 (m, 1H), 2.84 (m, 2H), 2.30 (m, 4H), 2.00-1.70 (m, 7H), 1.64 (m, 1H). Mass spectrum (ESI, m/z): Calcd. for C₂₇H₂₉N₇O₂, 484.2 (M+H), found 484.2.

### Example 34

### 4-Cyano-1H-imidazole-2-carboxylic acid {2-cyclohex-1-enyl-4-[1-(2-morpholin-4-yl-ethyl)-piperidin-4-yl]-phenyl}-amide di-trifluoroacetic acid salt

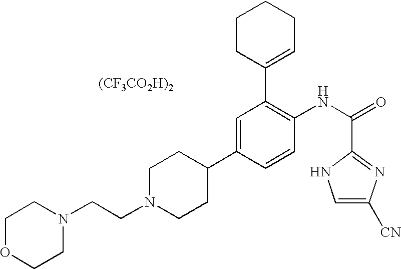

### a) 4-Cyano-1-(2-trimethylsilanyl-ethoxymethyl)-1H-imidazole-2-carboxylic acid {2-cyclohex-1-enyl-4-[1-(2-morpholin-4-yl-ethyl)-piperidin-4-yl]-phenyl}-amide

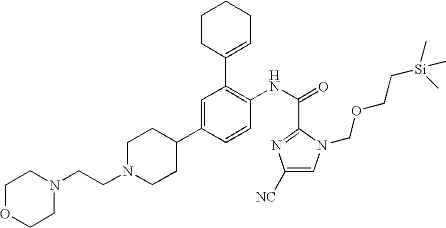

A flask was charged with 4-cyano-1-(2-trimethylsilanyl-ethoxymethyl)-1H-imidazole-2-carboxylic acid (2-cyclohex-1-enyl-4-piperidin-4-yl-phenyl)-amide TFA salt (830 mg, 1.34 mmol) (as prepared in Example 39, step (a)), K₂CO₃ (600 mg, 4.34 mmol), sodium iodide (40 mg, 0.27 mmol), 4-(2-chloro-ethyl)-morpholine hydrochloride (260 mg, 1.40 mmol), and 5.0 mL of N,N-dimethylacetamide and stirred for 8 h at 80° C. The reaction was diluted with EtOAc (50 mL) and washed with NaHCO₃ (2×50 mL), brine (50 mL) and concentrated. The title compound was purified by flash chomatography (silica gel, 5% MeOH/DCM) to give 650 mg (78%) of a white solid. Mass spectrum (ESI, m/z): Calcd. for C₃₄H₅₀N₆O₃Si, 619.4 (M+H), found 619.3.

### b) 4-Cyano-1H-imidazole-2-carboxylic acid {2-cyclohex-1-enyl-4-[1-(2-morpholin-4-yl-ethyl)-piperidin-4-yl]-phenyl}-amide trifluoroacetic acid salt To a solution of 4-cyano-1-(2-trimethylsilanyl-ethoxymethyl)-1H-imidazole-2-carboxylic acid {2-cyclohex-1-enyl-4-[1-(2-morpholin-4-yl-ethyl)-piperidin-4-yl]-phenyl}-amide (650 mg, 1.05 mmol) (as prepared in the previous step) in 10 mL of DCM was added 0.3 mL of EtOH and 3.0 mL of TFA, and the reaction was allowed to proceed for 2 h at 25° C. The reaction was diluted with 10 mL of EtOH and concentrated. The title compound was purified by RP-HPLC (C18), eluting with 30-50% CH₃CN in 0.1% TFA/H₂O over 9 min to give 600 mg (80%) of a white solid. ¹H-NMR (400 MHz, CD₃OD): δ 8.18 (d, 1H), 8.04 (s, 1H), 7.24 (dd, 1H), 7.14 (d, 1H), 5.84 (m, 1H), 3.84 (m, 4H), 3.76 (m, 2H), 3.50 (m, 2H), 3.30-3.10 (m, 4H), 2.92 (m, 5H), 2.30 (m, 4H), 2.20-2.00 (m, 4H), 1.90-1.74 (m, 4H). Mass spectrum (ESI, m/z): Calcd. for C₂₈H₃₆N₆O₂, 489.2, found 489.2.

### Example 35

### 4-Cyano-1H-imidazole-2-carboxylic acid [2-(1,1-dioxo-1,2,3,6-tetrahydro-1λ⁶-thiopyran-4-yl)-4-piperidin-4-yl-phenyl]-amide

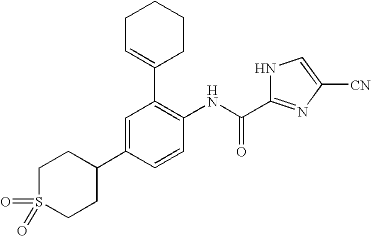

### a) Trifluoromethanesulfonic acid 3,6-dihydro-2H-thiopyran-4-yl ester

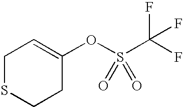

A solution of tetrahydro-thiopyran-4-one (1.00 g, 8.61 mmol) in 10 ml of THF was added to a solution of LDA (2.0 M, 4.52 ml, 9.04 mmol) in 20 ml of THF at −78° C. under Ar. The mixture was warmed to RT and stirred for 0.5 h, then cooled to −78° C. again. A solution of N-phenyltrifluoromethanesulfonimide (3.42 g, 9.47 mmol) in 10 ml of THF was added. The resulting mixture was warmed to RT and stirred for 0.5 h under Ar. Treated with 200 ml of EtOAc, the mixture was washed with H₂O (3×50 mL), brine (50 mL) and dried (Na₂SO₄). Removal of the solvent under reduced pressure followed by flash chromatography of the residue on silica gel (hexane-3% EtOAc/hexane) gave 810 mg (38%) of the title compound as a colorless oil. ¹H-NMR (CDCl₃; 400 MHz): δ 6.01 (m, 1H), 3.30 (m, 2H), 2.86 (dd, 2H, J=5.7, 5.7 Hz), 2.58-2.64 (m, 2H). Mass spectrum (ESI, m/z): Calcd. for C₆H₇F₃O₃S₂, 249.0 (M+H), found 249.3.

### b) 4-(4-Nitro-phenyl)-3,6-dihydro-2H-thiopyran

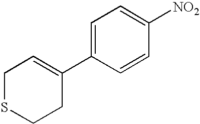

To a mixture of 4-nitrophenylboronic acid (418 mg, 2.50 mmol), trifluoro-methanesulfonic acid 3,6-dihydro-2H-thiopyran-4-yl ester (as prepared in the previous step, 931 mg, 3.75 mmol), Pd(PPh₃)₄ (433 mg, 0.375 mmol) and lithium chloride (LiCl) (212 mg, 5.0 mmol) in 20 mL of 1,4-dioxane was added 2.0 M aq Na₂CO₃ solution (3.13 mL, 6.25 mmol). The resulting mixture was stirred at 80° C. for 2 h and then cooled to RT. Treated with 200 mL of EtOAc, the mixture was washed with H₂O (2×30 mL), brine (30 mL) and dried (Na₂SO₄). Removal of the solvent under reduced pressure followed by flash chromatography of the residue on silica gel (1-3% EtOAc/hexane) gave 470 mg (85%) of the title compound as a light brown oil. ¹H-NMR (CDCl₃; 400 MHz): δ 8.19 (d, 2H, J=9.1 Hz), 7.48 (d, 2H, J=9.1 Hz), 6.36 (m, 1H), 3.39 (m, 2H), 2.91 (t, 2H, J=5.7 Hz), 2.72 (m, 2H). Mass spectrum (ESI, m/z): Calcd. for C₁₁K₁NO₂S, 222.1 (M+H), found 222.3.

### c) 4-(4-Nitro-phenyl)-3,6-dihydro-2H-thiopyran 1,1-dioxide

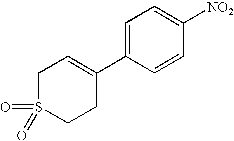

A solution of 3-chloroperoxybenzoic acid (1.04 g, 4.62 mmol, 77%) in 15 mL of dichloromethane (DCM) was added slowly to a solution of 4-(4-nitro-phenyl)-3,6-dihydro-2H-thiopyran (as prepared in the previous step, 465 mg, 2.10 mmol) in 15 mL of DCM at −78° C. under Ar. The mixture was stirred at −78° C. for 0.5 h, and then warmed to RT. Treated with 100 mL of EtOAc, the mixture was washed with 10% Na₂SO₃ (2×15 mL), satd aq NaHCO₃ solution (20 mL), H₂O (20 mL), brine (20 mL) and dried (Na₂SO₄). Removal of the solvent under reduced pressure followed by flash chromatography of the residue on silica gel (2-5% EtOAc/DCM) gave 518 mg (97%) of the title compound as a white solid. ¹H-NMR (CDCl₃; 400 MHz): δ 8.23 (d, 2H, J=9.0 Hz), 7.52 (d, 2H, J=9.0 Hz), 6.04 (m, 1H), 3.86 (m, 2H), 3.26-3.31 (m, 2H), 3.18-3.23 (m, 2H).

### d) 4-(1,1-Dioxo-hexahydro-1λ⁶-thiopyran-4-yl)-phenylamine

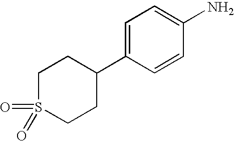

A mixture of 4-(4-nitro-phenyl)-3,6-dihydro-2H-thiopyran 1,1-dioxide (as prepared in the previous step, 502 mg, 1.98 mmol) and 10% Pd/C (250 mg, 50 wt %) in 15 mL of MeOH was stirred at RT under H₂ (balloon pressure) for 2 h. The Pd catalyst was removed by filtration on Celite, and the filtrate was concentrated to give 314 mg (70%) of the title compound as a slightly yellow solid. ¹H-NMR (CDCl₃; 400 MHz): δ 7.03 (d, 2H, J=8.3 Hz), 6.67 (d, 2H, J=8.3 Hz), 3.51-3.79 (br s, 2H), 3.11-3.17 (m, 4H), 2.70 (dddd, 1H, J=12.3, 12.3, 2.9, 2.9 Hz), 2.31-2.43 (m, 2H), 2.15-2.23 (m, 2H).

### e) 2-Bromo-4-(1,1-dioxo-hexahydro-1λ⁶-thiopyran-4-yl)-phenylamine

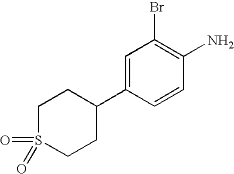

To a suspension of 4-(1,1-dioxo-hexahydro-1λ⁶-thiopyran-4-yl)-phenylamine (as prepared in the previous step, 174 mg, 0.77 mmol) in 20 mL of 3:1 DCM/MeOH at 0° C. was added N-bromosuccinimide (NBS) (137 mg, 0.77 mmol) in 5 mL of DCM under Ar. The mixture was warmed to RT and stirred for 1 h under Ar. Treated with 100 mL of EtOAc, the mixture was washed with H₂O (2×20 mL), brine (20 mL) and dried (Na₂SO₄). Removal of the solvent under reduced pressure followed by flash chromatography of the residue on silica gel (2-3% EtOAc/DCM) gave 155 mg (66%) of the title compound as a white solid. ¹H-NMR (CDCl₃; 400 MHz): δ 7.28 (d, 1H, J=2.0 Hz), 6.97 (dd, 1H, J=8.3, 2.0 Hz), 6.73 (d, 1H, J=8.3 Hz), 4.07 (br s, 2H), 3.09-3.14 (m, 4H), 2.66 (dddd, 1H, J=12.1, 12.1, 3.3, 3.3 Hz), 2.26-2.39 (m, 2H), 2.12-2.21 (m, 2H). Mass spectrum (ESI, m/z): Calcd. for C₁₁H₁₄BrNO₂S, 304.0 (M+H), found 304.1.

### f) 2-Cyclohex-1-enyl-4-(1,1-dioxo-hexahydro-1λ⁶-thiopyran-4-yl)-phenylamine

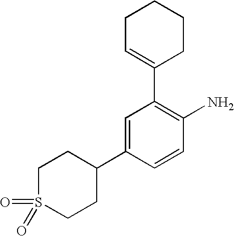

To a mixture of 2-bromo-4-(1,1-dioxo-hexahydro-1λ⁶-thiopyran-4-yl)-phenylamine (as prepared in the previous step, 150 mg, 0.493 mmol), cyclohexen-1-yl boronic acid (70 mg, 0.542 mmol) and Pd(PPh₃)₄ (57 mg, 0.0493 mmol) in 5 mL of 1,4-dioxane was added 2.0 M aq Na₂CO₃ solution (2.0 mL, 4.0 mmol). The resulting mixture was stirred at 80° C. for 8 h under Ar, and then cooled to RT. Treated with 50 mL of EtOAc, the mixture was washed with H₂O (3×15 mL), brine (20 mL) and dried (Na₂SO₄). Removal of the solvent under reduced pressure followed by flash chromatography of the residue on silica gel (2-5% EtOAc/DCM) gave 130 mg (86%) of the title compound as a brown solid. ¹H-NMR (CDCl₃; 400 MHz): δ 6.89 (dd, 1H, J=8.4, 2.3 Hz), 6.84 (d, 1H, J=2.3 Hz), 6.65 (d, 1H, J=8.4 Hz), 5.74 (m, 1H), 3.74 (br s, 2H), 3.08-3.17 (m, 4H), 2.66 (dddd, 1H, J=12.1, 12.1, 3.1, 3.1 Hz), 2.29-2.42 (m, 2H), 2.13-2.25 (m, 6H), 1.73-1.81 (m, 2H), 1.65-1.73 (m, 2H). Mass spectrum (ESI, m/z): Calcd. for C₁₂H₂₃NO₂S, 306.1 (M+H), found 306.1.

### g) 4-Cyano-1-(2-trimethylsilanyl-ethoxymethyl)-1H-imidazole-2-carboxylic acid [2-cyclohex-1-enyl-4-(1,1-dioxo-hexahydro-1λ⁶-thiopyran-4-yl)-phenyl]-amide

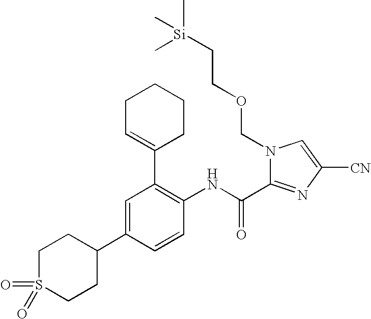

To a mixture of 2-cyclohex-1-enyl-4-(1,1-dioxo-hexahydro-1λ⁶-thiopyran-4-yl)-phenylamine (as prepared in the previous step, 122 mg, 0.50 mmol), potassium 4-cyano-1-(2-trimethylsilanyl-ethoxymethyl)-1H-imidazole-2-carboxylate (as prepared in Example 3, step (d), 134 mg, 0.44 mmol) and bromotri(pyrrolidino)phosphonium hexafluorophosphate (PyBroP) (205 mg, 0.44 mmol) in 5 mL of DMF was added DIEA (209 μL, 1.20 mmol). The resulting mixture was stirred at RT for 18 h under Ar, cooled to RT. Treated with 50 mL of EtOAc, the mixture was washed with H₂O (3×10 mL), brine (10 mL) and dried (Na₂SO₄). Removal of the solvent under reduced pressure followed by flash chromatography of the residue on silica gel (1-3% EtOAc/DCM) gave 161 mg (73%) of the title compound as a colorless oil. ¹H-NMR (CDCl₃; 400 MHz): δ 9.69 (s, 1H), 8.29 (d, 1H, J=8.4 Hz), 7.78 (s, 1H), 7.14 (dd, 1H, J=8.4, 2.2 Hz), 7.04 (d, 1H, J=2.2 Hz), 5.95 (s, 2H), 5.83 (m, 1H), 3.66 (t, 2H, J=8.2 Hz), 3.11-3.20 (m, 4H), 2.77 (dddd, 1H, J=12.1, 12.1, 3.2, 3.2 Hz), 2.35-2.47 (m, 2H), 2.17-2.33 (m, 6H), 1.74-1.89 (m, 4H), 0.97 (t, 2H, J=8.2 Hz), 0.00 (s, 9H). Mass spectrum (ESI, m/z): Calcd. for C₂₈H₃₈N₄O₄SSi, 555.2 (M+H), found 555.3.

### h) 4-Cyano-1H-imidazole-2-carboxylic acid [2-cyclohex-1-enyl-4-(1,1-dioxo-hexahydro-1λ⁶-thiopyran-4-yl)-phenyl]-amide

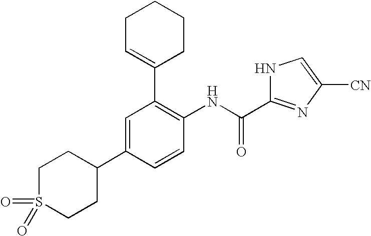

To a solution of 4-cyano-1-(2-trimethylsilanyl-ethoxymethyl)-1H-imidazole-2-carboxylic acid [2-cyclohex-1-enyl-4-(1,1-dioxo-hexahydro-1λ⁶-thiopyran-4-yl)-phenyl]-amide (as prepared in the previous step, 145 mg, 0.261 mmol) in 6 mL of DCM was added 0.20 mL of EtOH followed by 2 mL of TFA. The resulting solution was stirred at RT for 3 h. Removal of the solvent under reduced pressure followed by flash chromatography of the residue on silica gel (20-25% EtOAc/DCM) gave 83 mg (90%) of the title compound as a white solid. ¹H-NMR (CDCl₃; 400 MHz): δ 12.34 (s, 1H), 9.60 (s, 1H), 8.35 (d, 1H, J=8.4 Hz), 7.75 (s, 1H), 7.30 (dd, 1H, J=8.4, 2.2 Hz), 7.08 (d, 1H, J=2.2 Hz), 5.86 (m, 1H), 3.11-3.23 (m, 4H), 2.80 (dddd, 1H, J=12.2, 12.2, 2.8, 2.8 Hz), 2.40-2.57 (m, 2H), 2.17-2.35 (m, 6H), 1.74-1.91 (m, 4H). Mass spectrum (ESI, m/z): Calcd. for C₂₂H₂₄N₄O₃S, 425.2 (M+H), found 425.6.

### Example 36

### 4-Cyano-1H-imidazole-2-carboxylic acid [2-(1,1-dioxo-1,2,3,6-tetrahydro-1λ⁶-thiopyran-4-yl)-4-piperidin-4-yl-phenyl]-amide trifluoroacetic acid salt

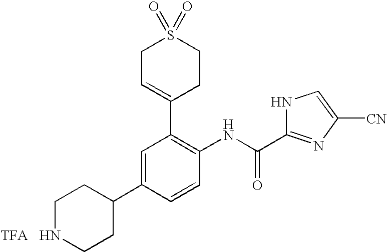

### a) 2-(3,6-Dihydro-2H-thiopyran-4-yl)-5,5-dimethyl-[1,3,2]dioxaborinane

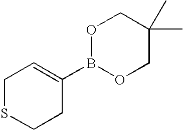

A mixture of trifluoromethanesulfonic acid 3,6-dihydro-2H-thiopyran-4-yl ester (as prepared in Example 35, step (a), 500 mg, 2.01 mmol), bis(neopentyl glycolato)diboron (478 mg, 2.11 mmol), Pd(dppf)Cl₂ (147 mg, 0.20 mmol) and KOAc (592 mg, 6.03 mmol) in 8 mL of 1,4-dioxane was stirred at 80° C. for 8 h under Ar, and then cooled to RT. Treated with 50 mL of EtOAc, the mixture was washed with H₂O (2×10 mL), brine (10 mL) and dried (Na₂SO₄). Removal of the solvent under reduced pressure followed by flash chromatography of the residue on silica gel (0-5% EtOAc/DCM) gave 351 mg (82%) of the title compound as a colorless oil. ¹H-NMR (CDCl₃; 400 MHz): δ 6.62 (m, 1H), 3.63 (s, 4H), 3.21 (m, 2H), 2.68 (t, 2H, J=5.8 Hz), 2.37 (m, 2H), 0.96 (s, 6H). Mass spectrum (ESI, m/z): Calcd. for C₁₀H₁₇BO₂S, 213.1 (M+H), found 213.1.

### b) 4-[4-Amino-3-(3,6-dihydro-2H-thiopyran-4-yl)-phenyl]-piperidine-1-carboxylic acid tert-butyl ester

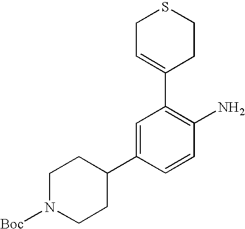

To a mixture of 4-(4-amino-3-bromo-phenyl)-piperidine-1-carboxylic acid tert-butyl ester (as prepared in Example 13, step (c), 200 mg, 0.563 mmol), 2-(3,6-dihydro-2H-thiopyran-4-yl)-5,5-dimethyl-[1,3,2]dioxaborinane (as prepared in the previous step, 131 mg, 0.619 mmol) and Pd(PPh₃)₄ (65 mg, 0.056 mmol) in 5 mL of 1,4-dioxane was added 2.0 M aq Na₂CO₃ solution (2.25 mL, 4.5 mmol). The resulting mixture was stirred at 80° C. for 7 h under Ar, and then cooled to RT. Treated with 50 mL of EtOAc, the mixture was washed with H₂O (3×15 mL), brine (20 mL) and dried (Na₂SO₄). Removal of the solvent under reduced pressure followed by flash chromatography of the residue on silica gel (15-30% EtOAc/hexane) gave 141 mg (67%) of the title compound as a colorless oil. ¹H-NMR (CDCl₃; 400 MHz): δ 6.91 (dd, 1H, J=8.2, 2.2 Hz), 6.81 (d, 1H, J=2.2 Hz), 6.65 (d, 1H, J=8.2 Hz), 5.91 (m, 1H), 4.22 (br s, 2H), 3.66 (br s, 2H), 3.29-3.31 (m, 2H), 2.87 (dd, 2H, J=5.7, 5.7 Hz), 2.77 (m, 2H), 2.47-2.56 (m, 3H), 1.78 (d, 2H, J=12.6 Hz), 1.50-1.63 (m, 2H), 1.48 (s, 9H). Mass spectrum (ESI, m/z): Calcd. for C₂₁H₃₀N₂O₂S, 375.2 (M+H), found 375.2.

### c) 4-[4-{[4-Cyano-1-(2-trimethylsilanyl-ethoxymethyl)-1H-imidazole-2-carbonyl]-amino}-3-(3,6-dihydro-2H-thiopyran-4-yl)-phenyl]-piperidine-1-carboxylic acid tert-butyl ester

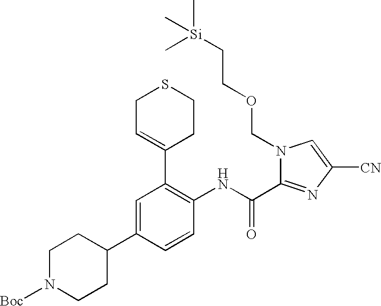

To a mixture of 4-[4-amino-3-(3,6-dihydro-2H-thiopyran-4-yl)-phenyl]-piperidine-1-carboxylic acid tert-butyl ester (as prepared in the previous step, 45 mg, 0.12 mmol), potassium 4-cyano-1-(2-trimethylsilanyl-ethoxymethyl)-1H-imidazole-2-carboxylate (as prepared in Example 3, step (d), 44 mg, 0.144 mmol) and PyBroP (67 mg, 0.144 mmol) in 2 mL of DMF was added DIEA (42 μL, 0.24 mmol). The resulting mixture was stirred at RT for 4 h under Ar. Treated with 30 mL of EtOAc, the mixture was washed with H₂O (3×10 mL), brine (10 mL) and dried (Na₂SO₄). Removal of the solvent under reduced pressure followed by flash chromatography of the residue on silica gel (1-2% EtOAc/DCM) gave 64 mg (85%) of the title compound as a light yellow oil. ¹H-NMR (CDCl₃; 400 MHz): δ 9.51 (s, 1H), 8.21 (d, 1H, J=8.5 Hz), 7.78 (s, 1H), 7.16 (dd, 1H, J=8.5, 2.1 Hz), 7.02 (d, 1H, J=2.1 Hz), 6.00 (m, 1H), 5.92 (s, 2H), 4.25 (br s, 2H), 3.66 (t, 2H, J=8.2), 3.42 (m, 2H), 2.93 (dd, 2H, J=5.7, 5.7 Hz), 2.79 (m, 2H), 2.63 (dddd, 1H, J=12.3, 12.3, 3.3, 3.3 Hz), 2.49-2.56 (m, 2H), 1.82 (d, 2H, J=12.8 Hz), 1.56-1.66 (m, 2H), 1.49 (s, 9H), 0.97 (t, 2H, J=8.2 Hz), 0.00 (s, 9H).

### d) 4-[4-{[4-Cyano-1-(2-trimethylsilanyl-ethoxymethyl)-1H-imidazole-2-carbonyl]-amino}-3-(1,1-dioxo-1,2,3,6-tetrahydro-1λ⁶-thiopyran-4-yl)-phenyl]-piperidine-1-carboxylic acid tert-butyl ester

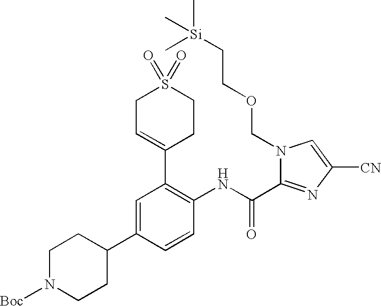

A solution of 3-chloroperoxybenzoic acid (91 mg, 0.404 mmol, 77%) in 1 mL of DCM was added slowly to 4-[4-{[4-cyano-1-(2-trimethylsilanyl-ethoxymethyl)-1H-imidazole-2-carbonyl]-amino}-3-(3,6-dihydro-2H-thiopyran-4-yl)-phenyl]-piperidine-1-carboxylic acid tert-butyl ester (as prepared in the previous step, 120 mg, 0.192 mmol) in 3 mL of DCM at −78° C. under Ar. The mixture was stirred at −78° C. for 15 min, and then warmed to RT. Treated with 40 mL of EtOAc, the mixture was washed with 15% Na₂SO₃ (5 mL), satd aq NaHCO₃ solution (2×10 mL), H₂O (10 mL), brine (10 mL) and dried (Na₂SO₄). Removal of the solvent under reduced pressure followed by flash chromatography of the residue on silica gel (2-10% EtOAc/DCM) gave 85 mg (67%) of the title compound as a colorless oil. ¹H-NMR (CDCl₃; 400 MHz): δ 9.23 (s, 1H), 8.03 (d, 1H, J=8.3 Hz), 7.80 (s, 1H), 7.21 (dd, 1H, J=8.3, 2.0 Hz), 7.06 (d, 1H, J=2.0 Hz), 5.93 (s, 2H), 5.75 (t, 1H, J=4.1 Hz), 4.25 (br s, 2H), 3.86 (br s, 2H), 3.66 (t, 2H, J=8.2 Hz), 3.29 (t, 2H, J=6.3 Hz), 3.03 (t, 2H, J=5.4 Hz), 2.74-2.86 (m, 2H), 2.64 (dddd, 1H, J=12.3, 12.3, 3.3, 3.3 Hz), 1.82 (d, 2H, J=12.3 Hz), 1.55-1.65 (m, 2H), 1.49 (s, 9H), 0.98 (t, 2H, J=8.2 Hz), 0.01 (s, 9H). Mass spectrum (ESI, m/z): Calcd. for C₃₂H₄₅N₅O₆SSi, 656.3 (M+H), found 656.7.

### e) 4-Cyano-1H-imidazole-2-carboxylic acid [2-(1,1-dioxo-1,2,3,6-tetrahydro-1λ⁶-thiopyran-4-yl)-4-piperidin-4-yl-phenyl]-amide, trifluoroacetic acid salt

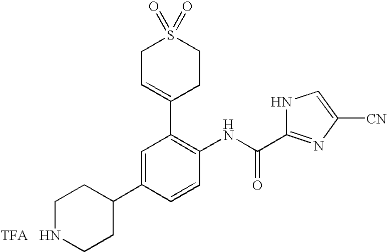

To a solution of 4-[4-{[4-cyano-1-(2-trimethylsilanyl-ethoxymethyl)-1H-imidazole-2-carbonyl]-amino}-3-(1,1-dioxo-1,2,3,6-tetrahydro-1λ⁶-thiopyran-4-yl)-phenyl]-piperidine-1-carboxylic acid tert-butyl ester (as prepared in the previous step, 81 mg, 0.123 mmol) in 6 mL of DCM was added 0.20 mL of EtOH followed by 2 mL of TFA. The resulting solution was stirred at RT for 3 h. Removal of the solvent under reduced pressure gave 64 mg (96%) of the title compound as a white solid. ¹H-NMR (CD₃OD; 400 MHz): δ 8.02 (s, 1H), 7.78 (d, 1H, J=8.3 Hz), 7.29 (dd, 1H, J=8.3, 2.0 Hz), 7.21 (d, 1H, J=2.0 Hz), 5.71 (t, 1H, J=4.2 Hz), 3.83 (br s, 2H), 3.51 (d, 2H, J=12.4 Hz), 3.33 (t, 2H, J=6.0 Hz), 3.15 (td, 2H, J=13.1, 2.6 Hz), 3.01 (m, 2H), 2.94 (dddd, 1H, J=12.2, 12.2, 3.5, 3.5 Hz), 2.08 (d, 2H, J=12.9 Hz), 1.91 (m, 2H, J=13.3, 13.3, 13.3, 3.8 Hz). Mass spectrum (ESI, m/z): Calcd. for C₂₁H₂₃N₅O₃S, 426.2 (M+H), found 426.2.

### Example 37

### 4-Cyano-1H-imidazole-2-carboxylic acid [4-(1-acetyl-piperidin-4-yl)-2-(1,1-dioxo-1,2,3,6-tetrahydro-1λ⁶-thiopyran-4-yl)-phenyl]-amide

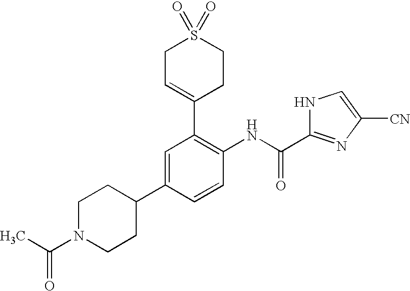

To a suspension of 4-cyano-1H-imidazole-2-carboxylic acid [2-(1,1-dioxo-1,2,3,6-tetrahydro-1λ⁶-thiopyran-4-yl)-4-piperidin-4-yl-phenyl]-amide trifluoroacetic acid salt (as prepared in Example 36, step (e), 62 mg, 0.115 mmol) in 4 mL of 1:1 DCM/DMF at RT was added DIEA (60 μL, 0.345 mmol). The mixture was stirred for 5 min, then acetic anhydride (11 μL, 0.121 mmol) was added slowly to the mixture, and the resulting mixture was stirred at RT for 0.5 h. Treated with 40 mL of EtOAc, the mixture was washed with H₂O (2×20 mL). The aqueous layers were extracted with EtOAc (4×10 mL). The combined organic layers were concentrated in vacuo. The residue was purified by flash chromatography on silica gel (1-4% MeOH/DCM) yielding 50.9 mg (95%) of the title compound as a white solid. ¹H-NMR (CDCl₃; 400 MHz): δ 13.0 (s, 1H), 9.10 (s, 1H), 8.13 (d, 1H, J=8.4 Hz), 7.77 (d, 1H, J=2.3 Hz), 7.26 (dd, 1H, J=8.4, 2.0 Hz), 7.08 (d, 1H, J=2.0 Hz), 5.77 (t, 1H, J=4.3 Hz), 4.84 (dt, 1H, J=13.3, 2.1 Hz), 4.00 (dt, 1H, J=13.3, 2.1 Hz), 3.89 (br s, 2H), 3.31 (t, 2H, J=6.2 Hz), 3.23 (td, 1H, J=13.2, 2.5 Hz), 3.02 (m, 2H), 2.77 (dddd, 1H, J=11.9, 11.9, 3.4, 3.4 Hz), 2.68 (ddd, 1H, J=12.6, 12.6, 2.9 Hz), 2.18 (s, 3H), 1.70-1.97 (m, 4H). Mass spectrum (ESI, m/z): Calcd. for C₂₃H₂₅N₅O₄S, 468.2 (M+H), found 468.1.

### Example 38a

### 4-Cyano-1H-imidazole-2-carboxylic acid {2-cyclohex-1-enyl-4-[1-(2-dimethylamino-acetyl)-piperidin-4-yl]-phenyl}-amide

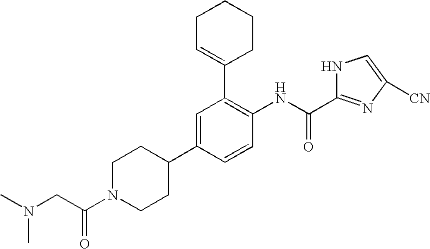

A mixture of 4-cyano-1H-imidazole-2-carboxylic acid (2-cyclohex-1-enyl-4-piperidin-4-yl-phenyl)-amide trifluoroacetic acid salt (as prepared in Example 14, step (b), 655 mg, 1.30 mmol) in DCM (15 mL) was cooled to 0° C. and DIEA (0.92 mL, 5.2 mmol) was added. Dimethylaminoacetyl chloride hydrochloride (211 mg, 1.3 mol) was then added portion wise over 10 min. The reaction mixture was stirred at 0° C. for 30 min and allowed to warm to RT and stirred for 2 h. Solvent was removed in vacuo and the resulting residue was partitioned between brine and DCM. The organic layer was separated, dried (Na₂SO₄) and concentrated. The residue obtained was purified on silica (5% MeOH: DCM) to obtain 432 mg (70%) of the title compound as a white solid. ¹H-NMR (CDCl₃; 400 MHz): δ 9.49 (s, 1H), 8.24 (d, 1H, J=2.3 Hz), 7.70 (s, 1H), 7.12 (dd, 1H, J=8.4, 2.1 Hz), 7.01 (s, 1H), 5.82 (m, 1H), 4.75 (d, 1H, J=13.4 Hz), 4.13 (d, 1H, J=13.4 Hz), 3.57 (d, 1H, J=14.2 Hz), 3.18 (d, 1H, J=14.2 Hz), 3.12 (td, 1H, J=13.3, 2.4 Hz), 2.73 (dddd, 1H, J=11.9, 11.9, 3.8, 3.8 Hz), 2.65 (ddd, 1H, J=13.3, 13.3, 2.4 Hz), 2.40 (s, 6H), 2.18-2.32 (m, 4H), 1.60-1.98 (m, 8H). Mass spectrum (ESI, m/z): Calcd. for C₂₆H₃₂N₆O₂, 461.3 (M+H), found 461.2.

### Example 38b

### 4-Cyano-1H-imidazole-2-carboxylic acid {2-cyclohex-1-enyl-4-[1-(2-methylamino-acetyl)-piperidin-4-yl]-phenyl}-amide

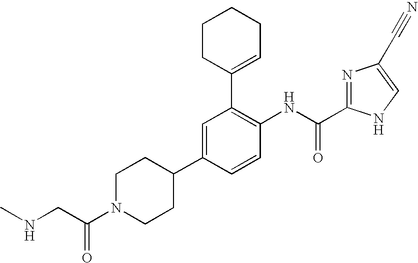

HPLC purification of Example 38a also afforded a small amount of 4-cyano-1H-imidazole-2-carboxylic acid {2-cyclohex-1-enyl-4-[1-(2-methylamino-acetyl)-piperidin-4-yl]-phenyl}-amide. ¹H-NMR (CD₃OD; 400 MHz): δ 8.02 (d, 1H, J=8.4 Hz), 7.92 (s, 1H), 7.07 (dd, 1H, J=8.4 Hz, J=2.4 Hz), 6.98 (d, 1H, J=2.4 Hz), 5.73-5.68 (m, 1H), 4.60-4.51 (m, 1H), 3.76-3.68 (m, 1H), 3.20-3.11 (m, 1H), 2.81-2.70 (m, 2H), 2.67 (s, 3H), 2.22-2.13 (m, 4H), 1.88-1.66 (m, 6H), 1.66-1.46 (m, 2H). Mass spectrum (ESI, m/z): Calcd. for C₂₅H₃₀N₆O₂, 447.2 (M+H), found 447.3.

### Example 39

### 4-{4-[(4-Cyano-1H-imidazole-2-carbonyl)-amino]-3-cyclohex-1-enyl-phenyl}-piperidine-1-carboxylic acid (2-hydroxy-ethyl)-amide

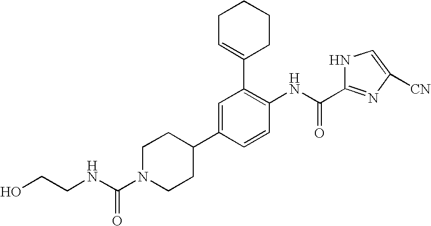

### a) 4-Cyano-1-(2-trimethylsilanyl-ethoxymethyl)-1H-imidazole-2-carboxylic acid (2-cyclohex-1-enyl-4-piperidin-4-yl-phenyl)-amide, trifluoroacetic acid salt

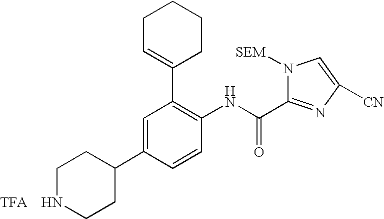

To a solution of 4-(4-{[4-cyano-1-(2-trimethylsilanyl-ethoxymethyl)-1H-imidazole-2-carbonyl]-amino}-3-cyclohex-1-enyl-phenyl)-piperidine-1-carboxylic acid tert-butyl ester (as prepared in Example 14, step (a), 81 mg, 0.123 mmol) in 18 mL of DCM was added 1 mL of EtOH followed by 5 mL of TFA at 0° C. The resulting solution was stirred at RT for 0.5 h, treated with 20 mL of EtOH followed by 20 mL of n-PrOH and 5 mL of H₂O, the mixture was then concentrated under reduced pressure to give a slightly yellow solid. Flash chromatography of the compound on silica gel (2-4% MeOH/DCM) gave 0.87 g (85%) of the title compound as a white solid. ¹H-NMR (CDCl₃; 400 MHz): δ 9.70 (s, 1H), 9.66 (br s, 1H), 9.15 (br s, 1H), 8.29 (d, 1H, J=8.3 Hz), 7.78 (s, 1H), 7.13 (dd, 1H, J=8.3, 2.2 Hz), 7.03 (d, 1H, J=2.2 Hz), 5.95 (s, 2H), 5.83 (m, 1H), 3.66 (t, 2H, J=8.4 Hz), 3.55 (d, 2H, J=12.3 Hz), 2.95-3.11 (m, 2H), 2.76 (m, 1H), 2.18-2.33 (m, 4H), 1.99-2.15 (m, 4H), 1.82 (m, 4H), 0.97 (t, 2H, J=8.3 Hz), 0.00 (s, 9H). Mass spectrum (ESI, m/z): Calcd. for C₂₈H₃₉N₅O₂Si, 506.3 (M+H), found 506.1.

### b) 4-(4-{[4-Cyano-1-(2-trimethylsilanyl-ethoxymethyl)-1H-imidazole-2-carbonyl]-amino}-3-cyclohex-1-enyl-phenyl)-piperidine-1-carboxylic acid (2-hydroxy-ethyl)-amide

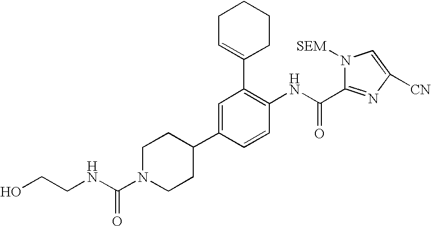

A solution of 4-cyano-1-(2-trimethylsilanyl-ethoxymethyl)-1H-imidazole-2-carboxylic acid (2-cyclohex-1-enyl-4-piperidin-4-yl-phenyl)-amide trifluoroacetic acid salt (as prepared in the previous step, 116 mg, 0.192 mmol) and DIEA (134 μL, 0.770 mmol) in 4 mL of DCM was added slowly to solution of triphosgene (23 mg, 0.0768 mmol) in 4 mL of DCM at −78° C. under Ar. The mixture was stirred at −78° C. for 15 min, warmed to RT and stirred for 15 min and cooled to −78° C. again. A suspension of 2-amino-ethanol (350 μL, 5.77 mmol) in 4 mL of THF was added and the resulting mixture was warmed to RT and stirred for 20 h under Ar. Treated with 100 mL of EtOAc, the mixture was washed with H₂O (3×20 mL), brine (20 mL) and dried (Na₂SO₄). Removal of the solvent in vacuo followed by flash chromatography of the residue on silica gel (10% EtOAc/DCM then 5% MeOH/DCM) gave 95 mg (83%) of the title compound as a colorless oil. ¹H-NMR (CDCl₃; 400 MHz): δ 9.68 (s, 1H), 8.25 (d, 1H, J=8.4 Hz), 7.77 (s, 1H), 7.12 (dd, 1H, J=8.4, 2.2 Hz), 7.01 (d, 1H, J=2.2 Hz), 5.94 (s, 2H), 5.83 (m, 1H), 4.96 (t, 1H, J=5.6 Hz), 4.11 (d, 2H, J=13.3 Hz), 3.75 (ddd, 2H, J=4.4 Hz), 3.66 (t, 2H, J=8.3 Hz), 3.44 (ddd, 2H, J=5.0 Hz), 3.36 (t, 1H, J=4.6 Hz), 2.91 (ddd, 2H, J=13.0, 2.2 Hz), 2.66 (dddd, 1H, J=12.2, 12.2, 3.3, 3.3 Hz), 2.18-2.33 (m, 4H), 1.75-1.91 (m, 6H), 1.67 (dddd, 2H, J=12.9, 12.9, 12.9, 4.0 Hz), 0.97 (t, 2H, J=8.3 Hz), 0.00 (s, 9H). Mass spectrum (ESI, m/z): Calcd. for C₃₁H₄₄N₆O₄Si, 593.3 (M+H), found 593.1.

### c) 4-{4-[(4-Cyano-1H-imidazole-2-carbonyl)-amino]-3-cyclohex-1-enyl-phenyl}-piperidine-1-carboxylic acid (2-hydroxy-ethyl)-amide

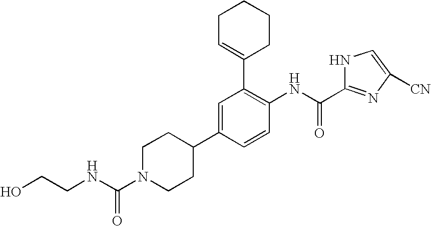

To a solution of 4-(4-{[4-cyano-1-(2-trimethylsilanyl-ethoxymethyl)-1H-imidazole-2-carbonyl]-amino}-3-cyclohex-1-enyl-phenyl)-piperidine-1-carboxylic acid (2-hydroxy-ethyl)-amide (as prepared in the previous step, 95 mg, 0.16 mmol) in 3 mL of DCM was added 0.10 mL of EtOH followed by 1.0 mL of TFA. The resulting solution was stirred at RT for 6 h. Removal of the solvent under reduced pressure followed by flash chromatography of the residue on silica gel (2-8% MeOH/DCM) gave 68 mg (92%) of the title compound as a white solid. ¹H-NMR (CD₃OD; 400 MHz): δ 8.09 (d, 1H, J=8.4 Hz), 8.00 (s, 1H), 7.15 (dd, 1H, J=8.4, 2.2 Hz), 5.79 (m, 1H), 4.15 (dd, 2H, J=13.3, 1.1 Hz), 3.61 (t, 2H, J=5.9 Hz), 3.27-3.32 (m, 2H), 2.90 (ddd, 2H, J=13.0, 13.0, 2.5 Hz), 2.73 (dddd, 1H, J=12.1, 12.1, 2.6, 2.6 Hz), 2.26 (m, 4H), 1.73-1.88 (m, 6H), 1.62 (dddd, 2H, J=12.6, 12.6, 12.6, 4.0 Hz). Mass spectrum (ESI, m/z): Calcd. for C₂₅H₃₀N₆O₃, 463.2 (M+H), found 463.2.

### Example 40

### 4-Cyano-1H-imidazole-2-carboxylic acid {2-cyclohex-1-enyl-4-[1-(2-methanesulfonyl-ethyl)-piperidin-4-yl]-phenyl}-amide

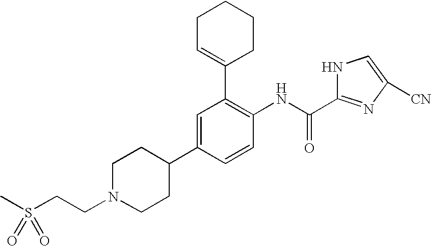

### a) Methanesulfonic acid 2-methanesulfonyl-ethyl ester

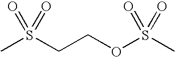

To a solution of methanesulfonyl chloride (484 mg, 4.23 mmol) in 15 mL of DCM at 0° C. was added 2-methanesulfonyl-ethanol (500 mg, 4.03 mmol) in 10 mL of DCM followed by DIEA (1.05 mL, 6.05 mmol) under Ar. The mixture was warmed to RT and stirred for 20 h under Ar. The mixture was treated with 100 mL of EtOAc and washed with H₂O (3×20 mL), brine (20 mL) and dried (Na₂SO₄). Removal of the solvent in vacuo gave 534 mg (66%) of the title compound as a brown oil. ¹H-NMR (CDCl₃; 400 MHz): δ 4.67 (d, 2H, J=5.5 Hz), 3.46 (d, 2H, J=5.5 Hz), 3.11 (s, 3H), 3.04 (s, 3H).

### b) 4-Cyano-1H-imidazole-2-carboxylic acid {2-cyclohex-1-enyl-4-[1-(2-methanesulfonyl-ethyl)-piperidin-4-yl]-phenyl}-amide

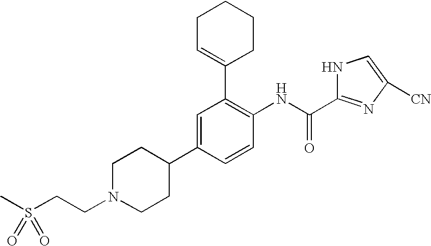

To a solution of 4-cyano-1H-imidazole-2-carboxylic acid (2-cyclohex-1-enyl-4-piperidin-4-yl-phenyl)-amide trifluoroacetic acid salt (as prepared in Example 14, step (b), 85 mg, 0.174 mmol) and DIEA (91 μL, 0.521 mmol) in 3 mL of DCM at RT was added 2-methanesulfonic acid 2-methanesulfonyl-ethyl ester (as prepared in the previous step, 42 mg, 0.208 mmol). The resulting mixture was stirred at RT for 3 h. Treated with 50 mL of EtOAc, the mixture was washed with H₂O (2×20 mL), brine (10 mL) and dried (Na₂SO₄). Removal of the solvent in vacuo followed by flash chromatography of the residue on silica gel (1-3% MeOH/DCM) gave 54 mg (65%) of the title compound as a white solid. ¹H-NMR (CDCl₃; 400 MHz): δ 9.54 (s, 1H), 8.25 (d, 1H, J=8.4 Hz), 7.72 (s, 1H), 7.15 (dd, 1H, J=8.4, 2.0 Hz), 7.04 (d, 1H, J=2.0 Hz), 5.85 (m, 1H), 3.21 (t, 1H, J=6.5 Hz), 3.09 (s, 3H), 3.02-3.11 (m, 2H), 2.92 (t, 2H, J=6.5 Hz), 2.52 (dddd, 1H, J=12.1, 12.1, 3.3, 3.3 Hz), 2.18-2.34 (m, 4H), 2.18 (t, 2H, J=10.8 Hz), 1.64-1.94 (m, 8H). Mass spectrum (ESI, m/z): Calcd. for C₂₅H₃₁N₅O₃S, 482.2 (M+H), found 482.2.

The following compounds have been prepared according to the examples as indicated:

| | | Mass | | | |
|---|---|---|---|---|---|
| | | Spectrum | | | |
| Example | Structure | [M + H]⁺Calcd. | Found | Formula | Proc. Of Ex |
| 41 | 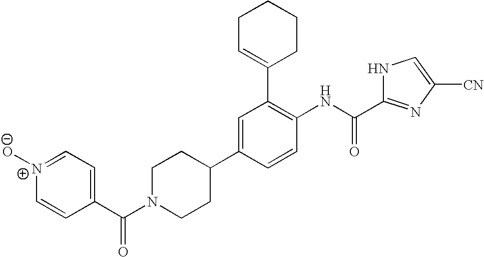 | 497.2 | 497.2 | C₂₈H₂₈N₆O₃ | 29 |
| 42 | 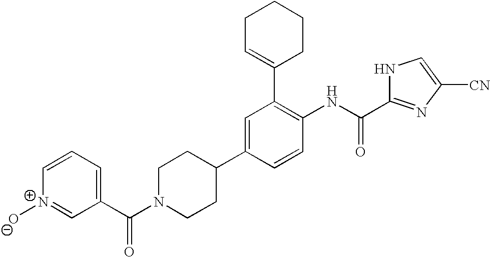 | 497.2 | 497.3 | C₂₈H₂₈N₆O₃ | 29 |

### Example 43

### 4-Cyano-1H-imidazole-2-carboxylic acid{2-cyclohex-1-enyl-4-[1-(pyridine-3-carbonyl)-piperidin-4-yl]-phenyl}-amide

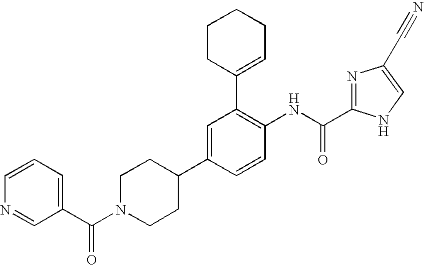

A solution of 4-cyano-1H-imidazole-2-carboxylic acid (2-cyclohex-1-enyl-4-piperidin-4-yl-phenyl)-amide trifluoroacetic acid salt (as prepared in Example 14, step (b), 75.0 mg, 0.15 mmol) in CH₂Cl₂ (10 mL) was treated with Et₃N (64.1 μL, 0.46 mmol) and cooled to 0° C. The mixture was treated with nicotinoyl chloride hydrochloride (0.030 g, 0.17 mmol) and stirred at 0° C. for 15 min then at room temperature for 17 h. The reaction mixture was adsorbed directly onto silica gel. Silica gel chromatography (10% MeOH in EtOAc) afforded the title compound (61.0 mg, 83%) as a white solid. ¹H-NMR (CDCl₃; 400 MHz): δ 9.51 (br s, 1H), 8.77 (s, 1H), 8.70-8.66 (m, 1H), 8.32 (d, 1H, J=8.4 Hz), 7.86-7.81 (m, 1H), 7.70 (s, 1H), 7.42-7.37 (m, 1H), 7.17 (d, 1H, J=8.4 Hz), 7.06-7.04 (m, 1H), 5.87-5.82 (m, 1H), 4.98-4.87 (m, 1H), 3.94-3.84 (m, 1H), 3.29-3.18 (m, 1H), 2.98-2.86 (m, 1H), 2.86-2.76 (m, 1H), 2.34-2.20 (m, 4H), 1.94-1.72 (m, 9H). LC-MS (ESI, m/z): Calcd. for C₂₈H₂₈N₆O₂, 481.2 (M+H), found 481.3.

### Example 44

### 4-Cyano-1H-imidazole-2-carboxylic acid (2-cyclohex-1-enyl-4-{1-[2-(2-hydroxy-ethylamino)-acetyl]-piperidin-4-yl}-phenyl)-amide trifluoroacetic acid salt

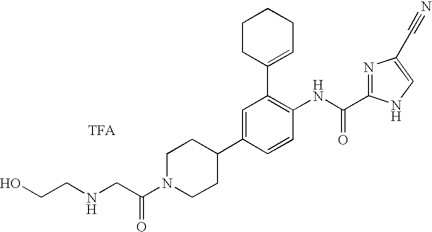

### a) [2-(4-{4-[(4-Cyano-1H-imidazole-2-carbonyl)-amino]-3-cyclohex-1-enyl-phenyl}-piperidin-1-yl)-2-oxo-ethyl]-carbamic acid tert-butyl ester

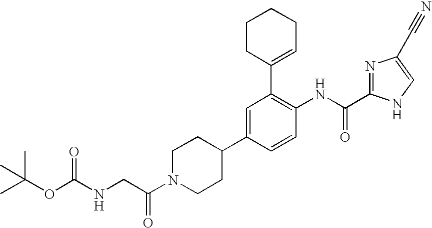

A solution of N—BOC-glycine (0.29 g, 1.63 mmol) in CH₂Cl₂ (10 mL) was treated with DIEA (0.85 mL, 4.90 mmol), HOBt (0.26 g, 1.96 mmol), and EDCI (0.38 g, 1.96 mmol). The mixture was stirred at room temperature for 10 min and added to a suspension of 4-cyano-1H-imidazole-2-carboxylic acid (2-cyclohex-1-enyl-4-piperidin-4-yl-phenyl)-amide trifluoroacetic acid salt (as prepared in Example 14, step (b), 0.80 g, 1.63 mmol) in CH₂Cl₂ (20 mL). The solution was stirred at room temperature for 17 h. Solvents were evaporated in vacuo. Silica gel chromatography (50% EtOAc in hexanes) afforded the title compound (0.41 g, 47%) as a white solid. ¹H-NMR (CDCl₃; 400 MHz): δ 9.53 (s, 1H), 8.26 (d, 1H, J=8.4 Hz), 7.80-7.78 (m, 1H), 7.71 (s, 1H), 7.45-7.43 (m, 1H), 7.06 (d, 1H, J=8.4 Hz), 7.00 (s, 1H), 5.83 (br s, 1H), 5.76 (br s, 1H), 4.78-4.68 (m, 1H), 3.96-3.85 (m, 2H), 3.17-3.03 (m, 1H), 2.78-2.63 (m, 2H), 2.29 (br s, 2H), 2.22 (br s, 2H), 1.95-1.87 (m, 2H), 1.86-1.72 (m, 4H), 1.70-1.55 (m, 2H), 1.44 (s, 9H). LC-MS (ESI, m/z): Calcd. for C₂₉H₃₆N₆O₄ 533.3 (M+H), found 532.9.

### b) 4-Cyano-1H-imidazole-2-carboxylic acid {4-[1-(2-amino-acetyl)-piperidin-4-yl]-2-cyclohex-1-enyl-phenyl]-amide trifluoroacetic acid salt

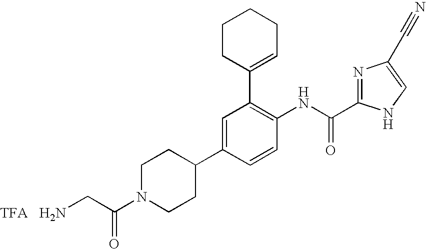

A solution of [2-(4-{4-[(4-cyano-1H-imidazole-2-carbonyl)-amino]-3-cyclohex-1-enyl-phenyl}-piperidin-1-yl)-2-oxo-ethyl]-carbamic acid tert-butyl ester (as prepared in the previous step, 0.41 g, 0.77 mmol) in CH₂Cl₂ (20 mL) was treated with EtOH (0.2 mL) and TFA (6 mL). The mixture stirred at room temperature for 45 min, and the solvents were evaporated in vacuo. The crude material was used directly in the next step. LC-MS (ESI, m/z): Calcd. for C₂₄H₂₈N₆O₂ 433.2 (M+H), found 433.2.

### c) 4-Cyano-1H-imidazole-2-carboxylic acid (2-cyclohex-1-enyl-4-{1-[2-(2-hydroxy-ethylamino)-acetyl]-piperidin-4-yl}-phenyl)-amide trifluoroacetic acid salt

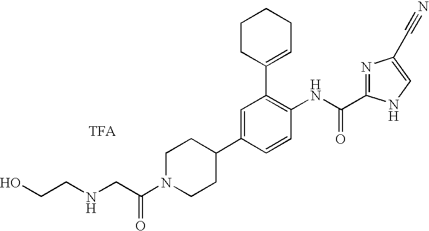

A suspension of 4-cyano-1H-imidazole-2-carboxylic acid {4-[1-(2-amino-acetyl)-piperidin-4-yl]-2-cyclohex-1-enyl-phenyl]-amide trifluoroacetic acid salt (as prepared in the previous step, 0.42 g, 0.77 mmol) in CH₂Cl₂ (20 mL) was treated with Na(OAc)₃BH (0.33 g, 1.54 mmol) and solid glyoxal (44.6 mg, 0.77 mmol). The mixture stirred at room temperature for 1 h, and the solvent was evaporated in vacuo. The residue was taken up in MeOH and the solids filtered off, and the filtrate was concentrated in vacuo. Reverse phase HPLC (C-18 column) (20% to 60% acetonitrile in water with 0.1% TFA over 30 min) afforded the title compound (83 mg, 19% over two steps) as a white solid. ¹H-NMR (CD₃OD; 400 MHz): δ 8.16-8.09 (m, 1H), 8.05-8.01 (m, 1H), 7.22-7.15 (m, 1H), 7.11-7.06 (m, 1H), 5.84-5.79 (m, 1H), 4.72-4.62 (m, 1H), 4.24-3.91 (m, 2H), 3.89-3.80 (m, 2H), 3.28-3.18 (m, 2H), 2.92-2.79 (m, 2H), 2.28 (br s, 4H), 1.98-1.89 (m, 2H), 1.89-1.76 (m, 4H), 1.76-1.57 (m, 2H). LC-MS (ESI, m/z): Calcd. for C₂₆H₃₂N₆O₃ 477.2 (M+H), found 477.2.

### Example 45

### 4-Cyano-1H-imidazole-2-carboxylic acid (2-cyclohex-1-enyl-4-{1-[2-(2-hydroxy-ethyl)-methyl-amino-acetyl]-piperidin-4-yl}-phenyl)-amide trifluoroacetic acid salt

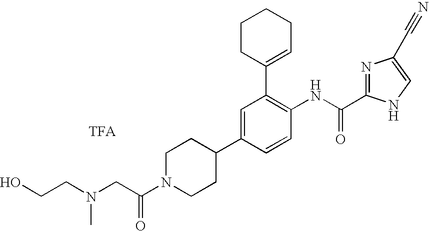

A solution of 4-cyano-1H-imidazole-2-carboxylic acid (2-cyclohex-1-enyl-4-{1-[2-(2-hydroxy-ethylamino)-acetyl]-piperidin-4-yl}-phenyl)-amide trifluoroacetic acid salt (as prepared in Example 44, step (c), 50.0 mg, 0.085 mmol) in MeOH (3 mL) was treated with Na(OAc)₃BH (39.5 mg, 0.19 mmol) and 37% aqueous formaldehyde (8.2 μL, 0.10 mmol). The mixture was stirred at room temperature for 5.5 h, and the solvents were removed in vacuo. Reverse phase HPLC (C-18 column) (10% to 50% acetonitrile in water with 0.1% TFA over 30 min) afforded the title compound (19.5 mg, 47%) as a white solid. ¹H-NMR (CD₃OD; 400 MHz): δ 8.12 (d, 1H, J=8.4 Hz), 8.02 (s, 1H), 7.19 (dd, 1H, J=8.4, 2.0 Hz), 7.09 (d, 1H, J=2.0 Hz), 5.84-5.79 (m, 1H), 4.72-4.64 (m, 1H), 4.39-4.23 (m, 2H), 3.84-3.79 (m, 1H), 3.31-3.21 (m, 1H), 3.03-2.94 (m, 6H), 2.92-2.80 (m, 2H), 2.32-2.24 (m, 4H), 2.00-1.90 (m, 2H), 1.90-1.76 (m, 5H), 1.78-1.59 (m, 2H). LC-MS (ESI, m/z): Calcd. for C₂₂H₃₄N₆O₃ 491.3 (M+H), found 491.2.

### Example 46

### 4-Cyano-1H-imidazole-2-carboxylic acid [4-(1-acetyl-piperidin-4-yl)-2-(1,2,5,6-tetrahydro-pyridin-3-yl)-phenyl]-amide trifluoroacetic acid salt

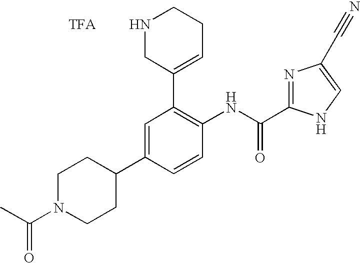

### a) 5-Trifluoromethanesulfonyloxy-3,6-dihydro-2H-pyridine-1-carboxylic acid tert-butyl ester

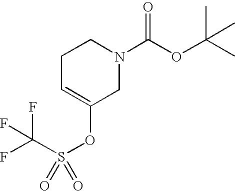

A solution of LDA (23.4 mL, 35.1 mmol, 1.5 M in cyclohex) in THF (50 mL) was cooled to −78° C. under Ar. The solution was treated with 3-oxo-piperidine-1-carboxylic acid tert-butyl ester (5.00 g, 25.1 mmol) as a solution in THF (15 mL) via drop wise addition and stirred for 15 min. The mixture was treated with 1,1,1-trifluoro-N-phenyl-N-[(trifluoromethyl)sulfonyl]methanesulfonimide (12.5 g, 35.1 mmol) as a solution in THF (40 mL). The mixture was allowed to warm to room temperature and stir 2.5 h. The reaction was quenched with saturated aqueous NaHCO₃, diluted with Et₂O, and washed with water. The organic layer was dried over MgSO₄ and concentrated in vacuo. Silica gel chromatography (5% EtOAc in hexanes) afforded the title compound (2.45 g, 30%) as a colorless oil. ¹H-NMR (CDCl₃; 400 MHz): δ 5.97-5.89 (m, 1H), 4.09-4.01 (m, 2H), 3.54-3.45 (m, 2H), 2.36-2.26 (m, 2H), 1.48 (s, 9H). LC-MS (ESI, m/z): Calcd. for C₁₁H₁₆F₃NO₅S 332.1 (M+H), found 332.1.

### b) 5-(4,4,5,5-Tetramethyl-[1,3,2]dioxaborolan-2-yl)-3,6-dihydro-2H-pyridine-1-carboxylic acid tert-butyl ester

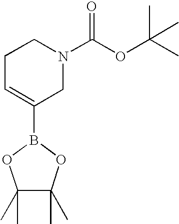

PdCl₂dppf (0.16 g, 0.22 mmol), KOAc (2.18 g, 22.2 mmol), 4,4,5,5,4′,4′,5′,5′-octamethyl-[2,2′]bi[[1,3,2]dioxaborolanyl] (2.07 g, 8.13 mmol), and dppf (0.12 g, 0.22 mmol) were placed in a round-bottomed flask, and the flask was flushed with Ar. A degassed solution of 5-trifluoromethanesulfonyloxy-3,6-dihydro-2H-pyridine-1-carboxylic acid tert-butyl ester (as prepared in the previous step, 2.45 g, 7.40 mmol) in dioxane (70 mL) was added to the flask and heated to 80° C. for 16 h. The mixture was filtered through a glass-fritted funnel to remove the solid KOAc, and the filtrate was concentrated in vacuo. Silica gel chromatography (5% EtOAc in hexanes) afforded the title compound (1.62 g, 71%) as a colorless oil. ¹H-NMR (CDCl₃; 400 MHz): δ 6.69-6.60 (m, 1H), 3.98 (br s, 2H), 3.49-3.42 (m, 2H), 2.24-2.16 (m, 2H), 1.47 (s, 9H), 1.27 (s, 12H). LC-MS (ESI, m/z): Calcd. for C₁₈H₂₈BNO₄ 310.2 (M+H), found 311.0.

### c) 4-(4-Nitro-phenyl)-3,6-dihydro-2H-pyridine-1-carboxylic acid tert-butyl ester

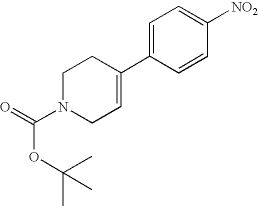

The title compound was prepared by the Suzuki coupling procedure of Example 35, step (b) using 4-nitrophenylboronic acid (167 mg, 1.00 mmol) and 4-trifluoromethanesulfonyloxy-3,6-dihydro-2H-pyridine-1-carboxylic acid tert-butyl ester (as prepared in Example 13, step (a), 295 mg, 1.00 mmol). Silica gel chromatography (10% EtOAc in hexanes) afforded the title compound (273 mg, 90%) as an oil. ¹H-NMR (CDCl₃; 400 MHz): δ 8.19 (d, 2H, J=8.8 Hz), 7.50 (d, 2H, J=8.8 Hz), 6.23 (m, 1H), 4.12 (m, 2H), 3.66 (m, 2H), 2.54 (m, 2H), 1.49 (s, 9H).

### d) 1-[4-(4-Amino-phenyl)-piperidin-1-yl]-ethanone

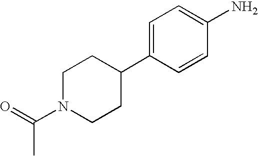

A solution of 4-(4-nitro-phenyl)-3,6-dihydro-2H-pyridine-1-carboxylic acid tert-butyl ester (as prepared in the previous step, 304 mg, 1.00 mmol) in a 1:1 mixture of DCM/TFA (10 mL) was stirred at room temperature for 3 h and concentrated. The residue was dried in vacuo overnight, was taken up in CH₂Cl₂ (10 mL) and was cooled to 0° C. To this solution, Et₃N (280 μL, 2 mmol) was added drop wise, followed by acetic anhydride (102 μL, 1 mmol). The resulting mixture was stirred at 0° C. for 1 h and allowed to warm to room temperature. The reaction mixture was washed with brine, and the organic layer was separated, dried and concentrated. The resulting product was reduced to obtain the title compound (143 mg, 65%) using a procedure similar to Example 4, step (d). ¹H-NMR (CDCl₃; 400 MHz): δ 6.97 (d, 2H, J=8.4 Hz), 6.64 (d, 2H, J=8.4 Hz), 4.75 (m, 1H), 3.93 (m, 1H), 3.13 (m, 3H), 2.66 (m, 2H), 2.12 (s, 3H), 1.84 (m, 2H), 1.57 (m, 2H).

### e) 1-[4-(4-Amino-3-bromo-phenyl)-piperidin-1-yl]-ethanone

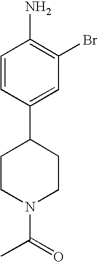

A solution of 1-[4-(4-amino-phenyl)-piperidin-1-yl]-ethanone (as prepared in the previous step, 0.36 g, 1.66 mmol) in CH₂Cl₂ (10 mL) was cooled to −78° C. and treated with NBS (0.28 g, 1.58 mmol) as a suspension in CH₂Cl₂ (4 mL). The reaction was allowed to warm to room temperature and stir for 30 min. The reaction was diluted with CH₂Cl₂ and washed with saturated aqueous NaHCO₃. The organic layer was dried over MgSO₄ and concentrated in vacuo. The crude material was used directly in the next reaction. LC-MS (ESI, m/z): Calcd. for C₁₃H₁₇BrN₂O 297.1 (M+H), found 297.1.

### f) 5-[5-(1-Acetyl-piperidin-4-yl)-2-amino-phenyl]-3,6-dihydro-2H-pyridine-1-carboxylic acid tert-butyl ester

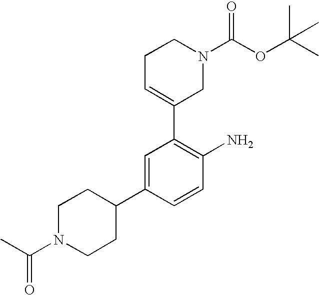

A solution of 5-(4,4,5,5-tetramethyl-[1,3,2]dioxaborolan-2-yl)-3,6-dihydro-2H-pyridine-1-carboxylic acid tert-butyl ester (as prepared in Example 46, step (b), 0.62 g, 2.02 mmol) and 1-[4-(4-amino-3-bromo-phenyl)-piperidin-1-yl]-ethanone (as prepared in the previous step, 0.20 g, 0.67 mmol) in toluene:EtOH (2:1, 9 mL) was treated with 2.0 M aqueous Na₂CO₃ (2.7 mL, 5.38 mmol) and was degassed with sonication under Ar. The mixture was heated to 80° C., treated with Pd(PPh₃)₄ (54 mg, 0.05 mmol), and stirred at 80° C. for 4.5 h. The reaction was cooled to room temperature, diluted with EtOAc, and washed with saturated aqueous NaHCO₃. The organic layer was dried over MgSO₄ and concentrated in vacuo to afford the title compound (0.25 g, 93%) as an off-white solid. LC-MS (ESI, m/z): Calcd. for C₂₃H₃₃N₃O₃ 422.2 (M+Na), found 422.0.

### g) 5-(5-(1-Acetyl-piperidin-4-yl)-2-{[4-cyano-1-(2-trimethylsilanyl-ethoxymethyl)-1H-imidazole-2-carbonyl]-amino}-phenyl)-3,6-dihydro-2H-pyridine-1-carboxylic acid tert-butyl ester

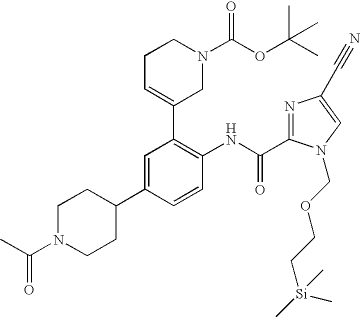

A solution of 5-[5-(1-acetyl-piperidin-4-yl)-2-amino-phenyl]-3,6-dihydro-2H-pyridine-1-carboxylic acid tert-butyl ester (as prepared in the previous step, 0.25 g, 0.63 mmol) in CH₂Cl₂ was treated with PyBroP (0.44 g, 0.94 mmol) and 4-cyano-1-(2-trimethylsilanyl-ethoxymethyl)-1H-imidazole-2-carboxylic acid, potassium salt (as prepared in Example 3, step (d), 0.21 g, 0.69 mmol). The resulting slurry was cooled to 0° C. and treated with DIEA (0.33 mL, 1.88 mmol). The ice bath was removed and the mixture stirred at room temperature for 18 h. The reaction was diluted with CH₂Cl₂ and washed with saturated aqueous NaHCO₃. The organic layer was dried over MgSO₄ and concentrated in vacuo. Silica gel chromatography (25-45% EtOAc in hexanes then 100% EtOAc) afforded the title compound (399 mg, 98%) as a white solid. LC-MS (ESI, m/z): Calcd. for C₃₄H₄₈N₆O₅Si 649.4 (M+H), found 649.9.

### h) 4-Cyano-1H-imizazole-2-carboxylic acid [4-(1-acetyl-piperidin-4-yl)-2-(1,2,5,6-tetrahydro-pyridin-3-yl)-phenyl]-amide trifluoroacetic acid salt

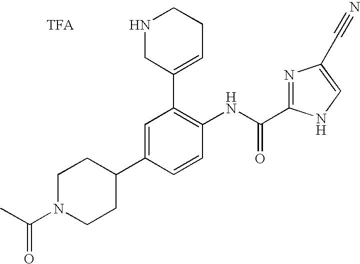

A solution of 5-(5-(1-acetyl-piperidin-4-yl)-2-{[4-cyano-1-(2-trimethylsilanyl-ethoxymethyl)-1H-imidazole-2-carbonyl]-amino}-phenyl)-3,6-dihydro-2H-pyridine-1-carboxylic acid tert-butyl ester (as prepared in the previous step, 0.40 g, 0.61 mmol) in CH₂Cl₂ (20 mL) and EtOH (0.4 mL) was treated with TFA (3 mL). The solution was stirred at room temperature for 0.5 h. The solvents were evaporated in vacuo, and the residue was immediately taken up in EtOH (25 mL) and stored at 5° C. for 11 h. The solution was concentrated in vacuo, and the residue was taken up in CH₂Cl₂ (20 mL) and EtOH (0.4 mL) then treated with TFA (6 mL). The reaction was stirred at room temperature for 2 h, and the solvents were evaporated in vacuo. Reverse phase HPLC (C-18 column) (10 to 80% acetonitrile in water with 0.1% TFA over 30 min) afforded the title compound (56.9 mg, 22%) as a white solid. ¹H-NMR (CDCl₃; 400 MHz): δ 8.06 (s, 1H), 7.81 (d, 1H, J=8.4 Hz), 7.32 (d, 1H, J=8.4 Hz), 7.22 (s, 1H), 6.10-6.03 (m, 1H), 4.74-4.64 (m, 2H), 4.11-4.02 (m, 1H), 3.95 (s, 2H), 3.50-3.37 (m, 2H), 3.29-3.20 (m, 1H), 2.93-2.82 (m, 1H), 2.80-2.69 (m, 1H), 2.62-2.53 (m, 2H), 2.16 (s, 3H), 1.98-1.84 (m, 2H), 1.78-1.54 (m, 2H). LC-MS (ESI, m/z): Calcd. for C₂₃H₂₆N₆O₂ 419.2 (M+H), found 419.2.

### Example 47

(4-{4-[(4-Cyano-1H-imidazole-2-carbonyl)-amino]-3-cyclohex-1-enyl-phenyl}-piperidin-1-yl)-acetic acid trifluoroacetic acid salt

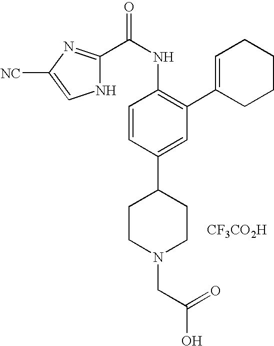

A flask was charged with 4-cyano-1H-imidazole-2-carboxylic acid (2-cyclohex-1-enyl-4-piperidin-4-yl-phenyl)-amide TFA salt (33 mg, 0.067 mmol) (as prepared in Example 14, step (b)), t-butyl bromoacetate (10 μL, 0.067 mmol), NEt₃ (20 μL, 0.135 mmol) and 0.25 mL of DCM and stirred for 10 h at 25° C. The reaction mixture was loaded on a 5 g SPE cartridge (silica) and 23 mg (70%) of (4-{4-[(4-cyano-1H-imidazole-2-carbonyl)-amino]-3-cyclohex-1-enyl-phenyl}-piperidin-1-yl)-acetic acid tert-butyl ester was eluted with 25% EtOAc/DCM. This compound was dissolved in 1 mL of DCM and 20 μL of EtOH and 1 mL of TFA were added and the reaction stirred for 3 h at 25° C. The title compound was purified by RP-HPLC (C18), eluting with 30-50% CH₃CN in 0.1% TFA/H₂O over 12 min to give 10 mg (40%) of a white solid. ¹H-NMR (400 MHz, CD₃OD): δ 8.16 (d, 1H), 8.02 (s, 1H), 7.22 (dd, 1H), 7.10 (d, 1H), 5.72 (m, 1H), 4.04 (s, 2H), 3.76 (m, 2H), 3.22 (m, 2H), 2.90 (m, 1H), 2.29 (m, 4H), 2.10 (m, 4H), 1.82 (m, 4H). Mass spectrum (ESI, m/z): Calcd. for C₂₄H₂₇N₅O₃, 434.2 (M+H), found 434.2.

### Example 48

### 4-Cyano-1H-imidazole-2-carboxylic acid {4-[1-(3-amino-3-methyl-butyryl)-piperidin-4-yl]-2-cyclohex-1-enyl-phenyl}-amide trifluoroacetic acid salt

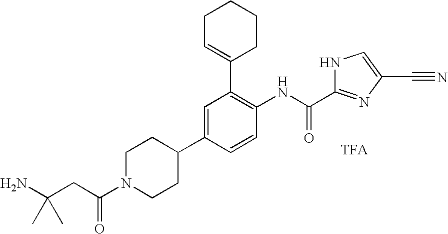

### a) [3-(4-{4-[(4-Cyano-1H-imidazole-2-carbonyl)-amino]-3-cyclohex-1-enyl-phenyl}-piperidin-1-yl)-1,1-dimethyl-3-oxo-propyl]-carbamic acid tert-butyl ester

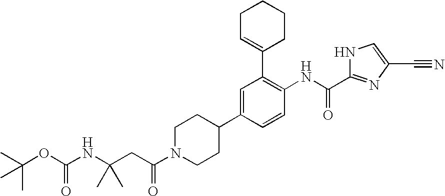

To a mixture of 4-cyano-1H-imidazole-2-carboxylic acid (2-cyclohex-1-enyl-4-piperidin-4-yl-phenyl)-amide trifluoroacetic acid salt (as prepared in Example 14, step (b), 40.0 mg, 0.0818 mmol), 3-tert-butoxycarbonylamino-3-methyl-butyric acid (J. Med. Chem., 34(2), 633-642, (1991), 21.4 mg, 0.0981 mmol) and PyBroP (55.0 mg, 0.0981 mmol) in dichloroethane (2 mL) was added DIEA (43 μL, 0.25 mmol) and the resulting mixture was stirred at RT for 1 day under Ar. The mixture was diluted with EtOAc (30 mL) and washed with H₂O (2×10 mL), brine (10 mL), dried over Na₂SO₄ and then concentrated in vacuo. The residue was purified by flash chromatography (silica gel, 10-40% EtOAc/hexane) to give 33.0 mg (70%) of the title compound as a colorless oil. Mass spectrum (ESI, m/z): Calcd. for C₃₂H₄₂N₆O₄, 575.3 (M+H), found 574.8.

### b) 4-Cyano-1H-imidazole-2-carboxylic acid {4-[1-(3-amino-3-methyl-butyryl)-piperidin-4-yl]-2-cyclohex-1-enyl-phenyl}-amide trifluoroacetic acid salt

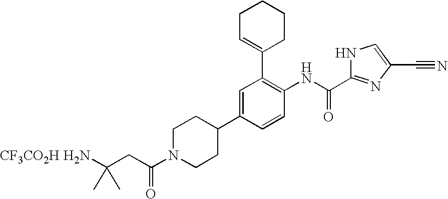

To a solution of [3-(4-{4-[(4-cyano-1H-imidazole-2-carbonyl)-amino]-3-cyclohex-1-enyl-phenyl}-piperidin-1-yl)-1,1-dimethyl-3-oxo-propyl]-carbamic acid tert-butyl ester (33.0 mg, 0.0574 mmol) (as prepared in the previous step) in 3 mL of DCM and 0.10 mL EtOH at 0° C. was added 1.0 mL of TFA, the mixture was warmed to RT and stirred for 3 h. The reaction was diluted with 3 mL of n-PrOH and then concentrated in vacuo. The residue was purified by flash chromatography (silica gel, 3-8% MeOH/DCM) to give 33.5 mg (99%) of the title compound as a white solid. ¹H-NMR (400 MHz, CDCl₃): δ 13.3 (s, 1H), 9.52 (s, 1H), 8.57 (br s, 3H), 8.26 (d, 1H, J=8.6 Hz), 7.69 (s, 1H), 7.02 (dd, 1H, J=8.6, 1.7 Hz), 6.98 (d, 1H, J=1.7 Hz), 5.78 (m, 1H), 4.67 (br d, 1H, J=13.4 Hz), 3.88 (br d, 1H, J=13.4 Hz), 3.10 (m, 1H), 2.55-2.85 (m, 4H), 2.23 (m, 4H), 1.72-2.01 (m, 8H), 1.50 (s, 6H). Mass spectrum (ESI, m/z): Calcd. for C₂₇H₃₄N₆O₂, 475.3 (M+H), found 475.1.

### Example 49

### 4H-[1,2,4]-triazole-3-carboxylic acid (2-cyclohex-1-enyl-4-piperidin-4-yl-phenyl)-amide bis trifluoroacetic acid salt

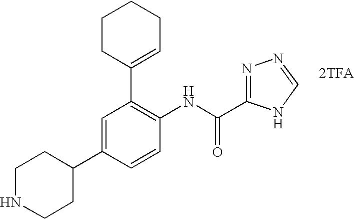

### a) 1-(2-Trimethylsilanyl-ethoxymethyl)-1H-[1,2,4]-triazole-3-carboxylic acid methyl ester

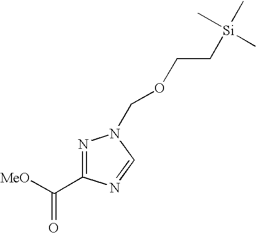

To a suspension of NaH (60% dispersion) (200 mg, 5.00 mmol) in DMF (5 mL) at 0° C., a solution of methyl-1H-1,2,4-triazolecarboxylate (635 mg, 5.00 mmol) in DMF (5 mL) was added dropwise. The resulting suspension was stirred at the same temperature for 30 min and treated with SEMCl (0.90 mL, 5.0 mmol). The resulting solution was stirred at RT for 30 min and poured onto ice. The product was extracted with ether (3×20 mL). The ether layers were combined, dried (Na₂SO₄) and concentrated in vacuo. The residue obtained was chromatographed on silica (10% EtOAc/hexane) to obtain the title compound (530 mg, 41%). Mass spectrum (ESI, m/z): Calcd. for C₁₀H₁₉N₃O₃Si, 258.1 (M+H), found 258.2.

### b) 4-(3-Cyclohex-1-enyl-4-{[1-(2-trimethylsilanyl-ethoxymethyl)-1H-[1,2,4]triazole-3-carbonyl]-amino}-phenyl)-piperidine-1-carboxylic acid tert-butyl ester

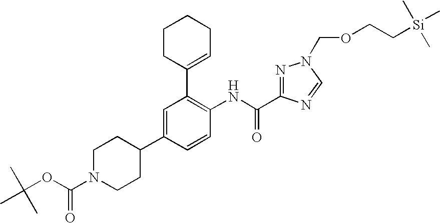

To a solution of 1-(2-trimethylsilanyl-ethoxymethyl)-1H-[1,2,4]-triazole-3-carboxylic acid methyl ester (as prepared in the previous step, 257 mg, 1.00 mmol) in EtOH (2 mL), 2 N KOH (0.5 mL, 1 mmol) was added. The resulting solution was stirred at RT for 20 min and concentrated in vacuo. The residue obtained was suspended in ether (10 mL) and sonicated for 5 min. The ether was then removed in vacuo and the resulting residue was dried for 4 hr to obtain 1-(2-trimethylsilanyl-ethoxymethyl)-1H-[1,2,4]-triazole-3-carboxylic acid potassium salt (273 mg, 97%) which was directly used in the next step without any further purification.

A mixture of 1-(2-trimethylsilanyl-ethoxymethyl)-1H-[1,2,4]-triazole-3-carboxylic acid potassium salt (as prepared above, 28 mg, 0.10 mmol), DIEA (34 μL, 0.20 mmol), 4-(4-amino-3-cyclohex-1-enyl-phenyl)-piperidine-1-carboxylic acid tert-butyl ester (as prepared in Example 14, step (b), 35.6 mg, 0.100 mmol) and PyBroP (69.9 mg, 0.150 mmol) in DCM (2 mL) was stirred at RT for 12 h. The reaction mixture was diluted with DCM (5 mL) and washed with saturated aqueous NaHCO₃ (10 mL) and water (10 mL). The organic layer was separated, dried (Na₂SO₄) and concentrated in vacuo. The product was chromatographed on silica (20-40% EtOAc/hexane) to obtain the title compound (31.9 mg, 55%). Mass spectrum (ESI, m/z): Calcd. for C₃₁H₄₇N₅O₄Si, 481.2 (M-BOC+2H), found. 481.2.

### c) 4H-[1,2,4]-triazole-3-carboxylic acid (2-cyclohex-1-enyl-4-piperidin-4-yl-phenyl)-amide bis trifluoroacetic acid salt

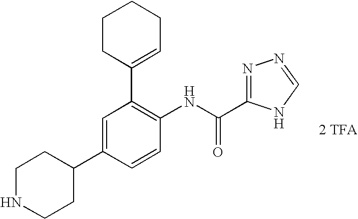

To a solution of 4-(3-cyclohex-1-enyl-4-{[1-(2-trimethylsilanyl-ethoxymethyl)-1H-[1,2,4]-triazole-3-carbonyl]-amino}-phenyl)-piperidine-1-carboxylic acid tert-butyl ester (as prepared in the previous step, 81.9 mg, 0.140 mmol) in DCM (0.4 mL) and EtOH (13 μL), was added TFA (0.13 mL). The resulting solution was stirred at RT for 3 h and concentrated in vacuo. The residue obtained was dried under vacuum for 1 h, suspended in ether (10 mL) and sonicated for 5 min. The solid formed was collected by suction filtration to obtain the title compound (56 mg, 68%). ¹H-NMR (CD₃OD; 400 MHz): δ 8.53 (br s, 1H), 8.20 (d, 1H, J=8.4 Hz), 7.21 (dd, 1H, J=8.4, 2.1 Hz), 7.11 (d, 1H, J=2.1 Hz), 5.83 (br s, 1H), 3.45 (m, 2H), 3.19 (m, 2H), 2.98 (m, 1H), 2.28 (m, 4H), 2.14 (m, 2H), and 1.95-1.75 (m, 6H). Mass spectrum (ESI, m/z): Calcd. for C₂₀H₂₅N₅O, 352.4 (M+H), found 352.2.

### Example 50

### 5-Chloro-4H-1,2,4′-triazole-3-carboxylic acid (2-cyclohex-1-enyl-4-piperidin-4-yl-phenyl)-amide trifluoroacetic acid salt

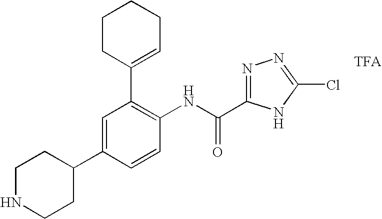

### a) 5-Chloro-1-(2-trimethylsilanyl-ethoxymethyl)-1H-[1,2,4]-triazole-3-carboxylic acid methyl ester

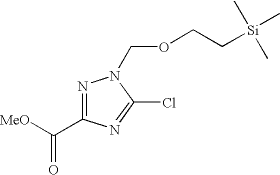

To a suspension of NaH (60% dispersion, 53.9 mg, 1.34 mmol) in DMF (5 mL) at 0° C., a solution of 5-chloro-1H-[1,2,4]-triazole-3-carboxylic acid methyl ester (Bull. Pharm. Sci., 20(1): 47-61, (1997), 218 mg, 1.35 mmol) in DMF (10 mL) was added dropwise. The resulting suspension was stirred at the same temperature for 30 min and then treated with SEMCl (0.24 mL, 1.4 mmol). The resulting solution was stirred at RT for 30 min and poured onto ice. The mixture was extracted with ether (3×20 mL) and the ether layers were combined, dried (Na₂SO₄) and concentrated in vacuo. The residue obtained was chromatographed on silica (10% EtOAc/hexane) to obtain the title compound (227 mg, 58%). Mass spectrum (ESI, m/z): Calcd. for C₁₀H₁₈ClN₃O₃Si, 292.0 and 294.0 (M+H), found 291.5 and 293.6.

### b) 4-(4-{[5-Chloro-1-(2-trimethylsilanyl-ethoxymethyl)-1H-[1,2,4]-triazole-3-carbonyl]-amino}-3-cyclohex-1-enyl-phenyl)-piperidine-1-carboxylic acid tert-butyl ester

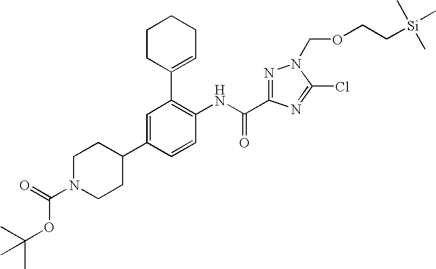

To a solution of 4-(4-{[5-chloro-1-(2-trimethylsilanyl-ethoxymethyl)-1H-[1,2,4]triazole-3-carboxylic acid methyl ester (as prepared in the previous step, 227 mg, 0.780 mmol) in EtOH (2 mL), 2 N KOH (0.4 mL, 0.8 mmol) was added. The resulting solution was stirred at RT for 20 min and concentrated in vacuo. The residue obtained was suspended in ether (10 mL) and sonicated for 5 min. The ether was then removed and the resulting residue was dried in vacuo for 4 h to obtain 4-(4-{[5-chloro-1-(2-trimethylsilanyl-ethoxymethyl)-1H-[1,2,4]triazole-3-carboxylic acid potassium salt (223 mg, 91%) which was directly used in the next step without any further purification.

A mixture of 4-(4-{[5-chloro-1-(2-trimethylsilanyl-ethoxymethyl)-1H-[1,2,4]-triazole-3-carboxylic acid potassium salt (as prepared above, 35 mg, 0.10 mmol), DIEA (34 μL, 0.10 mmol), 4-(4-amino-3-cyclohex-1-enyl-phenyl)-piperidine-1-carboxylic acid tert-butyl ester (as prepared in Example 14, step (b), 35.6 mg, 0.100 mmol) and PyBroP (69.9 mg, 0.150 mmol) in DCM (2 mL) was stirred at RT for 12 h. The reaction mixture was diluted with DCM (5 mL) and washed with saturated aqueous NaHCO₃ (10 mL) and water (10 mL). The organic layer was separated, dried (Na₂SO₄) and concentrated in vacuo. The product was chromatographed on silica (20-40% EtOAc/hexane) to obtain the title compound (52 mg, 85%). ¹H-NMR (CDCl₃; 400 MHz): δ 9.60 (s, 1H), 8.29 (d, 1H, J=8.4 Hz), 7.18 (dd, 1H, J=8.4, 2.2 Hz), 7.13 (d, 1H, J=2.2 Hz), 5.99 (s, 2H), 5.84 (br s, 1H), 4.18-4.25 (m, 2H), 3.72-3.76 (m, 2H), 2.58-2.67 (m, 2H), 2.51-2.64 (m, 1H), 2.18-2.33 (m, 4H), 1.78-1.92 (m, 6H), 1.55-1.65 (m, 2H), 1.49 (s, 9H), 0.93-0.98 (m, 2H), 0.10 (s, 9H).

### c) 5-Chloro-1H-[1,2,4]-triazole-3-carboxylic acid (2-cyclohex-1-enyl-4-piperidin-4-yl-phenyl)-amide trifluoroacetic acid salt

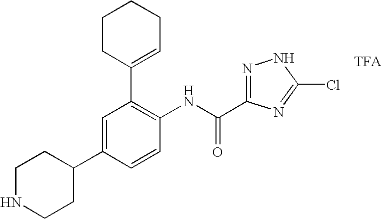

To a solution of 4-(4-{[5-chloro-1-(2-trimethylsilanyl-ethoxymethyl)-1H-[1,2,4]-triazole-3-carbonyl]-amino}-3-cyclohex-1-enyl-phenyl)-piperidine-1-carboxylic acid tert-butyl ester (as prepared in the previous step, 63.3 mg, 0.102 mmol) in DCM (0.5 mL) and EtOH (11 μL) was added TFA (0.1 mL). After stirring the resulting mixture at RT for 12 h, another 0.1 mL of TFA was added. The reaction mixture was stirred for an additional 5 h at RT, the solvents were evaporated, and the title compound was purified by RP-HPLC (C18) eluting with 20-70% CH₃CN in 0.1% TFA/H₂O over 20 min to obtain the title compound (30 mg, 58%). ¹H-NMR (CD₃OD; 400 MHz): δ 8.14 (d, 1H, J=8.4 Hz), 7.20 (dd, 1H, J=8.4, 2.1 Hz), 7.13 (d, 1H, J=2.1 Hz), 5.82 (br s, 1H), 3.45 (m, 2H), 3.19 (m, 2H), 2.98 (m, 1H), 2.28 (m, 4H), 2.14 (m, 2H), and 1.95-1.75 (m, 6H). Mass spectrum (ESI, m/z): Calcd. for C₂₀H₂₄ClN₅O, 386.1 and 388.1 (M+H), found 386.2 and 388.1.

### Example 51

### 5-Cyano-1H-imidazole-2-carboxylic acid [2-cyclohex-1-enyl-4-(cis-2,6-dimethyl-piperidin-4-yl)-phenyl]-amide bis trifluoroacetic acid salt, and 5-cyano-1H-imidazole-2-carboxylic acid [2-cyclohex-1-enyl-4-(trans-2,6-dimethyl-piperidin-4-yl)-phenyl]-amide bis trifluoroacetic acid salt

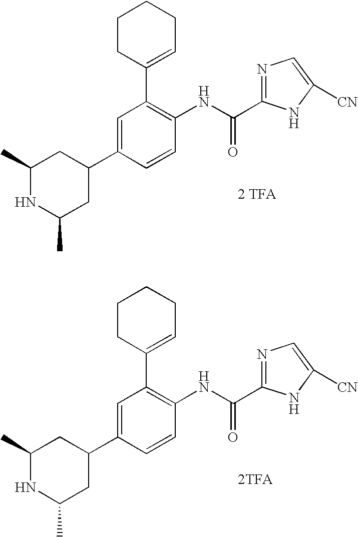

### a) Cis/trans 2,6-Dimethyl-4-oxo-piperidine-1-carboxylic acid tert-butyl ester

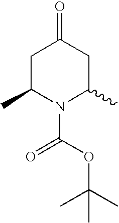

A solution of cis/trans-2,6-dimethylpiperidinone (Coll. Czech. Chem. Commun.: 31(11), 4432-41, (1966), 1.27 g, 10.0 mmol) in ether (100 mL) was treated with aq 1 N NaOH (11 mL, 11 mmol) and (BOC)₂O (2.18 g, 10.0 mmol). The resulting mixture as stirred at RT for 48 hr. The ether layer was separated, dried and concentrated. The residue was chromatographed on silica (10% EtOAc-hexane) to obtain the title compound (1.10 g, 50%): LC-MS (ESI, m/z): Calcd. for C₁₂H₂₁NO₃, 128.1 (M-BOC+2H), found 128.1.

### b) 4-(4-Amino-phenyl)-cis/trans 2,6-dimethyl-piperidine-1-carboxylic acid tert-butyl ester

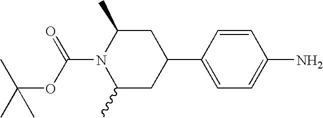

A solution of cis/trans N-Boc-2,6-dimethylpiperidinone (as prepared in the previous step, 1.14 g, 5.00 mmol) in THF (20 mL) was cooled to −78° C. and treated with LDA (1.5 M solution in cyclohex, THF and ethylbenzene, 4.4 mL, 6.5 mmol) under Ar. The resulting mixture was stirred at the same temperature for 30 min and treated with N-phenyltrifluoromethanesulfonimide (2.34 g, 6.55 mmol) in THF (20 mL). The reaction mixture was stirred for another 30 min and allowed to warm to RT. After 30 min. at RT the reaction mixture was concentrated in vacuo and the residue was taken up in ether (20 mL) and washed with cold water (2×10 mL). The ether layer was dried (Na₂SO₄) and concentrated to afforded cis/trans-2,6-dimethyl-4-trifluoromethanesulfonyloxy-3,6-dihydro-2H-pyridine-1-carboxylic acid tert-butyl ester (890 mg, 49%) which was directly used in next step.

The title compound was then prepared according to the Suzuki coupling procedure of Example 35, step (b) using 4-aminophenylboronic acid (219 mg, 1.00 mmol) and cis/trans-2,6-dimethyl-4-trifluoromethanesulfonyloxy-3,6-dihydro-2H-pyridine-1-carboxylic acid tert-butyl ester (as prepared above, 321 mg, 1.00 mmol). Silica gel chromatography (10-20% EtOAc/hexanes) afforded 4-(4-amino-phenyl)-2,6-dimethyl-3,6-dihydro-2H-pyridine-1-carboxylic acid tert-butyl ester (172 mg, 57%): Mass spectrum (ESI, m/z): Calcd. for C₁₈H₂₆N₂O₂, 303.2 (M+H) found 303.1. A solution of 4-(4-amino-phenyl)-2,6-dimethyl-3,6-dihydro-2H-pyridine-1-carboxylic acid tert-butyl ester (as prepared above, 380 mg, 1.25 mmol) in MeOH (10 mL) was hydrogenated over 10% Pd/C (190 mg) at 20 psi for 1 h. The solution was filtered through a pad of Celite and concentrated to give the title compound (360 mg, 94%). Mass spectrum (ESI, m/z): Calcd. for C₁₈H₂₈N₂O₂, 305.2 (M+H), found 305.6.

### c) 4-(4-Amino-3-cyclohex-1-enyl-phenyl)-cis/trans 2,6-dimethyl-piperidine-1-carboxylic acid tert-butyl ester

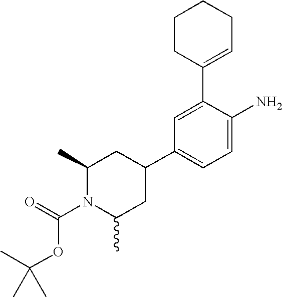

To a solution of 4-(4-amino-phenyl)-2,6-dimethyl-piperidine-1-carboxylic acid tert-butyl ester (as prepared in previous step, 334 mg, 1.09 mmol) in DCM (10 mL) was added NBS (195 mg, 1.09 mmol) and the reaction mixture was stirred at RT for 12 h. The reaction mixture was diluted with DCM (10 mL) and washed with saturated aqueous NaHCO₃ (10 mL) and water (10 mL). The organic layer was separated, dried (Na₂SO₄) and concentrated in vacuo to obtain 4-(4-amino-3-bromo-phenyl)-cis/trans-2,6-dimethyl-piperidine-1-carboxylic acid tert-butyl ester (367 mg, 87%). Mass spectrum (ESI, m/z): Calcd. for C₁₈H₂₂BrN₂O₂, 327.0 and 329.0 (M-t-Bu+H), found 327.0 and 328.9.

The title compound was then prepared according to the Suzuki coupling procedure of Example 12, step (d) using cyclohexan-1-enyl boronic acid (157 mg, 1.25 mmol) and 4-(4-amino-3-bromo-phenyl)-2,6-dimethyl-piperidine-1-carboxylic acid tert-butyl ester (as prepared above, 382 mg, 1.00 mmol) and chromatographed on silica (20% EtOAc/hexanes) to afford 254 mg (66%). Mass spectrum (ESI, m/z): Calcd. for C₂₄H₃₆N₂O₂, 384.2 (M+H), found 385.1.

### d) 4-(4-{[4-Cyano-1-(2-trimethylsilanyl-ethoxymethyl)-1H-imidazole-2-carbonyl]-amino}-3-cyclohex-1-enyl-phenyl)-cis-2,6-dimethyl-piperidine-1-carboxylic acid tert-butyl ester; and 4-(4-{[4-cyano-1-(2-trimethylsilanyl-ethoxymethyl)-1H-imidazole-2-carbonyl]-amino}-3-cyclohex-1-enyl-phenyl)-trans-2,6-dimethyl-piperidine-1-carboxylic acid tert-butyl ester

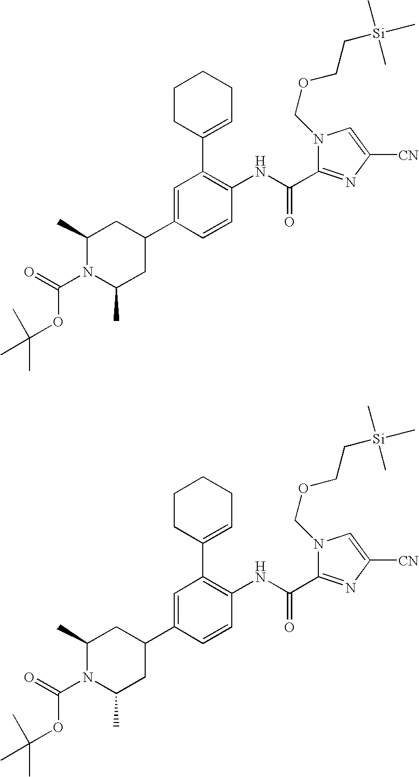

A mixture of 4-cyano-1-(2-trimethylsilanyl-ethoxymethyl)-1H-imidazole-2-carboxylic acid, potassium salt (as prepared in Example 3, step (d), 384 mg, 1.00 mmol), DIEA (0.34 μL, 2.0 mmol), 4-(4-amino-3-cyclohex-1-enyl-phenyl)-2,6-dimethyl-piperidine-1-carboxylic acid tert-butyl ester (as prepared in the previous step, 384 mg, 1.00 mmol) and PyBroP (699 mg, 1.50 mmol) in DCM (20 mL) was stirred at RT for 12 h. The reaction mixture was diluted with DCM (10 mL) and washed with saturated aqueous NaHCO₃ (10 mL) and water (10 mL). The organic layer was separated, dried (Na₂SO₄) and concentrated in vacuo to obtained a mixture of the above two title compounds (321 mg, 50.7%). The mixture was chromatographed on silica (10-20% EtOAc/hexane) to obtain the individual title compounds.

4-(4-{[4-cyano-1-(2-trimethylsilanyl-ethoxymethyl)-1H-imidazole-2-carbonyl]-amino}-3-cyclohex-1-enyl-phenyl)-trans-2,6-dimethyl-piperidine-1-carboxylic acid tert-butyl ester (31 mg). Mass spectrum (ESI, m/z): Calcd. for C₃₅H₅₁N₅O₄Si, 634.3 (M+H), found 634.1.

4-(4-{[4-cyano-1-(2-trimethylsilanyl-ethoxymethyl)-1H-imidazole-2-carbonyl]-amino}-3-cyclohex-1-enyl-phenyl)-cis-2,6-dimethyl-piperidine-1-carboxylic acid tert-butyl ester contaminated with 10% of 4-(4-{[4-cyano-1-(2-trimethylsilanyl-ethoxymethyl)-1H-imidazole-2-carbonyl]-amino}-3-cyclohex-1-enyl-phenyl)-trans-2,6-dimethyl-piperidine-1-carboxylic acid tert-butyl ester (290 mg). Mass spectrum (ESI, m/z): Calcd. for C₃₅H₅₁N₅O₄Si, 634.3 (M+H), found 634.1.

### e) 5-Cyano-1H-imidazole-2-carboxylic acid [2-cyclohex-1-enyl-4-(cis-2,6-dimethyl-piperidin-4-yl)-phenyl]-amide bis trifluoroacetic acid salt and 5-cyano-1H-imidazole-2-carboxylic acid [2-cyclohex-1-enyl-4-(trans-2,6-dimethyl-piperidin-4-yl)-phenyl]-amide bis trifluoroacetic acid salt

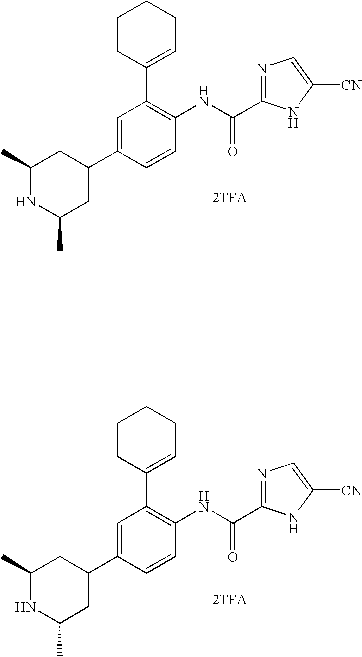

The title compounds were prepared from 290 mg (0.457 mmol) of 4-(4-{[4-cyano-1-(2-trimethylsilanyl-ethoxymethyl)-1H-imidazole-2-carbonyl]-amino}-3-cyclohex-1-enyl-phenyl)-cis-2,6-dimethyl-piperidine-1-carboxylic acid tert-butyl ester and 31 mg (0.048 mmol) of 4-(4-{[4-cyano-1-(2-trimethylsilanyl-ethoxymethyl)-1H-imidazole-2-carbonyl]-amino}-3-cyclohex-1-enyl-phenyl)-trans-2,6-dimethyl-piperidine-1-carboxylic acid tert-butyl ester according to the procedure in Example 14, step (b).

5-Cyano-1H-imidazole-2-carboxylic acid [2-cyclohex-1-enyl-4-(cis-2,6-dimethyl-piperidin-4-yl)-phenyl]-amide bis trifluoroacetic acid salt (93 mg, 32%): ¹H-NMR (CD₃OD; 400 MHz): δ 8.17 (d, 1H, J=8.4 Hz), 8.03 (s, 1H), 7.22 (d, 1H, J=8.4 Hz), 7.11 (s, 1H), 5.72 (br s, 1H), 3.87 (m, 1H), 3.78 (m, 1H), 3.45 (m, 1H), 3.23 (m, 1H), 3.07 (m, 1H), 2.22 (m, 4H), 2.19 (m, 2H), 1.75-1.92 (m, 4H), 1.56 (m, 3H), 1.37 (m, 6H). Mass spectrum, ESI, m/z): Calcd. for C₂₄H₂₉N₅O, 404.2 (M+H), found 404.2.

5-Cyano-1H-imidazole-2-carboxylic acid [2-cyclohex-1-enyl-4-(trans-2,6-dimethyl-piperidin-4-yl)-phenyl]-amide bis trifluoroacetic acid salt (17.3 mg, 56%). ¹H-NMR (CDCl₃; 400 MHz): δ 13.9 (br s, 1H), 10.3 (br s, 1H), 9.98 (s, 1H), 8.41 (d, 1H, J=8.4 Hz), 7.75 (br s, 1H), 7.26 (dd, 1H, J=8.4, 2.0 Hz), 7.15 (d, 1H, J=2 Hz), 5.92 (br s, 1H), 4.12 (m, 1H), 3.59 (m, 1H), 3.1-3.3 (m, 4H), 2.25-2.42 (m, 6H), 2.05-1.78 (m, 6H), 1.62 (d, 3H, J=7.1 Hz), 1.43 (d, 3H, J=6.3 Hz). Mass spectrum (ESI, m/z): Calcd. for C₂₄H₂₉N₅O, 404.2 (M+H), found 404.2.

### Example 52

### 5-Cyano-1H-imidazole-2-carboxylic acid {2-cyclohex-1-enyl-4-[1-(R)-(+)-(2,3-dihydroxy-propionyl)-piperidin-4-yl]-phenyl}-amide

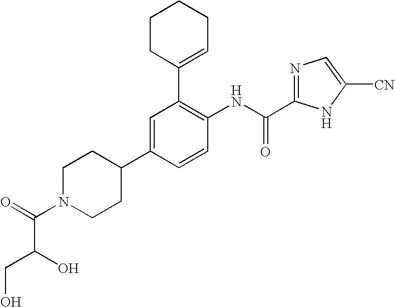

### a) 5-Cyano-1H-imidazole-2-carboxylic acid {2-cyclohex-1-enyl-4-[1-(R)-(+)2,2-dimethyl-[1,3]dioxolane-4-carbonyl)-piperidin-4-yl]-phenyl}-amide

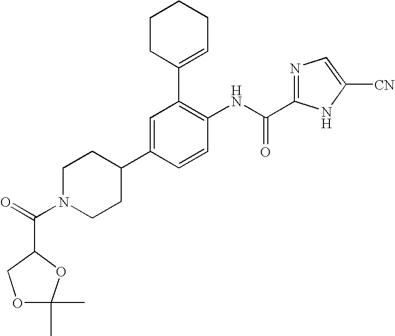

To a solution of methyl (R)-(+)-2,2-dimethyl-1,3-dioxolane-4-carboxylate (0.16 mL, 1.0 mmol) in MeOH (2 mL), 2 N KOH (0.5 mL, 1 mmol) was added. The resulting solution was stirred at RT for 20 min and concentrated in vacuo. The residue obtained was suspended in ether (10 mL) and sonicated for 5 min. The ether was then removed and the resulting residue was dried in vacuo for 4 h to obtain (R)-(+)-2,2-dimethyl-1,3-dioxolane-4-carboxylic acid potassium salt (173 mg, 94%) which was directly used in the next step without purification.

To a solution of 4-cyano-1H-imidazole-2-carboxylic acid (2-cyclohex-1-enyl-4-piperidin-4-yl-phenyl)-amide, trifluoroacetic acid salt (as prepared in Example 14, step (b), 40 mg, 0.08 mmol) in DCM (1.5 mL) was added to a mixture of (R)-(+)-2,2-dimethyl-1,3-dioxalane-4-carboxylic acid potassium salt (as prepared above, 18 mg, 0.090 mmol), EDCI (18.8 mg, 0.0900 mmol), HOBt (13.2 mg, 0.0900 mmol) and DIEA (42 μL, 0.24 mmol). The resulting mixture was stirred at RT for 6 h. Water (10 mL) was added and DCM layer was separated, dried (Na₂SO₄) and concentrated. The residue obtained was chromatographed on silica (2% MeOH/DCM) to obtain title compound (47 mg, 97%). Mass spectrum (ESI, m/z): Calcd. for C₂₈H₃₃N₅O₄, 504.2 (M+H), found 503.9.

### b) 5-Cyano-1H-imidazole-2-carboxylic acid {2-cyclohex-1-enyl-4-[1-(R)-(+)-(2,3-dihydroxy-propionyl)-piperidin-4-yl]-phenyl}-amide

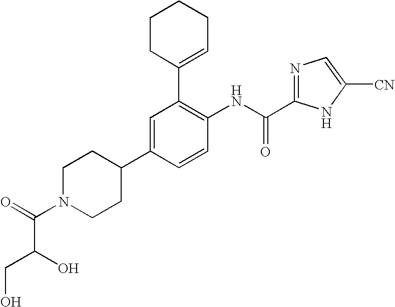

To a solution of 5-cyano-1H-imidazole-2-carboxylic acid {2-cyclohex-1-enyl-4-[1-(R)-(2,2-dimethyl-[1,3]dioxolane-4-carbonyl)-piperidin-4-yl]-phenyl}-amide (as prepared in the previous step, 45 mg, 0.090 mmol) in MeOH (1 mL) was added aq 2 N HCl (2 mL). The resulting mixture was stirred at RT for 12 hr. Solvents were removed in vacuo and the resulting residue was dried for 4 h. The ether (10 mL) was added and sonicated for 5 min. The ether was removed in vacuo and the residue was dried for 12 h to obtain the title compound (21.3 mg, 52%). ¹H-NMR (DMSO; 400 MHz): δ 14.1 (br s, 1H), 9.85 (s, 1H), 8.32 (s, 1H), 7.92 (d, 1H, J=8.4 Hz), 7.18 (dd, 1H, J=8.4, 2.1 Hz), 7.13 (d, 1H, J=2.1 Hz), 5.72 (br s, 1H), 4.51 (m, 1H), 4.33 (m, 1H), 4.15 (m, 1H), 3.55 (m, 1H), 3.43 (m, 1H), 3.08 (m, 1H), 2.81 (m, 1H), 2.63 (m, 1H), 2.12-2.24 (m, 4H), 1.31-1.38 (m, 10H). mass spectrum (ESI, m/z): Calcd. for C₂₅H₂₉N₅O₄, 464.2 (M+H), found 464.1.

### Example 53

### 5-Cyano-1H-imidazole-2-carboxylic acid [2-cyclohex-1-enyl-4-(1-methoxy-piperidin-4-yl)-phenyl]-amide trifluoroacetic acid salt

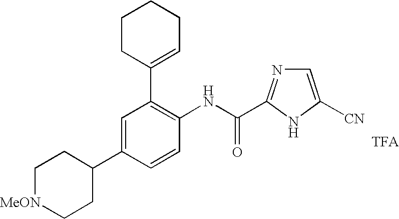

### a) 4-(1-Methoxy-1,2,3,6-tetrahydro-pyridin-4-yl)-phenylamine

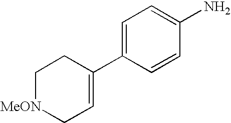

A solution of N-methoxypiperidinone (J. Org. Chem., 26, 1867, (1961), 650 mg, 5.00 mmol) in THF (20 mL)) was cooled to −78° C. and treated with LDA (1.5 M solution in cyclohex, THF and ethylbenzene, 4.3 mL, 6.4 mmol) under Ar. The resulting mixture was stirred at same temperature for 30 min and treated with N-phenyltrifluoromethanesulfonimide (2.3 g, 6.4 mmol) in THF (20 mL). The reaction mixture was stirred for another 30 min and allowed to warm to RT. After 30 min at RT, the reaction mixture was concentrated in vacuo and the residue obtained was taken up in EtOAc (20 mL) and washed with cold water (2×10 mL). EtOAc layer was dried (Na₂SO₄) and concentrated to afforded trifluoromethanesulfonic acid 1-methoxy-1,2,3,6-tetrahydro-pyridin-4-yl ester (980 mg, 71%) as a white foam which was directly used in next step.

The title compound was then prepared according to Suzuki coupling procedure of Example 35, step (b) using 4-aminophenylboronic acid (219 mg, 1.00 mmol) and trifluoromethanesulfonic acid 1-methoxy-1,2,3,6-tetrahydro-pyridin-4-yl ester (as prepared above, 261 mg, 1.00 mmol). Silica gel chromatography (20-50% EtOAc/hexanes) afforded 60 mg (29%). Mass spectrum (ESI, m/z): Calcd. for C₁₂H₁₆N₂O, 205.1 (M+H), found 205.2.

### b) 2-Cyclohex-1-enyl-4-(1-methoxy-piperidin-4-yl)-phenylamine

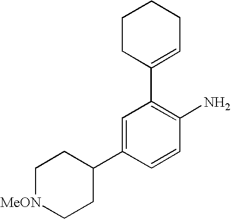

A solution of 4-(1-methoxy-1,2,3,6-tetrahydro-pyridin-4-yl)-phenylamine (as prepared in previous step) (40.8 mg, 0.200 mmol) in MeOH (5 mL) was hydrogenated over 10% Pd/C (20.4 mg) at 20 psi for 1 h. The solution was filtered through a pad of Celite and concentrated to give 4-(1-methoxy-piperidin-4-yl)-phenylamine (38 mg, 92%) which was directly used in the next step without purification.

To a solution of 4-(1-methoxy-piperidin-4-yl)-phenylamine (as prepared above, 42 mg, 0.20 mmol) in DCM (2 mL) was added NBS (36.2 mg, 0.20 mmol) and the reaction mixture was stirred at RT for 12 h. The reaction mixture was diluted with DCM (10 mL) and washed with saturated aqueous NaHCO₃ (10 mL) and water (10 mL). The organic layer was separated, dried (Na₂SO₄) and concentrated in vacuo to obtain 2-bromo-4-(1-methoxy-1,2,3,6-tetrahydro-pyridin-4-yl)-phenylamine (43 mg, 74.5%) which was used in the next step without purification.

The title compound was then prepared according to Suzuki coupling procedure of Example 12, step (d) using cyclohex-1-enyl boronic acid (27.9 mg, 1.00 mmol) and 2-bromo-4-(1-methoxy-1,2,3,6-tetrahydro-pyridin-4-yl)-phenylamine (as prepared above, 44 mg, 0.15 mmol) and chromatographed on silica (20-50% EtOAc/hexanes) afforded 2-cyclohex-1-enyl-4-(1-methoxy-piperidin-4-yl)-phenylamine (33 mg, 74%). Mass spectrum, (ESI, m/z): Calcd. for C₁₈H₂₆N₂O, 287.2 (M+H), found 286.8.

### c) 4-Cyano-1-(2-trimethylsilanyl-ethoxymethyl)-1H-imidazole-2-carboxylic acid [2-cyclohex-1-enyl-4-(1-methoxy-piperidin-4-yl)-phenyl]-amide

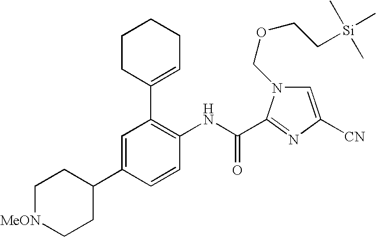

A mixture of 4-cyano-1-(2-trimethylsilanyl-ethoxymethyl)-1H-imidazole-2-carboxylic acid, potassium salt (as prepared in Example 3, step (d), 35.6 mg, 0.100 mmol), DIEA (0.34 μL, 0.20 mmol), 2-cyclohex-1-enyl-4-(1-methoxy-piperidin-4-yl)-phenylamine (as prepared in previous step, 28.6 mg, 0.1 mmol) and PyBroP (69.9 mg, 0.150 mmol) in DCM (2 mL) was stirred at RT for 12 h. The reaction mixture was diluted with DCM (10 mL) and washed with saturated aqueous NaHCO₃ (10 mL) and water (10 mL). The organic layer was separated, dried (Na₂SO₄) and concentrated in vacuo. The product was chromatographed on silica (20-40% EtOAc/hexane) to obtain the title compound (26 mg, 48%). Mass spectrum (ESI, m/z): Calcd. for C₂₉H₄₁N₅O₃Si, 536.3 (M+H), found 536.2.

### d) 5-Cyano-1H-imidazole-2-carboxylic acid [2-cyclohex-1-enyl-4-(1-methoxy-piperidin-4-yl)-phenyl]-amide trifluoroacetic acid salt

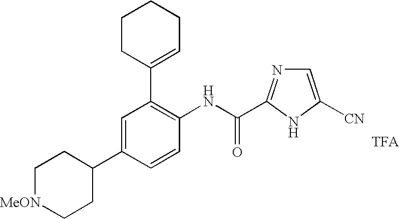

To a solution of 4-cyano-1-(2-trimethylsilanyl-ethoxymethyl)-1H-imidazole-2-carboxylic acid [2-cyclohex-1-enyl-4-(1-methoxy-piperidin-4-yl)-phenyl]-amide (as prepared in previous step, 31 mg, 0.020 mmol) in DCM (0.5 mL) and EtOH (11 μL) was added TFA (0.1 mL). The resulting solution was stirred at RT for 6 h. The reaction mixture was concentrated in vacuo and the resulting residue was dried for 1 h, suspended in ether (10 mL) and sonicated for 5 min. The solid formed was collected by suction filtration to obtain the title compound (17.3 mg, 58%). ¹H-NMR (DMSO; 400 MHz): δ 9.70 (s, 1H), 8.30 (s, 1H), 7.83 (d, 1H, J=8.4 Hz), 7.14 (d, 1H, J=8.4 Hz), 7.05 (s, 1H), 5.71 (br s, 1H), 3.30-3.55 (m, 5H), 2.41-2.62 (m, 2H), 2.12-2.19 (m, 4H), 1.60-1.85 (m, 8H). Mass spectrum (ESI, m/z): Calcd. for C₂₃H₂₂N₅O₂, 406.2 (M+H), found 406.1.

### Example 54

### 4-Cyano-1H-imidazole-2-carboxylic acid [6-(4,4-dimethyl-cyclohex-1-enyl)-1′,2′,3′,4′,5′,6′-hexahydro-[2,4′]bipyridinyl-5-yl]-amide trifluoroacetic acid salt

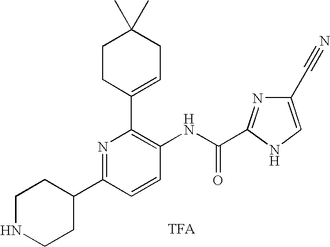

### a) 5-Nitro-3′,6′-dihydro-2′H-[2,4′]bipyridinyl-1′-carboxylic acid tert-butyl ester

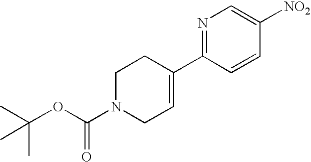

A solution of 202 mg (0.994 mmol) 2-bromo-5-nitropyridine in 4 mL of toluene and 2 mL of EtOH was treated with 338 mg (1.09 mmol) 4-trifluoromethane-sulfonyloxy-3,6-dihydro-2H-pyridine-1-carboxylic acid tert-butyl ester (Synthesis, 993, (1991)) and 1.49 mL (2.981 mmol) 2 M aqueous Na₂CO₃. The mixture was degassed via sonication, placed under argon, treated with 80.3 mg (0.00700 mmol) Pd(PPh₃)₄ and heated to 80° C. for 4 h. The mixture was diluted with EtOAc and washed with water. The organic layer was dried over MgSO₄ and concentrated in vacuo. The resulting residue was chromatographed on a 50-g silica Varian MegaBond Elut column with 10-25% EtOAc-hexane to afford 226 mg (75%) of the title compound as a light yellow solid: Mass spectrum (ESI, m/z): Calcd. for C₁₅H₁₉N₃O₄, 306.1 (M+H), found 305.7.

### b) 5-Amino-3′,4′,5′,6′-tetrahydro-2′H-[2,4′]bipyridinyl-1′-carboxylic acid tert-butyl ester

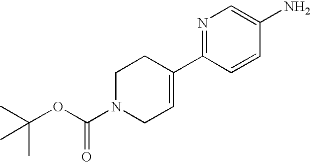

A solution of 226 mg (0.740 mmol) 5-nitro-3′,6′-dihydro-2′H-[2,4′]bipyridinyl-1′-carboxylic acid tert-butyl ester (as prepared in the previous step) in 15 mL MeOH was treated with 110 mg 10% Pd/C (Degussa type E101-NE/W, Aldrich, 50% by weight water) and 1 atm H₂ at room temperature for 18 h. The mixture was filtered through Celite, and the filter cake was washed with MeOH. Concentration afforded 220 mg (107%) of the title compound as a colorless glassy solid. Mass spectrum (ESI, m/z): Calcd. for C₁₅H₂₃N₃O₂, 278.2 (M+H), found 278.0.

### c) 5-Amino-6-bromo-3′,4′,5′,6′-tetrahydro-2′H-[2,4′]bipyridinyl-1′-carboxylic acid tert-butyl ester

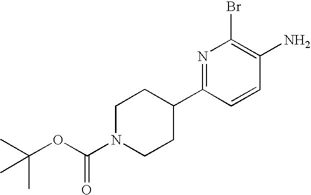

A solution of 220 mg (0.793 mmol) 5-amino-3′,4′,5′,6′-tetrahydro-2′H-[2,4′]bipyridinyl-1′-carboxylic acid tert-butyl ester (as prepared in the previous step) in 10 mL CH₂Cl₂ was treated with 134 mg (0.753 mmol) N-bromosuccinimide at room temperature for 20 min. The mixture was diluted with CH₂Cl₂ and washed with saturated aqueous NaHCO₃. The organic layer was dried over MgSO₄ and concentrated in vacuo. Chromatography of the residue on a 50-g silica Varian MegaBond Elut column with 10-35% EtOAc-hexanes afforded 209 mg (74%) of the title compound as a colorless glassy solid. ¹H-NMR (CDCl₃; 400 MHz): δ 6.97 (d, 1H, J=8.0 Hz), 6.91 (d, 1H, J=8.0 Hz), 4.28-4.15 (br s, 2H), 4.06-3.90 (m, 2H), 2.85-2.75 (m, 2H), 2.77-2.68 (m, 1H), 1.92-1.83 (m, 2H), 1.68-1.54 (m, 2H), 1.47 (s, 9H).

### d) 5-Amino-6-(4,4-dimethyl-cyclohex-1-enyl)-3′,4′,5′,6′-tetrahydro-2′H-[2,4′]bipyridinyl-1′-carboxylic acid tert-butyl ester

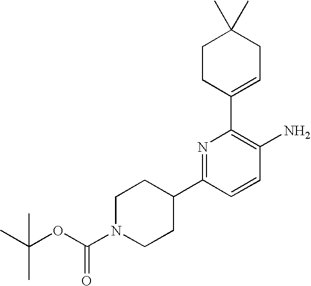

A solution of 209 mg (0.587 mmol) 5-amino-6-bromo-3′,4′,5′,6′-tetrahydro-2′H-[2,4′]bipyridinyl-1′-carboxylic acid tert-butyl ester (as prepared in the previous step) in 5 mL of toluene and 2.5 mL of EtOH was treated with 99.3 mg (0.645 mmol) 4,4-dicyclohex-1-enylboronic acid and 2.34 mL (4.69 mmol) 2 M aqueous Na₂CO₃. The mixture was degassed via sonication, placed under argon, treated with 47.4 mg (0.0410 mmol) Pd(PPh₃)₄, and heated to 80° C. for 16 h. The mixture was diluted with EtOAc and washed with water. The aqueous layer was extracted with additional EtOAc, and the combined organic layers were dried over MgSO₄ and concentrated in vacuo. Chromatography of the residue on a 50-g silica Varian MegaBond Elut column with 25% EtOAc-hexanes afforded 150 mg (66%) of the title compound as a white foamy solid. Mass spectrum (ESI, m/z): Calcd. for C₂₃H₃₅N₃O₂, 386.3 (M+H), found 386.3.

### e) 5-{[4-Cyano-1-(2-trimethylsilanyl-ethoxymethyl)-1H-imidazole-2-carbonyl]-amino}-6-(4,4-dimethyl-cyclohex-1-enyl)-3′,4′,5′,6′-tetrahydro-2′H-[2,4′]bipyridinyl-1′-carboxylic acid tert-butyl ester

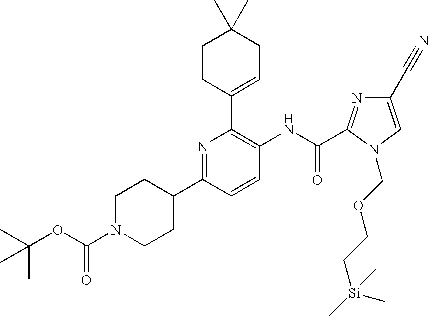

A solution of 150 mg (0.389 mmol) 5-amino-6-(4,4-dimethyl-cyclohex-1-enyl)-3′,4′,5′,6′-tetrahydro-2′H-[2,4′]bipyridinyl-1′-carboxylic acid tert-butyl ester (as prepared in the previous step) in 15 mL of CH₂Cl₂ was treated with 131 mg (0.428 mmol) of 4-cyano-1-(2-trimethylsilanyl-ethoxymethyl)-1H-imidazole-2-carboxylate potassium salt (as prepared in Example 3, step (b)), 272 mg (0.584 mmol) PyBroP, and 203 μL (1.17 mmol) DIEA at room temperature for 3 h. The mixture was diluted with CH₂Cl₂ and washed with saturated aqueous NaHCO₃. The organic layer was dried over MgSO₄ and concentrated in vacuo. Chromatography of the residue on a 50-g silica Varian MegaBond Elut column with 50% EtOAc-hexanes afforded 215 mg (87%) of the title compound as a white solid. Mass spectrum (ESI, m/z): Calcd. for C₃₄H₅₀N₆O₄Si, 635.4 (M+H), found 635.3.

### f) 4-Cyano-1H-imidazole-2-carboxylic acid [6-(4,4-dimethyl-cyclohex-1-enyl)-1′,2′,3′,4′,5′,6′-hexahydro-[2,4′]bipyridinyl-5-yl]-amide trifluoroacetic acid salt

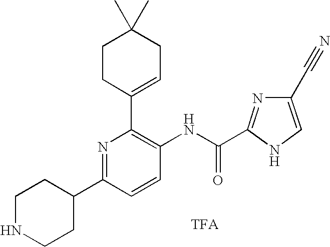

A solution of 215 mg (0.339 mmol) 5-{[4-cyano-1-(2-trimethylsilanyl-ethoxymethyl)-1H-imidazole-2-carbonyl]-amino}-6-(4,4-dimethyl-cyclohex-1-enyl)-3′,4′,5′,6′-tetrahydro-2′H-[2,4′]bipyridinyl-1′-carboxylic acid tert-butyl ester (as prepared in the previous step) in 10 mL of CH₂Cl₂ was treated with three drops MeOH and 3 mL TFA at room temperature for 4 h. MeOH (10 mL) was added and the solvents evaporated in vacuo. Chromatography of the residue on a 50-g silica Varian MegaBond Elut column with 10% MeOH—CH₂Cl₂ afforded 210 mg (97%) of the title compound as a white solid. ¹H-NMR (CD₃OD; 400 MHz): δ 8.59 (d, 1H, J=8.4 Hz), 8.04 (s, 1H), 7.28 (d, 1H, J=8.4 Hz), 6.02-5.93 (m, 1H), 3.58-3.48 (m, 2H), 3.32-3.03 (m, 3H), 2.54-2.42 (m, 2H), 2.23-2.02 (m, 6H), 1.11 (s, 6H). Mass spectrum (ESI, m/z): Calcd. for C₂₃H₂₈N₆O, 405.2 (M+H), found 405.2.

### Example 55

### 4-Cyano-1H-imidazole-2-carboxylic acid [1′-(2-dimethylamino-acetyl)-6-(4,4-dimethyl-cyclohex-1-enyl)-1′,2′,3′,4′,5′,6′-hexahydro-[2,4′]bipyridinyl-5-yl]-amide trifluoroacetic acid salt

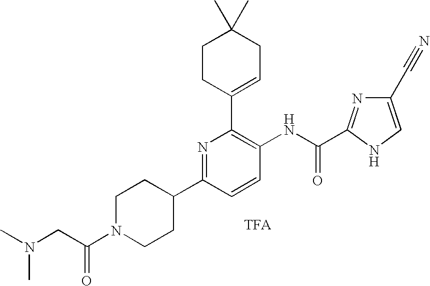

A suspension of 20.9 mg (0.203 mmol) N,N-dimethylglycine in 4 mL CH₂Cl₂ was treated with 49.8 mg (0.197 mmol) bis(2-oxo-3-oxazolidinyl)phosphinic chloride (BOP-Cl) and 75 μL (0.54 mmol) Et₃N at room temperature for 1 h. The mixture was then treated with 70.0 mg (0.135 mmol) 4-cyano-1H-imidazole-2-carboxylic acid [6-(4,4-dimethyl-cyclohex-1-enyl)-1′,2′,3′,4′,5′,6′-hexahydro-[2,4′]bipyridinyl-5-yl]-amide trifluoroacetate (as prepared in Example 54, step (f)) at room temperature for 18 h. The mixture was diluted with CH₂Cl₂ and washed with water. The organic layer was dried over MgSO₄ and concentrated in vacuo. The residue was purified by RP-HPLC (C18) with 10-80% CH₃CN in 0.1% TFA/H₂O over 30 min to afford 34.9 mg (53%) of the title compound as a white solid. ¹H-NMR (CD₃OD; 400 MHz): δ 8.38 (d, 1H, J=8.4 Hz), 8.05 (s, 1H), 7.33 (d, 1H, J=8.4 Hz), 6.05-5.98 (m, 1H), 4.68 (d, 1H, J=15.2 Hz), 3.82 (d, 1H, J=15.2 Hz), 3.16-3.05 (m, 1H), 3.01-2.94 (m, 6H), 2.52-2.40 (m, 2H), 2.39 (s, 6H), 2.17-2.10 (m, 2H), 2.09-1.87 (m, 2H), 1.67-1.59 (m, 2H), 1.12 (s, 6H). Mass spectrum (ESI, m/z): Calcd. for C₂₇H₃₅N₇O₂, 490.3 (M+H), found 490.4.

### Example 56

### 4-Cyano-1H-imidazole-2-carboxylic acid [6-(4,4-dimethyl-cyclohex-1-enyl)-1′-(2-methanesulfonyl-ethyl)-1′,2′,3′,4′,5′,6′-hexhydro-[2,4′]bipyridinyl-5-yl]-amide trifluoroacetic acid salt

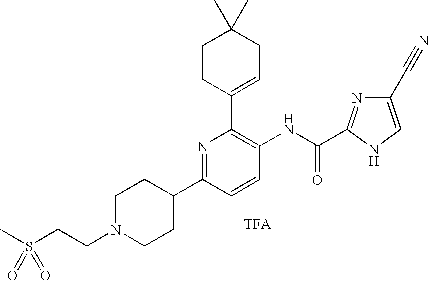

A solution of 70.0 mg (0.135 mmol) 4-cyano-1H-imidazole-2-carboxylic acid [6-(4,4-dimethyl-cyclohex-1-enyl)-1′,2′,3′,4′,5′,6′-hexahydro-[2,4′]bipyridinyl-5-yl]-amide (as prepared in Example 54, step (f)) in 10 mL of CH₂Cl₂ was treated with 32.7 mg (0.162 mmol) methanesulfonic acid 2-methanesulfonyl-ethyl ester (as prepared in Example 40, step (a)) and 70.5 μL (0.405 mmol) DIEA at room temperature for 6 h. The mixture was diluted with CH₂Cl₂ and washed with water. The organic layer was dried over MgSO₄ and concentrated in vacuo. The residue was purified by RP-HPLC (C18) with 20-60% CH₃CN in 0.1% TFA/H₂O over 30 min to afford 48 mg (85%) of the title compound as a white solid. ¹H-NMR (CD₃OD; 400 MHz): δ 8.65 (d, 1H, J=8.4 Hz), 8.05 (s, 1H), 7.34 (d, 1H, J=8.4 Hz), 6.05-5.98 (m, 1H), 3.85-3.66 (m, 6H), 3.29-3.21 (m, 2H), 3.20-3.01 (m, 1H), 3.14 (s, 3H), 2.53-2.45 (m, 2H), 2.30-2.15 (m, 4H), 2.15-2.10 (m, 2H), 1.62 (t, 2H, J=6.4 Hz), 1.11 (s, 6H). Mass spectrum (ESI, m/z): Calcd. for C₂₆H₃₄N₆O₃S, 511.2 (M+H), found 511.3.

### Example 57

### 5-Cyano-1H-imidazole-2-carboxylic acid {4-[1-(2-amino-2-methyl-propionyl)-piperidin-4-yl]-2-cyclohex-1-enyl-phenyl}-amide trifluoroacetic acid salt

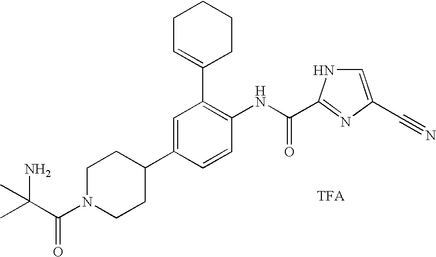

### a) {2-[4-(4-{[4-Cyano-1-(2-trimethylsilanyl-ethoxymethyl)-1H-imidazole-2-carbonyl]-amino}-3-cyclohex-1-enyl-phenyl)-piperidin-1-yl]-1,1-dimethyl-2-oxo-ethyl}-carbamic acid tert-butyl ester

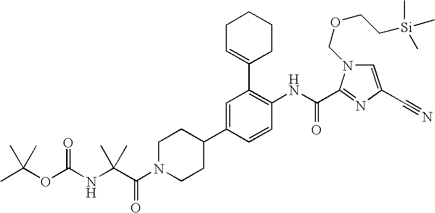

To a solution of 4-(4-{[4-cyano-1-(2-trimethylsilanyl-ethoxymethyl)-1H-imidazole-2-carbonyl]-amino}-3-cyclohex-1-enyl-phenyl)-piperidine-1-carboxylic acid tert-butyl ester (231 mg, 0.380 mmol) (as prepared in Example 14, step (a)) in 2.5 mL of DCM and 0.4 mL EtOH was added 700 μL of TFA and the solution stirred for 3 h at 25° C. The reaction was diluted with 4 mL of EtOH and then concentrated to give ca. a 2:1 mixture of 5-cyano-1-(2-trimethylsilanyl-ethoxymethyl)-1H-imidazole-2-carboxylic acid (2-cyclohex-1-enyl-4-piperidin-4-yl-phenyl)-amide trifluoroacetic acid salt to starting material by ¹H-NMR and LC/MS which was used in the following step without further purification. The mixture in 3 mL of DCM was added to a solution of 2-tert-butoxycarbonylamino-2-methyl-propionic acid (53 mg, 0.70 mmol), DIEA (122 μL, 0.700 mmol) and PyBroP (144 mg, 0.300 mmol) in 3 mL of DCM and the reaction was stirred at 25° C. overnight. The reaction was diluted with EtOAc (25 mL) and washed with satd aq NaHCO₃ (1×25 mL) and brine (25 mL) and the organic layer was dried over Na₂SO₄ and then concentrated. Purification of the residue by preparative TLC (50% EtOAc-hexanes) afforded 40 mg (15%) of the title compound as a white solid. Mass Spectrum (ESI, m/z): Calcd. for C₃₂H₅₅N₆O₅Si, 691.3 (M+H), found 691.1.

### b) 5-Cyano-1H-imidazole-2-carboxylic acid {4-[1-(2-amino-2-methyl-propionyl)-piperidin-4-yl]-2-cyclohex-1-enyl-phenyl}-amide trifluoroacetic acid salt

To a solution of {2-[4-(4-{[4-cyano-1-(2-trimethylsilanyl-ethoxymethyl)-1H-imidazole-2-carbonyl]-amino}-3-cyclohex-1-enyl-phenyl)-piperidin-1-yl]-1,1-dimethyl-2-oxo-ethyl}-carbamic acid tert-butyl ester (40 mg, 0.050 mmol) in 2 mL of DCM and 20 μL of EtOH was added 1.5 mL of TFA. The solution was stirred for 3 h at 25° C., diluted with 2 mL of EtOH and concentrated in vacuo. Trituration of the residue with ether afforded 8.4 mg (29%) of the title compound as a white solid. ¹H-NMR (CD₃OD; 400 MHz): δ 8.10 (d, 1H, J=8.4 Hz), 8.00 (s, 1H), 7.16 (d, 1H, J=8.4 Hz), 7.07 (s, 1H), 5.79 (s, 1H), 4.55-4.48 (m, 1H), 3.30 (s, 6H), 2.89-2.87 (m, 2H), 2.40-2.25 (m, 4H), 1.96-1.93 (m, 2H), 1.86-1.83 (m, 6H), 1.64-1.61 (m, 2H). Mass Spectrum (ESI, m/z): Calcd. for C₂₆H₃₃N₆O₂, 461.2 (M+H), found 461.3.

### Example 58

### 5-Cyano-1H-imidazole-2-carboxylic acid [6-cyclohex-1-enyl-1′-(2-methanesulfonyl-ethyl)-1′,2′,3′,4′,5′,6′-hexahydro-[2,4′]bipyridinyl-5-yl]-amide

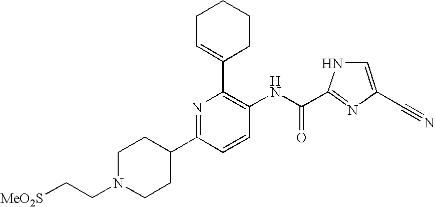

### a) 5-Amino-6-cyclohex-1-enyl-3′,4′,5′,6′-tetrahydro-2′H-[2,4′]bipyridinyl-1′-carboxylic acid tert-butyl ester

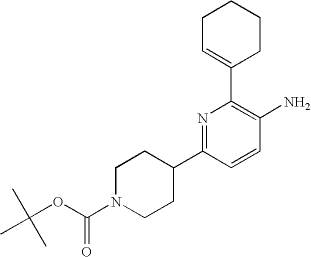

To a mixture of 5-amino-6-bromo-3′,4′,5′,6′-tetrahydro-2′H-[2,4′]bipyridinyl-1′-carboxylic acid tert-butyl ester (331 mg, 0.93 mmol) (as prepared in Example 54, step (c)) and cyclohexen-1-yl boronic acid (141 mg, 1.11 mmol) in 5 mL of EtOH, 10 mL of toluene and 5 mL of 2 M Na₂CO₃, was added Pd(PPh₃)₄ (107 mg, 0.0930 mmol) and the result was heated at 80° C. for 16 h. The reaction was diluted with 100 mL of ether and 100 mL of brine and the layers were separated. The organic layer was dried (Na₂SO₄) and concentrated in vacuo. Purification of the residue by column chromatography (silica gel, 30-60% ether-hexanes) afforded 248 mg (74%) the title compound as an light brown oil LC-MS (ESI, m/z): Calcd. for C₂₁H₃₂N₃O₂ (M+H), 358.2, found 358.1.

### b) 5-{[4-Cyano-1-(2-trimethylsilanyl-ethoxymethyl)-1H-imidazole-2-carbonyl]-amino}-6-cyclohex-1-enyl-3′,4′,5′,6′-tetrahydro-2′H-[2,4′]bipyridinyl-1′-carboxylic acid tert-butyl ester

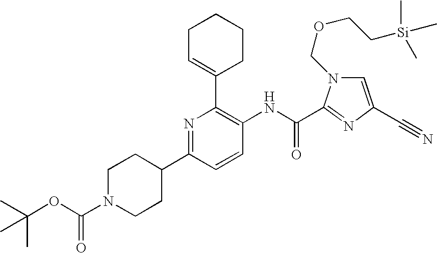

To a solution of 4-cyano-1-(2-trimethylsilanyl-ethoxymethyl)-1H-imidazole-2-carboxylate potassium salt (296 mg, 0.970 mmol) (as prepared in Example 3, step (d)) in 8 mL DCM was added DIEA (291 μL, 1.72 mmol) and PyBroP (512 mg, 1.10 mmol), and the reaction was stirred at 25° C. for 15 min. A solution of 5-amino-6-cyclohex-1-enyl-3′,4′,5′,6′-tetrahydro-2′H-[2,4′]bipyridinyl-1′-carboxylic acid tert-butyl ester (233 mg, 0.65 mmol) (as prepared in the previous step) in 4 mL DCM was added and the reaction stirred overnight at 25° C. The reaction was diluted with EtOAc (25 mL) and washed with NaHCO₃ (1×25 mL) and brine (25 mL) and the organic layer was dried over Na₂SO₄ and then concentrated. The residue was purified by flash chromatography (silica gel, 5% MeOH—CHCl₃) to afford 167 mg (40%) of the title compound as a white solid. Mass Spectrum (ESI, m/z): Calcd. for C₃₂H₄₆N₆O₄Si, 607.3 (M+H), found 607.3.

### c) 5-Cyano-1H-imidazole-2-carboxylic acid (6-cyclohex-1-enyl-1′,2′,3′,4′,5′,6′-hexahydro-[2,4′]bipyridinyl-5-yl)-amide trifluoroacetic acid salt

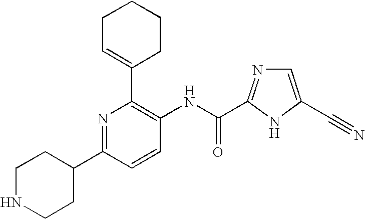

The title compound was prepared from 5-{[4-cyano-1-(2-trimethylsilanyl-ethoxymethyl)-1H-imidazole-2-carbonyl]-amino}-6-cyclohex-1-enyl-3′,4′,5′,6′-tetrahydro-2′H-[2,4]bipyridinyl-1′-carboxylic acid tert-butyl ester (167 mg, 0.27 mmol) using a procedure similar to Example 14, step (b) to afford 57 mg (43%) of the title compound as a white solid. LC-MS (ESI, m/z): Calcd. for C₂M₂₄N₆O, 377.2 (M+H), found 377.2.

### d) 5-Cyano-1H-imidazole-2-carboxylic acid [6-cyclohex-1-enyl-1′-(2-methanesulfonyl-ethyl)-1′,2′,3′,4′,5′,6′-hexahydro-[2,4′]bipyridinyl-5-yl]-amide

To a slurry of 5-cyano-1H-imidazole-2-carboxylic acid (6-cyclohex-1-enyl-1′,2′,3′,4′,5′,6′-hexahydro-[2,4]bipyridinyl-5-yl)-amide trifluoroacetic acid salt (57 mg, 0.11 mmol) in 5 mL of DCM was added DIEA (50.4 μL, 0.290 mmol) followed by 30.5 mg (0.150 mmol) of methanesulfonic acid 2-methanesulfonyl-ethyl ester (as prepared in Example 40, step (a)). The reaction was allowed to stir overnight, diluted with 20 mL of DCM, washed with satd aq NaHCO₃ (1×20 mL) and dried over Na₂SO₄. Purification by preparative TLC (silica gel, 40% EtOAc-hexanes) afforded 22.3 mg (40%) of the title compound as a white solid. ¹H-NMR (DMSO; 400 MHz): δ 10.02 (s, 1H), 8.24 (s, 1H), 8.11 (d, 1H, J=8.4 Hz), 7.18 (d, 1H, J=8.4 Hz), 5.96 (s, 1H), 3.04 (s, 3H), 3.02-2.99 (m, 3H), 2.73 (t, 2H, J=2.7 Hz), 2.39-2.37 (m, 2H), 2.11-2.05 (m, 4H), 1.85-1.64 (m, 10H). Mass Spectrum (ESI, m/z): Calcd. for C₂₄H₃₁N₆O₃S, 483.2 (M+H), found 483.3.

### Example 59

An alternate method for the synthesis of the intermediate described in Example 3 is described below.

### 4-Cyano-1-(2-trimethylsilanyl-ethoxymethyl)-1H-imidazole-2-carboxylic acid potassium salt

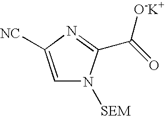

### a) 1H-Imidazole-4-carbonitrile

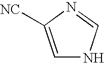

A 22-L, four-neck, round-bottom flask equipped with a mechanical stirrer, a temperature probe, a condenser, and an addition funnel with a nitrogen inlet was charged with 1H-imidazole-4-carboxaldehyde (Aldrich, 1.10 kg, 11.5 mol) and pyridine (3.0 L, 3.0 mol). The reaction flask was cooled to 8° C. with an ice bath and hydroxylamine hydrochloride (871 g, 12.5 mol) was added slowly in portions to maintain the internal temperature below 30° C. The reaction was allowed to cool to ambient temperature and stirred for 2 h at ambient temperature. The resulting thick yellow solution was heated to 80° C. with a heating mantle and acetic anhydride (2.04 L, 21.6 mol) was added dropwise over 200 min to maintain the temperature below 110° C. during the addition. The reaction mixture was heated at 100° C. for 30 min, after which time it was allowed to cool to ambient temperature and then further cooled in an ice bath. The pH was adjusted to 8.0 (pH meter) by the addition of 25 wt % NaOH (5.5 L) at such a rate that the internal temperature was maintained below 30° C. The reaction mixture was then transferred into a 22-L separatory funnel and extracted with ethyl acetate (6.0 L). The combined organic layer was washed with brine (2×4.0 L), dried over MgSO₄, filtered, and concentrated to dryness under reduced pressure at 35° C. to give the crude product as a yellow semisolid. The resulting semisolid was suspended in toluene (3.0 L) and stirred for 1 h, after which time it was filtered to give a light yellow solid, which was resuspended in toluene (3.0 L) and stirred for 1 h. The resulting slurry was filtered and the filter cake washed with toluene (2×500 mL) to give the title compound as a light yellow solid [870 g, 82%). The ¹H and ¹³C NMR spectra were consistent with the assigned structure.

### b) 1-(2-Trimethylsilanyl-ethoxymethyl)-1H-imidazole-4-carbonitrile and 3-(2-trimethylsilanyl-ethoxymethyl)-3H-imidazole-4-carbonitrile

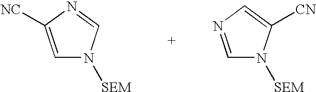

A 22-L, four-neck, round-bottom flask equipped with a mechanical stirrer, a temperature probe, and an addition funnel with a nitrogen inlet was charged with 1H-imidazole-4-carbonitrile (830 g, 8.91 mol, as prepared in the previous step), potassium carbonate (2.47 kg, 17.8 mol), and acetone (6.0 L). Agitation was initiated and the mixture was cooled to 10° C. with an ice bath. SEMCl (1.50 kg, 9.00 mol) was added through the addition funnel over 210 min to maintain the internal temperature below 15° C. The reaction was then allowed to warm to ambient temperature and stirred at ambient temperature overnight (20 h). The reaction mixture was then cooled in an ice bath to 10° C. and quenched by the slow addition of water (8.0 L) over 30 min to maintain the internal temperature below 30° C. The resulting mixture was transferred to a 22-L separatory funnel and extracted with ethyl acetate (2×7.0 L). The combined organics were concentrated under reduced pressure at 35° C. to give the crude product as a dark brown oil, which was purified through a plug of silica gel (16.5×20 cm, 2.4 kg silica gel) using 2:1 heptane/ethyl acetate (15 L) as eluent. The fractions containing the product were combined and concentrated under reduced pressure at 35° C. to afford a mixture of the title compounds as a light brown oil [1785 g, 90%). The ¹H NMR spectrum was consistent with the assigned structure and indicated the presence of a 64:36 ratio of regioisomers.

### c) 2-Bromo-1-(2-trimethylsilanyl-ethoxymethyl)-1H-imidazole-4-carbonitrile

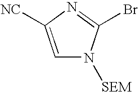

A 22-L, four-neck, round-bottom flask equipped with a mechanical stirrer, a temperature probe, and a condenser with a nitrogen inlet was charged with a mixture of 1-(2-trimethylsilanyl-ethoxymethyl)-1H-imidazole-4-carbonitrile and 3-(2-trimethylsilanyl-ethoxymethyl)-3H-imidazole-4-carbonitrile [600 g, 2.69 mol, as prepared in the previous step) and carbon tetrachloride (1.8 L). Agitation was initiated and the mixture was heated to 60° C. At this point N-bromosuccinimide (502 g, 2.82 mol) was added in several portions over 30 min, which resulted in an exotherm to 74° C. The reaction was allowed to cool to 60° C. and further stirred at 60° C. for 1 h. The reaction was allowed to cool slowly to ambient temperature and the resulting slurry was filtered and the filtrate washed with satd NaHCO₃ solution (4.0 L). The organics were passed through a plug of silica gel (8×15 cm, silica gel; 600 g) using 2:1 heptane/ethyl acetate (6.0 L) as eluent. The fractions containing the product (based on TLC analysis) were combined and concentrated under reduced pressure to give a crystalline light yellow solid, which was then filtered and washed with heptane (500 mL) to give the title compound as a crystalline white solid [593 g, 73%). The ¹H and ¹³C NMR spectra were consistent with the assigned structure and showed no evidence of the minor regioisomer.

### d) 4-Cyano-1-(2-trimethylsilanyl-ethoxymethyl)-1H-imidazole-2-carboxylic acid ethyl ester

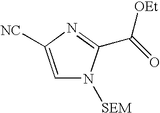

A 12-L, four-neck, round-bottom flask equipped with a mechanical stirrer, a temperature probe, and an addition funnel with a nitrogen inlet was charged with 2-bromo-1-(2-trimethylsilanyl-ethoxymethyl)-1H-imidazole-4-carbonitrile [390 g, 1.29 mol, as prepared in the previous step) and anhydrous tetrahydrofuran (4.0 L). Agitation was initiated and the reaction mixture was cooled to −50° C. using a dry ice/acetone bath. Isopropylmagnesium chloride (2.0 M in THF, 760 mL, 1.52 mol) was added through the addition funnel over 30 min to maintain the internal temperature below −40° C. The reaction was stirred for a further 30 min at −43° C., after which time it was cooled to −78° C. Ethyl chloroformate (210 mL, 2.20 mol) was added through the addition funnel over 10 min to maintain the internal temperature below −60° C. The reaction was stirred for a further 40 min at −70° C., at which point the dry ice/acetone bath was removed and the reaction was allowed to warm to ambient temperature over 1.5 h. The reaction mixture was cooled in an ice bath to 0° C. and quenched by the slow addition of satd ammonium chloride solution (1.8 L) at such a rate that the internal temperature was maintained below 10° C. The reaction mixture was transferred into a 12-L separatory funnel, diluted with ethyl acetate (4.0 L), and the layers were separated. The organic layer was washed with brine (2×2.0 L) and concentrated under reduced pressure at 35° C. to give a brown oil. The crude oil was dissolved in dichloromethane (300 mL) and purified by chromatography (15×22 cm, 1.5 kg of silica gel, 10:1 to 4:1 heptane/ethyl acetate) to give a yellow oil, which was dissolved in EtOAc (100 mL), diluted with heptane (2.0 L), and stored in a refrigerator for 5 h. The resulting slurry was filtered to give the title compound as a crystalline white solid (141 g, 37%). The ¹H and ¹³C NMR spectra were consistent with the assigned structure.

### e) 4-Cyano-1-(2-trimethylsilanyl-ethoxymethyl)-1H-imidazole-2-carboxylic acid potassium salt

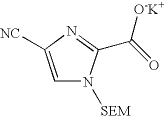

A 5-L, three-neck, round-bottom flask equipped with a mechanical stirrer, a temperature probe, and an addition funnel with a nitrogen inlet was charged with 5 [400 g, 1.35 mol) and ethanol (4.0 L). Agitation was initiated and a water bath was applied after all of the solid had dissolved. A solution of 6 N KOH (214.0 mL, 1.29 mol) was added through the addition funnel over 15 min to maintain the internal temperature below 25° C. and the reaction was stirred for 5 min at room temperature. The solution was then concentrated to dryness under reduced pressure at 20° C. to give a white solid. The resulting solid was suspended in methyl t-butyl ether (MTBE, 4.0 L) and stirred for 30 min, after which time the slurry was filtered and the filter cake washed with MTBE (1.0 L) to give the title compound as a white solid, which was further dried under vacuum at ambient temperature for 4 d [366 g, 89%). The ¹H NMR, ¹³C NMR, and mass spectra were consistent with the assigned structure. Anal. Calcd for C₁₁H₁₆KN₃O₃Si: C, 43.25; H, 5.28; N, 13.76. Found: C, 42.77; H, 5.15; N, 13.37. Karl Fisher: 1.3% H₂O.

### Experimentals

### Materials and Methods.

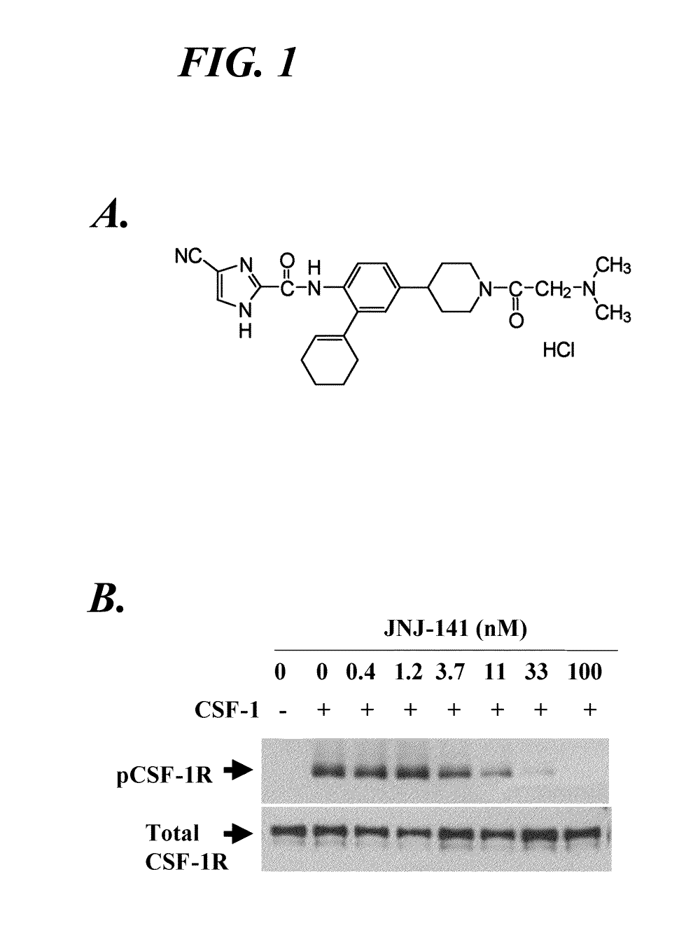

Example 38a, 4-cyano-N-[2-(1-cyclohexen-1-yl)-4-[1-[(dimethylamino)acetyl]-4-piperidinyl]phenyl]-1H-imidazole-2-carboxamide monohydrochloride, shown in FIG. 1A herein (referred to as “JNJ-141” herein), was prepared as described herein.

### Kinase Assays.

The full cytoplasmic regions of CSF-1R (CSF-1R 538-972) and CSF-1R-like tyrosine kinase 3 (FLT3 [FLT3 571-993]) encompassing the tyrosine kinase domains were expressed and purified from a baculovirus system as described in Schalk-Hihi C, et al. J Biol Chem 2007; 208:4085 4093. Stem cell factor receptor tyrosine kinase (KIT) was purchased from ProQinase (Hamburg, Germany). AXL receptor tyrosine kinase (AXL) was purchased from Upstate (Lake Placid, N.Y.). Neurotrophin receptor tyrosine kinase A (TRKA) was purchased from Invitrogen (Carlsbad, Calif.). CSF-1R 555-568 peptide (SYEGNSYTFIDPTQ) was synthesized and purified by AnaSpec (San Jose, Calif.). CSF-1R was assayed using a fluorescence polarization competition immunoassay that measured CSF-1R phosphorylation of CSF-1R 555-568 peptide at Y561. The reaction mixture (10 μL) contained 100 mM HEPES, pH 7.5, 1 mM DTT, 0.01% Tween-20 (v/v), 2% DMSO, 308 μM CSF-1R 555-568 peptide, 1 mM ATP, 5 mM MgCl₂, and 0.7 nM CSF-1R. The reaction was initiated with ATP, incubated 80 minutes at room temperature, and quenched by the addition of 5.4 mM EDTA. Ten μL of fluorescence polarization buffer/tracer/phospho-Y antibody mix (Tyrosine kinase assay kit, Green P2837, Invitrogen, Madison Wis.) were added to the quenched reaction, and fluorescence polarization was measured after 30 minutes using an Analyst reader (Molecular Devices) at excitation/emission of 485/530 nm. FLT3, KIT, TRKA, and AXL were assayed using the fluorescence polarization competition format as described for CSF-1R except that poly Glu4Tyr (Sigma, St Louis, Mo.) was used as a universal substrate. Prior to use, AXL was phosphorylated by incubation with 1 mM ATP, 10 mM MgCl₂, 100 mM HEPES, pH 7.5 for 60 minutes at room temperature, and stored at −70° C. FLT3 reactions contained 10 nM FLT3, 113 μM ATP, and 20 μg/ml poly Glu4Tyr, for 25 minutes. KIT reactions contained 1 nM KIT, 50 μM ATP, and 100 μg/ml poly Glu4Tyr, for 30 minutes. TRKA reactions contained 5 nM TRKA, 20 μM ATP, and 20 μg/ml poly Glu4Tyr, for 30 minutes. AXL reactions contained 0.5 nM AXL, 20 μM ATP, and 25 μg/ml poly Glu4Tyr, for 11 minutes. ATP Kₘ values (Michaelis-Menten constant) for FLT3, KIT, TRKA, and AXL were 50 μM, 44 μM, 29 μM, and 16 μM, respectively. The LCK IC₅₀ and inhibition of sixty kinases at 1 and 0.1 μM were determined using the Invitrogen SelectScreen™ Kinase Profiling Service. Another fifty-one kinases were assayed using the Millipore KinaseProfiler Assay Service.

### Cellular Assays.

Inhibition of CSF-1-induced CSF-1R phosphorylation was measured using HEK293 cells transfected to overexpress wild-type CSF-1R and an ELISA and immunoblot analysis as described previously in Baumann C A, et al., J Biochem Biophys Methods 2004; 60:69-79. A similar approach was used to measure inhibition of FLT3-ligand-induced FLT3 phosphorylation in Baf3 cells transfected to overexpress wild-type FLT3. Baf3 cells transfected to overexpress wild-type FLT3 were used to investigate the inhibition of FLT3 kinase activity in cells. (Yee, K W H, et al., Blood, 15 Oct. 2002, Vol. 100, No. 8, pp. 2941-2949). The phosphorylation state of FLT3 was assessed following stimulation with FLT3-L. Cells were plated in RPMI 1640 with 0.5% serum and 0.01 ng/mL IL-3 for 16 hours prior to a 1 hour incubation with graded concentrations of JNJ-141 or DMSO vehicle. Cells were treated with 100 ng/mL FLT3-L for 10 min. at 37° C. and immediately lysed. Phosphorylated FLT3 was quantified using a sandwich-type ELISA. Cleared lysates were transferred to microtiter plates coated with 50 ng/well FLT3 antibody (Santa Cruz Biotechnology Corp, Santa Cruz, Calif.; sc-480) and blocked with SeaBlock reagent (Pierce Chemicals, Rockford, Ill.). Lysates were incubated at 4° C. for 2 hours. Washed plates were incubated with 1:8000 dilution of HRP-conjugated phosphotyrosine antibody (Clone 4G10, Upstate Biotechnology) for 1 hour at room temperature. Following a final wash, signal detection with SuperSignal® Pico reagent (Pierce Chemical, Rockford, Ill.) was completed according to manufacturer's instructions on a Berthold Orion microplate luminometer Inhibition and ICₛₒ data analysis was done with GraphPad Prism® software using a nonlinear regression fit with a multiparameter, sigmoidal dose-response (variable slope) equation.

GAS6-induced AXL phosphorylation was measured using HEK293 cells transfected to overexpress AXL. HEK293E cells were engineered to express full length Axl and subsequently used to assay JNJ-141 inhibition of Gas6 mediated Axl phosphorylation. The episomal expression vector pCEP4-His6 was used to overexpress full-length human Axl in HEK293E cells. Human GAS6 was purified from conditioned media generated from the GAS6/HEK293E cell line (Fisher P W, et al., Biochem. J. (2005) 387, 727-735.). Axl/HEK293E cells were pretreated for 40 minutes with JNJ-141 prior to stimulation for 10 minutes with 200 ng/ml human GAS6. Cells were lysed with RIPA buffer (Santa Cruz sc-24948) and Axl was immunoprecipitated overnight with human Axl antibody (Santa Cruz, sc-1096) and collected onto A/G agarose (Santa Cruz sc-2003) Immunoprecipitates were resolved on 4-12% NuPAGE gels and transferred to nitrocellulose. Replicate blots of washed immunoprecipitates were probed using either an HRP-conjugated phosphotyrosine antibody (clone 4G10, Upstate) or a human Axl antibody to confirm equal loading of total Axl. Proteins were detected with SuperSignal® West Chemiluminescent substrate. Quantitation of x-ray films was by scanning densitometry using a UVP bioimaging system and LabWorks software. Inhibition and IC₅₀ data analysis was done with GraphPad Prism® software using a nonlinear regression fit with a multiparameter, sigmoidal dose-response (variable slope) equation.

Functional inhibition of CSF-1R was examined using assays of CSF-1-driven mouse macrophage proliferation and CSF-1-induced MCP-1 production by human monocytes. Monocytes isolated from human blood by negative selection using RosetteSep® human monocyte enrichment cocktail from StemCell Technologies (Cat. #15068) were cultured (2×10⁵/well) in round bottomed 96-well polypropylene plates (Corning 3790) with RMPI 1640 containing 10% heat-inactivated FBS and graded concentrations of JNJ-141 for 30 minutes. Cells were then stimulated 16 hrs with 100 ng/ml recombinant human CSF-1 (R&D Systems) and a specific ELISA (R&D Systems) was used to assay culture supernatants for MCP-1. Mouse macrophages were derived from bone marrow flushed from the femurs of B6C3F1 mice (Harlan Industries, Indianapolis, Ind.). Bone marrow cells suspended (1×10⁶ cells/ml) in culture medium (EMEM containing 10% FBS, 2 mM glutamine, 100 IU/ml penicillin and 100 ug/ml streptomycin, and 50 ng/ml recombinant murine CSF-1 (R&D Systems)) were cultured in tissue culture flasks (Falcon) at 37° C. and 5% CO₂ overnight. The non-adherent cells were re-plated into 100 mm bacteriological dishes (Falcon 35 1029) (10 ml/dish) and media was replaced after three and six days. On the seventh day, bone marrow-derived macrophages (BMDM) were harvested using Cellstripper™ (CellGro, Mediatech, Inc., Herndon, Va.), resuspended in culture media without CSF-1, and plated at a density of 5000 cells/well into Costar 96-well tissue culture plates. After overnight culture, wells were adjusted to contain 5 ng/ml CSF-1, 1 μM indomethacin, and graded concentrations of JNJ-141. Twenty-four hours later, wells were further adjusted to contain bromodeoxyuridine (BrDU) for an additional 6 hrs. Incorporation of BrDU into the DNA of proliferating macrophages was quantified by ELISA (Exalpha Corp. Watertown, Mass.) and concentrations of JNJ-141 that inhibited BrDU incorporation by fifty percent were calculated using GraphPad Prism® software and a four parameter logistics equation.

Cell proliferation dependent on ITD-FLT3, KIT, and TRKA was assessed using the MV-4-11 AML cell line (ATCC Number: CRL-9591), the M07e erythroleukemia cell line (DSMZ Number: ACC 104), and the TF-1 myeloid leukemia line (ATCC Number: CRL-2003), respectively. MV-4-11 cells are growth factor-independent due to the expression of a constitutive active ITD-FLT3 mutant (Quentmeier H, et al., Leukemia 2003; 17:120-124). M07e cells express KIT and proliferate in response to SCF (B Lange, et al., Blood 1987; 70:192-199.) TF-1 cells express TRKA and proliferate in response to NGF (B Lange, et al., Blood 1987; 70:192-199.) Cells were dispensed into Costar 96-well tissue culture plates (10,000 cells/well) together with graded concentrations of JNJ-141. M07e and TF-1 cultures were adjusted to contain 25 ng/ml SCF or 1.4 ng/ml NGF, respectively. Following a culture period of 72 hours, relative cell numbers were determined using CellTiterGlo™ reagent (Promega). MV-4-11 growth was calculated based on the difference between luminescence on Day 3 vs. Day 0. M07e and TF-1 growth was calculated based on the difference in luminescence of cells cultured in the presence vs. the absence of growth factor. IC₅₀ values were determined with GraphPad Prism® software using a nonlinear regression fit with a multiparameter, sigmoidal dose-response (variable slope) equation.

Inhibition of cellular LCK was determined as described in Maier J A, et al., Bioorganic & Medicinal Chemistry Letters 2006; 16:3646-3650. Jurkat cells (10⁵/well) (ATCC TIB-152) (clone E6-1) were pretreated with graded concentrations of JNJ-141 in round bottom 96 well-polypropylene plates for one hour. Cells were transferred to dishes pre-coated with CDR antibody (MAB100 R&D Systems). PMA was then added to a final concentration of 10 ng/ml and cells were incubated overnight at 37° C. Twenty-four hour culture supernatants were harvested and IL-2 protein expression determined by ELISA (R&D Systems). Cell viability was confirmed with CellTiter-Glo™ reagent.

### Animal Studies.

Animals were housed in facilities fully accredited by the American Association for Assessment and Accreditation of Laboratory Animal Care (AAALAC), and procedures involving animals were conducted in compliance with the NIH Guide for the Care and Use of Laboratory Animals.

In vivo pharmacodynamic activity of JNJ-141. Groups of six B6C3F1 mice (Taconic Farms), 8 weeks of age, were given oral doses of vehicle (aqueous 20% hydroxypropyl-β-cyclodextrin (HPβCD)) or JNJ-141 at 10 or 20 mg/kg. Eight hours later, mice were administered saline or 0.8 μg recombinant mouse CSF-1 (Cell Biosciences Inc, Norwood, Mass.) via the tail vein. Fifteen minutes after the tail vein injection, mice were sacrificed and spleens were isolated and snap frozen on dry ice. The frozen tissue was homogenized in 1 ml of Trizol (Invitrogen) per 50 mg of tissue, and RNA was purified according to the Trizol instructions and treated with 6.8 Kunitz units of RNase-free DNase (Qiagen, Valencia, Calif.) to degrade contaminating genomic DNA. The RNA was purified further using RNeasy columns (Qiagen). RT-PCR was performed in 25 μL reaction volumes using Reverse Transciptase qPCR Master Mix (Eurogentec) and approximately 50 ng RNA. Applied Biosystems, Inc., (Foster City, Calif.) was the source for the primer probe set for mouse c-fos mRNA (part# Mm00487425) and 18S rRNA (part# 4333760F). Amplification and detection were performed using the ABI Prism 7000 Sequence Detector system. Standard curves were created for c-fos mRNA and for 18s rRNA using RNA isolated from a vehicle-treated, CSF-1-induced mouse and used to calculate relative expression levels in all other samples. c-fos mRNA values were normalized to 18S rRNA content. Averaged, normalized c-fos content in the saline (no CSF-1) group was assigned a value of one and all other groups were expressed as “fold-induced”.

NCI-H460 human lung tumor xenograft model. NCI-H460 human lung carcinoma cells (ATCC Number HTB-177) were suspended at 1×10⁷ cells/mL in sterile PBS and 100 uL were injected s.c. in the left inguinal region of female athymic nude mice (CD-1, nu/nu, 9 to 10 weeks old) from Charles River Laboratories (Wilmington, Mass.). Three days later, mice were randomized into four groups (15 per group) and oral gavage dosing was initiated with vehicle or with JNJ-141 at doses of 25, 50 and 100 mg/kg. Dosing was twice daily during the week and once daily on weekends for 25 consecutive days. Tumor volumes were determined using electronic Vernier calipers using the formula (L×W)²/2, where L=length (mm) and W=width (shortest distance in mm) of the tumor. At study termination, blood samples were collected by cardiac puncture under CO₂ anesthesia in lithium heparin-coated tubes. Plasma was obtained by centrifugation (3000 rpm) at 4° C. for 10 minutes and stored frozen at −80° C. until analyzed for human and mouse CSF-1 using specific ELISAs (R&D Systems). Half of each tumor was immersed in Tissue-Tek O.C.T. (optimal cutting temperature) media (VWR, West Chester, Pa.), snap frozen and processed for immunohistochemical staining of the tumor vasculature. The other half of each tumor was fixed in 10% formalin and embedded in paraffin for immunohistochemical quantization of TAMs. Five μm sections were stained using rat anti-mouse F4/80 (Clone C1:A3-1, Serotec) and an HRP detection system including biotinylated rabbit anti-rat immunoglobulins (Dako Cytomation, Catalog Number: E0468) and anti-rabbit Envision with labeled polymer-HRP (Dako Cytomation, Catalog Number: K4003) and DAB. For each tumor, the three areas of highest macrophage density were assessed at 200× magnification. The percentage of each field positive for F4/80 stained cells was determined with the aid of Image Pro Plus software and the three fields were averaged for each tumor. To assess tumor vessel density, 8 μm cryostat sections were fixed in cold acetone for 5 min and air-dried. The sections were washed in PBS, blocked with 5% goat serum in PBS, and blocked further with Avidin-Biotin solution (SP-2001, Vector Corporation, Burlingame, Calif.). After washing, the sections were covered with PBS containing 10 μg/ml rat anti-mouse CD31 (RM5200, Caltag Laboratories, Burlingame, Calif.) for 60 minutes, washed and stained using the ABC-AP Rat kit (AK-5004, Vector Corporation). Levamisole was mixed with substrate to inhibit endogenous alkaline phosphatase. Rat IgG (Caltag Labs, R2a00), was used as a negative control and was negative in all cases. The sections were lightly counterstained and photographs were taken using a 4× objective lens. The percentage of tumor cross-sectional area occupied by vessels was calculated using Image Pro Plus (Phase 3 Image).

Rat MRMT-1 bone metastasis model. Inoculation of rat mammary MRMT-1 adenocarcinoma cells into tibiae has been described as a bone metastasis model (Medhurst S J, et al., Pain 2002; 96:129-40. An adaptation of the model (Roudier M P, et al., Clin Exp Metastasis 2006, 23:167-75) was performed by MDS Pharma Services (Bothel, Wash.). Female Sprague-Dawley rats (Harlan Sprague Dawley, Inc., Indianapolis, Ind.) approximately 125-150 grams were acclimated one week. The animals were anesthetized with ketamine/xylazine, and the right leg area was shaved and scrubbed with chlorhexadine and 70% ethanol (SOP-SUR026). A 1-cm rostral-caudal incision was made in the skin over the top half of the tibia. Blunt-dissection exposed the proximal tibia, and a Hamilton syringe guided by a 23-gauge needle was used to inject 3 μl of saline (sham) or saline containing 3×10⁴ MRMT-1 cells into the medullary cavity of the right proximal tibia of each rat. The injection site was closed with bone wax. The wound was closed with surgical staples. Animals were dosed by oral gavage twice daily, 10 hours apart, beginning on day 3 with vehicle (0.5% hydroxypropyl-methylcellulose) or JNJ-141 (20 or 60 mg/kg). To serve as a control, a fourth group was dosed QOD sc with 0.03 mg/kg zoledronate in saline. Eight rats were dosed per group. Rats were sacrificed on Day 17. Right tibiae were excised, together with surrounding tumor tissue, and microradiographs prepared using an MX-20 x-ray system (Faxitron X-ray Corporation, Wheeling, Ill.). Microradiographs were scored for tumor-induced osteolysis as follows: 0, no signs of destruction; 1, one to three small radiolucent lesions; 2, three to six lesions and loss of medullary bone; 3, loss of medullary bone and erosion of cortical bone; 4, full thickness uni-cortical bone loss; 5, full thickness bi-cortical bone loss and/or displaced skeletal fracture. The radiographs were used to select representative bones from each group for microCT imaging. All tibia were fixed in 10% neutral buffered formalin for two days, decalcified and sectioned for histopathological evaluation. TRAP staining was performed as described previously in Liu, C., et al., 1987. Histochemistry 86: 559-565. For each tibia, the numbers of tumor-associated TRAP⁺ osteoclasts were counted in the three 200×-fields with highest osteoclast frequency. A semi-quantitative three-point scoring method was used to compare treatment groups with regards to trabecular bone volume (3, >40 area; 2, >10%<40% area (normal); 1, 1-10% area; 0, none) and tumor volume (3, large; 2, moderate; 1, small; 0, none).

Pain Behavioral Assessments. Prior to surgery and on days 5, 7, 10, 14 and 16 the animals underwent behavioral analysis for tactile allodynia. The behavioral test for tactile allodynia, measured prior to surgery, was used for randomization of animals to the treatment groups. Rats were habituated to the von Frey testing apparatus, and tactile (i.e. mechanical) allodynia was determined by applying a series of calibrated nylon fibers through the cage floor and pressed against the plantar surface of the hind paw. The rats were unrestrained and unhandled during the test. The diameters of the von Frey filaments correspond to a logarithmic scale of force exerted and thus a linear and interval scale of perceived intensity. The withdrawal threshold was determined according to Chaplan's “up-down” method (Chaplan S R et al., J Neurosci Methods 1994 53(1):55-63) involving the use of successively larger and smaller fibers to allow identification of the 50% withdrawal threshold. Briefly when the rat lifted its paw in response to the pressure, the filament size was recorded and a weaker filament was used next. Conversely, in the absence of a response, a stronger stimulus was used. Strings of similar responses were thus generated and the 50% response threshold was calculated using a response variable spreadsheet. Significant differences in tactile allodynia were based on the comparison of group mean values.

2472 sarcoma model of secondary bone tumor pain and osteolysis. Experiments were performed on adult male C3H/HeJ mice (Jackson Laboratories, Bar Harbor, Me., USA), approximately 7-8 weeks old, weighing 25-30 g at the time of tumor cell injection. An arthrotomy was performed following induction of general anesthesia with ketamine/xylazine (2:1 ratio; intramuscular (i.m.)). A needle was inserted into the intramedullary canal to create a pathway for the sarcoma cells. A depression was then made using a pneumatic dental high speed handpiece. Mice were injected with Hanks buffered saline (HBSS) (20 ul, Sigma, St. Louis, Mo., USA) or HBSS containing the 2472 sarcoma line (ATCC, Rockville, Md., USA). The injection site is sealed with a dental amalgam plug to confine the cells within the intramedullary canal and followed by irrigation with sterile water (hypotonic solution). Finally, incision closure is achieved with a wound clip. Clips are removed at day 5 as not to interfere with behavioral testing.

The extent of tumor-induced bone reorganization (osteolysis) was radiologically assessed using Faxitron analysis (Specimen Radiography System Model MX-20, Faxitron X-ray Corporation, Wheeling, Ill., and Kodak film Min-R 2000, Rochester, N.Y.). Radiographs of tumor-bearing femora were scored on a 0 to 5 scale: (0) normal bone with no signs of destruction; (1) small pits of bone destruction (1-3 in number); (2) increased pitted appearance (3-6) and loss of medullary bone; (3) loss of medullary bone and erosion of cortical bone; (4) full thickness unicortical bone loss; (5) full thickness bicortical bone loss and displaced skeletal fracture.

A variety of behavioral measurements were used to assess the extent of bone cancer pain.

Spontaneous nocifensive behaviors: The number of spontaneous flinches and guarding, representative of nociceptive behavior, were recorded during a 2-minute observation period. Flinches are defined as number of times the animal raises its hindpaw and guarding as the amount of time animals hold the hindpaw aloft while stationary.

Palpation-induced nocifensive behaviors: Mechanical allodynia at the knee joint was evaluated by normally non-noxious palpation of the distal femur every second for 2 minutes. Following the 2-minute palpation, the mice were placed in the observation box and their palpation-induced guarding and flinching behavior was measured for an additional 2 minutes, as discussed above.

Forced ambulatory guarding: Forced ambulatory guarding: was determined using a Roto-Rod (IITC, Woodland Hills, Calif.). The Roto-Rod machine has a revolving rod and is equipped with speed, acceleration, and sensitivity controls. The animals will be placed on the rod with X4 speed, 8.0 acceleration, and 2.5 sensitivity controls. Forced ambulatory guarding was rated on a scale of 5 to 0: (5) normal use, (4) some limp, but not pronounced, (3) pronounced limp, (2) pronounced limp and prolonged guarding of limb, (1) partial non-use of the limb, and (0) complete lack of use.

Statistical analysis. Differences between treated and control animals were analyzed statistically by ANOVA and Dunnett's t-test, with a p-value of 0.05 (2-tailed) considered statistically significant. GraphPad Prism Version 4.0 was used for all statistical analyses and graphical presentation of the data.

### Results.

JNJ-141 is a potent inhibitor of CSF-1R and FLT3 with a narrow kinase selectivity profile. In an enzyme assay, JNJ-141 inhibited human CSF-1R kinase with an ICₛₒ value of 0.00069 μM. Specificity for CSF-1R vs. 110 other kinases was examined Ninety-three kinases were inhibited less than fifty percent at 1 μM. Of the remaining seventeen kinases, five had IC₅₀ values less than 0.1 μM including KIT (0.005 μM), AXL (0.012 μM), TRKA (0.015 μM), FLT3 (0.030 μM), and LCK (0.088 μM).

JNJ-141 was characterized further in cellular assays. Results are presented in Table 1.

TABLE 1
Inhibitory Activity of JNJ-141 in Cellular Assays

| Cells/cell-line | Target | Stimulus | Endpoint | IC₅₀ (μM)^{a} |
|---|---|---|---|---|
| mouse bone marrow- | CSF-1R | CSF-1 | proliferation | 0.0026 |
| derived macrophages | | | | |
| CD14⁺ monocytes | CSF-1R | CSF-1 | Induced MCP-1 | 0.0030 |
| | | | protein | |
| CSF-1R/HEK | CSF-1R | CSF-1 | Phospho-CSF-1R | 0.012 |
| MV-4-11 | ITD-FLT | None | proliferation | 0.021 |
| M07e | KIT | SCF | proliferation | 0.041 |
| FLT3/Baf3 | FLT3 | FLT3-ligand | Phospho-FLT3 | 0.076 |
| TF-1 | TRKA | NGF | proliferation | 0.15 |
| Jurkat | LCK | Anti-CD3, | Induced IL-2 | 3.9 |
| | | PMA | protein | |
| AXL/HEK | AXL | GAS6 | Phospho-AXL | 2.6 |
| H460, MDA-MB-231, | General | None | proliferation | >5.0 |
| A3 75 | | | | |

^{a}Results are means of at least three independent determinations except for FLT3/Baf3, Jurkat, and H460, MDA-MD-231, and A375 assays which were determined once.

Low nanomolar concentrations inhibited CSF-1R autophosphorylation in recombinant HEK cells (FIG. 1B) and CSF-1R-dependent proliferation of mouse macrophages and MCP-1 expression by human monocytes (Table 1). Relative to CSF-1R inhibition, approximately seven-fold higher concentrations of JNJ-141 inhibited the FLT3-dependent proliferation of MV-4-11 cells and FLT3 autophosphorylation in recombinant Baf3 cells. Approximately fifteen-fold higher concentrations inhibited KIT-dependent proliferation of M07e cells. TRKA-dependent proliferation of TF-1 cells was also inhibited at sub-micromolar concentrations of JNJ-28312141. In contrast to CSF-1R, FLT3, KIT, and TRKA cell potencies, the cellular IC₅₀ values for AXL autophosphorylation and LCK-dependent IL-2 production were greater than one micromolar. JNJ-141 (5 μM) did not inhibit the growth factor-independent proliferation of H460, MDA-MB-231, or A375 adenocarcinoma cells. In total, the data identified JNJ-141 as a potent, selective inhibitor of CSF-1R with additional cellular inhibition of FLT3, KIT, and TRKA at nanomolar concentrations.

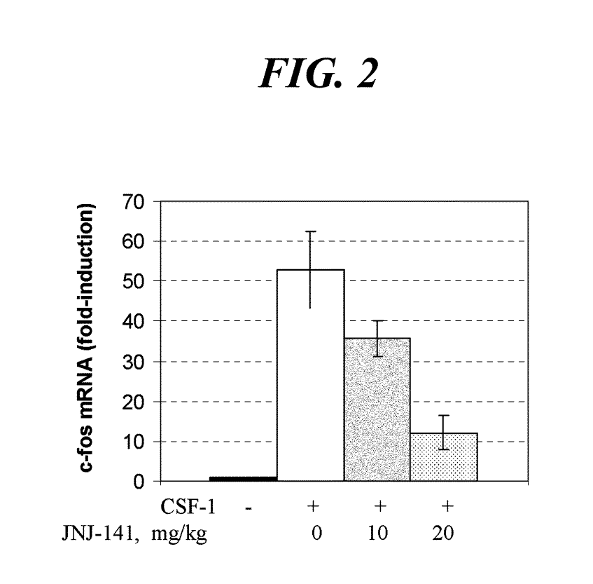

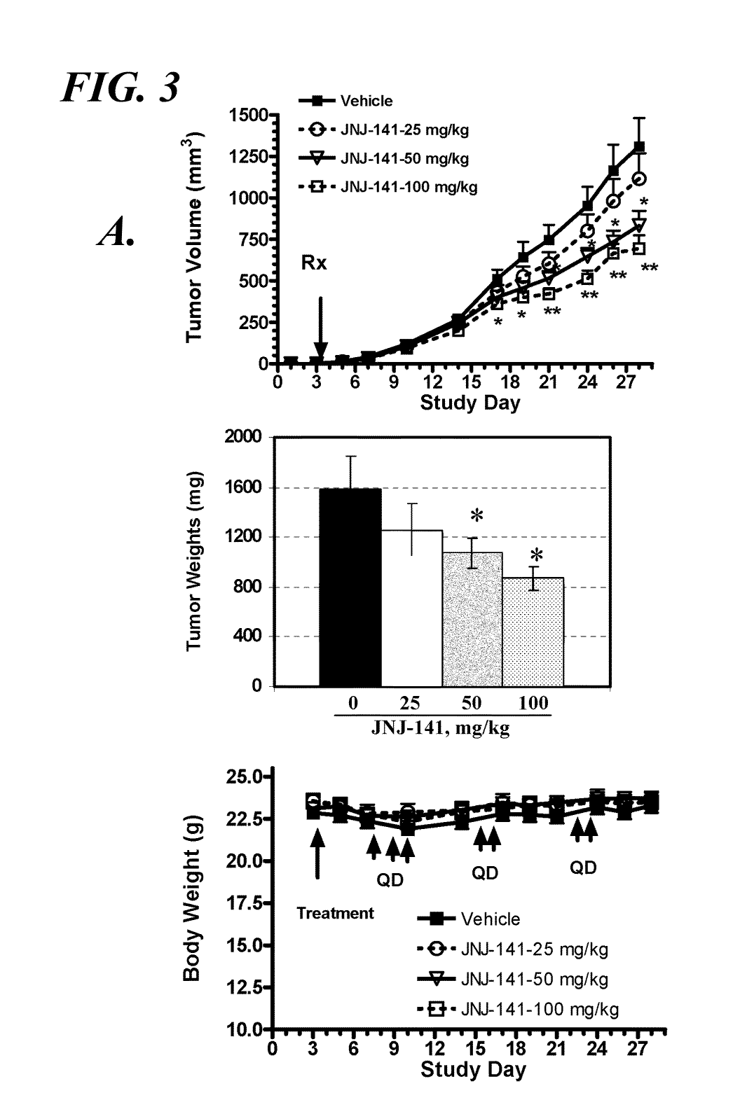

In vivo pharmacodynamic activity of JNJ-141. To confirm CSF-1R inhibition in vivo, a simple pharmacodynamic model was developed based on the reported ability of CSF-1 to elevate macrophage c-fos mRNA (Orlofsky A, et al., EMBO J 1987; 6: 2947-52.) Because macrophages are present in large numbers in spleens, c-fos mRNA in spleens of mice following intravenous recombinant CSF-1 was assessed. CSF-1 induced splenic c-fos mRNA 10- to 50-fold within fifteen minutes, but returned to baseline by 30 minutes. Induction was dose-dependent (ED₅₀ ca. 0.8 μg/mouse) and blocked 100% in mice dosed (i.p.) with 0.2 mg CSF-1-neutralizing monoclonal 5A1 antibody (BD Biosciences Pharmingen). When an oral dose of 10 or 20 mg/kg JNJ-141 was administered eight hours prior to the 0.8 μg CSF-1 challenge, c-fos mRNA induction was reduced 33% and 79%, respectively as shown in FIG. 2.

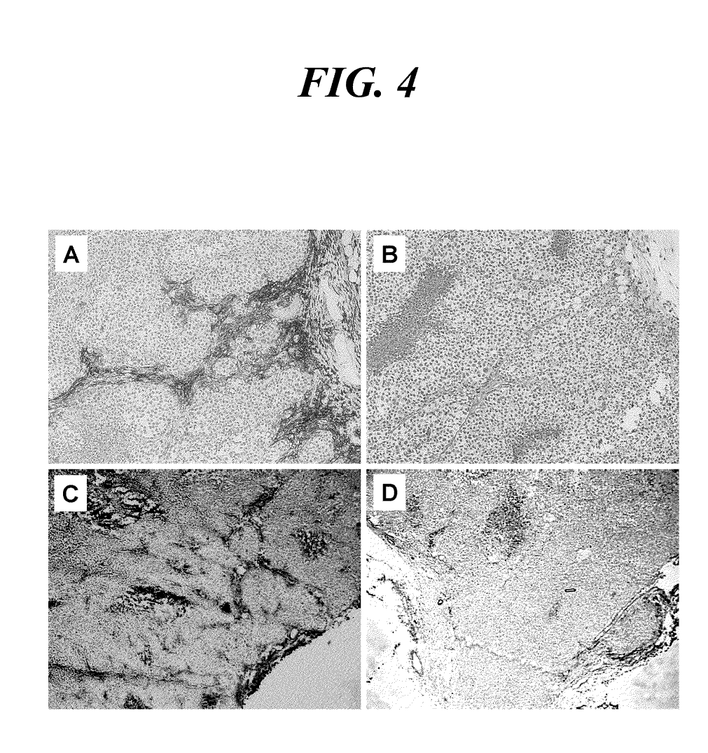

Growth inhibition of H460 lung adenocarcinoma xenografts by JNJ-141. JNJ-141 was used to test the hypothesis that CSF-1R-dependent macrophages support the growth of solid tumors. H460 lung adenocarcinoma xenografts were selected as a model based on three criteria. First, human CSF-1R expression was undetectable by RT-PCR in H460 cells or in xenografts and growth of H460 cells in culture was not suppressed by JNJ-141 (See Table 1, above). Second, lysates of H460 tumors contained ample quantities (35 ng/g wet weight) of human CSF-1, and H460 tumors developed a stroma well populated with macrophages (see FIG. 4). Lastly, viable H460 cells were limited to areas adjacent to a penetrating, serpentine vascular stroma, suggestive of stromal-dependent tumor growth. Together, these tumor characteristics provided an opportunity to investigate the putative contribution of CSF-1R-dependent macrophages to tumor growth.

JNJ-141 dose-dependently reduced H460 xenograft growth rates (see FIG. 3A). At study termination, final tumor weights were reduced by 21, 32 and 45% at 25, 50 and 100 mg/kg, respectively (See FIG. 3B). No overt toxicity or adverse effects on body weight were observed during the 25-day treatment period (See FIG. 3C).

### JNJ-141 Reduced Tumor-Associated Macrophages and Vascularity.

TABLE 2
JNJ-141 Reduced The Growth of H460 Tumorsand Reduced
Tumor-Associated Macrophages and Microvasculature

| Dose^{a} | Mean final | Macrophage | Microvascular |
|---|---|---|---|
| JNJ-141, | tumor | density, | density, |
| (mg/kg) | weight, mg | % area^{b} | % area^{c} |
| 0 | 1,584 ± 257 | 10.6 ± 1.83 | 3.66 ± 0.85 |
| 25 | 1,257 ± 209 | 4.3 ± 0.68** | 3.73 ± 0.81 |
| 50 | 1,070 ± 121 | 2.6 ± 0.57** | 2.10 ± 0.57 |
| 100 | 870 ± 94* | 0.31 ± 0.14** | 1.25 ± 0.16* |

Values represent means ± SEM
*p < 0.05 versus control;
**p < 0.01 versus control
^{a}Twice daily (accept qd on weekends) oral dosing commenced three days after subcutaneous inoculation of 1 × 10⁶ H460 lung adenocarcinom cells and continued until sacrifice on Day 25.
^{b}Percent area (200X-field) F4/80 positive.
^{c}Percent area CD31 positive.

To investigate the mechanism of action of JNJ-141 at the cellular level, TAMS were quantified by image analysis. F4/80 positive macrophages were abundant in the tumor stroma of vehicle-treated mice (FIG. 4A) and were present (albeit in lower numbers) within regions dominated by tumor cells. JNJ-141 efficiently reduced tumor-associated macrophages in a dose-dependent fashion (Table 2 and FIG. 4B) with ca. 97% reduction observed at the 100 mg/kg dose. The remaining positive cells were small and round and lacked the morphology of mature tissue macrophages.

To determine if reduced macrophage counts were associated with reduced tumor microvessel density, tumors were stained and quantified for CD31⁺ microvasculature (FIGS. 4C and 4D). In vehicle-treated mice, CD31⁺ microvasculature was present throughout the tumor stroma (FIG. 4C). Treatment with JNJ-141 resulted in a dose-dependent reduction in the tumor vascularity with a 66% reduction observed at the highest dose (Table 2 and FIG. 4D).

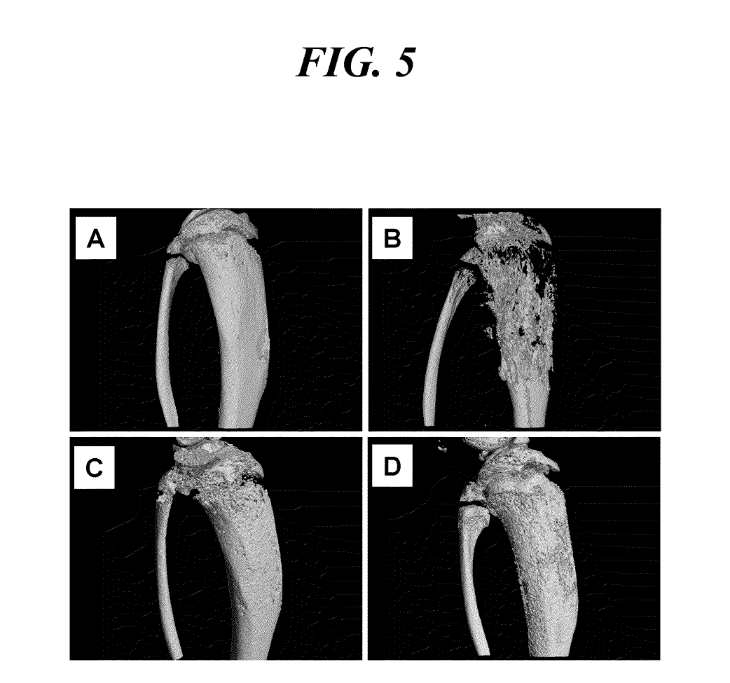

Inhibition of osteoclastogenesis and osteolysis by JNJ-141 in a rat model of bone metastasis. Lung and breast carcinoma are frequently associated with lytic skeletal metastases (Roodman G D., NEJM 2004; 350:1655-64.). Because CSF-1-null mice are deficient in osteoclasts, the effects of oral JNJ-141 in a well-characterized rat syngeneic MRMT mammary carcinoma model of bone metastasis were examined (Medhurst S J, et al., Pain 2002; 96:129-40.). The effects of JNJ-141 were compared with the bisphosphonate, zoledronate. Following inoculation of MRMT mammary carcinoma cells into tibiae, tumors formed both in the marrow cavity and the surrounding periosteum. By day 17, microradiography (results presented in Table 3) and micro-computed tomography (see FIG. 5) revealed extensive loss of trabecular bone and full thickness cortical lesions in vehicle-treated rats.

TABLE 3
JNJ-141 Inhibits MRMT-Induced Bone Erosions and
Inhibits Tumor-Associated Osteoclastogenesis

| | Microradiograph | Trabecular bone | Osteoclast | Tumor vol |
|---|---|---|---|---|
| Treatment^{a} | score^{b} | vol score^{c} | Counts^{d} | score^{c} |
| No cells | 0 | 2.29 ± 0.31 | — | — |
| MRMT + vehicle | 3.88 ± 0.24 | 0.88 ± 0.13 | 58.0 ± 5.6 | 3.00 ± 0 |
| MRMT + JNJ-141, | 0.75 ± 0.17** | 2.00 ± 0.0.2** | 3.7 ± 0.7** | 1.75 ± 0.48* |
| 20 mpk | | | | |
| MRMT + JNJ-141, | 0.83 ± 0.18** | 2.33 ± 0.23** | 2.6 ± 1.5** | 1.50 ± 0.55* |
| 60 mpk | | | | |
| MRMT + zoledronate, | 0.38 ± 0.20** | 2.63 ± 0.20** | 21.0 ± 3.2** | 1.63 ± 0.20** |
| 0.03 mpk | | | | |

Values represent means ± SEM
*p < 0.05 versus control;
**p < 0.01 versus control
^{a}On Day 0, saline (no cells) or saline containing 3 × 10⁴ MRMT-1 rat mammary gland carcinoma cells were injected into the medullary cavity of the right proximal tibia of each rat. Twice-daily oral dosing with vehicle or with 20 or 60 mg/kg JNJ-141 or QOD sc dosing with 0.03 mg/kg zoledronate commenced on Day 3 until sacrifice on Day 17.
^{b}Based on five point visual score (see Materials and Methods).
^{c}Based on three point visual score (see Materials and Methods).
^{d}Mean number of TRAP positive cells in three 200x fields with greatest numbers of tumor-associated osteoclasts.

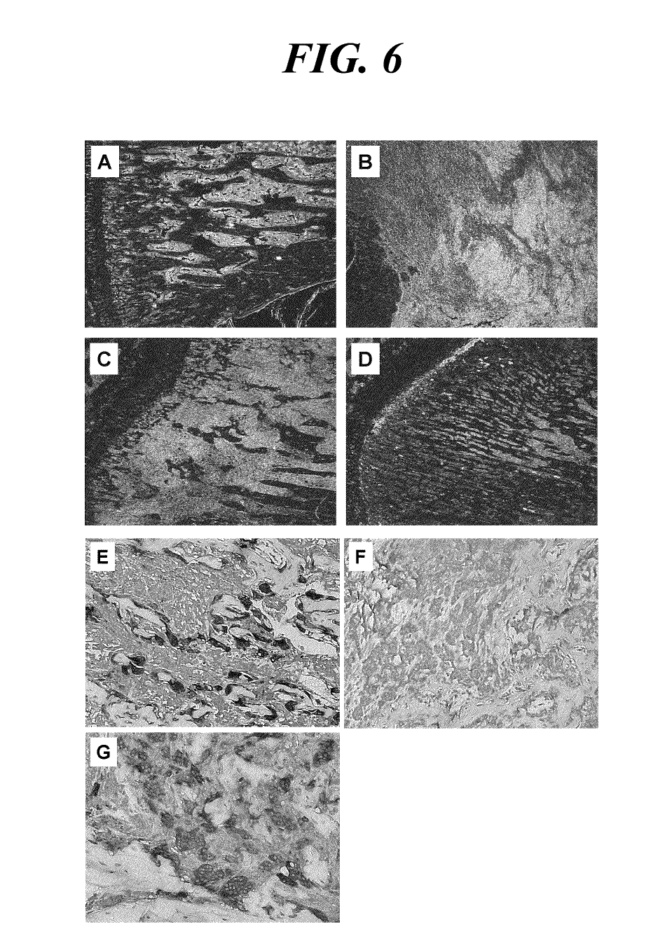

In marked contrast, treatment with JNJ-141 efficiently preserved bone. By day 17, erosion was still undetectable by microradiography in three of fourteen rats dose with either 20 or 60 mg/kg JNJ-141, while in eleven of fourteen rats, one to three small radiolucent lesions could be discerned. The impact of JNJ-141 on radiographic scores was similar to that following zoledronate. Histology confirmed the extensive loss of trabecular bone lesions in vehicle-treated rats bearing MRMT tumors (FIG. 6 and Table 3). JNJ-141 prevented tumor-associated erosions and overall trabecular bone scores were not different from sham (tumor-free) rats. Full thickness cortical bone lesions were routine in the vehicle-treated group but were not observed in the rats treated with JNJ-141 or zoledronate. Regardless of treatment, the marrow cavities of nearly all tumor-inoculated rats were filled with necrotic tumor, whereas viable tumor encapsulated the tibia. Both JNJ-141 and zoledronate reduced the overall size of the encapsulating tumors as assessed on day 17. In vehicle-treated rats, large, multinucleated TRAP⁺ osteoclasts were abundant in the tumors and lined residual cortical bone (FIG. 6). Tumor-associated osteoclasts were reduced by 64% by zoledronate and remained visible under low power magnification. In contrast, tumor-associated osteoclasts were difficult to find in rats treated with JNJ-141. JNJ-141 reduced tumor-associated osteoclasts by greater than 95% and the remaining few osteoclasts were small and often mononuclear.

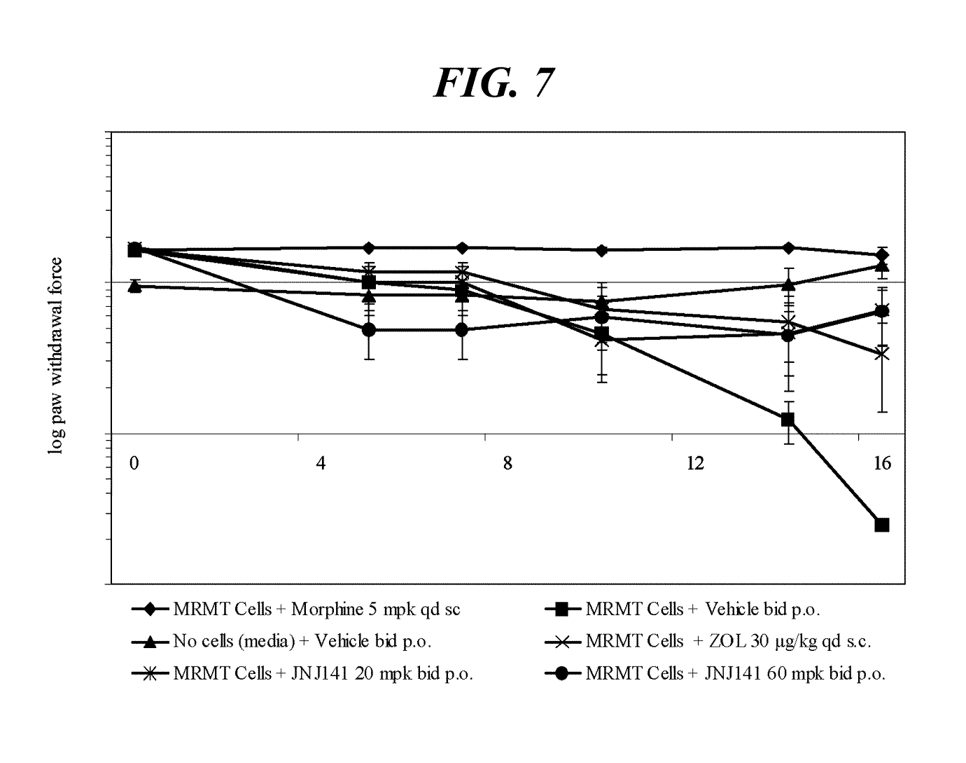

JNJ-141 prevented the onset of metastatic bone pain. Inoculation of MRMT-1 cells into the proximal tibia significantly increased mechanical allodynia in animals inoculated with MRMT-1 cells compared to animals inoculated with media at the final time point; p<0.01. Treatment of affected animals with morphine reversed allodynia from the 2nd time point forward, while treatment with either 20 mpk or 60 mpk of JNJ-141 decreased allodynia compared to tumor-inoculated animals at the final time point (p<0.05 and 0.01, respectively). Zoledronate treatment also decreased allodynia compared to tumor-inoculated animals but this effect did not reach statistical significance. Values in FIG. 7 represent group means±SEM.

Inhibition of osteolysis and pain related behaviors by JNJ-141 in a mouse model of bone metastasis. Inoculation of syngeneic NCTC 2472 osteolytic sarcoma cells into the femurs of C3H/HeJ mice provides a well-characterized model of bone metastasis with pain and osteolytic endpoints (Sevcik M A et al., Pain 2005; 115:128-41). JNJ-141 dose dependently prevented tumor associated bone erosions in this model (Table 4). Prevention of osteolysis was accompanied by reduced numbers of tartrate resistant acid phosphatase (TRACP) positive osteoclasts at the bone tumor interface. Pain related behaviors including spontaneous and palpation induced guarding (SG, PIG) and flinching (SF, PIF) were evident seven days after tumor inoculation and progressed with time in the vehicle treated group (FIG. 8 and Table 4). The progressive increase in pain related behaviors was prevented by JNJ-141 in a dose dependent fashion. By Day 15, palpation induced flinching was reduced by approximately 50% in mice treated with 120 mg/kg JNJ-141 (Table 4).

TABLE 4
Efficacy of JNJ-141 in a Rodent Model of Tumor-Induced Bone Pain

| | | | | | | | Pain-related Scores | | | | | | | |
|---|---|---|---|---|---|---|---|---|---|---|---|---|---|---|
| | | | Bone | Osteo- | Macro- | | | | PIF^{d} | | | SF^{d} | PIG^{e} | SG^{e} |
| Dose,^{a} | | | erosion | clast | phage | Tumor area % | Day | Day | Day | Day | Day | Day | Day | Day |
| (mg/kg) | | n | score^{b} | Counts^{c} | Counts^{c} | marrow | 7 | 9 | 11 | 13 | 15 | 15 | 15 | 15 |
| 0 | (sham) | 3 | 0.17 | 143 | 188 | NA | 2.5 | 1.8 | 1.7 | 2 | 1.7 | 1.7 | 0.7 | 0.7 |
| 120 | (sham) | 4 | 0.25 | 41 | 28 | NA | 2.5 | 1.3 | 1.3 | 1.8 | 1.8 | 1.3 | 0.9 | 0.6 |
| 0 | | 3 | 4.5 | 253 | 376 | 87 | 11.3 | 13.3 | 15.3 | 16.7 | 18.0 | 14.7 | 11.8 | 9.8 |
| 60 | | 3 | 3.5 | 79** | 84** | 100 | 10.3 | 14.3 | 16.3 | 12.7* | 11.7** | 10* | 9.1* | 7.9 |
| 120 | | 4 | 2.0** | 147 | 140* | 90 | 9.5 | 7.8** | 8.5** | 10.3** | 9.3** | 8.5** | 6.4** | 6.1** |

^{a}Following inoculation of femurs with saline (sham) or 10⁵ NCTC 2472 cells on Day 0, twice daily oral dosing was commenced on Day 3 and continued until study termination on Day 15.
^{b}Visual scores (scale 0-5) based on microradiographs.
^{c}Counts per high powered field. Osteoclasts defined as tartrate resistant acid phosphatase (TRACP) positive cells and macrophages defined as CD68 positive cells.
^{d}Number of palpation-induced (PIF) or spontaneous (SF) flinches over a 2-minute observation period.
^{e}Time (sec) animals hold the hindpaw aloft (guarding) while stationary over a 2-minute observation period;
SG = spontaneous guarding; PIG = palpation-induced guarding.
*p < 0.05 vs control;
**p < 0.01 vs control

### Discussion.

As demonstrated in the experimentals described herein, JNJ-141 is a potent CSF-1R inhibitor with the capacity to block proliferation and chemokine expression by monocyte/macrophages in vitro and to prevent CSF-1-induced expression of c-fos mRNA in vivo. Assay of 111 diverse recombinant kinases identified five additional potential tyrosine kinase targets (i.e., KIT, FLT3, TRKA, LCK, and AXL) inhibited by concentrations under 100 nM. However, of these, sub-micromolar concentrations of JNJ-141 inhibited cellular functions dependent on KIT, FLT3, and TRKA, but micromolar concentrations were required to inhibit AXL and LCK-dependent cell activities. The requirement for relatively high concentrations of JNJ-141 to impact cellular AXL and LCK assays may reflect the conformational differences between the purified recombinant kinases and their natural cellular counterparts. Overall, the kinase profile of JNJ-141 is attractive for the prevention and treatment of primary and secondary bone cancer because CSF-1R-dependent macrophages and osteoclasts are believed to support the growth of tumors and mediate osteolysis in metastatic bone disease, respectively. Furthermore, inhibition of FLT3, KIT, and TRKA may contribute to the desirable therapeutic activity of JNJ-141. Mast cells are dependent on KIT for survival and together with macrophages promote tumor angiogenesis and malignant progression (Soucek L et al., Nature Medicine 2007; 10: 1211-1218). Further, mast cells are associated with bone loss (Chiappetta N and Gruber B, Semin Arthritis Rheum 2006; 36: 32-6), and KIT is overexpressed on and may drive some osteosarcomas (Entz-Werle N et al., Int J Cancer 2007; 120: 2510-6) and gastrointestinal stromal tumors (Demetri G D., Seminars in Oncology 2001; 28:19-26). Thus, inhibition of KIT may contribute to reduced tumor growth and osteolysis and may cause regression of some primary and secondary bone tumors. Like FMS, FLT3 is highly expressed by macrophage and osteoclast progenitors and under some circumstances may augment or substitute for FMS (Lean J M et al., Blood 2001; 98: 2707-13). Inhibition of FLT3 might therefore contribute to the bone protective activity of JNJ-141 and to the inhibition of tumor angiogenesis. TRKA is the exclusive receptor for nerve growth factor (NGF). TRKA and NGF are essential to the growth and survival of nociceptors and antibodies that neutralize NGF dramatically reduced pain-related behaviours in a model of secondary bone cancer (Halvorson K G et al., Cancer Res 2005; 65: 9426-35). These data in mice suggest that inhibition of TRKA by JNJ-141 may have contributed to reducing tumor-mediated nociception in the MRMT-1 and 2472 sarcoma models and may provide pain relief to patients suffering severe pain associated with primary or secondary bone tumors. A direct effect of JNJ-141 on H460 cells was unlikely since the serum-dependent proliferation of H460 cells in culture was not affected by JNJ-141 at concentrations (5 μM) higher than achieved in vivo.

In the experimentals described herein, depletion of tumor-associated macrophages was profound (>97%) in JNJ-141 treated mice. CSF-1 inhibition might influence macrophage numbers by several mechanisms. CSF-1 has direct chemotactic activity for macrophages (Webb S E, et al., J Cell Science 1996; 109:793-803) and induces macrophage expression of the chemotactic peptide, monocyte chemotactic protein-1 (CCL2) (Baran C P, et al., Am J Respir Crit Care Med 2007; 176:78-89.) Recruited macrophages or their precursors proliferate in situ under the influence of CSF-1, and CSF-1 is a powerful macrophage survival and differentiation factor even at low concentrations (Chitu V et al., Curr Opin Immunol 2006; 18:39-48.) Consequently, JNJ-141 may have reduced recruitment of monocytes and macrophage progenitors to growing H460 tumors, and, once recruited, these cells failed to survive, proliferate, and differentiate. GM-CSF, IL-3, VEGF, CCL2, and other undefined growth factor pathways support macrophage recruitment and survival (Takahashi K., J Clin Exp Hematopathology 2001; 41:1-33.) Given the potential redundant pathways, the near quantitative reduction in TAMs by JNJ-141 is striking.

The reduction in TAMs and tumor growth was associated with a 66% percent reduction in tumor vessel density. TAMs are reported express a large number of growth factors and proteases that support angiogenesis (e.g., VEGF, bFGF, IL-8, urokinase, MMP-2 and MMP-9 and others) (Mantovani A. et al., Trends in Immunology 2002; 23:549-555). The results obtained with JNJ-141 are consistent with a growing body of literature that TAMs are required for optimal tumor angiogenesis. Parenteral liposomal clodronate, a selective macrophage toxin, was reported to reduce the growth of F9 teratocarcinoma and A673 rhadomyosarcoma xenografts by 75 and 66%, respectively (van Rooijen N et al., Methods Enzymol 2003; 373:3-16.) Growth suppression was accompanied by depletion of tumor-associated F4/80⁺ macrophages and marked reductions in tumor vasculature. In A673 tumors, the reduced density of CD1⁺ microvasculature was superior to that achieved using anti-VEGF.

More recently, a subpopulation of monocytes was identified that expresses Tie2 and that is required for optimal tumor angiogenesis (De Palma M et al., Cancer Cell 2005; 8:211-226.) Selective deletion of this subpopulation resulted in regression of glioma xenografts in nude mice. Other studies more specifically implicated CSF-1/CSF-1R in tumor growth and angiogenesis (see Background herein, and Nowickki A, et al., Int J Cancer 1996; 65: 112-119; Okazaki T, et al., J Immunol 2005; 174:7531-7538; Aharinejad S, et al., Cancer Res 2002; 62:5317-5324; Aharinejad S, et al., Cancer Res 2004; 64:5378-5384; Paulus P et al., Cancer Res 2006; 66:4349-56.)

The near absence of osteoclasts in CSF-1-deficient rodents identified a critical role for CSF-1 in normal osteoclastogenesis (Pollard, J. W. et al., Adv in Devel Biochem 1995; 4:153-193; Van Wesenbeeck L, et al., PNAS 2002; 99:14303-14308.)

Disregulated osteoclastogenesis occurs in arthritis and metastatic bone disease. A neutralizing CSF-1R antibody dramatically halted osteolysis in a mouse model of arthritis, identifying a role for CSF-1 in immune-mediated osteoclastogenesis (Kitaura H et al., J Clin Invest; 2005; 115:3418-27.)

As demonstrated in the experimentals described herein, nearly complete inhibition of osteoclastogenesis and bone erosion by JNJ-141 was seen in a syngeneic mammary carcinoma bone metastasis model. These data extend a previous report of bone protection by an alternative CSF-1R kinase inhibitor, Ki20227, in a melanoma model of bone metastasis and osteolysis (see Ohno H, et al., Mol Cancer Ther 2006; 5:2634-2643.)

The data presented in the experimentals described herein also support a critical role for CSF-1 in tumor-induced osteoclastogenesis and a therapeutic utility for JNJ-141 in the treatment of the nearly 85% of late stage breast cancer patients that will be diagnosed with bone metastases and are at risk of fracture, bone pain and hypercalcemia.

High-dose bisphosphonates (pamidronate and zoledronate) are indicated for the prevention of skeletal events in individuals with bone metastasis (see, Body J. J., Clin Cancer Res 2006; 12 (20 Suppl) 6258s-6263s), and denosumab, a RANKL antibody, had promising anti-resorptive activity in clinical trials (see, Body J. J, et al., Clin Cancer Res 2006; 12:1221-1228.) However, inhibitors of CSF-1R, such as JNJ-141, that are short acting and readily reversible may provide an attractive alternative to bisphosphonates that bind bone with a half-life of months, a feature that may contribute to osteonecrosis, especially as patient life expectancy increases.

JNJ-141 appears to reduce the growth rates of both soft tissue and skeletal metastases as evidenced by the growth suppression of H460 and the MRMT tumors seen in the experimentals described herein. This is in contrast to zoledronate that does not appear to effect the growth of soft tissue metastasis (Mundy G R et al., Semin Oncol 2001; 28(suppl 6):35-44).).

JNJ-141 reduced solid tumor growth and prevented bone erosion by skeletal metastases Inhibition of CSF-1R by JNJ-141 will be useful in the treatment of cancers in a variety of settings. For example, JNJ-141 will be useful in combination therapy because a neutralizing antibody to CSF-1 was shown to reduce tumor expression of chemoresistance genes (Paulus P et al., Cancer Res 2006; 66:4349-56) and removal of macrophage-derived growth and survival factors may delay tumor recovery following chemotherapy. Also, because CSF-1R has oncogenic potential (Kirma N et al., Cancer Res 2004; 64:4162-70), and is expressed on a variety of carcinomas (Kascinshi B., Cancer Treat Res 2002; 107:285-82), CSF-1R inhibitors may, in some instances, have direct anticancer activity. Further, intratumoral chemotaxis studies and live videomicroscopy have demonstrated that tumor cells move together with macrophages via a process that is dependent on both EGF and CSF-1 (Wyckoff J, et al., Cancer Res 2004; 64:7022-7029), and mouse genetics have identified a critical role for CSF-1 in spontaneous metastasis of mammary carcinomas to the lung (Lin E Y, et al., J Exp Med 2001; 193:727-739). No current therapies are directed toward the metastatic process even though circulating tumor cell numbers have been shown to accurately predict survival in some tumors (Budd G T et al., Clin Cancer Res 2006; 12:6403-6409.) Intervening CSF-1-dependent tumor-cell egress may ultimately slow the relentless metastatic dissemination that contributes to the death of end-stage cancer patients.

### Methods of Treatment/Prevention

As used herein, the term “cancer” refers to the unwanted cell proliferation of one or more subset of cells in a multicellular organism resulting in harm (i.e., discomfort or decreased life expectancy) to the multicellular organisms. As used herein “cell proliferative disorders” include neoplastic disorders. As used herein, a “neoplastic disorder” refers to a tumor resulting from abnormal or uncontrolled cellular growth.

As used herein, the terms “bone cancer” shall mean cancer that originates in bone tissue (referred to herein as “primary bone cancer”) and cancer cells that travel to the bone from elsewhere (referred to herein as “secondary bone cancer” or “metastatic bone cancer”).

Primary bone cancer cells include, but are not limited to, osteosarcoma cells, cells from Ewing's family of tumors, chondrosarcoma cells, malignant giant cell tumor cells, malignant fibrous histiocytoma cells and adamantinoma cells.

Types of bone cancer included (but are not limited to): Osteosarcoma, a cancerous tumor of the bone, usually of the arms, legs, or pelvis (the most common primary cancer); Chrondrosarcoma, cancer of the cartilage (the second most common primary cancer); Ewing's Sarcoma, tumors that usually develop in the cavity of the leg and arm bones, Fibrosarcoma and Malignant Fibrous Histiocytoma, cancers that develop in soft tissues such as the tendons, ligaments, fat, muscle and move to the bones of the legs, arms, and jaw; Giant Cell Tumor, a primary bone tumor that is malignant only about 10% of the time and is most common in the arm or leg bones; and Chordoma, a primary bone tumor that usually occurs in the skull or spine.

Secondary bone cancer or metastatic bone cancer cells (also referred to herein as “bone metastases”) are cancer cells that have metastasized from other tissues such as the breast, lung, prostate and kidney. Such cancers are additionally referred to as cancers of the organ or tissue in which the cancer begins that are “metastic to the bone”—for example, lung cancer metastatic to bone. Such cancers are intended to be within the definition of “bone cancer” herein.

The present invention provides therapeutic methods for treating a subject having, and prophylactic methods for preventing in a subject at risk of (or susceptible to) developing, bone cancer and the bone loss and bone pain associated with bone cancer said methods comprising the administration of a compound of Formula I, preferably Example 38a.

The term “subject” as used herein, refers to an animal, preferably a mammal, most preferably a human, who has been the object of treatment, observation or experiment.

In one embodiment, the present invention provides a method of treating bone cancer, including primary bone cancers and secondary bone cancers, preferably secondary bone cancer, in a subject in need thereof, said method comprising administering to the subject a therapeutically effective amount of a pharmaceutical composition comprising a compound of Formula I, preferably Example 38a, and a pharmaceutically acceptable carrier. Administration of said therapeutic agent can occur concurrently with the manifestation of symptoms characteristic of bone cancer. Preferably, said secondary bone cancer comprises bone metastases from breast, lung and prostate cancer, most preferably breast cancer metastases.

In another embodiment, the present invention provides a method of treating bone loss and bone pain associated with bone cancer, including primary bone cancers and secondary bone cancers, preferably secondary bone cancer, in a subject in need thereof, said method comprising administering to said subject a therapeutically effective amount of a pharmaceutical composition comprising a compound of Formula I, preferably Example 38a, and a pharmaceutically acceptable carrier. Administration of said therapeutic agent can occur concurrently with the manifestation of symptoms characteristic of bone cancer, a therapy to compensate for the bone loss and pain. Preferably, said secondary bone cancer comprises bone metastases from breast, lung and prostate cancer, most preferably breast cancer metastases.

In one embodiment, the present invention provides a method of preventing bone cancer, including primary bone cancers and secondary bone cancers, preferably secondary bone cancers, in a subject in need thereof, said method comprising administering to the subject a prophylactically effective amount of a pharmaceutical composition comprising a pharmaceutically acceptable carrier and a compound of Formula I, preferably Example 38a, and a pharmaceutically acceptable carrier. Administration of said prophylactic agent can occur prior to the manifestation of symptoms characteristic of bone cancer, such that the disease is prevented or, alternatively, delayed in its progression. Preferably, said secondary bone cancer comprises bone metastases from breast, lung and prostate cancer, most preferably breast cancer metastases.

In another embodiment, the present invention provides a method of preventing bone loss associated with bone cancer, including primary bone cancers and secondary bone cancers, preferably secondary bone cancers, in a subject in need thereof, said method comprising administering to the subject a prophylactically effective amount of a pharmaceutical composition comprising a pharmaceutically acceptable carrier and a compound of Formula I, preferably Example 38a, and a pharmaceutically acceptable carrier. Administration of said prophylactic agent can occur prior to the manifestation of the bone loss symptoms characteristic of bone cancer, such that the condition is prevented or, alternatively, delayed in its progression. Preferably, said secondary bone cancer comprises bone metastases from breast, lung and prostate cancer, most preferably breast cancer metastases.

In another embodiment, the present invention provides a method of preventing bone pain associated with bone cancer, including primary bone cancers and secondary bone cancers, preferably secondary bone cancers, in a subject in need thereof, said method comprising administering to the subject a prophylactically effective amount of a pharmaceutical composition comprising a pharmaceutically acceptable carrier and a compound of Formula I, preferably Example 38a, and a pharmaceutically acceptable carrier. Administration of said prophylactic agent can occur prior to the manifestation of the bone pain symptoms characteristic of bone cancer, such that the condition is prevented or, alternatively, delayed in its progression. Preferably, said secondary bone cancer comprises bone metastases from breast, lung and prostate cancer, most preferably breast cancer metastases.

The term “prophylactically effective amount” refers to an amount of active compound or pharmaceutical agent that inhibits or delays in a subject the onset of a disorder as being sought by a researcher, veterinarian, medical doctor or other clinician.

The term “therapeutically effective amount” as used herein, refers to an amount of active compound or pharmaceutical agent that elicits the biological or medicinal response in a subject that is being sought by a researcher, veterinarian, medical doctor or other clinician, which includes alleviation of the symptoms of the disease or disorder being treated.

Methods for determining therapeutically and prophylactically effective doses for pharmaceutical compositions comprising a compound of the present invention are disclosed herein and known in the art.

In a further aspect to the prophylactic and therapeutic methods of the present invention, the invention encompasses a combination therapy for treating a subject having, or preventing in a subject at risk of (or susceptible to) developing, bone cancer, including primary bone cancers and secondary bone cancers, preferably secondary bone cancers, and bone loss and bone pain associated with said bone cancer. Preferably, said secondary bone cancer comprises bone metastases from breast, lung and prostate cancer, most preferably breast cancer metastases.

The combination therapy comprises administering to the subject a therapeutically or prophylactically effective amount of a pharmaceutical composition comprising a compound of the present invention and a pharmaceutically acceptable carrier, and one or more other anti-cell proliferation therapy including chemotherapy, radiation therapy, gene therapy and immunotherapy.

As used herein, “chemotherapy” refers to a therapy involving a chemotherapeutic agent. A variety of chemotherapeutic agents may be used in the combined treatment methods disclosed herein. Chemotherapeutic agents contemplated as exemplary, include, but are not limited to: platinum compounds (e.g., cisplatin, carboplatin, oxaliplatin); taxane compounds (e.g., paclitaxcel, docetaxol); campotothecin compounds (irinotecan, topotecan); vinca alkaloids (e.g., vincristine, vinblastine, vinorelbine); anti-tumor nucleoside derivatives (e.g., 5-fluorouracil, leucovorin, gemcitabine, capecitabine); alkylating agents (e.g., cyclophosphamide, carmustine, lomustine, thiotepa); epipodophyllotoxins/podophyllotoxins (e.g. etoposide, teniposide); aromatase inhibitors (e.g., anastrozole, letrozole, exemestane); anti-estrogen compounds (e.g., tamoxifen, fulvestrant), antifolates (e.g., premetrexed disodium); hypomethylating agents (e.g., azacitidine); biologics (e.g., gemtuzamab, cetuximab, rituximab, pertuzumab, trastuzumab, bevacizumab, erlotinib); antibiotics/anthracyclines (e.g. idarubicin, actinomycin D, bleomycin, daunorubicin, doxorubicin, mitomycin C, dactinomycin, caminomycin, daunomycin); antimetabolites (e.g., aminopterin, clofarabine, cytosine arabinoside, methotrexate); tubulin-binding agents (e.g. combretastatin, colchicine, nocodazole); topoisomerase inhibitors (e.g., camptothecin). Further useful agents include verapamil, a calcium antagonist found to be useful in combination with antineoplastic agents to establish chemosensitivity in tumor cells resistant to accepted chemotherapeutic agents and to potentiate the efficacy of such compounds in drug-sensitive malignancies. (See Simpson W G, Cell Calcium. 1985 December; 6(6):449-67.) Additionally, yet to emerge chemotherapeutic agents are contemplated as being useful in combination with a compound of the present invention.

As used herein, “radiation therapy” refers to a therapy that comprises exposing the subject in need thereof to radiation. Such therapy is known to those skilled in the art. The appropriate scheme of radiation therapy will be similar to those already employed in clinical therapies wherein the radiation therapy is used alone or in combination with other chemotherapeutics.

As used herein, “gene therapy” refers to a therapy targeting on particular genes involved in tumor development. Possible gene therapy strategies include the restoration of defective cancer-inhibitory genes, cell transduction or transfection with antisense DNA corresponding to genes coding for growth factors and their receptors, RNA-based strategies such as ribozymes, RNA decoys, antisense messenger RNAs and small interfering RNA (siRNA) molecules and the so-called ‘suicide genes’.

As used herein, “immunotherapy” refers to a therapy targeting particular protein involved in tumor development via antibodies specific to such protein. For example, monoclonal antibodies against vascular endothelial growth factor have been used in treating cancers.

Where a second pharmaceutical composition is used in addition to a pharmaceutical composition comprising a compound of the present invention, the two pharmaceuticals may be administered simultaneously (e.g. in separate or unitary compositions) sequentially in either order, at approximately the same time, or on separate dosing schedules. In the latter case, the two pharmaceutical compositions will be administered within a period and in an amount and manner that is sufficient to ensure that an advantageous or synergistic effect is achieved. It will be appreciated that the preferred method and order of administration and the respective dosage amounts and regimes for each component of the combination will depend on the particular chemotherapeutic agent being administered in conjunction with the pharmaceutical composition of the present invention, their route of administration, the particular tumor being treated and the particular host being treated.

As will be understood by those of ordinary skill in the art, the appropriate doses of chemotherapeutic agents will be generally similar to or less than those already employed in clinical therapies wherein the chemotherapeutics are administered alone or in combination with other chemotherapeutics. The optimum method and order of administration and the dosage amounts and regime can be readily determined by those skilled in the art using conventional methods and in view of the information set out herein.

Dosages may be administered, for example once, twice or more per course of treatment, which may be repeated for example every 7, 14, 21 or 28 days.

The pharmaceutical composition of the present invention can be administered to a subject systemically, for example, intravenously, orally, subcutaneously, intramuscular, intradermal, or parenterally. The pharmaceutical composition of the present invention can also be administered to a subject locally. Non-limiting examples of local delivery systems include the use of intraluminal medical devices that include intravascular drug delivery catheters, wires, pharmacological stents and endoluminal paving.

The pharmaceutical composition of the present invention can further be administered to a subject in combination with a targeting agent to achieve high local concentration of a compound at the target site. In addition, the pharmaceutical composition of the present invention may be formulated for fast-release or slow-release with the objective of maintaining the drugs or agents in contact with target tissues for a period ranging from hours to weeks.

As used herein, the term “composition” is intended to encompass a product comprising the specified ingredients in the specified amounts, as well as any product which results, directly or indirectly, from combinations of the specified ingredients in the specified amounts.

As used herein, the phrase “pharmaceutically acceptable” refers to molecular entities and compositions that do not produce an adverse, allergic or other untoward reaction when administered to an animal, or a human, as appropriate. Veterinary uses are equally included within the invention and “pharmaceutically acceptable” formulations include formulations for both clinical and/or veterinary use.

The pharmaceutical compositions of the present invention comprise a compound of the Formula I, as the active ingredient, intimately admixed with a pharmaceutical carrier according to conventional pharmaceutical compounding techniques, which carrier may take a wide variety of forms depending on the form of preparation desired for administration, e.g., oral or parenteral such as intramuscular.

The pharmaceutical compositions of the present invention may contain between about 0.1 mg and 1000 mg, preferably about 100 to 500 mg, of the compound of Formula I, and may be constituted into any form suitable for the mode of administration selected.

Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques.

For parenterals, the carrier will usually comprise sterile water, though other ingredients, for example, for purposes such as aiding solubility or for preservation, may be included.

Injectable suspensions may also be prepared, in which case appropriate liquid carriers, suspending agents and the like may be employed.

The pharmaceutical compositions of the present invention also include pharmaceutical compositions for slow release of the compounds of Formula I. The composition includes a slow release carrier (typically, a polymeric carrier) and a compound of Formula I.

Slow release biodegradable carriers are well known in the art. These are materials that may form particles that capture therein an active compound(s) and slowly degrade/dissolve under a suitable environment (e.g., aqueous, acidic, basic, etc) and thereby degrade/dissolve in body fluids and release the active compound(s) therein. The particles are preferably nanoparticles (i.e., in the range of about 1 to 500 nm in diameter, preferably about 50-200 nm in diameter, and most preferably about 100 nm in diameter).

In preparation for slow release, a slow release carrier, typically a polymeric carrier, and a compound of the present invention are first dissolved or dispersed in an organic solvent. The obtained organic solution is then added into an aqueous solution to obtain an oil-in-water-type emulsion. Preferably, the aqueous solution includes surface-active agent(s). Subsequently, the organic solvent is evaporated from the oil-in-water-type emulsion to obtain a colloidal suspension of particles containing the slow release carrier and the compound of the present invention.

The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, from about 0.01 mg to 200 mg/kg of body weight per day, preferably, from about 0.03 to about 100 mg/kg of body weight per day, and most preferably, from about 2 to 40 mg/kg daily.

Most preferably the formulation of JNJ-141 comprises silicified microcrystalline cellulose, croscarmellose sodium, magnesium stearate, butylated hydroxyanisole, and butylated hydroxytoluene.

The pharmaceutical composition may be administered on a regimen of 1 to 5 times per day. The dosages, however, may be varied depending upon the requirement of the patients, the severity of the condition being treated and the particular compound of Formula I being employed. The use of either daily administration or post-periodic dosing may be employed.

Preferably the pharmaceutical compositions of the present invention are in unit dosage forms such as tablets, pills, capsules, powders, granules, sterile parenteral solutions or suspensions, metered aerosol or liquid sprays, drops, ampoules, auto-injector devices or suppositories; for oral parenteral, intranasal, sublingual or rectal administration, or for administration by inhalation or insufflation. Alternatively, the pharmaceutical composition may be presented in a form suitable for once-weekly or once-monthly administration; for example, an insoluble salt of the active compound, such as the decanoate salt, may be adapted to provide a depot preparation for intramuscular injection.

For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.1 to about 500 mg of the active ingredient of the present invention.

The tablets or pills of the pharmaceutical composition of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, acetyl alcohol and cellulose acetate.

The liquid forms in which a compound of Formula I may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin. The liquid forms in suitably flavored suspending or dispersing agents may also include the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

Advantageously, the pharmaceutical composition used in the present invention may be administered in a single daily dose, or the total daily dosage may be administered in divided doses of two, three or four times daily of 1 to 2 times per day. Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

Another alternative method for administering the compounds of the invention may be by conjugating a compound to a targeting agent which directs the conjugate to its intended site of action, i.e., to bone tumor cells, secondary bone tumor cells, or bone tumor associated macrophages or osteoclasts. Both antibody and non-antibody targeting agents may be used. Because of the specific interaction between the targeting agent and its corresponding binding partner, the compound of the present invention can be administered with high local concentrations at or near a target site and thus treats the disorder at the target site more effectively. For instance, a bisphosphonate group may be added to JNJ-141 in order to direct it to bone.

When proteins such as antibodies or growth factors, or polysaccharides are used as targeting agents, they are preferably administered in the form of injectable compositions. In the case of bone cancer, intracavitary administration may be preferable.

A therapeutically effective dose of a compound of the present invention conjugated to a targeting agent depends on the individual, the bone cancer type and state and other clinical variables. The effective dosages are readily determinable using data from animal models, including those presented herein.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

1. A method of treating or preventing bone cancer in a subject in need thereof, said method comprising administering to the subject a therapeutically effective amount of a pharmaceutical composition comprising a pharmaceutically acceptable carrier and a compound of Formula I: 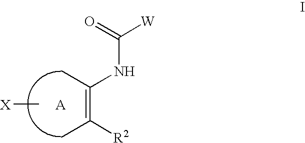
or a solvate, hydrate, tautomer or pharmaceutically acceptable salt thereof, wherein:
A is
phenyl or pyridyl, either of which may be substituted with one of chloro, fluoro, methyl, —N₃, —NH₂, —NH(alkyl), —N(alkyl)₂, —S(alkyl), —O(alkyl), or 4-aminophenyl;
W is
pyrrolyl, imidazolyl, isoxazolyl, oxazolyl, 1,2,4 triazolyl, or furanyl, any of which may be connected through any carbon atom, wherein the pyrrolyl, imidazolyl, isoxazolyl, oxazolyl, 1,2,4 triazolyl, or furanyl may contain one —Cl, —CN, —NO₂, —OMe, or —CF₃ substitution, connected to any other carbon;
R² is
cycloalkyl, thiophenyl, dihydrosulfonopyranyl, phenyl, furanyl, tetrahydropyridyl, or dihydropyranyl, any of which may be independently substituted with one or two of each of the following: chloro, fluoro, and C₍₁₋₃₎alkyl, with the proviso that tetrahydropyridyl is connected to the ring A through a carbon-carbon bond;
X is 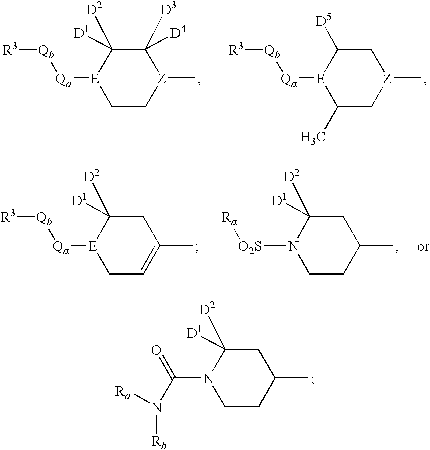
Z is
CH or N;
D¹ and D² are
each hydrogen or taken together form a double bond to an oxygen;
D³ and D⁴ are
each hydrogen or taken together form a double bond to an oxygen;
D⁵ is
hydrogen or —CH₃, wherein said —CH₃ may be relatively oriented syn or anti;
Rₐ and R_{b} are independently
hydrogen, cycloalkyl, haloalkyl, aryl, aralkyl, heteroaryl, or heteroaralkyl;
E is
N, S, O, SO or SO₂, with the proviso that E may not be N if the following three conditions are simultaneously met: Qₐ is absent, Q_{b} is absent, and R³ is an amino group or cyclic amino radical wherein the point of attachment to E is N;
Qₐ is
absent, —CH₂—, —CH₂CH₂—, or C(O);
Q_{b} is
absent, —NH—, —CH₂—, —CH₂CH₂—, or C(O), with the proviso that Q_{b} may not be C(O) if Qₐ is C(O), and further provided that Q_{b} may not be —NH— if E is N and Qₐ is absent, further provided that Q_{b} may not be —NH— if R³ is an amino group or cyclic amino radical wherein the point of attachment to Q_{b} is N;
R³ is
hydrogen, hydroxyalkylamino, (hydroxyalkyl)₂amino, alkylamino, aminoalkyl, dihydroxyalkyl, alkoxy, dialkylamino, hydroxyalkyl, —COOH, —CONH₂, —CN, —SO₂-alkyl-R⁴, —NH₂, or a 5 or six membered ring which contains at least one heteroatom N and may optionally contain an additional heteromoiety selected from S, SO₂, N, and O, and the 5 or 6 membered ring may be saturated, partially unsaturated or aromatic, wherein aromatic nitrogen in the 5 or 6 membered ring may be present as N-oxide, and the 5 or 6 membered ring may be optionally substituted with methyl, halogen, alkylamino, or alkoxy; R³ may also be absent, with the proviso that R³ is not absent when E is nitrogen;
R⁴ is
hydrogen, —OH, alkoxy, carboxy, carboxamido, or carbamoyl.

2. The method of claim 1, wherein
A is
phenyl or pyridyl;
X is 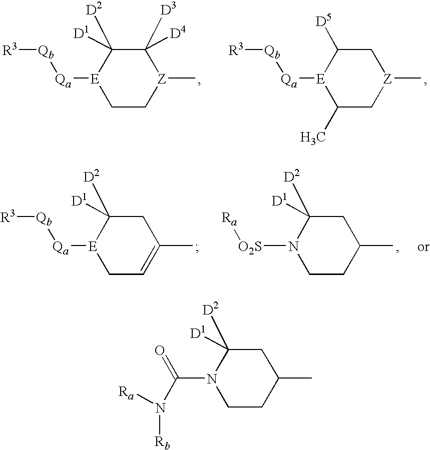
and is oriented para with respect to —NHCO—W.

3. The method of claim 2 wherein W is 3H-2-imidazolyl-4-carbonitrile.

4. The method of claim 3 wherein R² is cyclohexenyl which may be substituted with one or two methyl groups.

5. The method of claim 4 wherein:
X is 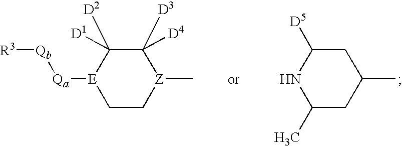
Z is
CH;
D¹ and D² are
each hydrogen;
D³ and D⁴ are
each hydrogen;
D⁵ is
—CH₃, wherein said —CH₃ may be relatively oriented syn or anti;
E is
N;
Q_{b} is
absent, —CH₂—, —CH₂CH₂—, or C(O), with the proviso that Q_{b} may not be C(O) if Qₐ is C(O), further provided that Q_{b} may not be —NH— if R³ is an amino group or cyclic amino radical wherein the point of attachment to Q_{b} is N; and
R³ is hydrogen, hydroxyalkylamino, (hydroxyalkyl)₂amino, alkylamino, aminoalkyl, dihydroxyalkyl, alkoxy, dialkylamino, hydroxyalkyl, —COOH, —CONH₂, —CN, —SO₂—CH₃, —NH₂, pyridyl, pyridyl-N-oxide, or morpholinyl.

6. The method of claim 5 wherein:
X is

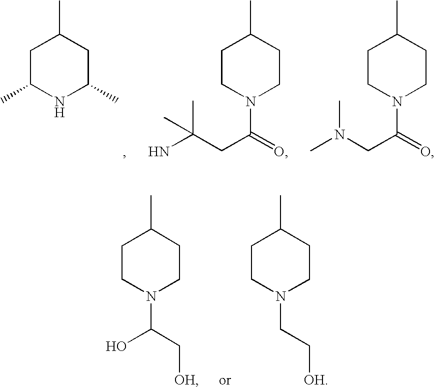

7. The method of claim 6, wherein the compound of Formula I is: 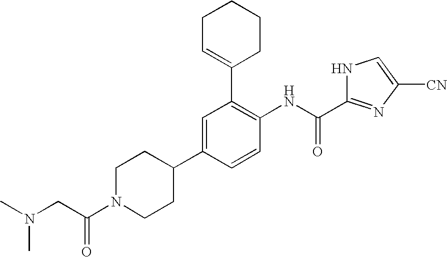
or a solvate, hydrate, tautomer or pharmaceutically acceptable salt thereof.

8. The method of claim 7, wherein the bone cancer is a secondary bone cancer.

9-16. (canceled)

17. A method of preventing or treating bone loss associated with bone cancer, in a subject in need thereof, said method comprising administering to the subject a prophylactically effective amount of a pharmaceutical composition comprising a pharmaceutically acceptable carrier and a compound of Formula I: 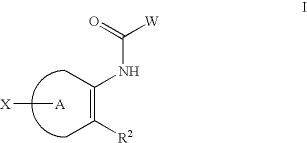
or a solvate, hydrate, tautomer or pharmaceutically acceptable salt thereof, wherein:
A is
phenyl or pyridyl, either of which may be substituted with one of chloro, fluoro, methyl, —N₃, —NH₂, —NH(alkyl), —N(alkyl)₂, —S(alkyl), —O(alkyl), or 4-aminophenyl;
W is
pyrrolyl, imidazolyl, isoxazolyl, oxazolyl, 1,2,4 triazolyl, or furanyl, any of which may be connected through any carbon atom, wherein the pyrrolyl, imidazolyl, isoxazolyl, oxazolyl, 1,2,4 triazolyl, or furanyl may contain one —Cl, —CN, —NO₂, —OMe, or —CF₃ substitution, connected to any other carbon;
R² is
cycloalkyl, thiophenyl, dihydrosulfonopyranyl, phenyl, furanyl, tetrahydropyridyl, or dihydropyranyl, any of which may be independently substituted with one or two of each of the following: chloro, fluoro, and C₍₁₋₃₎alkyl, with the proviso that tetrahydropyridyl is connected to the ring A through a carbon-carbon bond;
X is 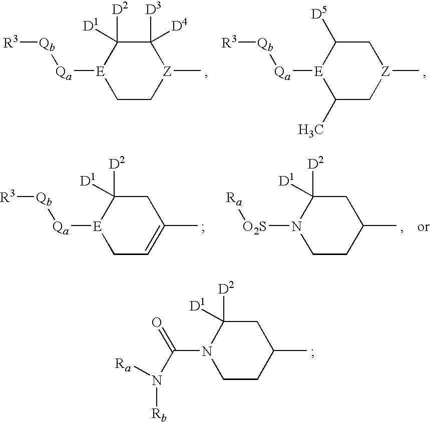
Z is
CH or N;
D¹ and D² are
each hydrogen or taken together form a double bond to an oxygen;
D³ and D⁴ are
each hydrogen or taken together form a double bond to an oxygen;
D⁵ is
hydrogen or —CH₃, wherein said —CH₃ may be relatively oriented syn or anti;
Rₐ and R_{b} are independently
hydrogen, cycloalkyl, haloalkyl, aryl, aralkyl, heteroaryl, or heteroaralkyl;
E is
N, S, O, SO or SO₂, with the proviso that E may not be N if the following three conditions are simultaneously met: Qₐ is absent, Q_{b} is absent, and R³ is an amino group or cyclic amino radical wherein the point of attachment to E is N;
Qₐ is
absent, —CH₂—, —CH₂CH₂—, or C(O);
Q_{b} is
absent, —NH—, —CH₂—, —CH₂CH₂—, or C(O), with the proviso that Q_{b} may not be C(O) if Qₐ is C(O), and further provided that Q_{b} may not be —NH— if E is N and Qₐ is absent, further provided that Q_{b} may not be —NH— if R³ is an amino group or cyclic amino radical wherein the point of attachment to Q_{b} is N;
R³ is
hydrogen, hydroxyalkylamino, (hydroxyalkyl)₂amino, alkylamino, aminoalkyl, dihydroxyalkyl, alkoxy, dialkylamino, hydroxyalkyl, —COOH, —CONH₂, —CN, —SO₂-alkyl-R⁴, —NH₂, or a 5 or six membered ring which contains at least one heteroatom N and may optionally contain an additional heteromoiety selected from S, SO₂, N, and O, and the 5 or 6 membered ring may be saturated, partially unsaturated or aromatic, wherein aromatic nitrogen in the 5 or 6 membered ring may be present as N-oxide, and the 5 or 6 membered ring may be optionally substituted with methyl, halogen, alkylamino, or alkoxy; R³ may also be absent, with the proviso that R³ is not absent when E is nitrogen;
R⁴ is
hydrogen, —OH, alkoxy, carboxy, carboxamido, or carbamoyl.

18. The method of claim 17, wherein
A is
phenyl or pyridyl;
X is 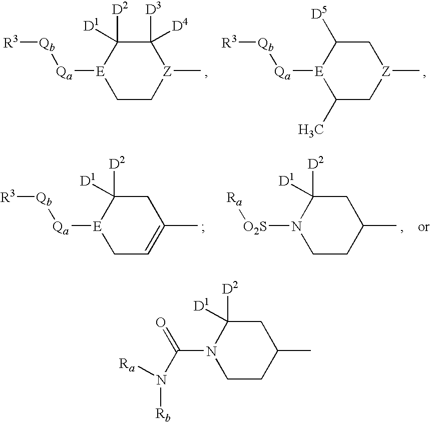
and is oriented para with respect to —NHCO—W.

19. The method of claim 18, wherein W is 3H-2-imidazolyl-4-carbonitrile.

20. The method of claim 19, wherein R² is cyclohexenyl which may be substituted with one or two methyl groups.

21. The method of claim 20, wherein:
X is 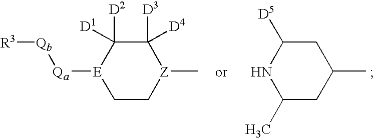
Z is
CH;
D¹ and D² are
each hydrogen;
D³ and D⁴ are
each hydrogen;
D⁵ is
—CH₃, wherein said —CH₃ may be relatively oriented syn or anti;
E is
N;
Q_{b} is
absent, —CH₂—, —CH₂CH₂—, or C(O), with the proviso that Q_{b} may not be C(O) if Qₐ is C(O), further provided that Q_{b} may not be —NH— if R³ is an amino group or cyclic amino radical wherein the point of attachment to Q_{b} is N; and
R³ is hydrogen, hydroxyalkylamino, (hydroxyalkyl)₂amino, alkylamino, aminoalkyl, dihydroxyalkyl, alkoxy, dialkylamino, hydroxyalkyl, —COOH, —CONH₂, —CN, —SO₂—CH₃, —NH₂, pyridyl, pyridyl-N-oxide, or morpholinyl.

22. The method of claim 21, wherein:
X is

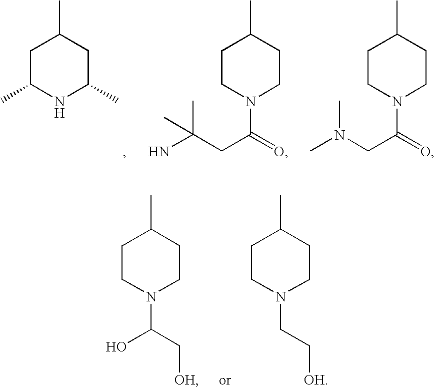

23. The method of claim 22, wherein the compound of Formula I is: 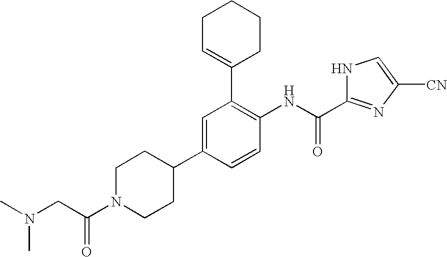
or a solvate, hydrate, tautomer or pharmaceutically acceptable salt thereof.

24. The method of claim 23, wherein the bone cancer is a secondary bone cancer.

25-32. (canceled)

33. A method of treating or preventing bone pain associated with bone cancer in a subject in need thereof, said method comprising administering to the subject a therapeutically effective amount of a pharmaceutical composition comprising a pharmaceutically acceptable carrier and a compound of Formula I: 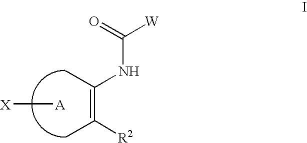
or a solvate, hydrate, tautomer or pharmaceutically acceptable salt thereof, wherein:
A is
phenyl or pyridyl, either of which may be substituted with one of chloro, fluoro, methyl, —N₃, —NH₂, —NH(alkyl), —N(alkyl)₂, —S(alkyl), —O(alkyl), or 4-aminophenyl;
W is
pyrrolyl, imidazolyl, isoxazolyl, oxazolyl, 1,2,4 triazolyl, or furanyl, any of which may be connected through any carbon atom, wherein the pyrrolyl, imidazolyl, isoxazolyl, oxazolyl, 1,2,4 triazolyl, or furanyl may contain one —Cl, —CN, —NO₂, —OMe, or —CF₃ substitution, connected to any other carbon;
R² is
cycloalkyl, thiophenyl, dihydrosulfonopyranyl, phenyl, furanyl, tetrahydropyridyl, or dihydropyranyl, any of which may be independently substituted with one or two of each of the following: chloro, fluoro, and C₍₁₋₃₎alkyl, with the proviso that tetrahydropyridyl is connected to the ring A through a carbon-carbon bond;
X is 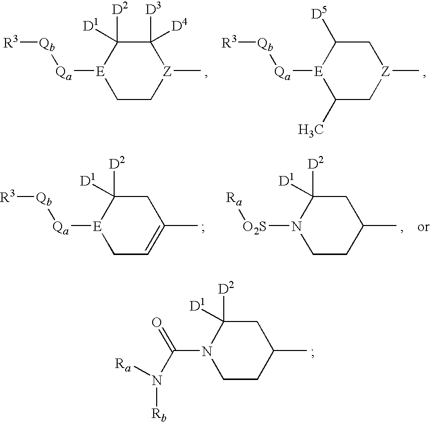
Z is
CH or N;
D¹ and D² are
each hydrogen or taken together form a double bond to an oxygen;
D³ and D⁴ are
each hydrogen or taken together form a double bond to an oxygen;
D⁵ is
hydrogen or —CH₃, wherein said —CH₃ may be relatively oriented syn or anti;
Rₐ and R_{b} are independently
hydrogen, cycloalkyl, haloalkyl, aryl, aralkyl, heteroaryl, or heteroaralkyl;
E is
N, S, O, SO or SO₂, with the proviso that E may not be N if the following three conditions are simultaneously met: Qₐ is absent, Q_{b} is absent, and R³ is an amino group or cyclic amino radical wherein the point of attachment to E is N;
Qₐ is
absent, —CH₂—, —CH₂CH₂—, or C(O);
Q_{b} is
absent, —NH—, —CH₂—, —CH₂CH₂—, or C(O), with the proviso that Q_{b} may not be C(O) if Qₐ is C(O), and further provided that Q_{b} may not be —NH— if E is N and Qₐ is absent, further provided that Q_{b} may not be —NH— if R³ is an amino group or cyclic amino radical wherein the point of attachment to Q_{b} is N;
R³ is
hydrogen, hydroxyalkylamino, (hydroxyalkyl)₂amino, alkylamino, aminoalkyl, dihydroxyalkyl, alkoxy, dialkylamino, hydroxyalkyl, —COOH, —CONH₂, —CN, —SO₂-alkyl-R⁴, —NH₂, or a 5 or six membered ring which contains at least one heteroatom Nand may optionally contain an additional heteromoiety selected from S, SO₂, N, and O, and the 5 or 6 membered ring may be saturated, partially unsaturated or aromatic, wherein aromatic nitrogen in the 5 or 6 membered ring may be present as N-oxide, and the 5 or 6 membered ring may be optionally substituted with methyl, halogen, alkylamino, or alkoxy; R³ may also be absent, with the proviso that R³ is not absent when E is nitrogen;
R⁴ is
hydrogen, —OH, alkoxy, carboxy, carboxamido, or carbamoyl.

34. The method of claim 33, wherein
A is
phenyl or pyridyl;
X is 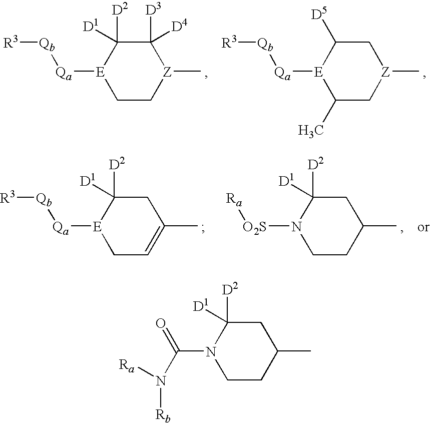
and is oriented para with respect to —NHCO—W.

35. The method of claim 34, wherein W is 3H-2-imidazolyl-4-carbonitrile.

36. The method of claim 35, wherein R² is cyclohexenyl which may be substituted with one or two methyl groups.

37. The method of claim 36, wherein:
X is 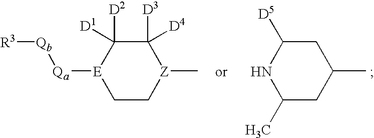
Z is
CH;
D¹ and D² are
each hydrogen;
D³ and D⁴ are
each hydrogen;
D⁵ is
—CH₃, wherein said —CH₃ may be relatively oriented syn or anti;
E is
N;
Q_{b} is
absent, —CH₂—, —CH₂CH₂—, or C(O), with the proviso that Q_{b} may not be C(O) if Qₐ is C(O), further provided that Q_{b} may not be —NH— if R³ is an amino group or cyclic amino radical wherein the point of attachment to Q_{b} is N; and
R³ is hydrogen, hydroxyalkylamino, (hydroxyalkyl)₂amino, alkylamino, aminoalkyl, dihydroxyalkyl, alkoxy, dialkylamino, hydroxyalkyl, —COOH, —CONH₂, —CN, —SO₂—CH₃, —NH₂, pyridyl, pyridyl-N-oxide, or morpholinyl.

38. The method of claim 37, wherein:
X is

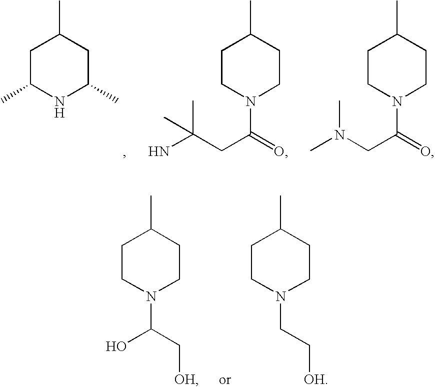

39. The method of claim 38, wherein the compound of Formula I is: 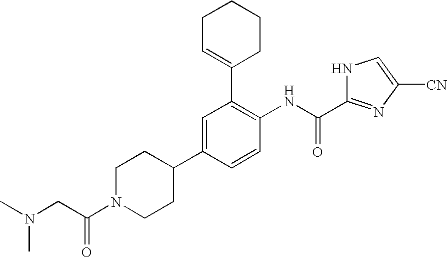
or a solvate, hydrate, tautomer or pharmaceutically acceptable salt thereof.

40. The method of claim 39, wherein the bone cancer is a secondary bone cancer.

41-48. (canceled)

49. A method of treating or preventing bone cancer in a subject in need thereof, said method comprising administering to the subject a therapeutically effective amount of a pharmaceutical composition comprising a pharmaceutically acceptable carrier and a compound that is: 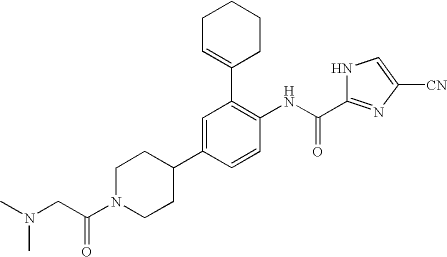
or a solvate, hydrate, tautomer or pharmaceutically acceptable salt thereof, and a pharmaceutically acceptable carrier.

50. The method of claim 49, further comprising administration of a chemotherapeutic agent.

51. The method of claim 49, wherein the pharmaceutical composition is administered by the controlled delivery by release from an intraluminal medical device of said compound.

52. The method of claim 49, wherein the pharmaceutical composition further comprises a targeting agent.

53-72. (canceled)
